(19) United States
(12) Patent Application Publication
(10) Pub. No.: US 2023/0181743 A1
(43) Pub. Date: Jun. 15, 2023

(54) FUNCTIONAL LIPID DERIVATIVES AND USES THEREOF

(71) Applicant: Ohio State Innovation Foundation, Columbus, OH (US)

(72) Inventors: Yizhou DONG, Dublin, OH (US); Yuebao ZHANG, Columbus, OH (US); Wenqing LI, Columbus, OH (US); Jingyue YAN, Columbus, OH (US)

(21) Appl. No.: 17/925,470

(22) PCT Filed: May 21, 2021
(86) PCT No.: PCT/US2021/033598

§ 371 Date: Nov. 15, 2022

Related U.S. Application Data
(60) Provisional application No. 63/028,171, filed on May 21, 2020.

(51) Int. Cl. A61K 47/54; A61K 47/69

(52) U.S. Cl. CPC A61K 47/543; A61K 47/6929; A61K 48/00

(57) ABSTRACT

The present disclosure relates to compounds, compositions, and methods for delivery of therapeutic, diagnostic, or prophylactic agents (for example, a nucleic acid).

### CROSS-REFERENCE TO RELATED APPLICATIONS

The application claims the benefit of U.S. Provisional Application No. 63/028,171, filed May 21, 2020, which is hereby incorporated herein by reference in its entirety.

### STATEMENT REGARDING FEDERALLY SPONSORED RESEARCH

This invention was made with government support under Grant No. R35GM119679 awarded by the National Institutes of Health. The Government has certain rights in the invention.

### FIELD

The present disclosure relates to compounds, compositions, and methods for delivery of therapeutic, diagnostic, or prophylactic agents (for example, a nucleic acid).

### BACKGROUND

Messenger RNA (mRNA) based therapeutics have shown great promise for expressing functional antibodies and proteins. Clinical studies have explored mRNA for use in immunotherapy as well as in vaccines through local administration of naked mRNA or mRNA-transfected dendritic cells in order to induce antigen-specific immune responses.

Efficient delivery of mRNA is a key step and challenge for mRNA therapeutics. Despite promising data from ongoing clinical trials, the clinical use of mRNA requires the discovery and development of more efficient delivery systems.

The compounds, compositions, and methods disclosed herein address these and other needs.

### SUMMARY

The present disclosure provides compounds and uses thereof. Also provided are compositions including a compound of the invention and an agent (e.g., an mRNA). The present disclosure also provides methods of using the compositions for delivering an agent to a subject.

In one aspect, the disclosure provides a compound of Formula A:

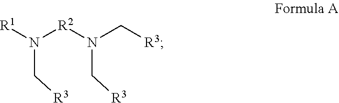

or a salt thereof,

wherein:

R¹ is

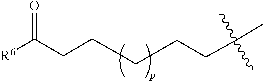

wherein p is an integer from 1 to 20.

R⁶ is a toll-like receptor (TLR) agonist or a derivative thereof;

R² is alkyl, cycloalkyl, heterocycloalkyl, alkylheterocycloalkyl, amine, amide, alkylamide, ether, or alkylether; and

each R³ is independently selected from alkyl, alkenyl, alkynyl, ester, or alkylester.

In some embodiments, R² is

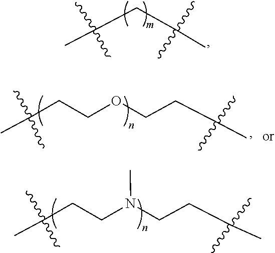

wherein m is an integer from 1 to 20,

wherein n is an integer from 1 to 3.

In one aspect, disclosed herein is a compound of Formula I:

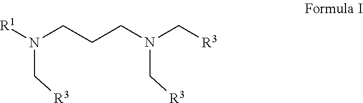

or a salt thereof, wherein:

R¹ is

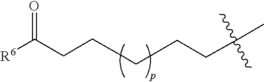

wherein p is an integer from 1 to 20;

R⁶ is a toll-like receptor (TLR) agonist or a derivative thereof; and

each R³ is independently selected from alkyl, alkenyl, alkynyl, ester, or alkylester.

In one aspect, the disclosure provides a composition comprising:

a compound of Formula A:

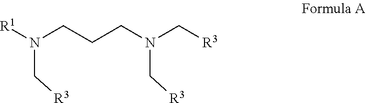

or a salt thereof, wherein:
R¹ is

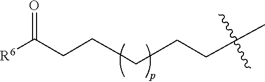

wherein p is an integer from 1 to 20;
R⁶ is a toll-like receptor (TLR) agonist or a derivative thereof;
R² is alkyl, cycloalkyl, heterocycloalkyl, alkylheterocycloalkyl, amide, alkylamide, amine, ether, or alkylether, and
each R³ is independently selected from alkyl, alkenyl, alkynyl, ester, or alkylester; and

an agent.

In some embodiments, the disclosure provides a composition comprising:

a compound of Formula I:

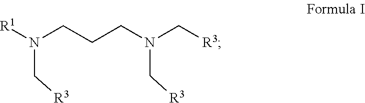

or a salt thereof, wherein:
R¹ is

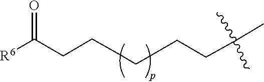

wherein p is an integer from 1 to 20;
R⁶ is a toll-like receptor (TLR) agonist or a derivative thereof; and
each R³ is independently selected from alkyl, alkenyl, alkynyl, ester, or

alkylester; and

an agent.

In some embodiments, R¹ is

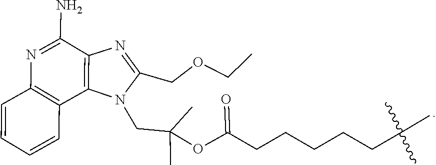 [SMILES: *CCCCCC(=O)OC(C)(C)Cn1c(COCC)nc2c(N)nc3ccccc3c21]

In some embodiments, the agent is a polynucleotide. In some embodiments, the agent is an RNA. In some embodiments, the agent is an mRNA.

In some embodiments, provided are methods for the delivery of nucleic acids. In some embodiments, provided herein are methods for the delivery of polynucleotides. In some embodiments, provided herein are methods to regulate the immune system for treating cancers and other immune disorders. In some embodiments, provided herein are methods for treating or preventing a respiratory infection. In some embodiments, provided herein are methods for inducing an immune response against respiratory viruses.

### BRIEF DESCRIPTION OF THE DRAWINGS

The accompanying figures, which are incorporated in and constitute a part of this specification, illustrate several aspects described below.

FIG. 1 illustrates a synthetic route for Compound 12.

FIG. 2 illustrates a synthetic route for Compound 23.

FIG. 3A-3G show mass spectra for (3A) compound 4, (3B) compound 8, (3C) compound 11, (3D) compound 12, (3E) compound 14, (3F) compound 18, and (3G) compound 23.

FIG. 4A-4D show the characterization of mRNA RAL nanoparticles. (4A) Delivery of luciferase mRNA in JAWS II cells. RAL-1 is formulated from compound 12. RAL-2 is formulated from compound 23. (4B) Representative cryo-TEM image of RAL CD40-LNPs. (4C) Particle size of RAL CD40-LNP. (4D) CD40 expression in JAWS II cells. Data are analyzed by unpaired two-tailed t test. ****P<0.0001. Data in 4A, 4D are presented as the mean±S.E.M.

FIG. 5A-5F show the anti-tumor activity in vivo. (5A) GFP expression in immune cells after intratumoral injection of RAL GFP-LNPs in subcutaneous B16 tumors. (5B) CD40 expression on the surface of dendritic cells after intratumoral injection of RAL GFP-LNPs in subcutaneous B16 tumors. (5C) Experiment setup. (5D) Tumor size after tumor implantation on day 0 (n=8-10). (5E) Tumor size of untreated B16 tumor after tumor rechallenge on opposite flank in cured mice on day 45 (n=5). (5F) Mice survival after the initial tumor implantation on day 0 (n=8-10). Data are analyzed by unpaired two-tailed t test or one-way ANOVA. *P<0.05, ** P<0.005 and *** P<0.0005. Data in 5A, 5D, and 5E are presented as the mean±S.E.M. Survival analysis was performed with the log-rank test.

FIG. 6A-6C show the anti-tumor activity in vivo. (6A) Experiment setup. RAL CD40-LNPs was administered through intravenous (i.v.) injection and anti-CD40 antibody was administered through intraperitoneal (i.p.) injection, respectively. (6B) Growth of B16 tumor after tumor implantation on day 0 (n=4-5). (6C) Overall survival after the initial tumor implantation on day 0 (n=4-5). Data are analyzed by unpaired two-tailed t test. * P<0.05, and ** P<0.005. Data in 6B are presented as the mean C±S.E.M. Survival analysis was performed with the log-rank test.

FIG. 7A-7B show RAL-LNP mediated mRNA delivery Ex vivo. (7A) RAL2 and RAL2-CD40 LNPs increased CD40 expression in primary DCs. (7B) RAL2 and RAL2-CD40 LNPs increased CD40 intensity in primary DCs. Data in 7A and 7B are presented as the mean±S.D. (n=3). Statistical significances are analyzed by one-way ANOVA followed by Dunnett's multiple comparison test. ****P<0.0001.

FIG. 8 shows exemplary compounds.

### DETAILED DESCRIPTION

The present disclosure provides new compounds, nanomaterials, and uses thereof. Also provided are compositions including a compound of the invention and an agent (e.g., an mRNA). The present disclosure also provides methods using the compositions for delivering an agent to a subject. These nanomaterials comprising a toll-like receptor (TLR) agonist or a derivative thereof are used in applications such as gene therapy and drug delivery. These compounds were designed and synthesized with different toll-like receptor (TLR) agonist or a derivative thereof heads and tunable lipid tails.

Reference will now be made in detail to the embodiments of the invention, examples of which are illustrated in the drawings and the examples. This invention may, however, be embodied in many different forms and should not be construed as limited to the embodiments set forth herein.

Unless defined otherwise, all technical and scientific terms used herein have the same meaning as commonly understood to one of ordinary skill in the art to which this invention belongs. The following definitions are provided for the full understanding of terms used in this specification.

### Definitions

General Definitions

Unless defined otherwise, all technical and scientific terms used herein have the same meaning as commonly understood to one of ordinary skill in the art to which this disclosure belongs. The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments and are also disclosed. The following definitions are provided for the full understanding of terms used in this specification.

As used herein, the article “a,” “an,” and “the” means “at least one,” unless the context in which the article is used clearly indicates otherwise.

The term “nucleic acid” as used herein means a polymer composed of nucleotides, e.g. deoxyribonucleotides or ribonucleotides.

The terms “ribonucleic acid” and “RNA” as used herein mean a polymer composed of ribonucleotides.

The terms “deoxyribonucleic acid” and “DNA” as used herein mean a polymer composed of deoxyribonucleotides.

The term “oligonucleotide” denotes single- or double-stranded nucleotide multimers of from about 2 to up to about 100 nucleotides in length. Suitable oligonucleotides may be prepared by the phosphoramidite method described by Beaucage and Carruthers, Tetrahedron Lett., 22:1859-1862 (1981), or by the triester method according to Matteucci, et al., J. Am. Chem. Soc., 103:3185 (1981), both incorporated herein by reference, or by other chemical methods using either a commercial automated oligonucleotide synthesizer or VLSIPS™ technology. When oligonucleotides are referred to as “double-stranded,” it is understood by those of skill in the art that a pair of oligonucleotides exist in a hydrogen-bonded, helical array typically associated with, for example, DNA. In addition to the 100% complementary form of double-stranded oligonucleotides, the term “double-stranded,” as used herein is also meant to refer to those forms which include such structural features as bulges and loops, described more fully in such biochemistry texts as Stryer, Biochemistry, Third Ed., (1988), incorporated herein by reference for all purposes.

The term “polynucleotide” refers to a single or double stranded polymer composed of nucleotide monomers. In some embodiments, the polynucleotide is composed of nucleotide monomers of generally greater than 100 nucleotides in length and up to about 8,000 or more nucleotides in length.

The term “polypeptide” refers to a compound made up of a single chain of D- or L-amino acids or a mixture of D- and L-amino acids joined by peptide bonds.

The term “complementary” refers to the topological compatibility or matching together of interacting surfaces of a probe molecule and its target. Thus, the target and its probe can be described as complementary, and furthermore, the contact surface characteristics are complementary to each other.

The term “hybridization” refers to a process of establishing a non-covalent, sequence-specific interaction between two or more complementary strands of nucleic acids into a single hybrid, which in the case of two strands is referred to as a duplex.

The term “anneal” refers to the process by which a single-stranded nucleic acid sequence pairs by hydrogen bonds to a complementary sequence, forming a double-stranded nucleic acid sequence, including the reformation (renaturation) of complementary strands that were separated by heat (thermally denatured).

The term “melting” refers to the denaturation of a double-stranded nucleic acid sequence due to high temperatures, resulting in the separation of the double strand into two single strands by breaking the hydrogen bonds between the strands.

The term “target” refers to a molecule that has an affinity for a given probe. Targets may be naturally-occurring or man-made molecules. Also, they can be employed in their unaltered state or as aggregates with other species.

The term “promoter” or “regulatory element” refers to a region or sequence determinants located upstream or downstream from the start of transcription and which are involved in recognition and binding of RNA polymerase and other proteins to initiate transcription. Promoters need not be of bacterial origin, for example, promoters derived from viruses or from other organisms can be used in the compositions, systems, or methods described herein. The term “regulatory element” is intended to include promoters, enhancers, internal ribosomal entry sites (IRES), and other expression control elements (e.g. transcription termination signals, such as polyadenylation signals and poly-U sequences). Such regulatory elements are described, for example, in Goeddel, Gene Expression Technology: Methods in Enzymology 185, Academic Press, San Diego, Calif. (1990). Regulatory elements include those that direct constitutive expression of a nucleotide sequence in many types of host cell and those that direct expression of the nucleotide sequence only in certain host cells (e.g., tissue-specific regulatory sequences). A tissue-specific promoter may direct expression primarily in a desired tissue of interest, such as muscle, neuron, bone, skin, blood, specific organs (e.g. liver, pancreas), or particular cell types (e.g. lymphocytes). Regulatory elements may also direct expression in a temporal-dependent manner, such as in a cell-cycle dependent or developmental stage-dependent manner, which may or may not also be tissue or cell-type specific. In some embodiments, a vector comprises one or more pol III promoter (e.g. 1, 2, 3, 4, 5, or more pol I promoters), one or more pol II promoters (e.g. 1, 2, 3, 4, 5, or more pol II promoters), one or more pol I promoters (e.g. 1, 2, 3, 4, 5, or more pol I promoters), or combinations thereof. Examples of pol III promoters include, but are not limited to, U6 and H1 promoters. Examples of pol II promoters include, but are not limited to, the retroviral Rous sarcoma virus (RSV) LTR promoter (optionally with the RSV enhancer), the cytomegalovirus (CMV) promoter (optionally with the CMV enhancer) [see, e.g., Boshart et al, Cell, 41:521-530 (1985)], the SV40 promoter, the dihydrofolate reductase promoter, the β-actin promoter, the phosphoglycerol kinase (PGK) promoter, and the EF1α promoter. Also encompassed by the term “regulatory element” are enhancer elements, such as WPRE; CMV enhancers; the R-U5′ segment in LTR of HTLV-I (Mol. Cell. Biol., Vol. 8(1), p. 466-472, 1988); SV40 enhancer; and the intron sequence between exons 2 and 3 of rabbit β-globin (Proc. Natl. Acad. Sci. USA., Vol. 78(3), p. 1527-31, 1981). It is appreciated by those skilled in the art that the design of the expression vector can depend on such factors as the choice of the host cell to be transformed, the level of expression desired, etc.

The term “recombinant” refers to a human manipulated nucleic acid (e.g. polynucleotide) or a copy or complement of a human manipulated nucleic acid (e.g. polynucleotide), or if in reference to a protein (i.e., a “recombinant protein”), a protein encoded by a recombinant nucleic acid (e.g. polynucleotide). In embodiments, a recombinant expression cassette comprising a promoter operably linked to a second nucleic acid (e.g. polynucleotide) may include a promoter that is heterologous to the second nucleic acid (e.g. polynucleotide) as the result of human manipulation (e.g., by methods described in Sambrook et al., Molecular Cloning A Laboratory Manual, Cold Spring Harbor Laboratory, Cold Spring Harbor, N.Y., (1989) or Current Protocols in Molecular Biology Volumes 1-3, John Wiley & Sons, Inc. (1994-1998)). In another example, a recombinant expression cassette may comprise nucleic acids (e.g. polynucleotides) combined in such a way that the nucleic acids (e.g. polynucleotides) are extremely unlikely to be found in nature. For instance, human manipulated restriction sites or plasmid vector sequences may flank or separate the promoter from the second nucleic acid (e.g. polynucleotide). One of skill will recognize that nucleic acids (e.g. polynucleotides) can be manipulated in many ways and are not limited to the examples above.

The term “expression cassette” refers to a nucleic acid construct, which when introduced into a host cell, results in transcription and/or translation of a RNA or polypeptide, respectively. In embodiments, an expression cassette comprising a promoter operably linked to a second nucleic acid (e.g. polynucleotide) may include a promoter that is heterologous to the second nucleic acid (e.g. polynucleotide) as the result of human manipulation (e.g., by methods described in Sambrook et al., Molecular Cloning A Laboratory Manual, Cold Spring Harbor Laboratory, Cold Spring Harbor, N.Y., (1989) or Current Protocols in Molecular Biology Volumes 1-3, John Wiley & Sons, Inc. (1994-1998)). In some embodiments, an expression cassette comprising a terminator (or termination sequence) operably linked to a second nucleic acid (e.g. polynucleotide) may include a terminator that is heterologous to the second nucleic acid (e.g. polynucleotide) as the result of human manipulation. In some embodiments, the expression cassette comprises a promoter operably linked to a second nucleic acid (e.g. polynucleotide) and a terminator operably linked to the second nucleic acid (e.g. polynucleotide) as the result of human manipulation. In some embodiments, the expression cassette comprises an endogenous promoter. In some embodiments, the expression cassette comprises an endogenous terminator. In some embodiments, the expression cassette comprises a synthetic (or non-natural) promoter. In some embodiments, the expression cassette comprises a synthetic (or non-natural) terminator.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or higher identity over a specified region when compared and aligned for maximum correspondence over a comparison window or designated region) as measured using a BLAST or BLAST 2.0 sequence comparison algorithms with default parameters described below, or by manual alignment and visual inspection (see, e.g., NCBI web site or the like). Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 10 amino acids or 20 nucleotides in length, or more preferably over a region that is 10-50 amino acids or 20-50 nucleotides in length. As used herein, percent (%) amino acid sequence identity is defined as the percentage of amino acids in a candidate sequence that are identical to the amino acids in a reference sequence, after aligning the sequences and introducing gaps, if necessary, to achieve the maximum percent sequence identity. Alignment for purposes of determining percent sequence identity can be achieved in various ways that are within the skill in the art, for instance, using publicly available computer software such as BLAST, BLAST-2, ALIGN, ALIGN-2 or Megalign (DNASTAR) software. Appropriate parameters for measuring alignment, including any algorithms needed to achieve maximal alignment over the full-length of the sequences being compared can be determined by known methods.

For sequence comparisons, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

One example of an algorithm that is suitable for determining percent sequence identity and sequence similarity are the BLAST and BLAST 2.0 algorithms, which are described in Altschul et al. (1977) Nuc. Acids Res. 25:3389-3402, and Altschul et al. (1990) J Mol. Biol. 215:403-410, respectively. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al. (1990) J Mol. Biol. 215:403-410). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) or 10, M=5, N=−4 and a comparison of both strands. For amino acid sequences, the BLASTP program uses as defaults a wordlength of 3, and expectation (E) of 10, and the BLOSUM62 scoring matrix (see Henikoff and Henikoff (1989) Proc. Natl. Acad. Sci. USA 89:10915) alignments (B) of 50, expectation (E) of 10, M=5, N=−4, and a comparison of both strands.

The BLAST algorithm also performs a statistical analysis of the similarity between two sequences (see, e.g., Karlin and Altschul (1993) Proc. Natl. Acad. Sci. USA 90:5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01.

The phrase “codon optimized” as it refers to genes or coding regions of nucleic acid molecules for the transformation of various hosts, refers to the alteration of codons in the gene or coding regions of polynucleic acid molecules to reflect the typical codon usage of a selected organism without altering the polypeptide encoded by the DNA. Such optimization includes replacing at least one, or more than one, or a significant number, of codons with one or more codons that are more frequently used in the genes of that selected organism.

Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are near each other, and, in the case of a secretory leader, contiguous and in reading phase. However, operably linked nucleic acids (e.g. enhancers and coding sequences) do not have to be contiguous. Linking is accomplished by ligation at convenient restriction sites. If such sites do not exist, the synthetic oligonucleotide adaptors or linkers are used in accordance with conventional practice. In embodiments, a promoter is operably linked with a coding sequence when it is capable of affecting (e.g. modulating relative to the absence of the promoter) the expression of a protein from that coding sequence (i.e., the coding sequence is under the transcriptional control of the promoter).

The term “nucleobase” refers to the part of a nucleotide that bears the Watson/Crick base-pairing functionality. The most common naturally-occurring nucleobases, adenine (A), guanine (G), uracil (U), cytosine (C), and thymine (T) bear the hydrogen-bonding functionality that binds one nucleic acid strand to another in a sequence specific manner.

As used throughout, by a “subject” (or a “host”) is meant an individual. Thus, the “subject” can include, for example, domesticated animals, such as cats, dogs, etc., livestock (e.g., cattle, horses, pigs, sheep, goats, etc.), laboratory animals (e.g., mouse, rabbit, rat, guinea pig, etc.) mammals, non-human mammals, primates, non-human primates, rodents, birds, reptiles, amphibians, fish, and any other animal. The subject can be a mammal such as a primate or a human.

The term “about” as used herein when referring to a measurable value such as an amount, a percentage, and the like, is meant to encompass variations of ±20%, ±10%, ±5%, or ±1% from the measurable value.

A nucleic acid sequence is “heterologous” to a second nucleic acid sequence if it originates from a foreign species, or, if from the same species, is modified by human action from its original form. For example, a heterologous promoter (or heterologous 5′ untranslated region (5′UTR)) operably linked to a coding sequence refers to a coding sequence from a species different from that from which the promoter was derived, or, if from the same species, a coding sequence which is different from naturally occurring allelic variants (for example, the 5′UTR or 3′UTR from a different gene is operably linked to a nucleic acid encoding for a co-stimulatory molecule).

As used herein, the terms “treating” or “treatment” of a subject includes the administration of a drug to a subject with the purpose of curing, healing, alleviating, relieving, altering, remedying, ameliorating, improving, stabilizing or affecting a disease or disorder, or a symptom of a disease or disorder. The terms “treating” and “treatment” can also refer to reduction in severity and/or frequency of symptoms, elimination of symptoms and/or underlying cause, and improvement or remediation of damage.

As used herein, the term “preventing” a disease, a disorder, or unwanted physiological event in a subject refers to the prevention of a disease, a disorder, or unwanted physiological event or prevention of a symptom of a disease, a disorder, or unwanted physiological event

“Effective amount” of an agent refers to a sufficient amount of an agent to provide a desired effect. The amount of agent that is “effective” will vary from subject to subject, depending on many factors such as the age and general condition of the subject, the particular agent or agents, and the like. Thus, it is not always possible to specify a quantified “effective amount.” However, an appropriate “effective amount” in any subject case may be determined by one of ordinary skill in the art using routine experimentation. Also, as used herein, and unless specifically stated otherwise, an “effective amount” of an agent can also refer to an amount covering both therapeutically effective amounts and prophylactically effective amounts. An “effective amount” of an agent necessary to achieve a therapeutic effect may vary according to factors such as the age, sex, and weight of the subject. Dosage regimens can be adjusted to provide the optimum therapeutic response. For example, several divided doses may be administered daily or the dose may be proportionally reduced as indicated by the exigencies of the therapeutic situation.

“Pharmaceutically acceptable” component can refer to a component that is not biologically or otherwise undesirable, i.e., the component may be incorporated into a pharmaceutical formulation of the invention and administered to a subject as described herein without causing significant undesirable biological effects or interacting in a deleterious manner with any of the other components of the formulation in which it is contained. When used in reference to administration to a human, the term generally implies the component has met the required standards of toxicological and manufacturing testing or that it is included on the Inactive Ingredient Guide prepared by the U.S. Food and Drug Administration.

“Pharmaceutically acceptable carrier” (sometimes referred to as a “carrier”) means a carrier or excipient that is useful in preparing a pharmaceutical or therapeutic composition that is generally safe and non-toxic, and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents. As used herein, the term “carrier” encompasses, but is not limited to, any excipient, diluent, filler, salt, buffer, stabilizer, solubilizer, lipid, stabilizer, or other material well known in the art for use in pharmaceutical formulations and as described further herein.

“Therapeutic agent” refers to any composition that has a beneficial biological effect. Beneficial biological effects include both therapeutic effects, e.g., treatment of a disorder or other undesirable physiological condition, and prophylactic effects, e.g., prevention of a disorder or other undesirable physiological condition. The terms also encompass pharmaceutically acceptable, pharmacologically active derivatives of beneficial agents specifically mentioned herein, including, but not limited to, salts, esters, amides, proagents, active metabolites, isomers, fragments, analogs, and the like. When the term “therapeutic agent” is used, or when a particular agent is specifically identified, it is to be understood that the term includes the agent per se as well as pharmaceutically acceptable, pharmacologically active salts, esters, amides, proagents, conjugates, active metabolites, isomers, fragments, analogs, etc.

As used herein, the term “controlled-release” or “controlled-release drug delivery” or “extended release” refers to release or administration of a drug from a given dosage form in a controlled fashion in order to achieve the desired pharmacokinetic profile in vivo. An aspect of “controlled” drug delivery is the ability to manipulate the formulation and/or dosage form in order to establish the desired kinetics of drug release.

The phrases “concurrent administration”, “administration in combination”, “simultaneous administration” or “administered simultaneously” as used herein, means that the compounds are administered at the same point in time or immediately following one another.

The term “antibodies” is used herein in a broad sense and includes both polyclonal and monoclonal antibodies. In addition to intact immunoglobulin molecules, also included in the term “antibodies” are fragments or polymers of those immunoglobulin molecules, and human or humanized versions of immunoglobulin molecules or fragments thereof. The antibodies can be tested for their desired activity using the in vitro assays described herein, or by analogous methods, after which their in vivo therapeutic and/or prophylactic activities are tested according to known clinical testing methods. There are five major classes of human immunoglobulins: IgA, IgD, IgE, IgG and IgM, and several of these may be further divided into subclasses (isotypes), e.g., IgG-1, IgG-2, IgG-3, and IgG-4; IgA-1 and IgA-2. One skilled in the art would recognize the comparable classes for mouse. The heavy chain constant domains that correspond to the different classes of immunoglobulins are called alpha, delta, epsilon, gamma, and mu, respectively.

The term “monoclonal antibody” as used herein refers to an antibody obtained from a substantially homogeneous population of antibodies, i.e., the individual antibodies within the population are identical except for possible naturally occurring mutations that may be present in a small subset of the antibody molecules. The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, as long as they exhibit the desired antagonistic activity.

The disclosed monoclonal antibodies can be made using any procedure which produces monoclonal antibodies. For example, disclosed monoclonal antibodies can be prepared using hybridoma methods, such as those described by Kohler and Milstein, Nature, 256:495 (1975). In a hybridoma method, a mouse or other appropriate host animal is typically immunized with an immunizing agent to elicit lymphocytes that produce or are capable of producing antibodies that will specifically bind to the immunizing agent. Alternatively, the lymphocytes may be immunized in vitro.

The monoclonal antibodies may also be made by recombinant DNA methods. DNA encoding the disclosed monoclonal antibodies can be readily isolated and sequenced using conventional procedures (e.g., by using oligonucleotide probes that are capable of binding specifically to genes encoding the heavy and light chains of murine antibodies). Libraries of antibodies or active antibody fragments can also be generated and screened using phage display techniques, e.g., as described in U.S. Pat. No. 5,804,440 to Burton et al. and U.S. Pat. No. 6,096,441 to Barbas et al.

In vitro methods are also suitable for preparing monovalent antibodies. Digestion of antibodies to produce fragments thereof, particularly, Fab fragments, can be accomplished using routine techniques known in the art. For instance, digestion can be performed using papain. Examples of papain digestion are described in WO 94/29348 published Dec. 22, 1994 and U.S. Pat. No. 4,342,566. Papain digestion of antibodies typically produces two identical antigen binding fragments, called Fab fragments, each with a single antigen binding site, and a residual Fc fragment. Pepsin treatment yields a fragment that has two antigen combining sites and is still capable of cross-linking antigen.

As used herein, the term “antibody or antigen binding fragment thereof” or “antibody or fragments thereof” encompasses chimeric antibodies and hybrid antibodies, with dual or multiple antigen or epitope specificities, and fragments, such as F(ab′)2, Fab′, Fab, Fv, sFv, scFv and the like, including hybrid fragments. Thus, fragments of the antibodies that retain the ability to bind their specific antigens are provided. For example, fragments of antibodies which maintain binding activity are included within the meaning of the term “antibody or antigen binding fragment thereof.” Such antibodies and fragments can be made by techniques known in the art and can be screened for specificity and activity according to the methods set forth in the Examples and in general methods for producing antibodies and screening antibodies for specificity and activity (See Harlow and Lane. Antibodies, A Laboratory Manual. Cold Spring Harbor Publications, New York, (1988)).

Also included within the meaning of “antibody or antigen binding fragment thereof” are conjugates of antibody fragments and antigen binding proteins (single chain antibodies). Also included within the meaning of “antibody or antigen binding fragment thereof” are immunoglobulin single variable domains, such as for example a nanobody.

The fragments, whether attached to other sequences or not, can also include insertions, deletions, substitutions, or other selected modifications of particular regions or specific amino acids residues, provided the activity of the antibody or antibody fragment is not significantly altered or impaired compared to the non-modified antibody or antibody fragment. These modifications can provide for some additional property, such as to remove/add amino acids capable of disulfide bonding, to increase its bio-longevity, to alter its secretory characteristics, etc. In any case, the antibody or antibody fragment must possess a bioactive property, such as specific binding to its cognate antigen. Functional or active regions of the antibody or antibody fragment may be identified by mutagenesis of a specific region of the protein, followed by expression and testing of the expressed polypeptide. Such methods are readily apparent to a skilled practitioner in the art and can include site-specific mutagenesis of the nucleic acid encoding the antibody or antibody fragment. (Zoller, M. J. Curr. Opin. Biotechnol. 3:348-354, 1992).

As used herein, the term “antibody” or “antibodies” can also refer to a human antibody and/or a humanized antibody. Many non-human antibodies (e.g., those derived from mice, rats, or rabbits) are naturally antigenic in humans, and thus can give rise to undesirable immune responses when administered to humans. Therefore, the use of human or humanized antibodies in the methods serves to lessen the chance that an antibody administered to a human will evoke an undesirable immune response.

The term “toll-like receptor” (TLR) refers to a member of a family of receptors that bind to pathogen associated molecular patterns (PAMPs) and facilitate an immune response in a mammal. Ten mammalian TLRs are known, e.g., TLR 1-10. The term “toll-like receptor agonist” (TLR agonist) refers to a molecule that binds to a TLR. Synthetic TLR agonists are chemical compounds that are designed to bind to a TLR and activate the receptor. Exemplary synthetic TLR agonists provided herein include “TLR-7 agonist”, “TLR” agonist”, “TLR-3 agonist” and “TLR-9 agonist.” TLR agonists include imiquimod, resiquimod, broprimine and loxoribine, gardiquimod, CL075, SM324405, UC1V150, CU-T12-9, or derivatives thereof.

### Chemical Definitions

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, the heteroatoms, such as nitrogen, can have hydrogen substituents and/or any permissible substituents of organic compounds described herein which satisfy the valences of the heteroatoms. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound, e.g., a compound that does not spontaneously undergo transformation such as by rearrangement, cyclization, elimination, etc.

“Z¹,” “Z²,” “Z³,” and “Z⁴” are used herein as generic symbols to represent various specific substituents. These symbols can be any substituent, not limited to those disclosed herein, and when they are defined to be certain substituents in one instance, they can, in another instance, be defined as some other substituents.

The term “aliphatic” as used herein refers to a non-aromatic hydrocarbon group and includes branched and unbranched, alkyl, alkenyl, or alkynyl groups.

The term “alkyl” as used herein is a branched or unbranched saturated hydrocarbon group of 1 to 24 carbon atoms, such as methyl, ethyl, n-propyl, isopropyl, n-butyl, isobutyl, t-butyl, pentyl, hexyl, heptyl, octyl, nonyl, decyl, dodecyl, tetradecyl, hexadecyl, eicosyl, tetracosyl, and the like. The alkyl group can also be substituted or unsubstituted. The alkyl group can be substituted with one or more groups including, but not limited to, alkyl, halogenated alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol, as described below.

Throughout the specification “alkyl” is generally used to refer to both unsubstituted alkyl groups and substituted alkyl groups; however, substituted alkyl groups are also specifically referred to herein by identifying the specific substituent(s) on the alkyl group. For example, the term “halogenated alkyl” specifically refers to an alkyl group that is substituted with one or more halide, e.g., fluorine, chlorine, bromine, or iodine. The term “alkoxyalkyl” specifically refers to an alkyl group that is substituted with one or more alkoxy groups, as described below. The term “alkylamino” specifically refers to an alkyl group that is substituted with one or more amino groups, as described below, and the like. When “alkyl” is used in one instance and a specific term such as “alkylalcohol” is used in another, it is not meant to imply that the term “alkyl” does not also refer to specific terms such as “alkylalcohol” and the like.

This practice is also used for other groups described herein. That is, while a term such as “cycloalkyl” refers to both unsubstituted and substituted cycloalkyl moieties, the substituted moieties can, in addition, be specifically identified herein; for example, a particular substituted cycloalkyl can be referred to as, e.g., an “alkylcycloalkyl.” Similarly, a substituted alkoxy can be specifically referred to as, e.g., a “halogenated alkoxy,” a particular substituted alkenyl can be, e.g., an “alkenylalcohol,” and the like. Again, the practice of using a general term, such as “cycloalkyl,” and a specific term, such as “alkylcycloalkyl,” is not meant to imply that the general term does not also include the specific term.

The term “alkoxy” as used herein is an alkyl group bound through a single, terminal ether linkage; that is, an “alkoxy” group can be defined as —OZ′ where Z′ is alkyl as defined above.

The term “alkenyl” as used herein is a hydrocarbon group of from 2 to 24 carbon atoms with a structural formula containing at least one carbon-carbon double bond. Asymmetric structures such as (Z¹Z²)C═C(Z³Z⁴) are intended to include both the E and Z isomers. This can be presumed in structural formulae herein wherein an asymmetric alkene is present, or it can be explicitly indicated by the bond symbol C═C. The alkenyl group can be substituted with one or more groups including, but not limited to, alkyl, halogenated alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol, as described below.

The term “alkynyl” as used herein is a hydrocarbon group of 2 to 24 carbon atoms with a structural formula containing at least one carbon-carbon triple bond. The alkynyl group can be substituted with one or more groups including, but not limited to, alkyl, halogenated alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol, as described below.

The term “aryl” as used herein is a group that contains any carbon-based aromatic group including, but not limited to, benzene, naphthalene, phenyl, biphenyl, phenoxybenzene, and the like. The term “heteroaryl” is defined as a group that contains an aromatic group that has at least one heteroatom incorporated within the ring of the aromatic group. Examples of heteroatoms include, but are not limited to, nitrogen, oxygen, sulfur, and phosphorus. The term “non-heteroaryl,” which is included in the term “aryl,” defines a group that contains an aromatic group that does not contain a heteroatom. The aryl or heteroaryl group can be substituted or unsubstituted. The aryl or heteroaryl group can be substituted with one or more groups including, but not limited to, alkyl, halogenated alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol as described herein. The term “biaryl” is a specific type of aryl group and is included in the definition of aryl. Biaryl refers to two aryl groups that are bound together via a fused ring structure, as in naphthalene, or are attached via one or more carbon-carbon bonds, as in biphenyl.

The term “cycloalkyl” as used herein is a non-aromatic carbon-based ring composed of at least three carbon atoms. Examples of cycloalkyl groups include, but are not limited to, cyclopropyl, cyclobutyl, cyclopentyl, cyclohexyl, etc. The term “heterocycloalkyl” is a cycloalkyl group as defined above where at least one of the carbon atoms of the ring is substituted with a heteroatom such as, but not limited to, nitrogen, oxygen, sulfur, or phosphorus. The cycloalkyl group and heterocycloalkyl group can be substituted or unsubstituted. The cycloalkyl group and heterocycloalkyl group can be substituted with one or more groups including, but not limited to, alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol as described herein.

The term “cycloalkenyl” as used herein is a non-aromatic carbon-based ring composed of at least three carbon atoms and containing at least one double bound, i.e., C═C. Examples of cycloalkenyl groups include, but are not limited to, cyclopropenyl, cyclobutenyl, cyclopentenyl, cyclopentadienyl, cyclohexenyl, cyclohexadienyl, and the like. The term “heterocycloalkenyl” is a type of cycloalkenyl group as defined above, and is included within the meaning of the term “cycloalkenyl,” where at least one of the carbon atoms of the ring is substituted with a heteroatom such as, but not limited to, nitrogen, oxygen, sulfur, or phosphorus. The cycloalkenyl group and heterocycloalkenyl group can be substituted or unsubstituted. The cycloalkenyl group and heterocycloalkenyl group can be substituted with one or more groups including, but not limited to, alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol as described herein.

The term “cyclic group” is used herein to refer to either aryl groups, non-aryl groups (i.e., cycloalkyl, heterocycloalkyl, cycloalkenyl, and heterocycloalkenyl groups), or both. Cyclic groups have one or more ring systems that can be substituted or unsubstituted. A cyclic group can contain one or more aryl groups, one or more non-aryl groups, or one or more aryl groups and one or more non-aryl groups.

The term “aldehyde” as used herein is represented by the formula —C(O)H. Throughout this specification “C(O)” or “CO” is a short hand notation for C═O.

The terms “amine” or “amino” as used herein are represented by the formula —NZ′Z², where Z¹ and Z² can each be substitution group as described herein, such as hydrogen, an alkyl, halogenated alkyl, alkenyl, alkynyl, aryl, heteroaryl, cycloalkyl, cycloalkenyl, heterocycloalkyl, or heterocycloalkenyl group described above.

The term “carboxylic acid” as used herein is represented by the formula —C(O)OH. A “carboxylate” or “carboxyl” group as used herein is represented by the formula —C(O)O⁻.

The term “ester” as used herein is represented by the formula —OC(O)Z′ or —C(O)OZ′, where Z′ can be an alkyl, halogenated alkyl, alkenyl, alkynyl, aryl, heteroaryl, cycloalkyl, cycloalkenyl, heterocycloalkyl, or heterocycloalkenyl group described above.

The term “ether” as used herein is represented by the formula Z¹OZ², where Z¹ and Z² can be, independently, an alkyl, halogenated alkyl, alkenyl, alkynyl, aryl, heteroaryl, cycloalkyl, cycloalkenyl, heterocycloalkyl, or heterocycloalkenyl group described above.

The term “ketone” as used herein is represented by the formula Z′C(O)Z², where Z¹ and Z² can be, independently, an alkyl, halogenated alkyl, alkenyl, alkynyl, aryl, heteroaryl, cycloalkyl, cycloalkenyl, heterocycloalkyl, or heterocycloalkenyl group described above.

The term “halide” or “halogen” as used herein refers to the fluorine, chlorine, bromine, and iodine.

The term “hydroxyl” as used herein is represented by the formula —OH.

The term “nitro” as used herein is represented by the formula —NO₂.

The term “silyl” as used herein is represented by the formula —SiZ¹Z²Z³, where Z¹, Z², and Z³ can be, independently, hydrogen, alkyl, halogenated alkyl, alkoxy, alkenyl, alkynyl, aryl, heteroaryl, cycloalkyl, cycloalkenyl, heterocycloalkyl, or heterocycloalkenyl group described above.

The term “sulfonyl” is used herein to refer to the sulfo-oxo group represented by the formula —S(O)₂Z¹, where Z′ can be hydrogen, an alkyl, halogenated alkyl, alkenyl, alkynyl, aryl, heteroaryl, cycloalkyl, cycloalkenyl, heterocycloalkyl, or heterocycloalkenyl group described above.

The term “sulfonylamino” or “sulfonamide” as used herein is represented by the formula —S(O)₂NH—.

The term “phosphonyl” is used herein to refer to the phospho-oxo group represented by the formula —P(O)(OZ¹)₂, where Z¹ can be hydrogen, an alkyl, halogenated alkyl, alkenyl, alkynyl, aryl, heteroaryl, cycloalkyl, cycloalkenyl, heterocycloalkyl, or heterocycloalkenyl group described above.

The term “thiol” as used herein is represented by the formula —SH.

The term “thio” as used herein is represented by the formula —S—.

“R¹,” “R²,” “R³,” “R”,” etc., where n is some integer, as used herein can, independently, possess one or more of the groups listed above. For example, if R¹ is a straight chain alkyl group, one of the hydrogen atoms of the alkyl group can optionally be substituted with a hydroxyl group, an alkoxyl group, an amine group, an alkyl group, a halide, and the like. Depending upon the groups that are selected, a first group can be incorporated within second group or, alternatively, the first group can be pendant (i.e., attached) to the second group. For example, with the phrase “an alkyl group comprising an amino group,” the amino group can be incorporated within the backbone of the alkyl group. Alternatively, the amino group can be attached to the backbone of the alkyl group. The nature of the group(s) that is (are) selected will determine if the first group is embedded or attached to the second group.

Unless stated to the contrary, a formula with chemical bonds shown only as solid lines and not as wedges or dashed lines contemplates each possible isomer, e.g., each enantiomer, diastereomer, and meso compound, and a mixture of isomers, such as a racemic or scalemic mixture.

Reference will now be made in detail to specific aspects of the disclosed materials, compounds, compositions, articles, and methods, examples of which are illustrated in the accompanying Examples and Figures.

### Compounds

In one aspect, disclosed herein is a compound of Formula A:

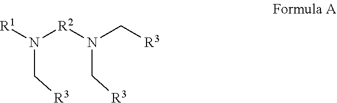

or a salt thereof, wherein:

R¹ is

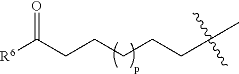

wherein p is an integer from 1 to 20;

R⁶ is a toll-like receptor (TLR) agonist or a derivative thereof;

R² is alkyl, cycloalkyl, heterocycloalkyl, alkylheterocycloalkyl, amide, alkylamide, amine, ether, or alkylether, and

each R³ is independently selected from alkyl, alkenyl, alkynyl, ester, or alkylester.

In some embodiments, R⁶ is

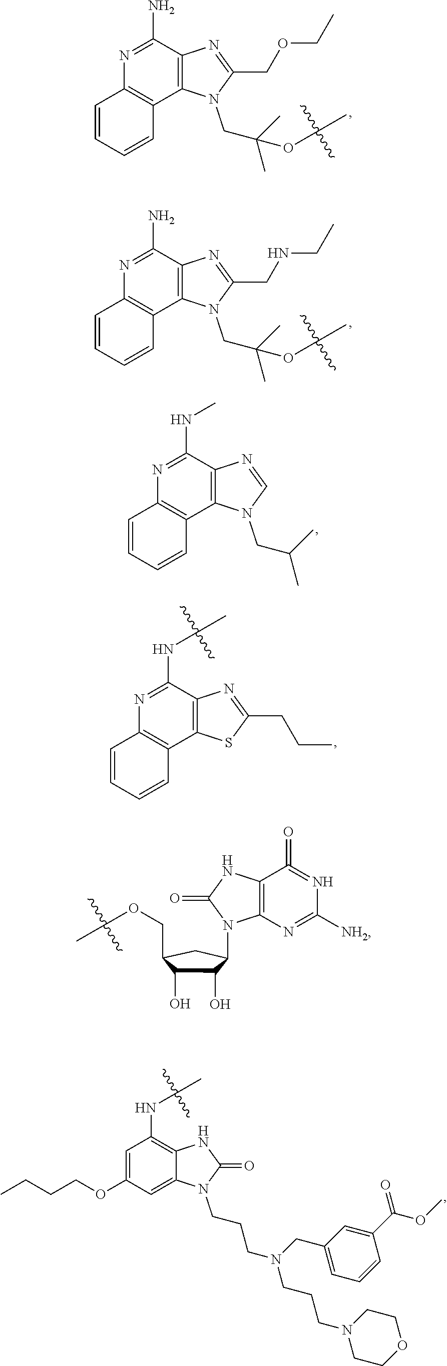

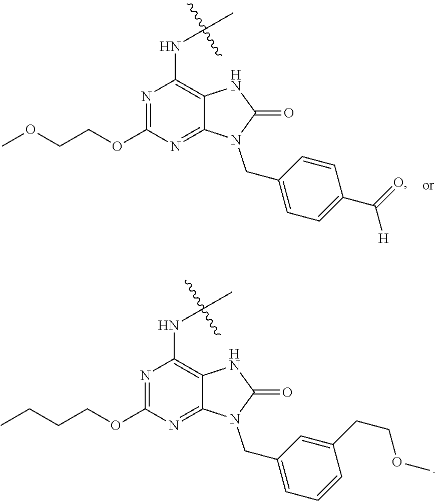 [SMILES: *Nc1nc(OCCOC)nc2c1[nH]c(=O)n2Cc1ccc(C=O)cc1 ; *Nc1nc(OCCCC)nc2c1[nH]c(=O)n2Cc1cccc(CCOC)c1]

In some embodiments, R⁶ is

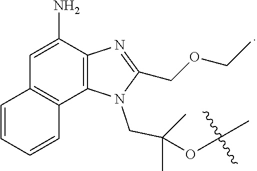 [SMILES: *OC(C)(C)Cn1c(COCC)nc2c(N)cc3ccccc3c21]

In some embodiments, R¹ is

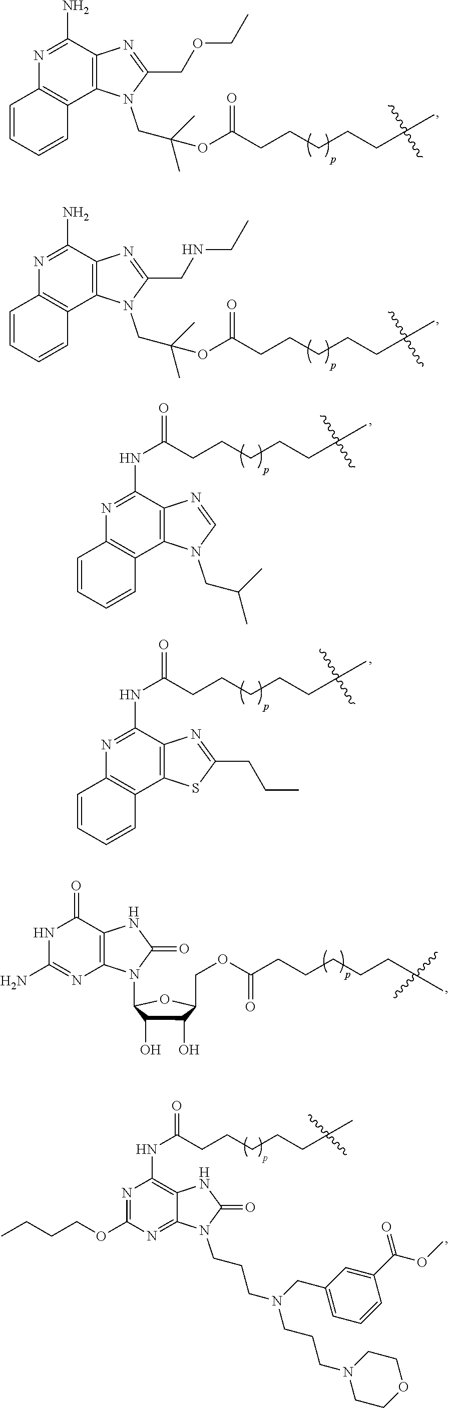

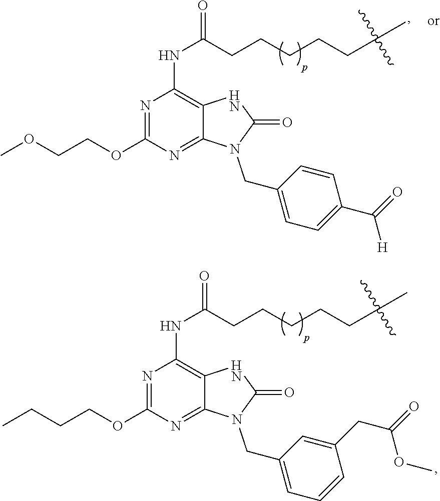

wherein p is an integer from 1 to 20.

In some embodiments, R¹ is

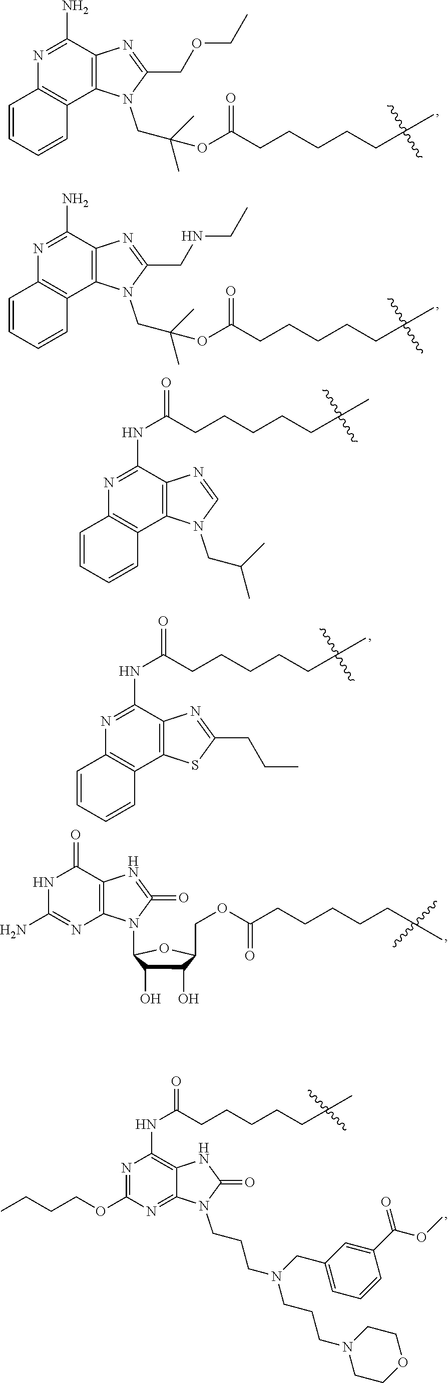

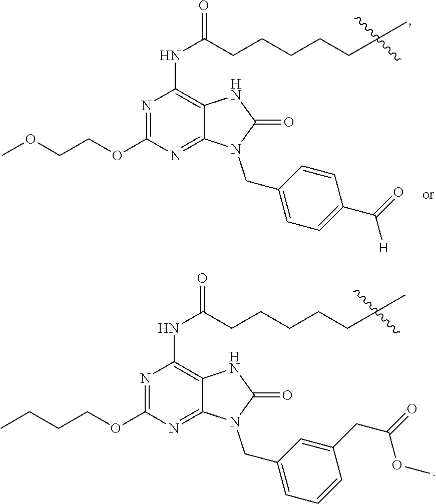 [SMILES: *CCCCCC(=O)Nc1nc(OCCOC)nc2c1[nH]c(=O)n2Cc1ccc(C=O)cc1 ; *CCCCCC(=O)Nc1nc(OCCCC)nc2c1[nH]c(=O)n2Cc1cccc(CC(=O)OC)c1]

In some embodiments, R¹ is

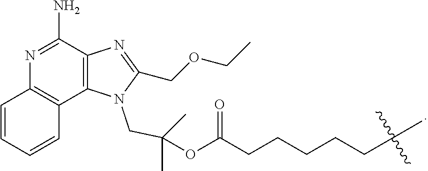 [SMILES: *CCCCCC(=O)OC(C)(C)Cn1c(COCC)nc2c(N)nc3ccccc3c21]

In some embodiments, R² is

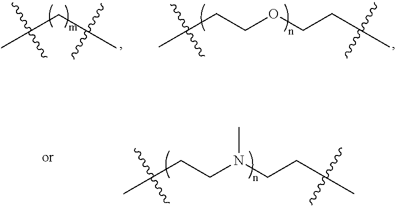

wherein m is an integer from 1 to 20,
wherein n is an integer from 1 to 3.

In some embodiments, disclosed herein is a compound of Formula A:

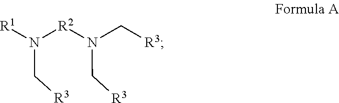

or a salt thereof, wherein:

R¹ is

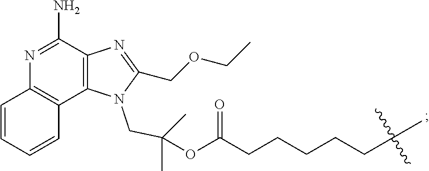 [SMILES: *CCCCCC(=O)OC(C)(C)Cn1c(COCC)nc2c(N)nc3ccccc3c21]

and

R² is alkyl, cycloalkyl, heterocycloalkyl, alkylheterocycloalkyl, amide, alkylamide, ether, alkylether,

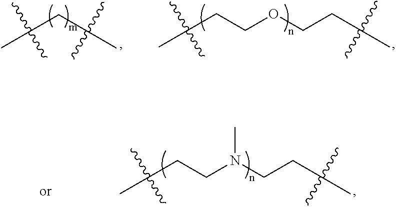

wherein m is an integer from 1 to 20,

wherein n is an integer from 1 to 3; and

each R³ is independently selected from alkyl, alkenyl, alkynyl, ester, or alkylester.

In one aspect, disclosed herein is a compound of Formula I, II, III, IV, V, VI, VII, VIII, IX or X:

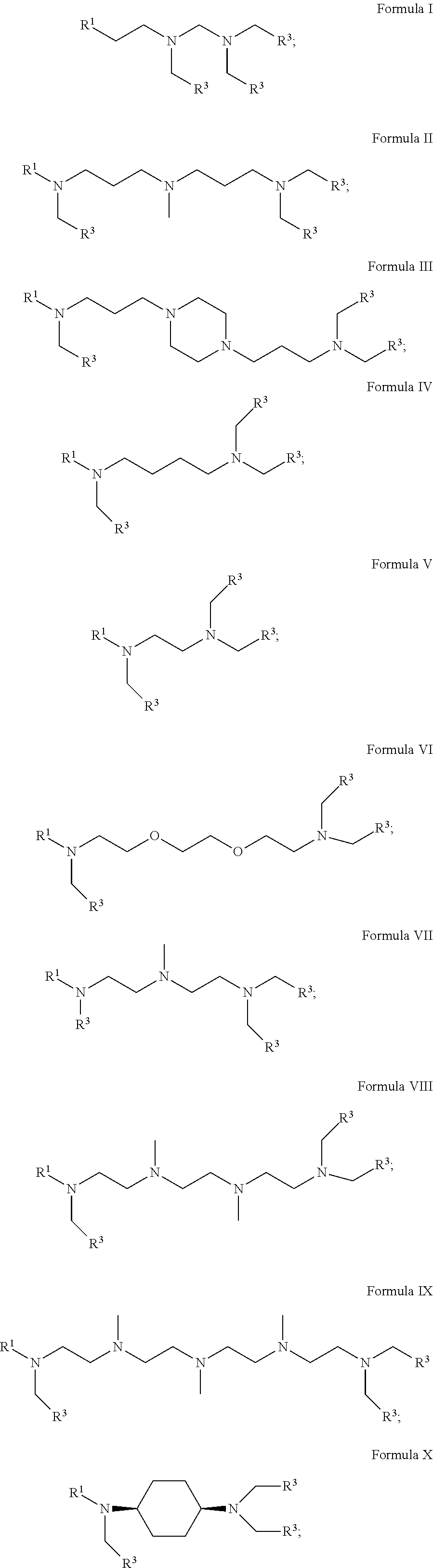

or a salt thereof, wherein:

R¹ is

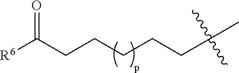

wherein p is an integer from 1 to 20;

R⁶ is a toll-like receptor (TLR) agonist or a derivative thereof; and

each R³ is independently selected from alkyl, alkenyl, alkynyl, ester, or alkylester.

In some embodiments, R⁶ is

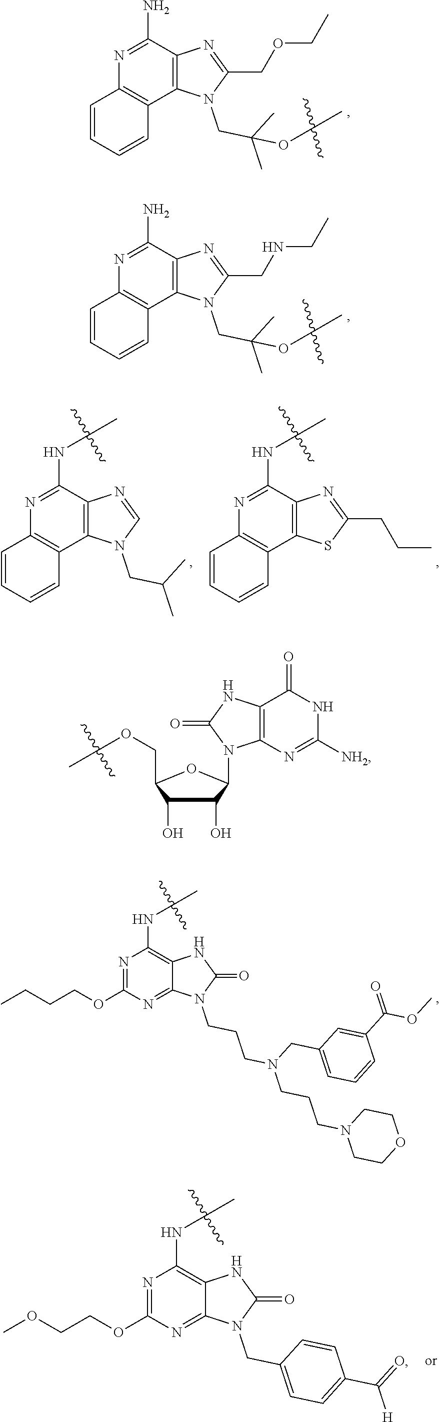

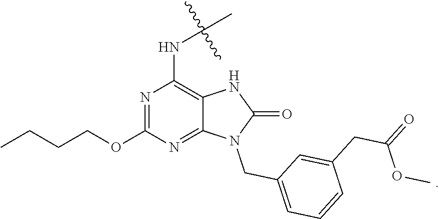 [SMILES: *Nc1nc(OCCCC)nc2c1[nH]c(=O)n2Cc1cccc(CC(=O)OC)c1]

In some embodiments, R⁶ is

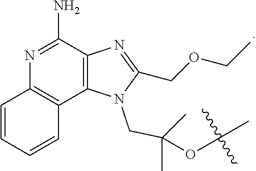 [SMILES: *OC(C)(C)Cn1c(COCC)nc2c(N)nc3ccccc3c21]

In some embodiments, R¹ is

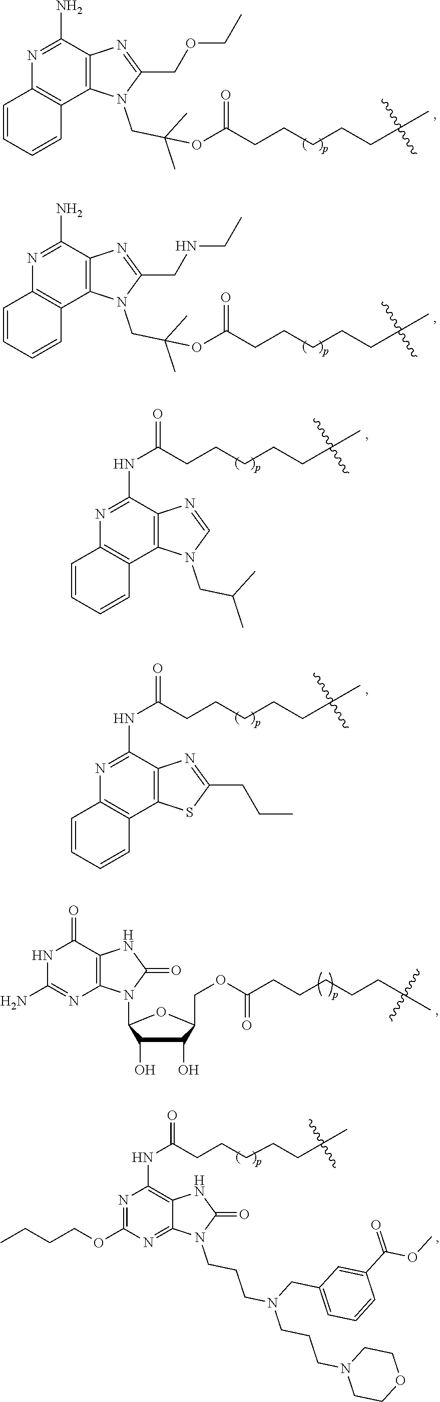

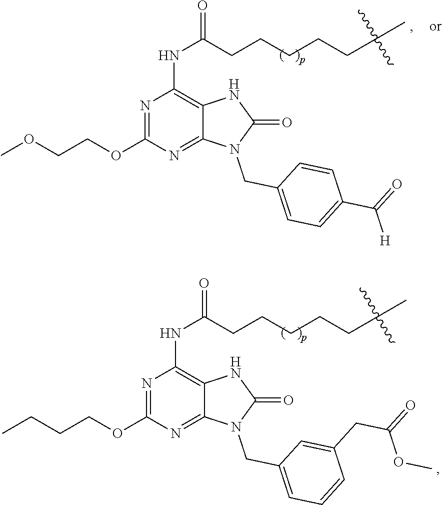

wherein p is an integer from 1 to 20.

In some embodiments, R¹ is

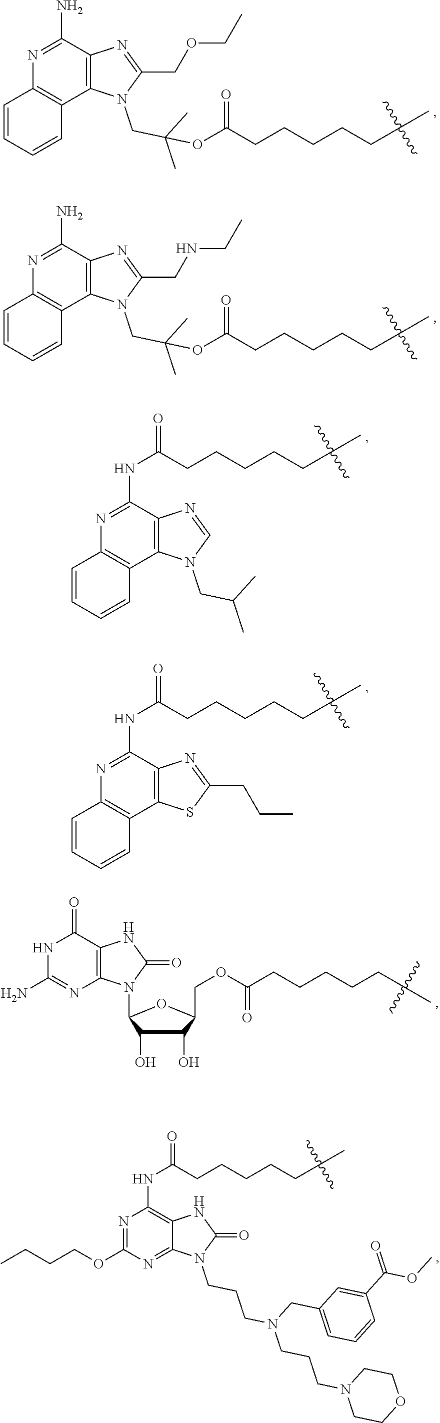

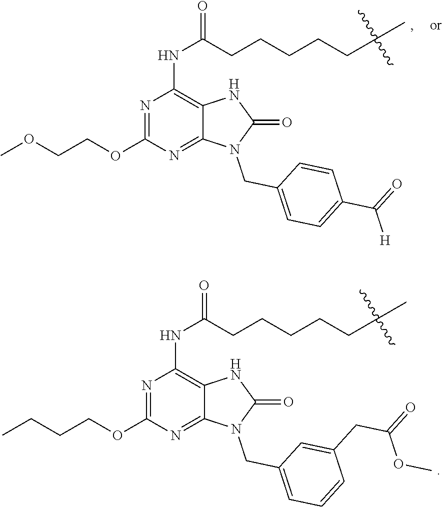 [SMILES: *CCCCCC(=O)Nc1nc(OCCOC)nc2c1[nH]c(=O)n2Cc1ccc(C=O)cc1 ; *CCCCCC(=O)Nc1nc(OCCCC)nc2c1[nH]c(=O)n2Cc1cccc(CC(=O)OC)c1]

In some embodiments, R¹ is

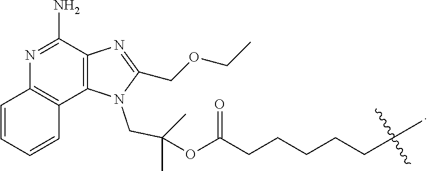 [SMILES: *CCCCCC(=O)OC(C)(C)Cn1c(COCC)nc2c(N)nc3ccccc3c21]

In one aspect, disclosed herein is a compound of Formula I:

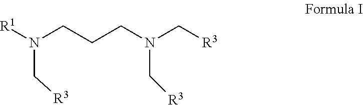

or a salt thereof, wherein:

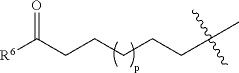

wherein p is an integer from 1 to 20;

R⁶ is a toll-like receptor (TLR) agonist or a derivative thereof; and

each R³ is independently selected from alkyl, alkenyl, alkynyl, ester, or alkylester.

In some embodiments, disclosed herein is a compound of Formula I:

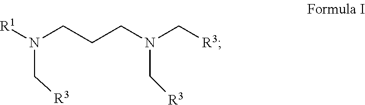

or a salt thereof, wherein:

R¹ is

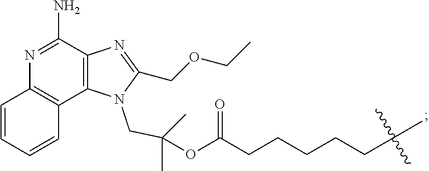 [SMILES: *CCCCCC(=O)OC(C)(C)Cn1c(COCC)nc2c(N)nc3ccccc3c21]

and

each R³ is independently selected from alkyl, alkenyl, alkynyl, ester, or alkylester.

In one aspect, disclosed herein is a compound of Formula II:

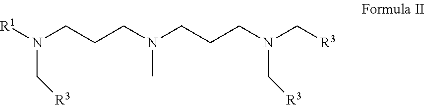

or a salt thereof, wherein:

R¹ is

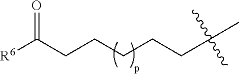

wherein p is an integer from 1 to 20;

R⁶ is a toll-like receptor (TLR) agonist or a derivative thereof; and

each R³ is independently selected from alkyl, alkenyl, alkynyl, ester, or alkylester.

In one aspect, disclosed herein is a compound of Formula III:

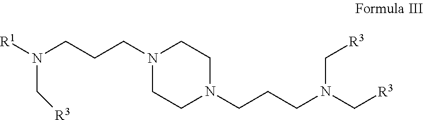

or a salt thereof, wherein:

R¹ is

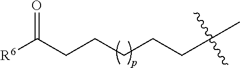

wherein p is an integer from 1 to 20;

R⁶ is a toll-like receptor (TLR) agonist or a derivative thereof; and

each R³ is independently selected from alkyl, alkenyl, alkynyl, ester, or alkylester.

In one aspect, disclosed herein is a compound of Formula IV:

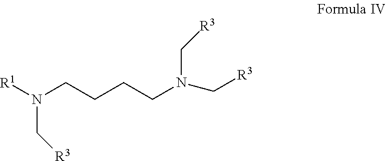

or a salt thereof, wherein:

R¹ is

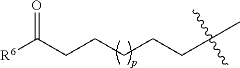

wherein p is an integer from 1 to 20;

R⁶ is a toll-like receptor (TLR) agonist or a derivative thereof; and

each R³ is independently selected from alkyl, alkenyl, alkynyl, ester, or alkylester.

In one aspect, disclosed herein is a compound of Formula V:

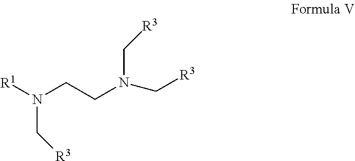

or a salt thereof, wherein:

R¹ is

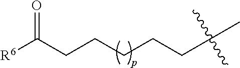

wherein p is an integer from 1 to 20;

R⁶ is a toll-like receptor (TLR) agonist or a derivative thereof; and

each R³ is independently selected from alkyl, alkenyl, alkynyl, ester, or alkylester.

In one aspect, disclosed herein is a compound of Formula VI:

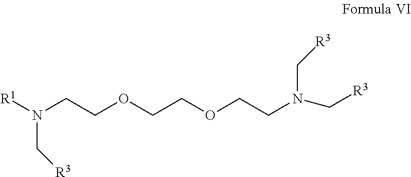

or a salt thereof, wherein:

R¹ is

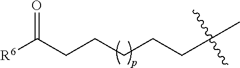

wherein p is an integer from 1 to 20;

R⁶ is a toll-like receptor (TLR) agonist or a derivative thereof; and

each R³ is independently selected from alkyl, alkenyl, alkynyl, ester, or alkylester.

In one aspect, disclosed herein is a compound of Formula VII:

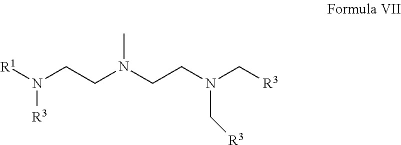

or a salt thereof, wherein:

R¹ is

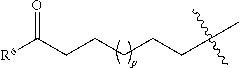

wherein p is an integer from 1 to 20;

R⁶ is a toll-like receptor (TLR) agonist or a derivative thereof; and

each R³ is independently selected from alkyl, alkenyl, alkynyl, ester, or alkylester.

In one aspect, disclosed herein is a compound of Formula VIII:

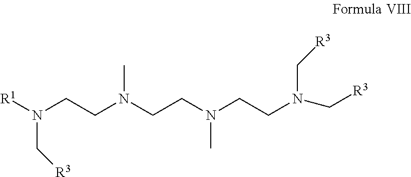

or a salt thereof, wherein:

R¹ is

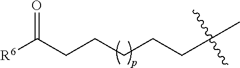

wherein p is an integer from 1 to 20;

R⁶ is a toll-like receptor (TLR) agonist or a derivative thereof; and

each R³ is independently selected from alkyl, alkenyl, alkynyl, ester, or alkylester.

In one aspect, disclosed herein is a compound of Formula IX:

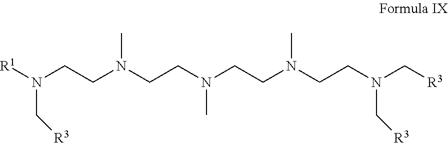

or a salt thereof, wherein:

R¹ is

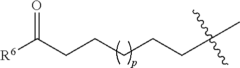

wherein p is an integer from 1 to 20;

R⁶ is a toll-like receptor (TLR) agonist or a derivative thereof; and

each R³ is independently selected from alkyl, alkenyl, alkynyl, ester, or alkylester.

In one aspect, disclosed herein is a compound of Formula X:

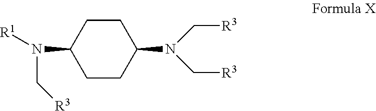

or a salt thereof, wherein:

R¹ is

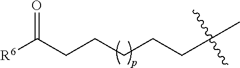

wherein p is an integer from 1 to 20;

R⁶ is a toll-like receptor (TLR) agonist or a derivative thereof; and

each R³ is independently selected from alkyl, alkenyl, alkynyl, ester, or alkylester.

In one aspect, the disclosure provides a compound of Formula A, I, II, III, IV, V, VI, VII, VIII, IX, X, or a salt thereof, wherein:

R⁶ is a toll-like receptor (TLR) agonist or a derivative thereof.

In one aspect, the disclosure provides a compound of Formula A, I, II, III, IV, V, VI, VII, VIII, IX, X, or a salt thereof, wherein:

R⁶ is

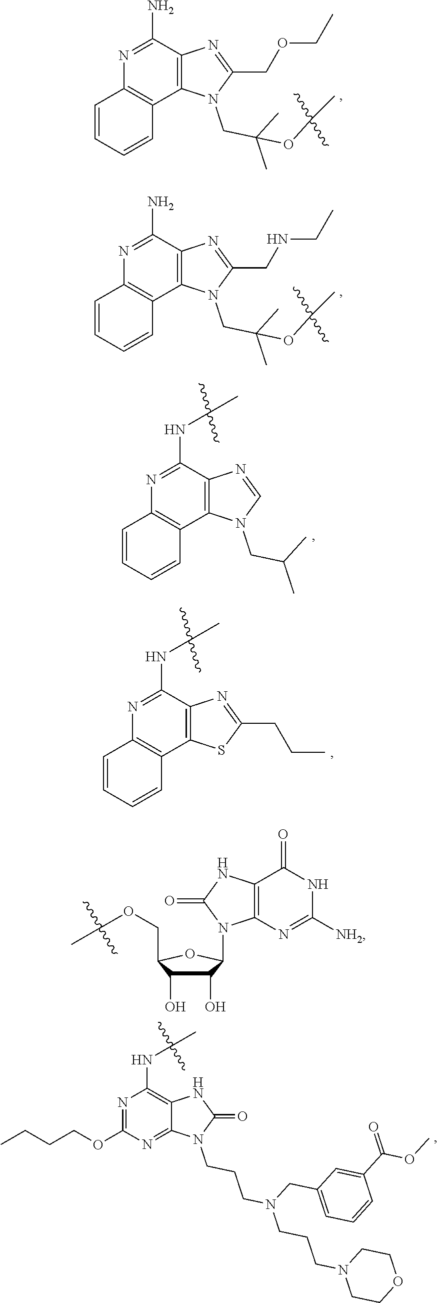

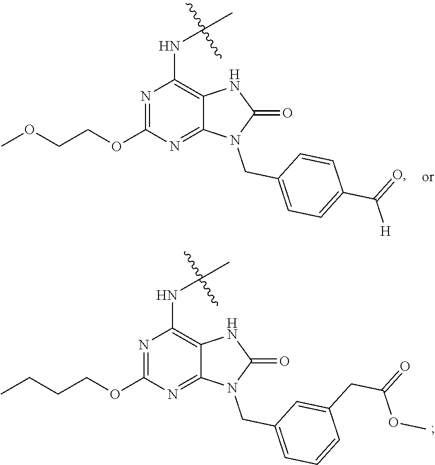 [SMILES: *Nc1nc(OCCOC)nc2c1[nH]c(=O)n2Cc1ccc(C=O)cc1 ; *Nc1nc(OCCCC)nc2c1[nH]c(=O)n2Cc1cccc(CC(=O)OC)c1]

and

each R³ is independently selected from alkyl, alkenyl, alkynyl, ester, or alkylester.

In some embodiments, the disclosure provides a compound of Formula A, I, II, III, IV, V, VI, VII, VIII, IX, X, or a salt thereof, wherein:

R⁶ is

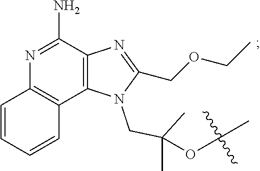 [SMILES: *OC(C)(C)Cn1c(COCC)nc2c(N)nc3ccccc3c21]

and

each R³ is independently selected from alkyl, alkenyl, alkynyl, ester, or alkylester.

In some embodiments, the disclosure provides a compound of Formula A, I, II, III, IV, V, VI, VII, VIII, IX, X, or a salt thereof, wherein:

R¹ is

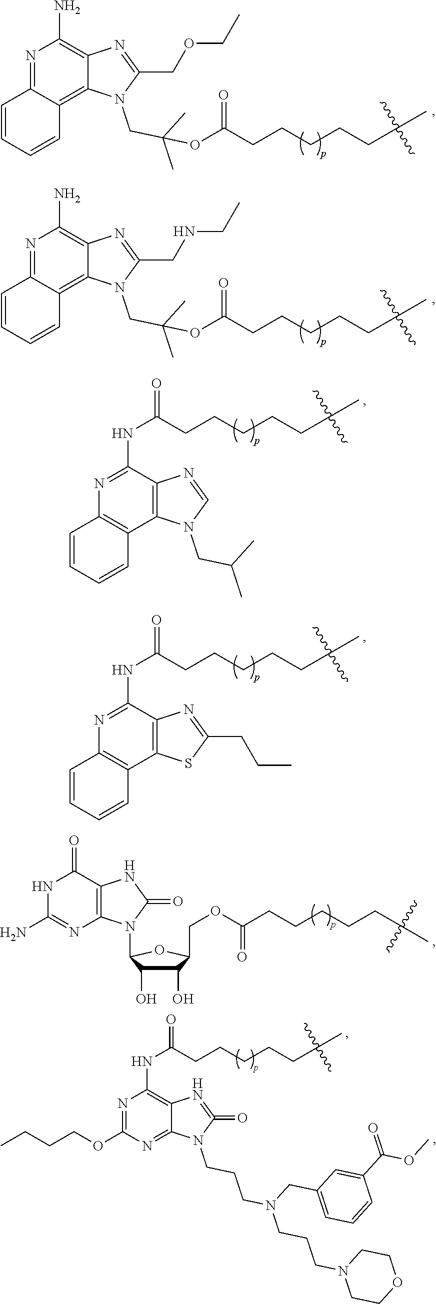

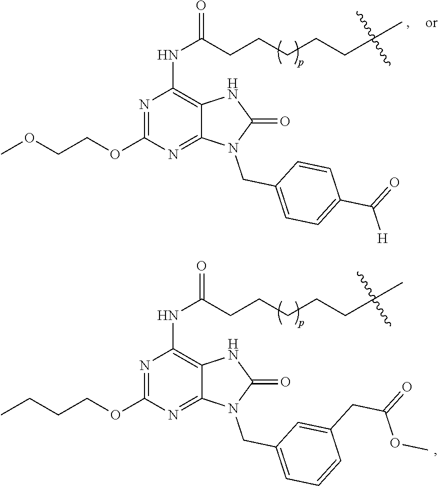

wherein p is an integer from 1 to 20; and

each R³ is independently selected from alkyl, alkenyl, alkynyl, ester, or alkylester.

In some embodiments, the disclosure provides a compound of Formula A, I, II, III, IV, V, VI, VII, VIII, IX, X, or a salt thereof, wherein:

R¹ is

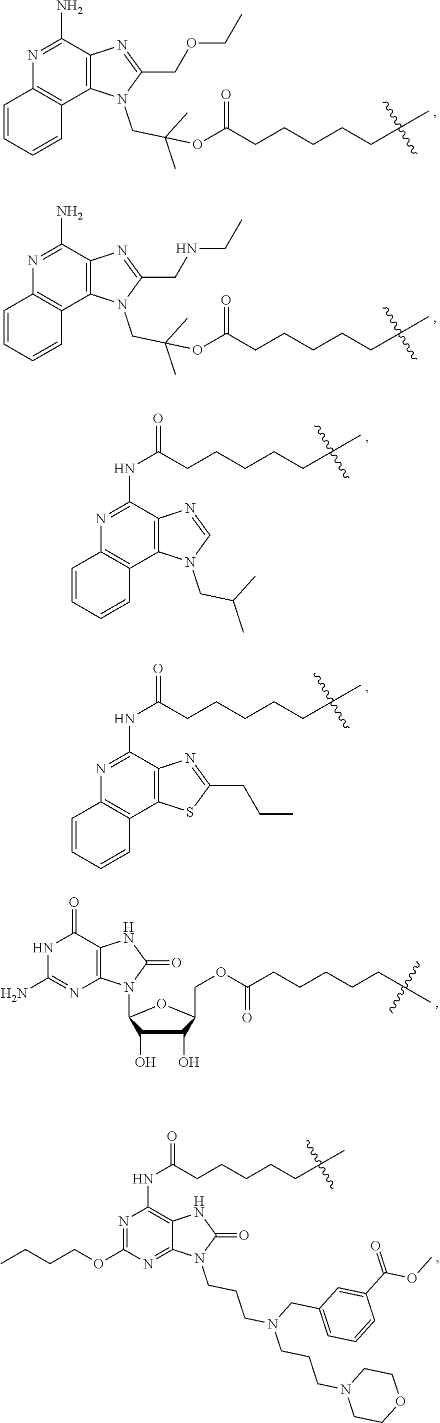

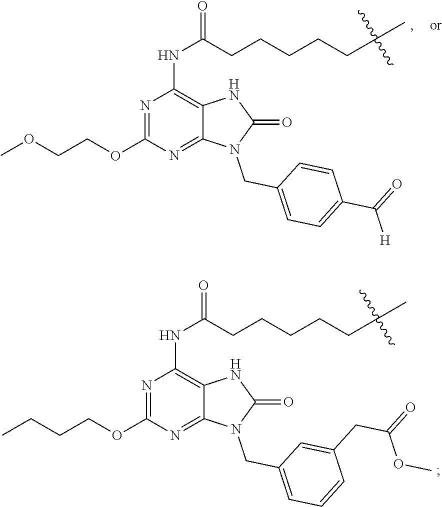 [SMILES: *CCCCCC(=O)Nc1nc(OCCOC)nc2c1[nH]c(=O)n2Cc1ccc(C=O)cc1 ; *CCCCCC(=O)Nc1nc(OCCCC)nc2c1[nH]c(=O)n2Cc1cccc(CC(=O)OC)c1]

and

each R³ is independently selected from alkyl, alkenyl, alkynyl, ester, or alkylester.

In some embodiments, the disclosure provides a compound of Formula A, I, II, III, IV, V, VI, VII, VIII, IX, X, or a salt thereof, wherein:

R¹ is

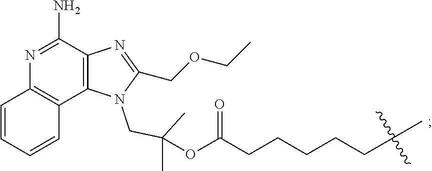 [SMILES: *CCCCCC(=O)OC(C)(C)Cn1c(COCC)nc2c(N)nc3ccccc3c21]

and each R³ is independently selected from alkyl, alkenyl, alkynyl, ester, or alkylester. In one aspect, the disclosure provides a compound of Formula A, I, II, III, IV, V, VI, VII, VIII, IX, X, or a salt thereof, wherein:

R¹ is

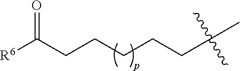

wherein p is an integer from 1 to 20;

R⁶ is a toll-like receptor (TLR) agonist or a derivative thereof; and

each R³ is independently selected from alkyl, alkenyl, alkynyl, ester, or alkylester.

In one aspect, the disclosure provides a compound of Formula A, I, II, III, IV, V, VI, VII, VIII, IX, X, or a salt thereof, wherein:

R¹ is

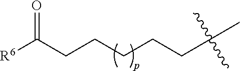

wherein p is an integer from 1 to 20;

R⁶ is a toll-like receptor (TLR) agonist or a derivative thereof; and

each R³ is independently selected from C₇₋₁₇alkyl, C₇₋₂₀alkenyl, or C₁₋₁₀alkylester, wherein the C₁₋₁₀alkylester is substituted with an alkyl or alkenyl group.

In one aspect, the disclosure provides a compound of Formula A, I, II, III, IV, V, VI, VII, VIII, IX, X, or a salt thereof, wherein:

R¹ is

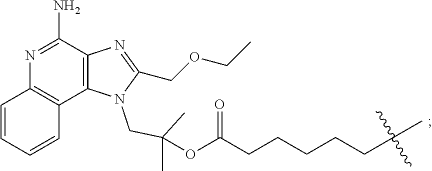 [SMILES: *CCCCCC(=O)OC(C)(C)Cn1c(COCC)nc2c(N)nc3ccccc3c21]

and

each R³ is independently selected from C₇₋₁₇alkyl, C₇₋₂₀alkenyl, or C₁₋₁₀alkylester, wherein the C₁₋₁₀alkylester is substituted with an alkyl or alkenyl group.

For Formula A in the compounds above, R² is alkyl, cycloalkyl, heterocycloalkyl, alkylheterocycloalkyl, amide, amine, alkylamide, ether, or alkylether.

In some embodiments, R² is

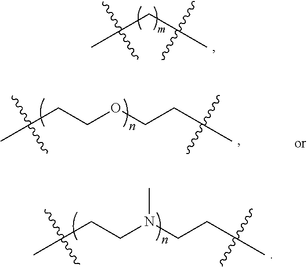

wherein m is an integer from 1 to 20,

wherein n is an integer from 1 to 3.

In some embodiments, R¹ is

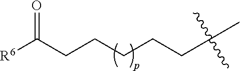

wherein p is an integer from 1 to 20.

In some embodiments, R′ is a toll-like receptor (TLR) agonist or a derivative thereof.

In some embodiments, R⁶ is

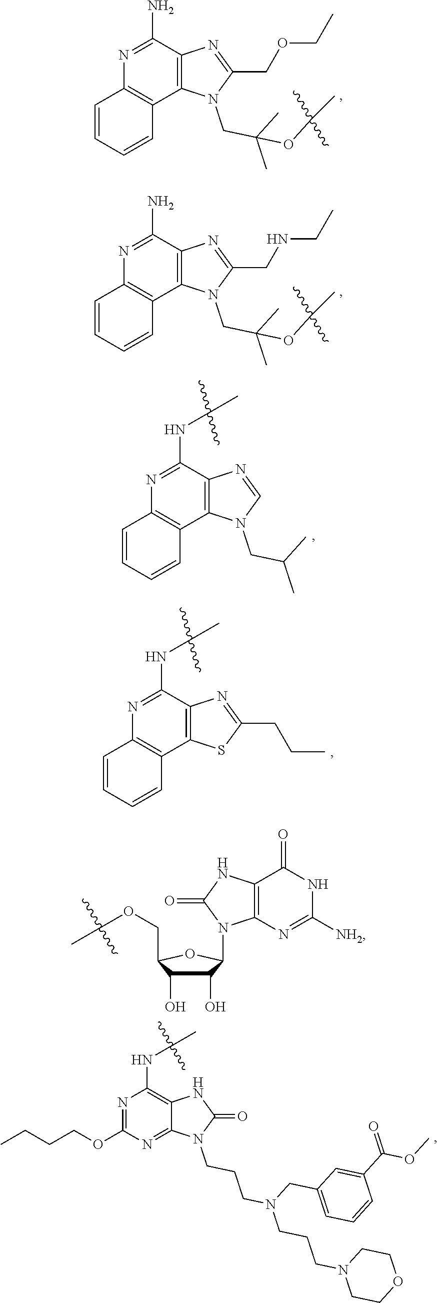

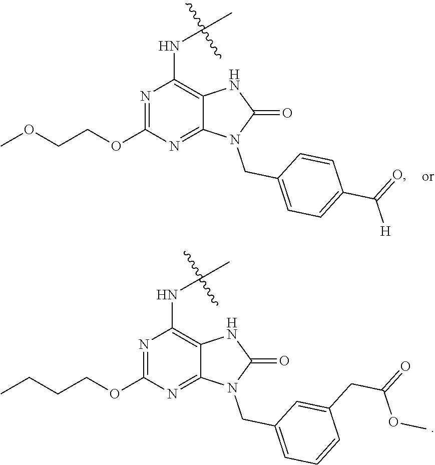 [SMILES: *Nc1nc(OCCOC)nc2c1[nH]c(=O)n2Cc1ccc(C=O)cc1 ; *Nc1nc(OCCCC)nc2c1[nH]c(=O)n2Cc1cccc(CC(=O)OC)c1]

In some embodiments, R⁶ is

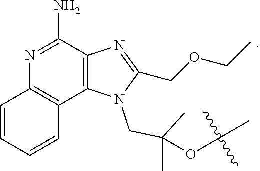 [SMILES: *OC(C)(C)Cn1c(COCC)nc2c(N)nc3ccccc3c21]

In some embodiments, R¹ is

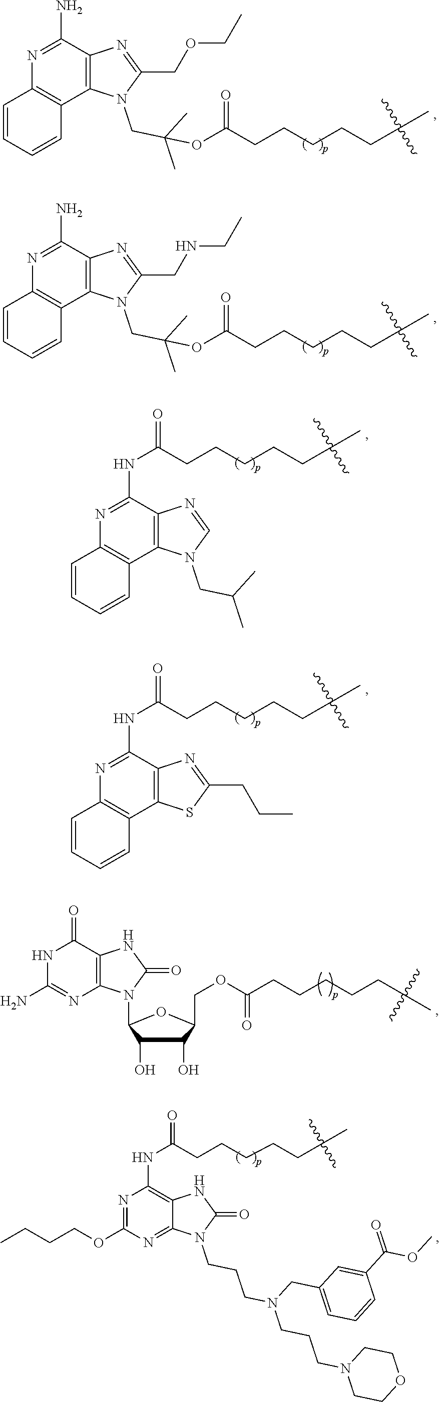

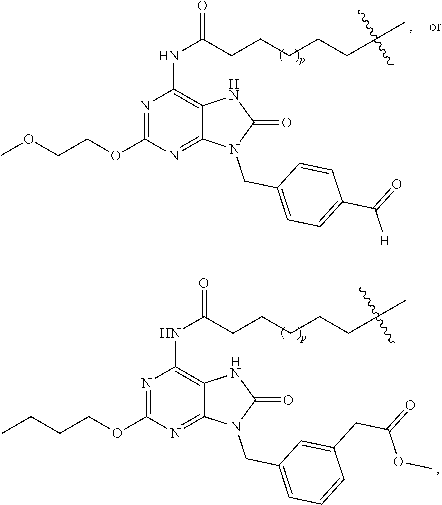

wherein p is an integer from 1 to 20.

In some embodiments, R¹ is

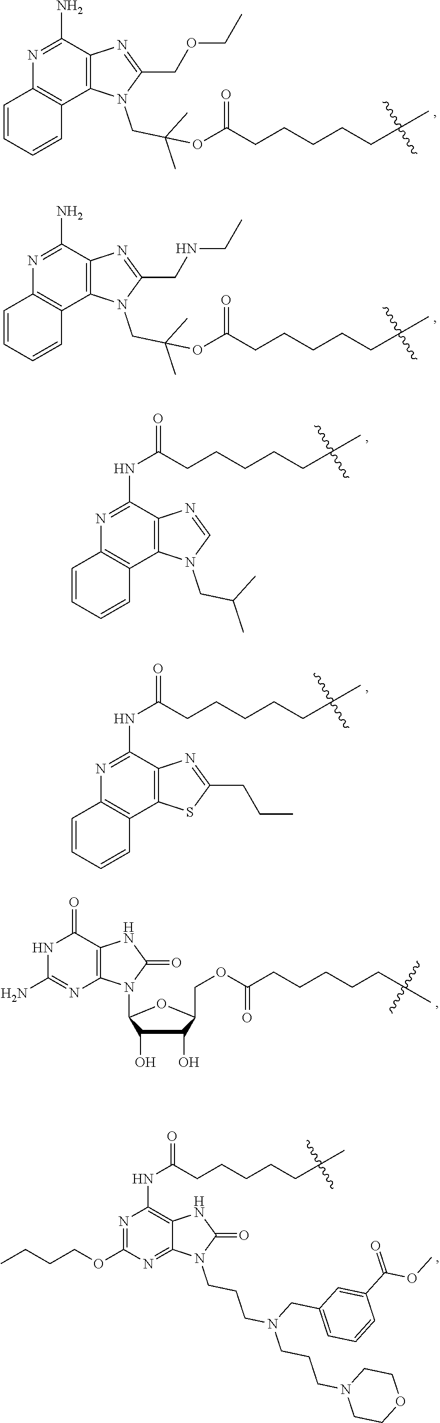

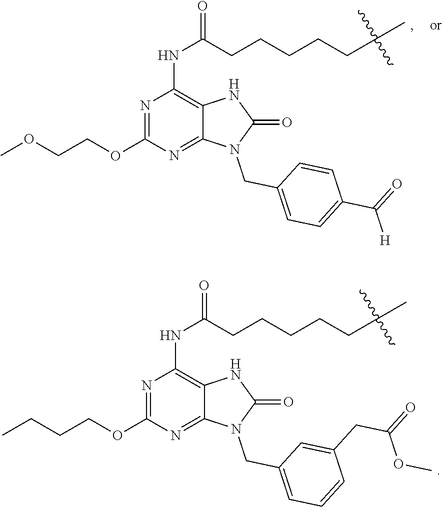 [SMILES: *CCCCCC(=O)Nc1nc(OCCOC)nc2c1[nH]c(=O)n2Cc1ccc(C=O)cc1 ; *CCCCCC(=O)Nc1nc(OCCCC)nc2c1[nH]c(=O)n2Cc1cccc(CC(=O)OC)c1]

In some embodiments, R′ is

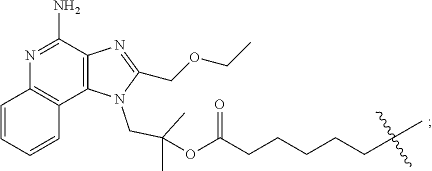 [SMILES: *CCCCCC(=O)OC(C)(C)Cn1c(COCC)nc2c(N)nc3ccccc3c21]

Various alkyl and ether linkers can be used to link the toll-like receptor (TLR) agonist to the lipid tail. Examples of linkers can include:

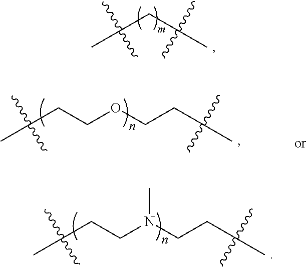

wherein m is an integer from 1 to 20; and

wherein n is an integer from 1 to 3.

In some embodiments, the toll-like receptor (TLR) agonist or a derivative thereof is selected from resiquimod, gardiquimod, imiquimod, CL075, SM324405, UC1V150, CU-T12-9, loxoribine, or derivatives thereof.

In some embodiments, each R³ is independently selected from alkyl, alkenyl, alkynyl, ester, or alkylester. In some embodiments, each R³ is independently selected from alkyl, alkenyl, or alkylester. In some embodiments, each R³ is independently selected from C₇₋₁₇alkyl, C₇₋₂₀alkenyl, or C₁₋₁₀alkylester, wherein the C₁₋₁₀alkylester is substituted with an alkyl or alkenyl group.

In some embodiments, each R³ is independently selected from an alkyl. In some embodiments, each R³ is independently selected from a C₇₋₁₇alkyl. In some embodiments, each R³ is independently selected from a C₉₋₁₂alkyl. In some embodiments, each R³ is independently selected from a C₉alkyl. In some embodiments, each R³ is independently selected from a C₁₀alkyl. In some embodiments, each R³ is independently selected from a C₁₁alkyl. In some embodiments, each R³ is independently selected from a C₁₀alkyl.

In some embodiments, each R³ is independently selected from an alkenyl. In some embodiments, each R³ is independently selected from a C₇₋₂₀alkenyl. In some embodiments, each R³ is independently selected from a C₉alkenyl.

In some embodiments, each R³ is independently selected from an alkylester. In some embodiments, each R³ is independently selected from an alkylester, wherein the alkylester is substituted with an alkyl or alkenyl group. In some embodiments, each R³ is independently selected from a C₁₋₁₀alkylester, wherein the C₁₋₁₀alkylester is substituted with an alkyl or alkenyl group. In some embodiments, each R³ is independently selected from a C₁₋₁₀alkylester, wherein the C₁₋₁₀alkylester is substituted with C₆₋₈alkyl group. In some embodiments, each R³ is independently selected from a C₁₋₁₀alkylester, wherein the C₁₋₁₀alkylester is substituted with C₆₋₁₀alkenyl group.

In some embodiments, each R³ is independently selected from a linear or branched saturated alkyl chain comprising 7 to 17 carbons. In some embodiments, each R³ is independently selected from

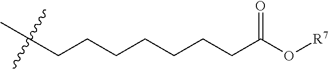

wherein each R⁷ is independently selected from a linear or branched saturated alkyl chain comprising 4 to 9 carbons.

In some embodiments, at least one R³ is C₇-C₁₇ alkyl. In some embodiments, at least one R³ is C₇₋₂₀alkenyl. In some embodiments, at least one R³ is C₁₋₁₀alkylester. In some embodiments, at least one R³ is C₁₋₁₀alkylester substituted with an alkyl or alkenyl group.

In some embodiments, at least one R³ is a branched alkyl. In some embodiments, at least one R³ is an unbranched alkyl. In some embodiments, at least one R³ is a branched alkenyl. In some embodiments, at least one R³ is an unbranched alkenyl.

In some embodiments, R³ are independently selected from:

C₇ alkyl, C₈ alkyl, C₉ alkyl, C₁₀ alkyl, C₁₁ alkyl, C₁₂ alkyl, C₁₃ alkyl, C₁₄ alkyl, Cis alkyl, C₁₆ alkyl, C₁₇ alkyl;

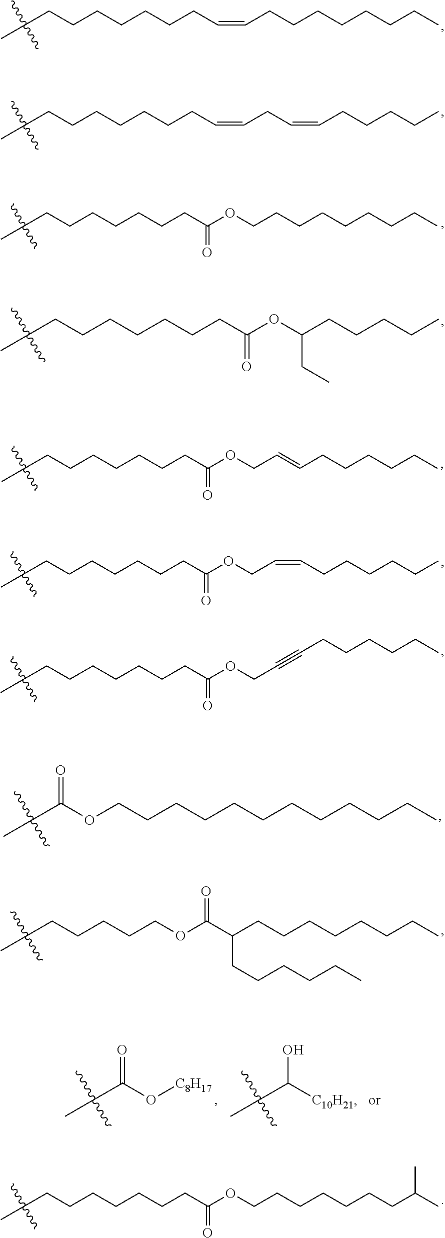

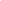

In some embodiments, the compound is selected from the following:

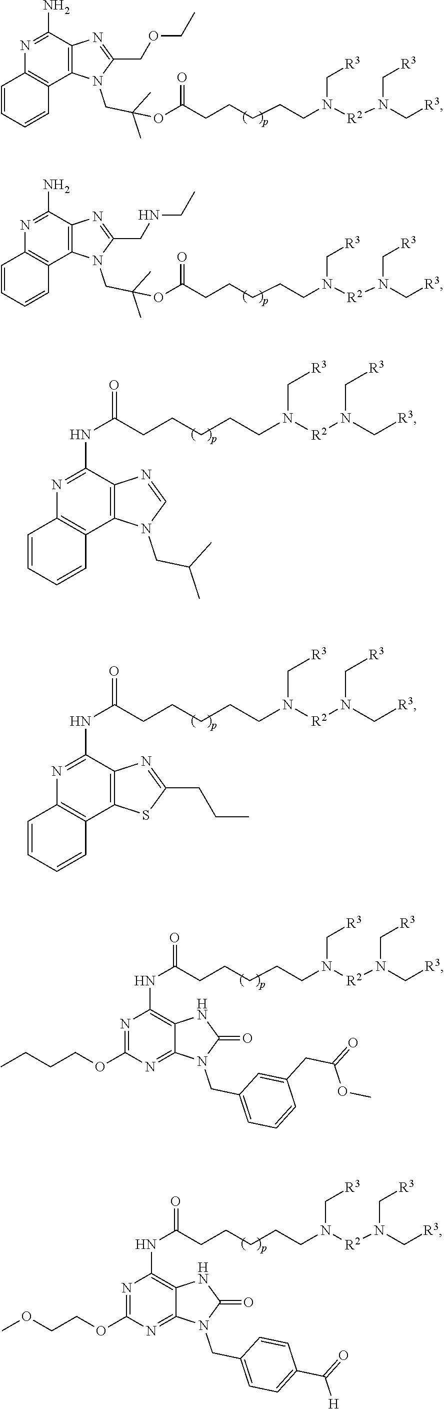

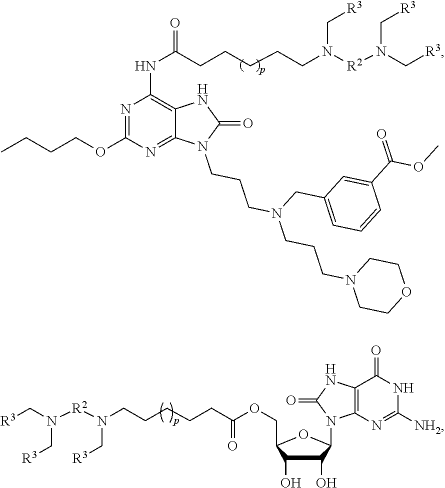

or a salt thereof;

wherein

p is an integer from 1 to 20;

R² is alkyl, cycloalkyl, heterocycloalkyl, alkylheterocycloalkyl, amide, alkylamide, amine, ether, or alkylether, and

each R³ is independently selected from alkyl, alkenyl, alkynyl, ester, or alkylester.

In some embodiments, R² is

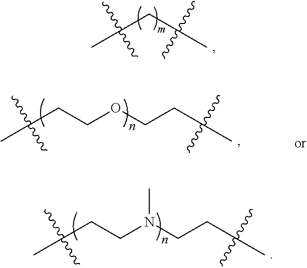

wherein m is an integer from 1 to 20,
wherein n is an integer from 1 to 3.

In some embodiments, each R³ is independently selected from alkyl, alkenyl, alkynyl, ester, or alkylester. In some embodiments, each R³ is independently selected from alkyl, alkenyl, or alkylester. In some embodiments, each R³ is independently selected from C₇₋₁₇alkyl, C₇₋₂₀alkenyl, or C₁₋₁₀alkylester, wherein the C₁₋₁₀alkylester is substituted with an alkyl or alkenyl group.

In some embodiments, each R³ is independently selected from an alkyl. In some embodiments, each R³ is independently selected from a C₇₋₁₇alkyl. In some embodiments, each R³ is independently selected from a C₉₋₁₂alkyl. In some embodiments, each R³ is independently selected from a C₉alkyl. In some embodiments, each R³ is independently selected from a C₁₀alkyl. In some embodiments, each R³ is independently selected from a C₁₁alkyl. In some embodiments, each R³ is independently selected from a C₁₀alkyl.

In some embodiments, each R³ is independently selected from an alkenyl. In some embodiments, each R³ is independently selected from a C₇₋₂₀alkenyl. In some embodiments, each R³ is independently selected from a C₉alkenyl.

In some embodiments, each R³ is independently selected from an alkylester. In some embodiments, each R³ is independently selected from an alkylester, wherein the alkylester is substituted with an alkyl or alkenyl group. In some embodiments, each R³ is independently selected from a C₁₋₁₀alkylester, wherein the C₁₋₁₀alkylester is substituted with an alkyl or alkenyl group. In some embodiments, each R³ is independently selected from a C₁₋₁₀alkylester, wherein the C₁₋₁₀alkylester is substituted with C₆₋₈alkyl group. In some embodiments, each R³ is independently selected from a C₁₋₁₀alkylester, wherein the C₁₋₁₀alkylester is substituted with C₆₋₁₀alkenyl group.

In some embodiments, each R³ is independently selected from a linear or branched saturated alkyl chain comprising 7 to 17 carbons. In some embodiments, each R³ is independently selected from

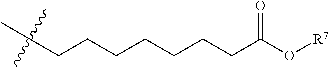

wherein each R⁷ is independently selected from a linear or branched saturated alkyl chain comprising 4 to 9 carbons.

In some embodiments, at least one R³ is C₇-C₁₇ alkyl. In some embodiments, at least one R³ is C₇₋₂₀alkenyl. In some embodiments, at least one R³ is C₁₋₁₀alkylester. In some embodiments, at least one R³ is C₁₋₁₀alkylester substituted with an alkyl or alkenyl group.

In some embodiments, at least one R³ is a branched alkyl. In some embodiments, at least one R³ is an unbranched alkyl. In some embodiments, at least one R³ is a branched alkenyl. In some embodiments, at least one R³ is an unbranched alkenyl.

In some embodiments, R³ are independently selected from:

C₇ alkyl, C₈ alkyl, C₉ alkyl, C₁₀ alkyl, C₁₁ alkyl, C₁₂ alkyl, C₁₃ alkyl, C₁₄ alkyl, Cis alkyl, C₁₆ alkyl, C₁₇ alkyl;

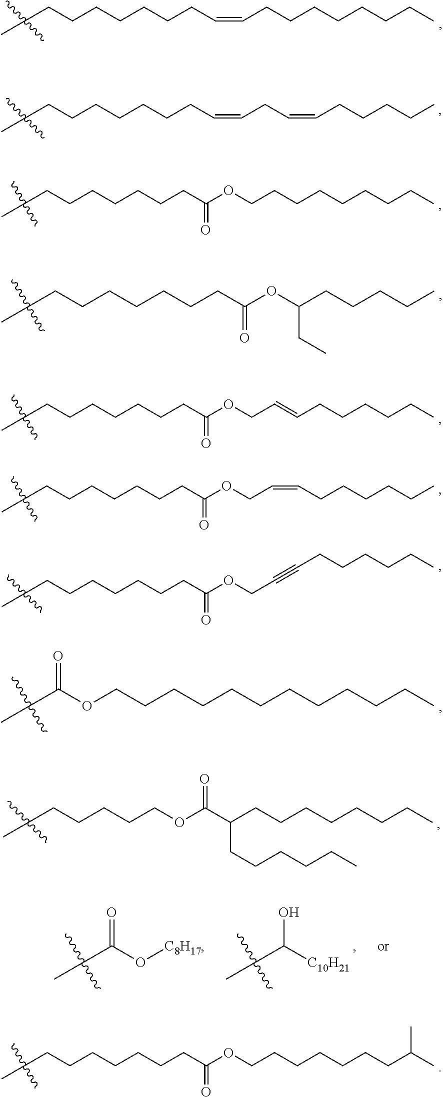

In some embodiments, the compound is selected from the following:

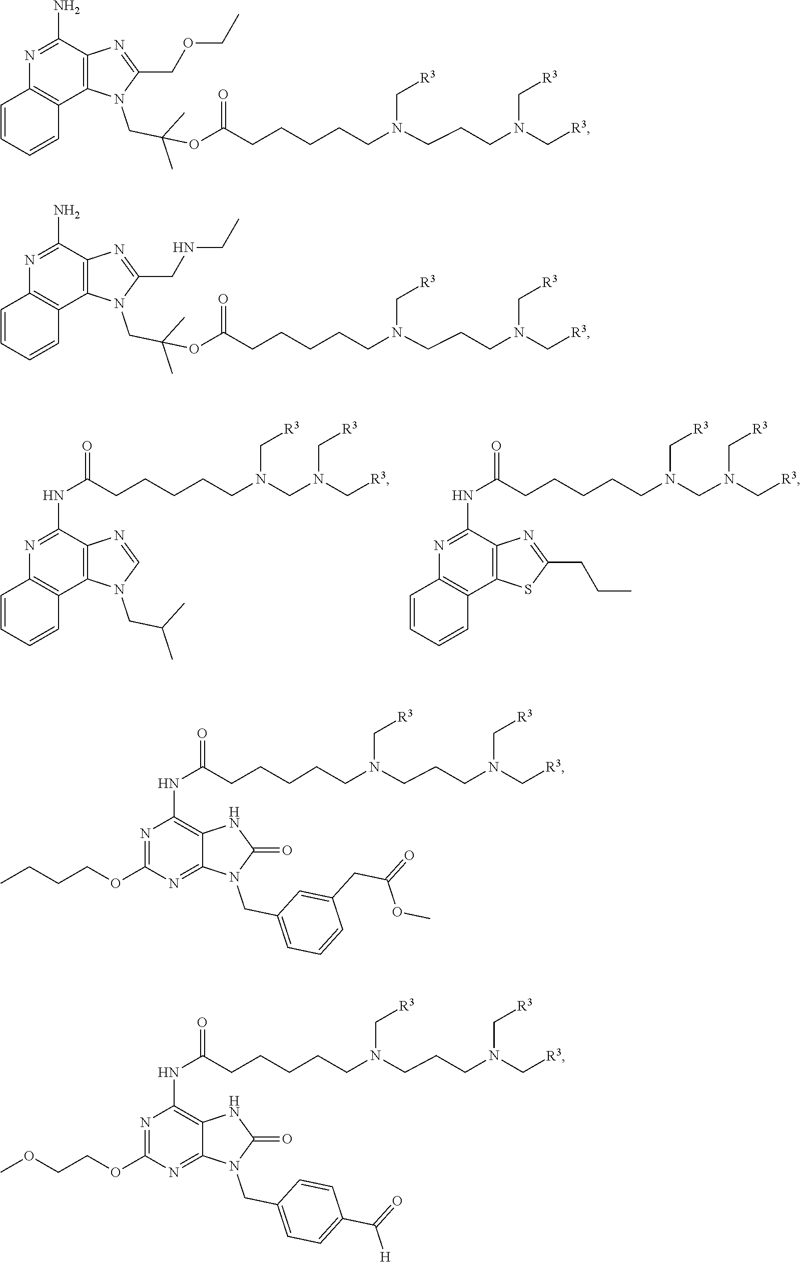

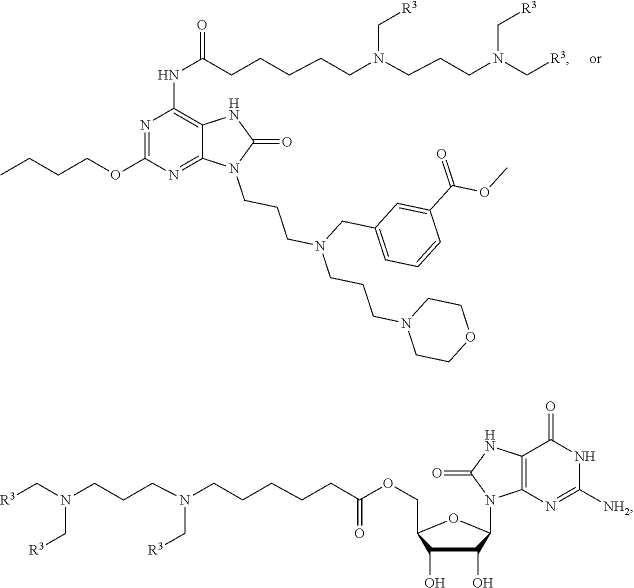

or a salt thereof.

In some embodiments, each R³ is independently selected from alkyl, alkenyl, alkynyl, ester, or alkylester. In some embodiments, each R³ is independently selected from alkyl, alkenyl, or alkylester. In some embodiments, each R³ is independently selected from C₇₋₁₇alkyl, C₇₋₂₀alkenyl, or C₁₋₁₀alkylester, wherein the C₁₋₁₀alkylester is substituted with an alkyl or alkenyl group.

In some embodiments, each R³ is independently selected from an alkyl. In some embodiments, each R³ is independently selected from a C₇₋₁₇alkyl. In some embodiments, each R³ is independently selected from a C₉₋₁₂alkyl. In some embodiments, each R³ is independently selected from a C₉alkyl. In some embodiments, each R³ is independently selected from a C₁₀alkyl. In some embodiments, each R³ is independently selected from a C₁₁alkyl. In some embodiments, each R³ is independently selected from a C₁₀alkyl.

In some embodiments, each R³ is independently selected from an alkenyl. In some embodiments, each R³ is independently selected from a C₇₋₂₀alkenyl. In some embodiments, each R³ is independently selected from a C₉alkenyl.

In some embodiments, each R³ is independently selected from an alkylester. In some embodiments, each R³ is independently selected from an alkylester, wherein the alkylester is substituted with an alkyl or alkenyl group. In some embodiments, each R³ is independently selected from a C₁₋₁₀alkylester, wherein the C₁₋₁₀alkylester is substituted with an alkyl or alkenyl group. In some embodiments, each R³ is independently selected from a C₁₋₁₀alkylester, wherein the C₁₋₁₀alkylester is substituted with C₆₋₈alkyl group. In some embodiments, each R³ is independently selected from a C₁₋₁₀alkylester, wherein the C₁₋₁₀alkylester is substituted with C₆₋₁₀alkenyl group.

In some embodiments, each R³ is independently selected from a linear or branched saturated alkyl chain comprising 7 to 17 carbons. In some embodiments, each R³ is independently selected from

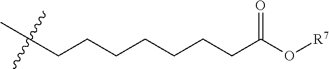

wherein each R⁷ is independently selected from a linear or branched saturated alkyl chain comprising 4 to 9 carbons.

In some embodiments, at least one R³ is C₇-C₁₇ alkyl. In some embodiments, at least one R³ is C₇₋₂₀alkenyl. In some embodiments, at least one R³ is C₁₋₁₀alkylester. In some embodiments, at least one R³ is C₁₋₁₀alkylester substituted with an alkyl or alkenyl group.

In some embodiments, at least one R³ is a branched alkyl. In some embodiments, at least one R³ is an unbranched alkyl. In some embodiments, at least one R³ is a branched alkenyl. In some embodiments, at least one R³ is an unbranched alkenyl.

In some embodiments, R³ are independently selected from:

C₇ alkyl, C₈ alkyl, C₉ alkyl, C₁₀ alkyl, C₁₁ alkyl, C₁₂ alkyl, C₁₃ alkyl, C₁₄ alkyl, Cis alkyl, C₁₆ alkyl, C₁₇ alkyl;

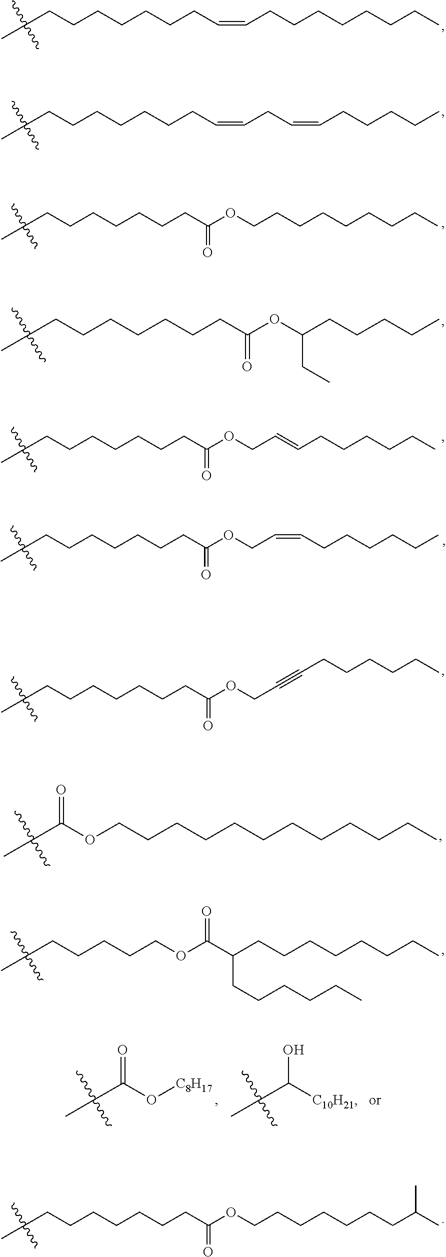

In some embodiments, the compound is selected from the following:

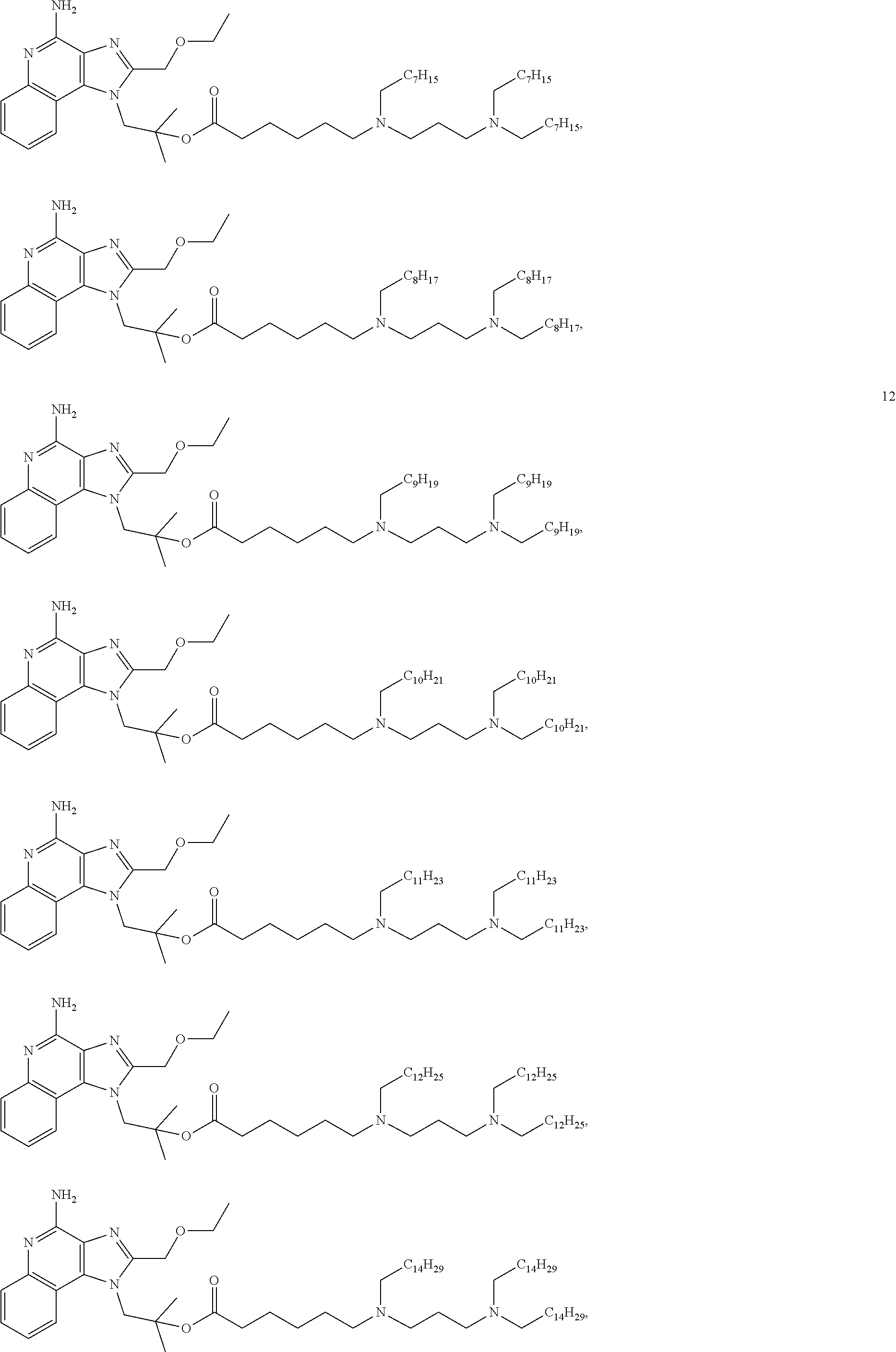

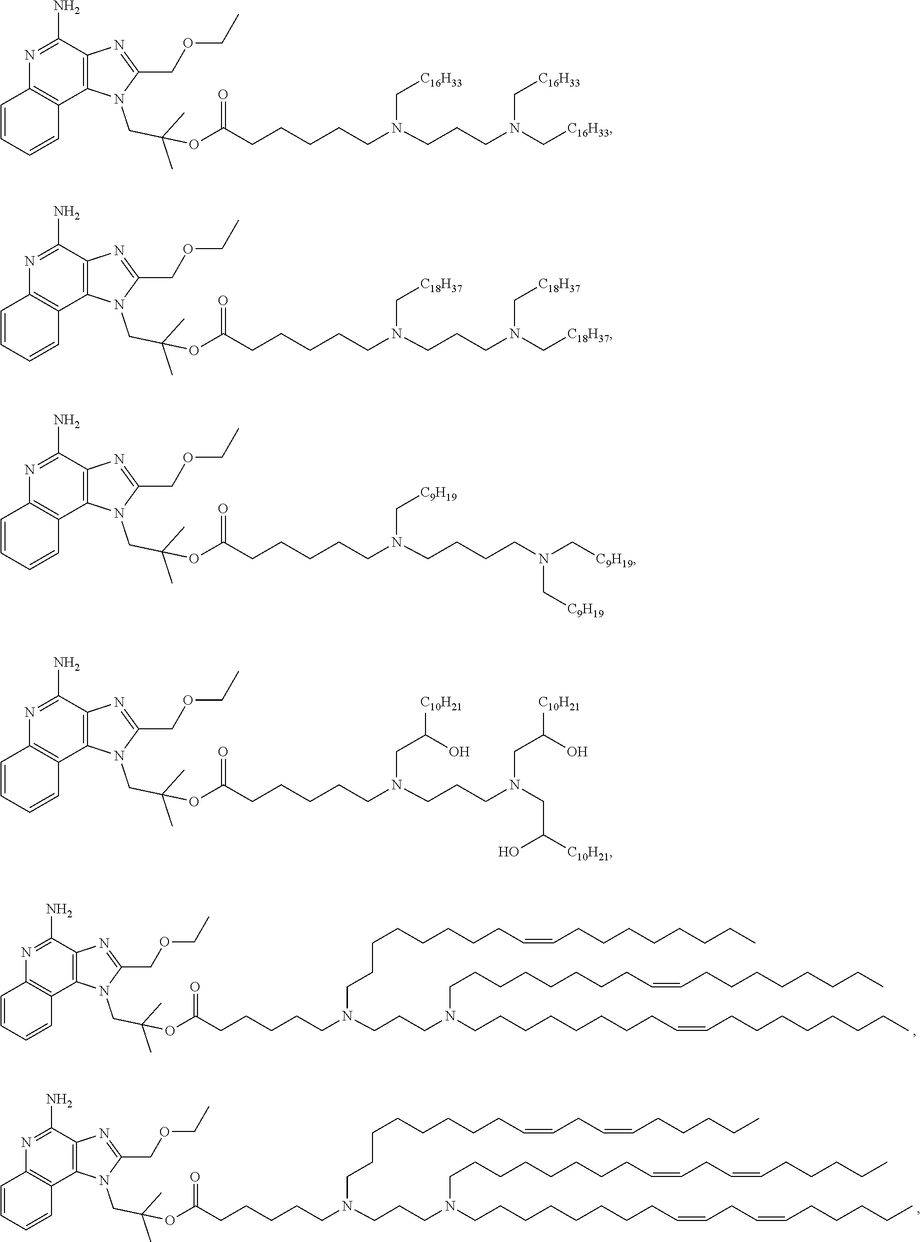

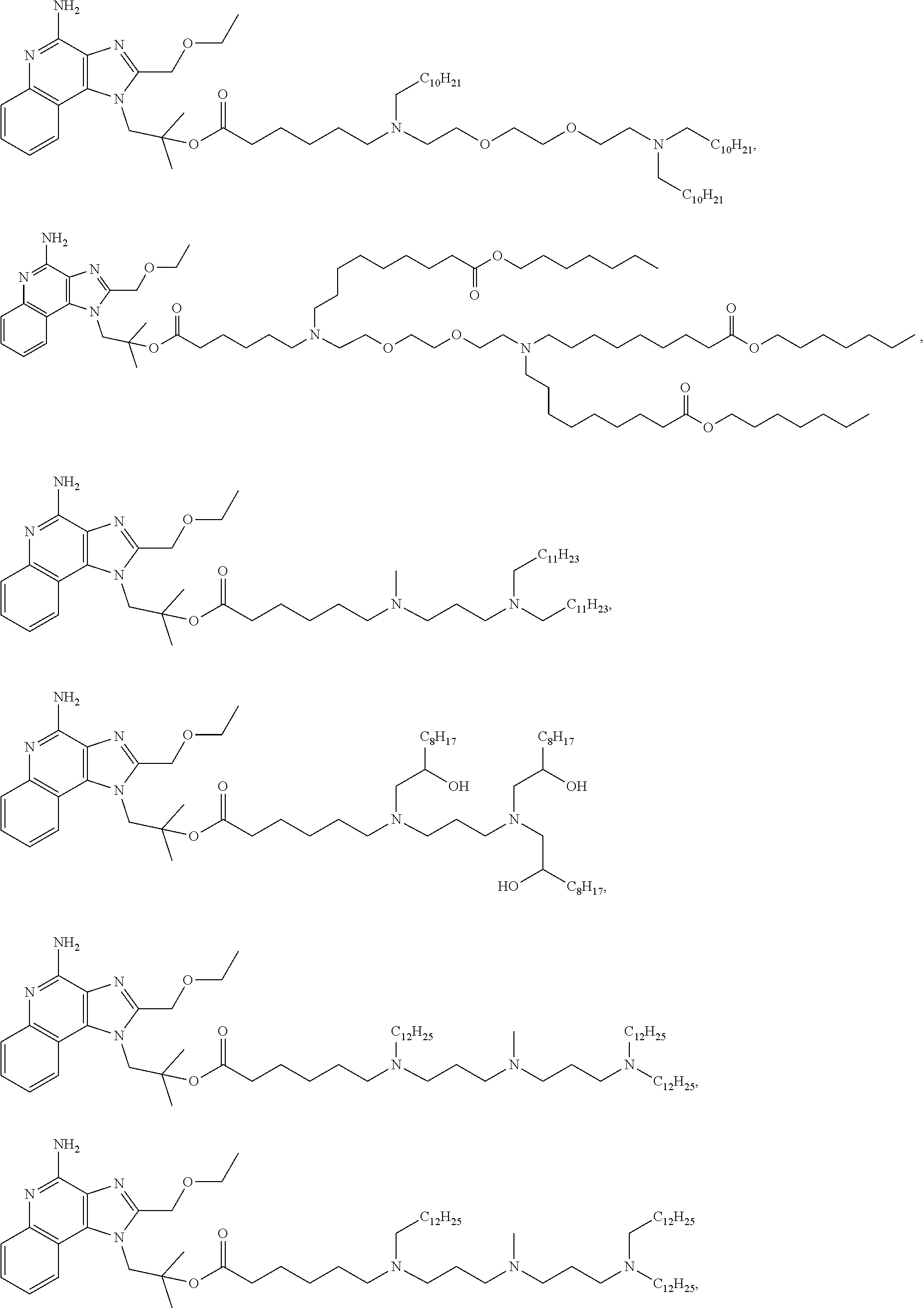

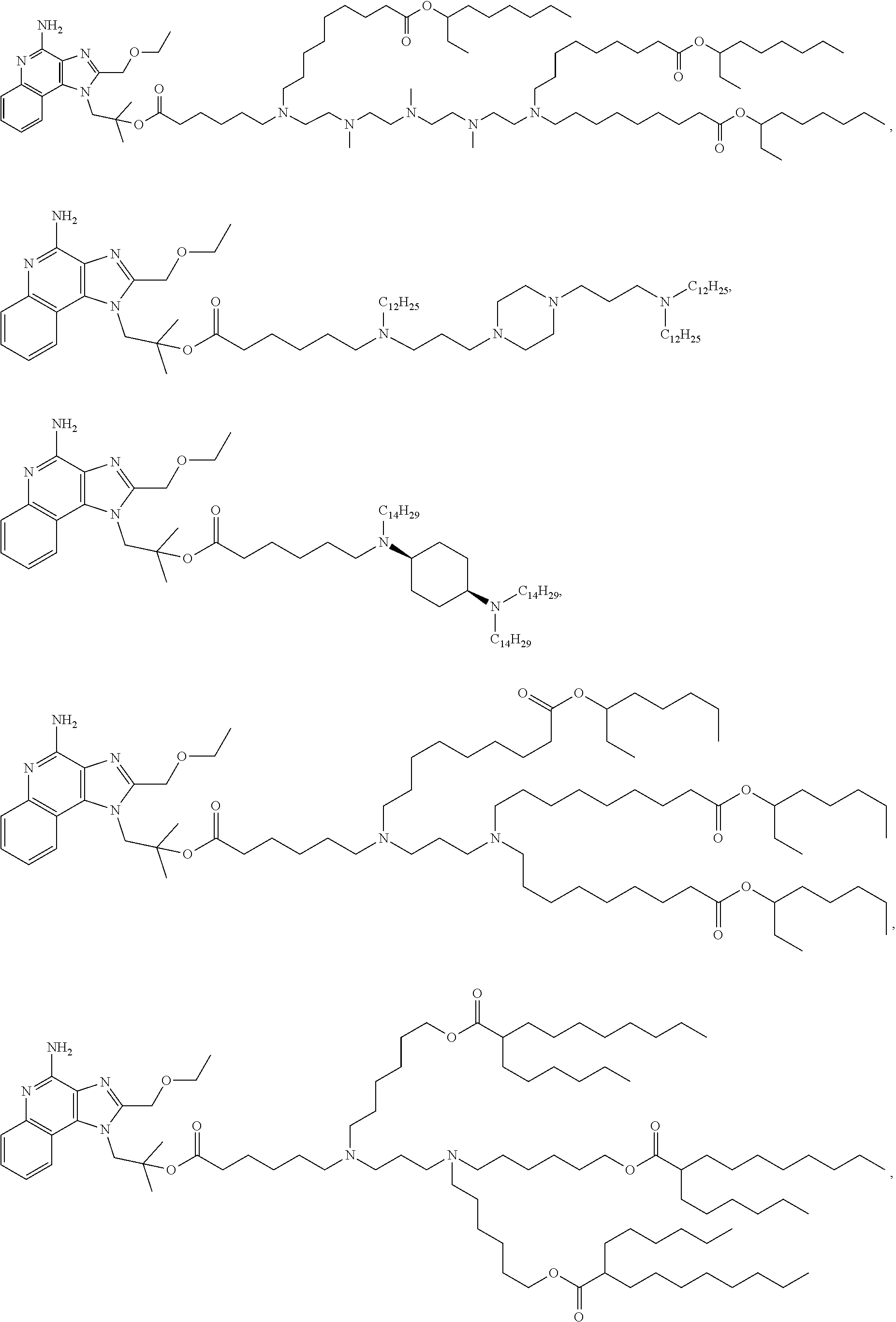

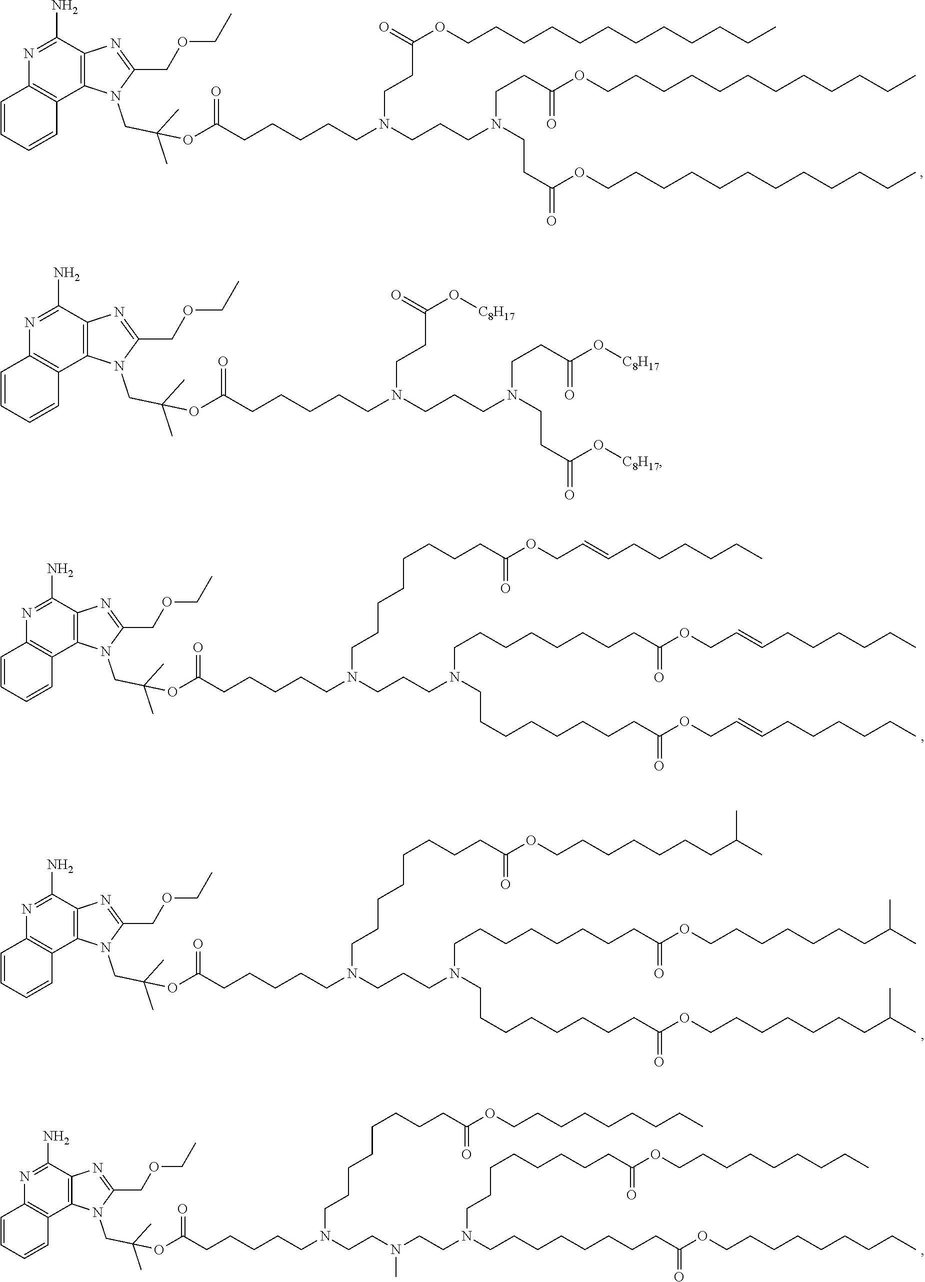

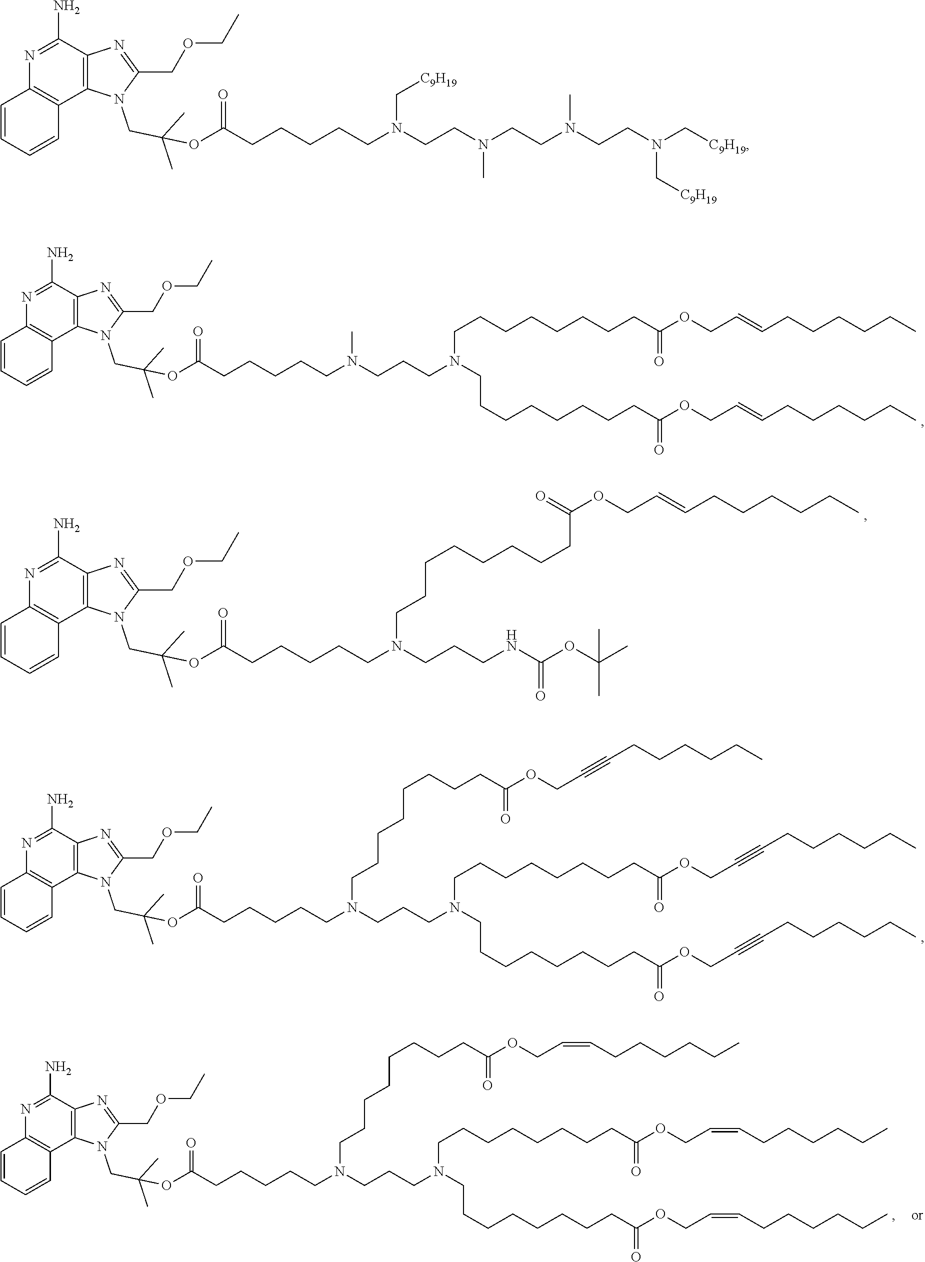

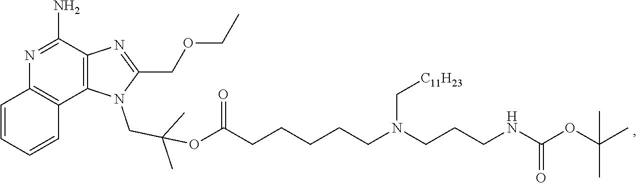

or a salt thereof.

In some embodiments, the compound is:

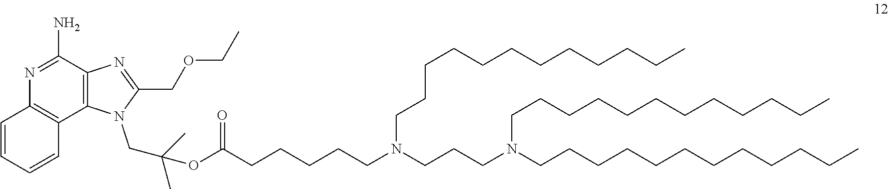 [SMILES: CCCCCCCCCCCCN(CCCCCCCCCCCC)CCCN(CCCCCCCCCCCC)CCCCCC(=O)OC(C)(C)Cn1c(COCC)nc2c(N)nc3ccccc3c21]

or a salt thereof.

In some embodiments, the compound is:

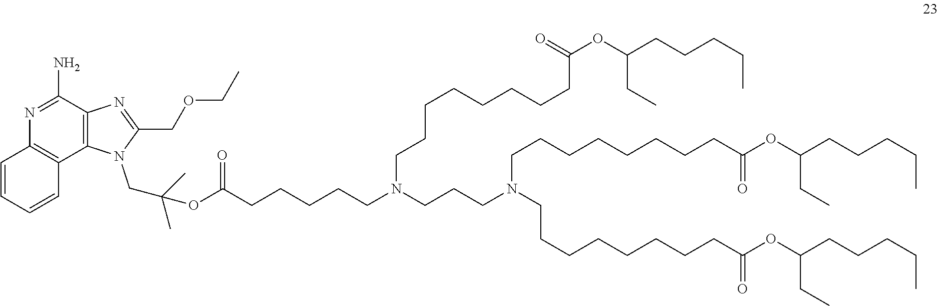 [SMILES: CCCCCC(CC)OC(=O)CCCCCCCCN(CCCCCCCCC(=O)OC(CC)CCCCC)CCCN(CCCCCCCCC(=O)OC(CC)CCCCC)CCCCCC(=O)OC(C)(C)Cn1c(COCC)nc2c(N)nc3ccccc3c21]

or a salt thereof.

In some embodiments, the compound is selected from the following:

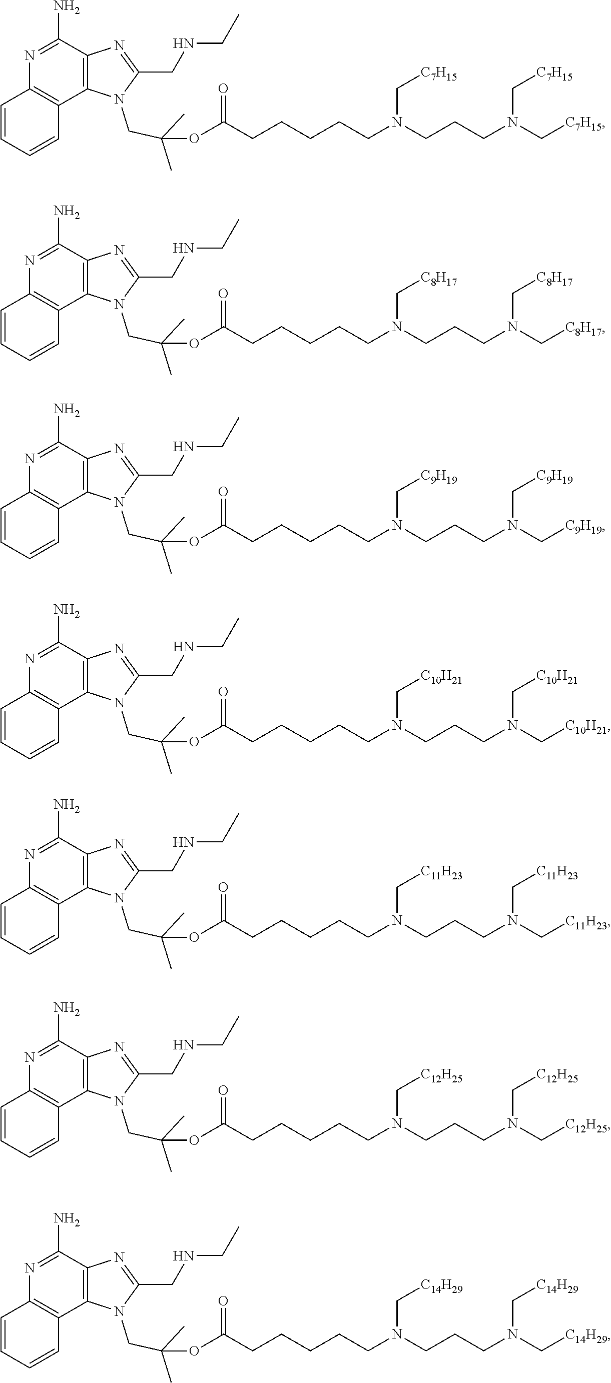

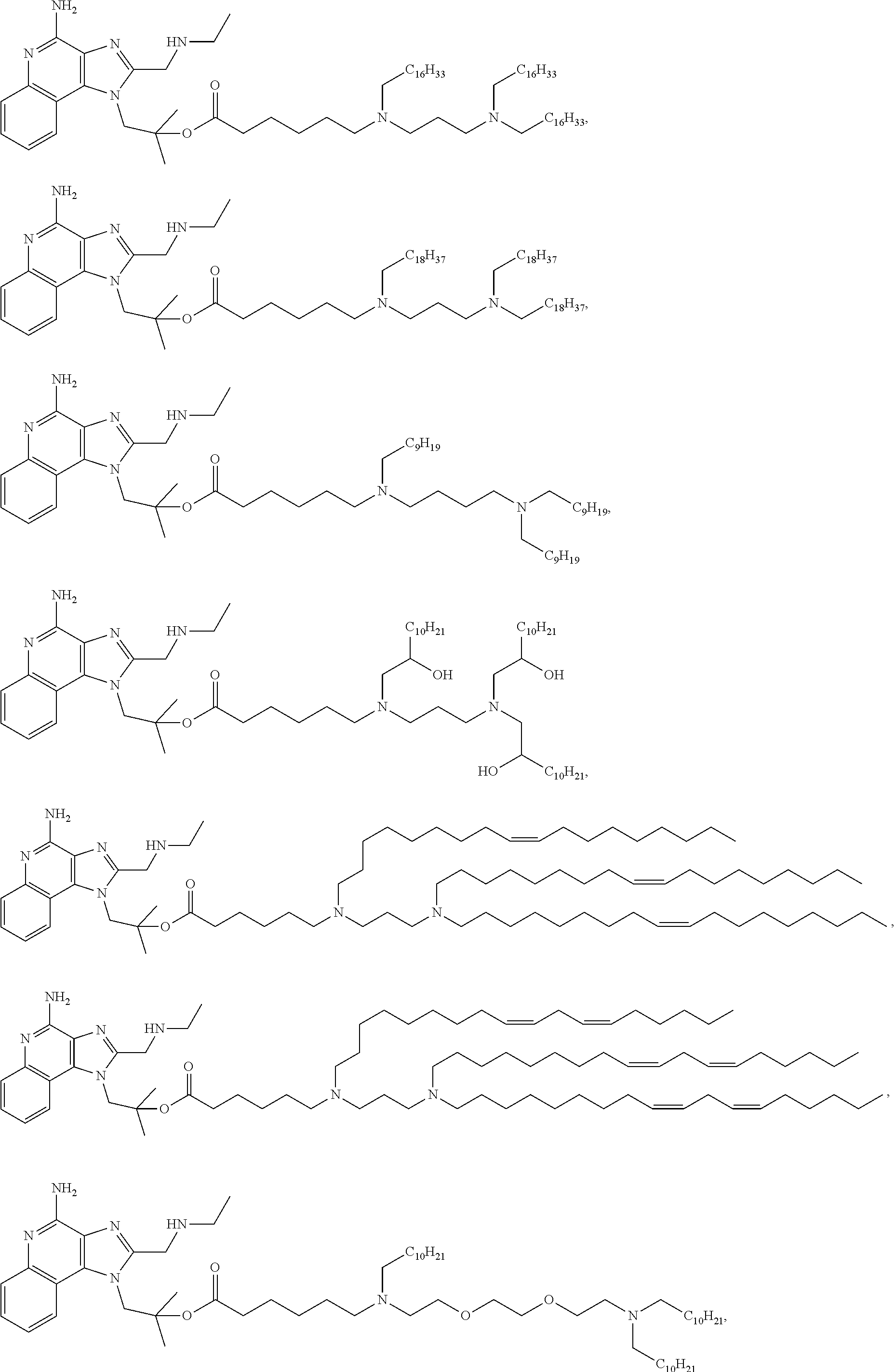

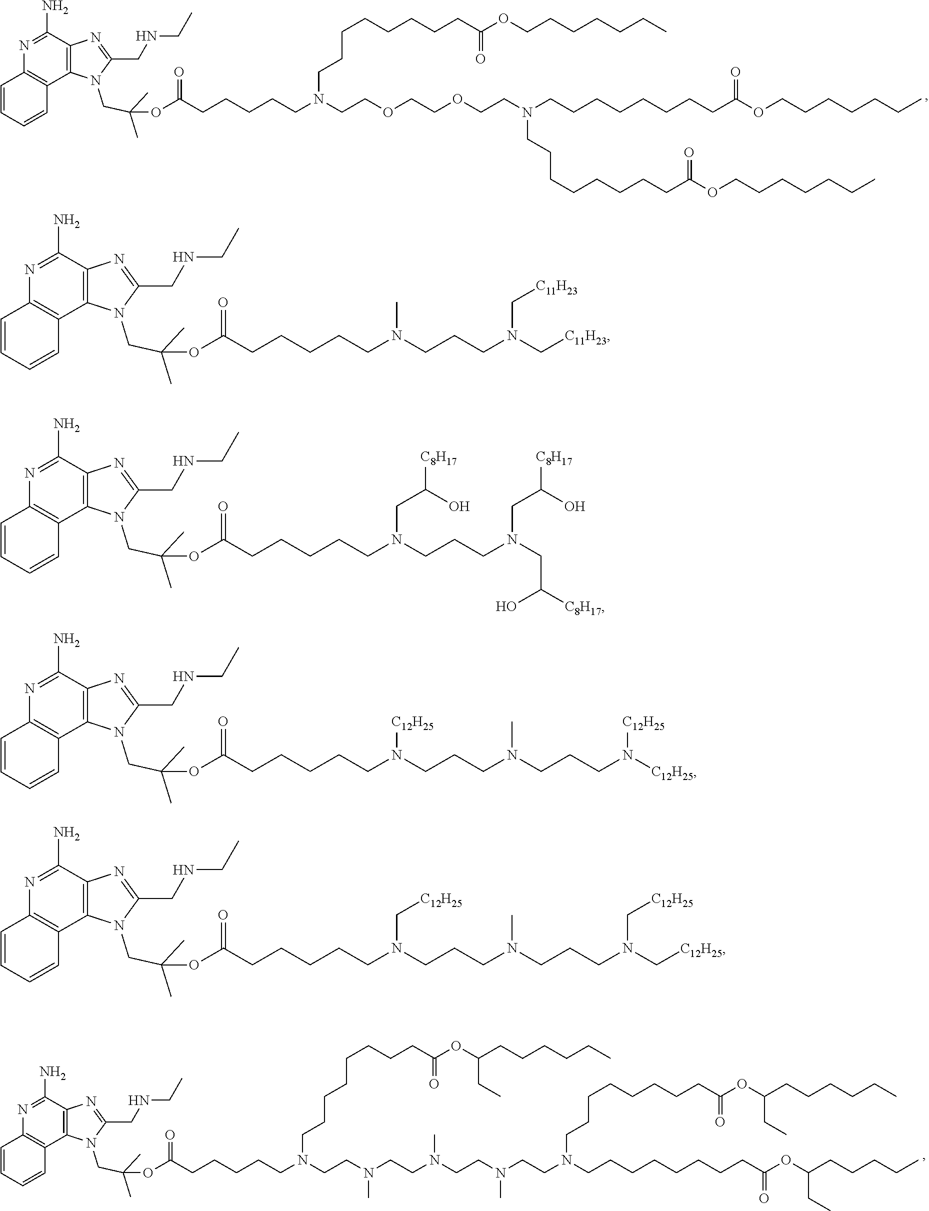

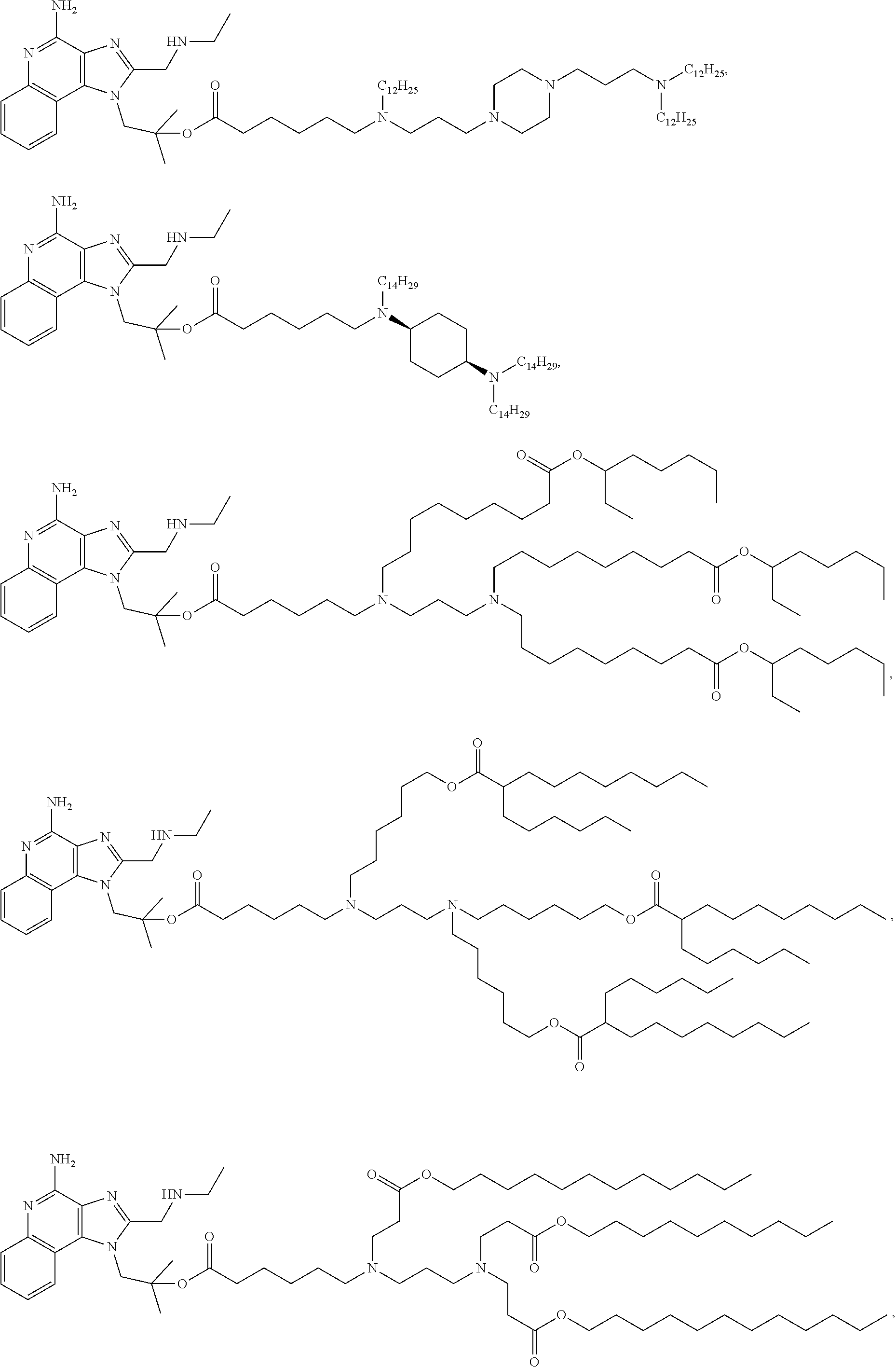

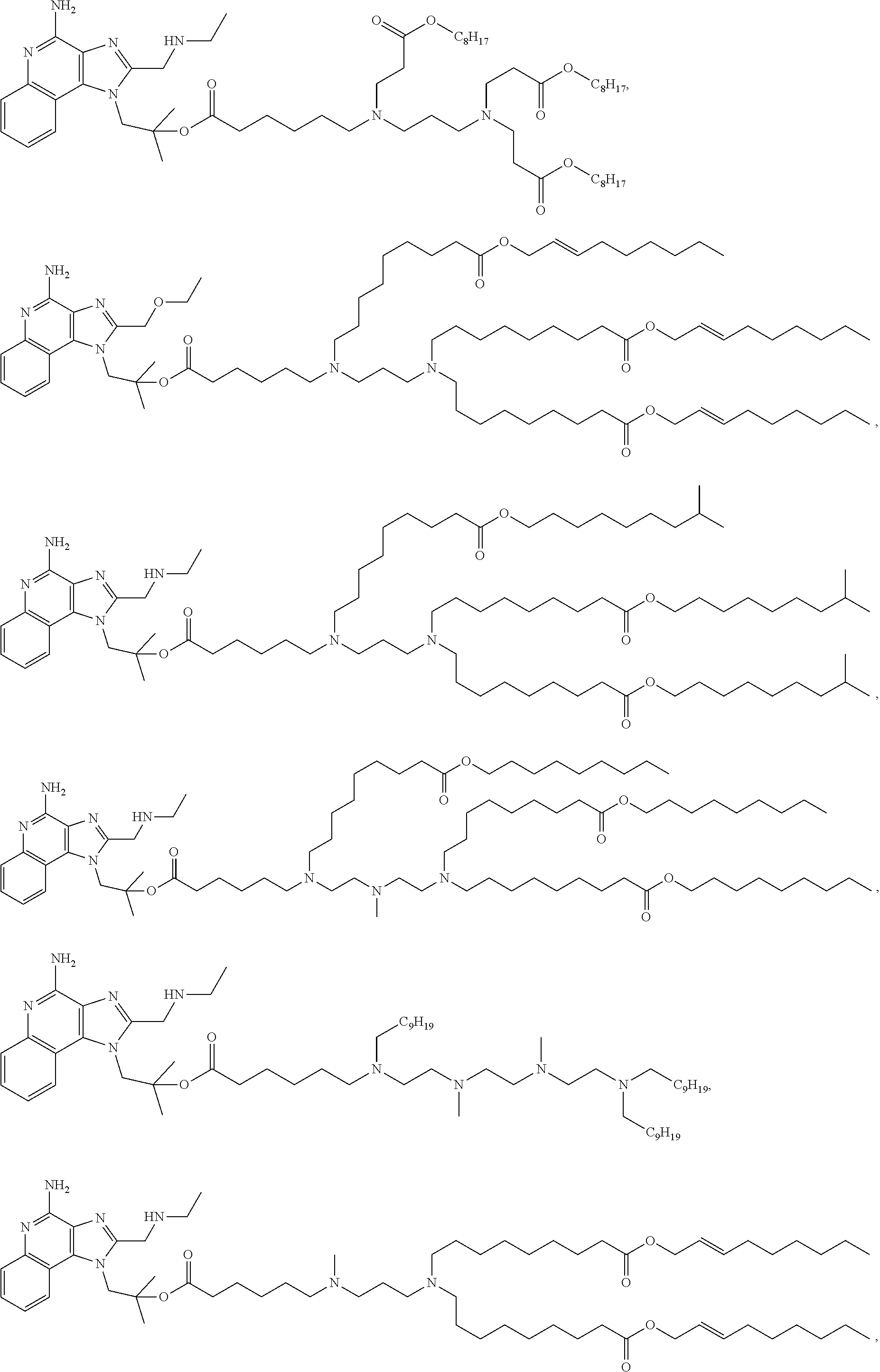

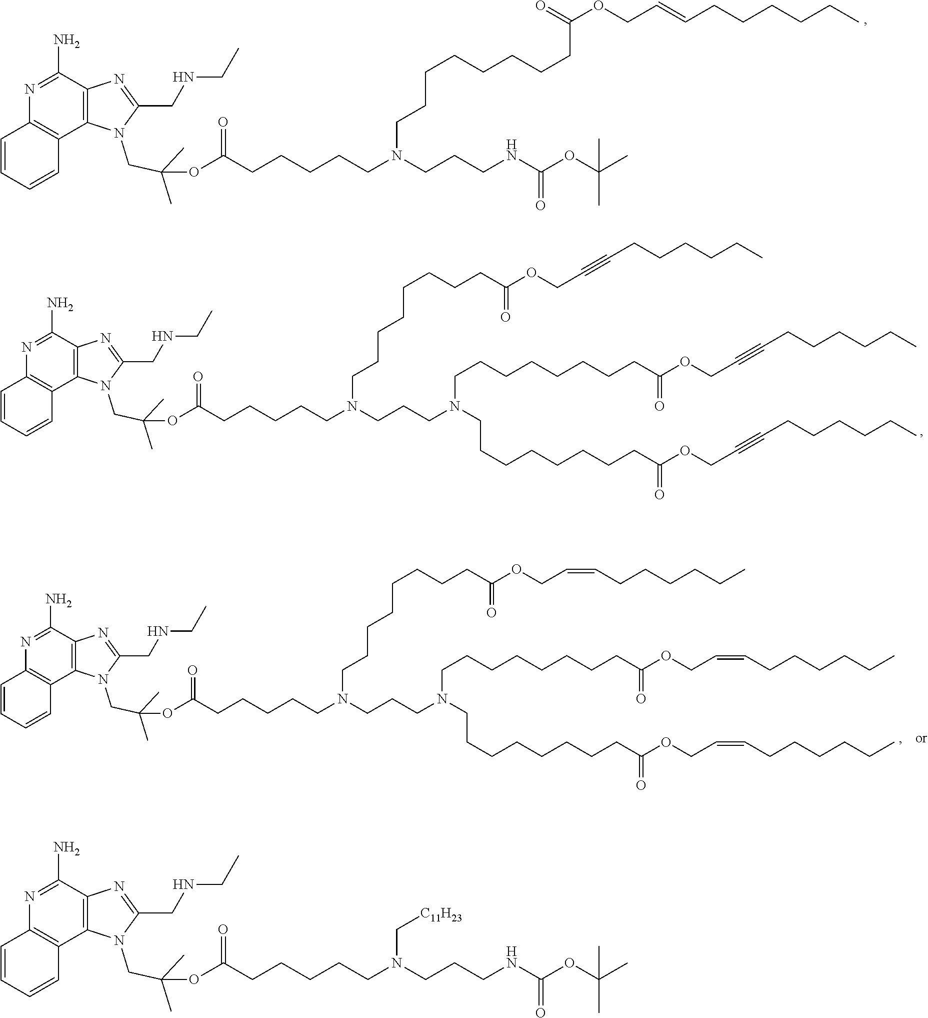

or a salt thereof.

In some embodiments, the compound is selected from the following:

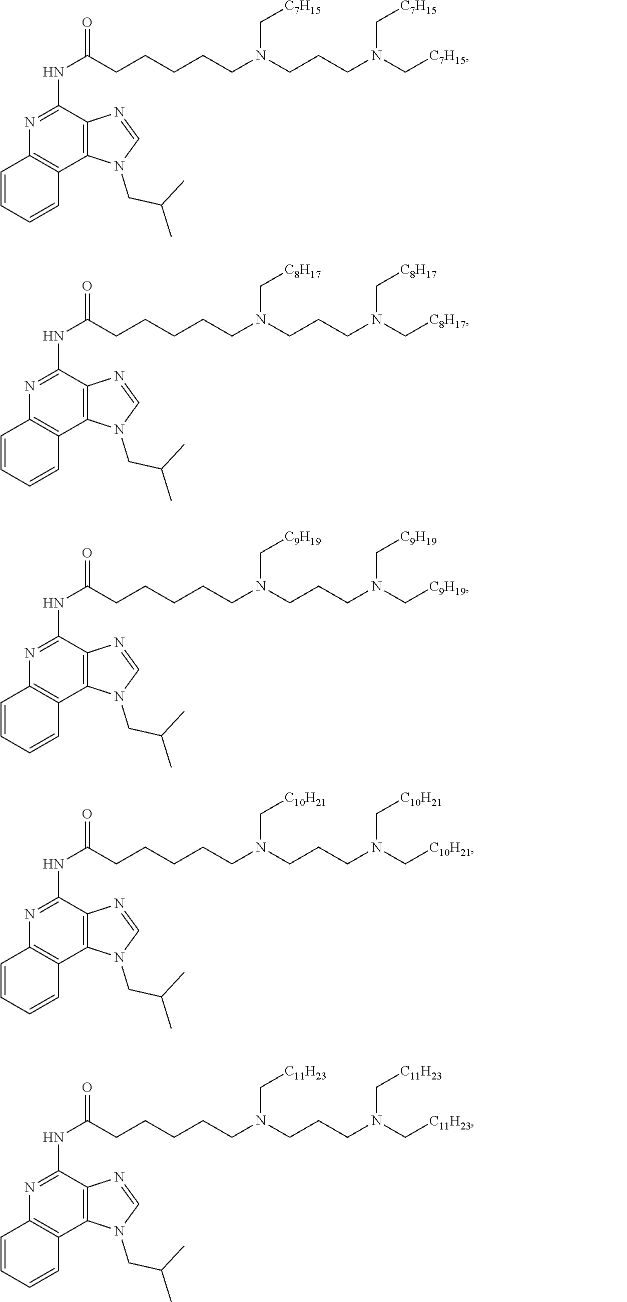

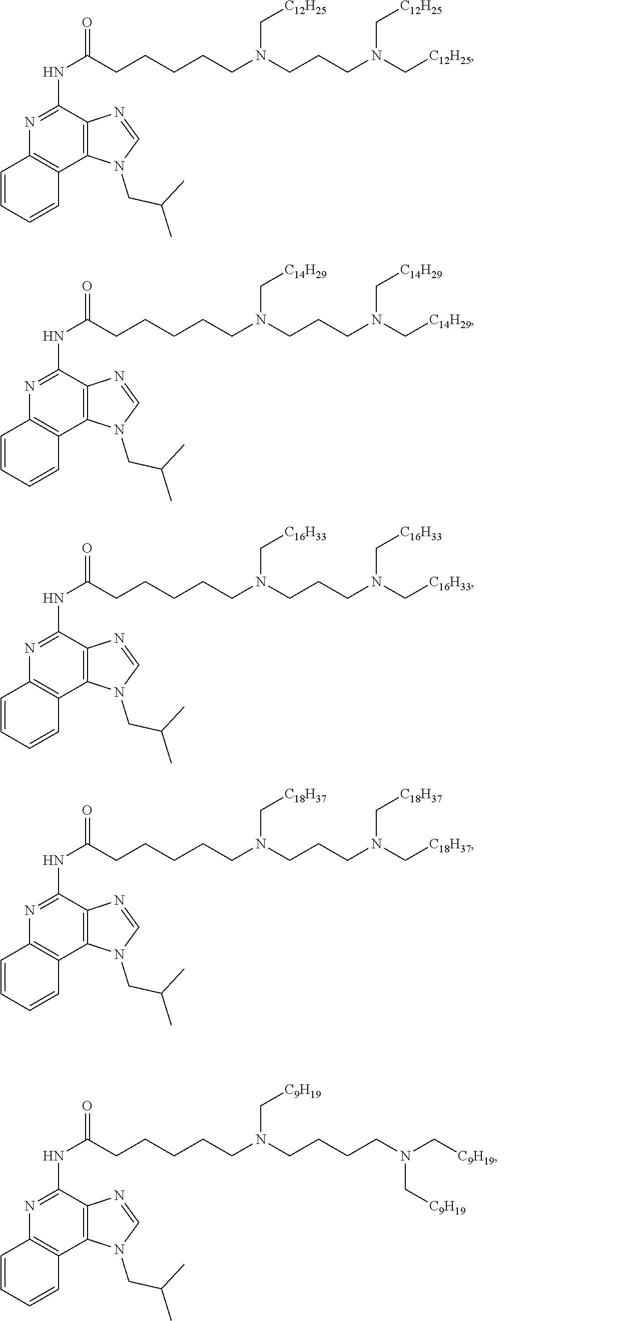

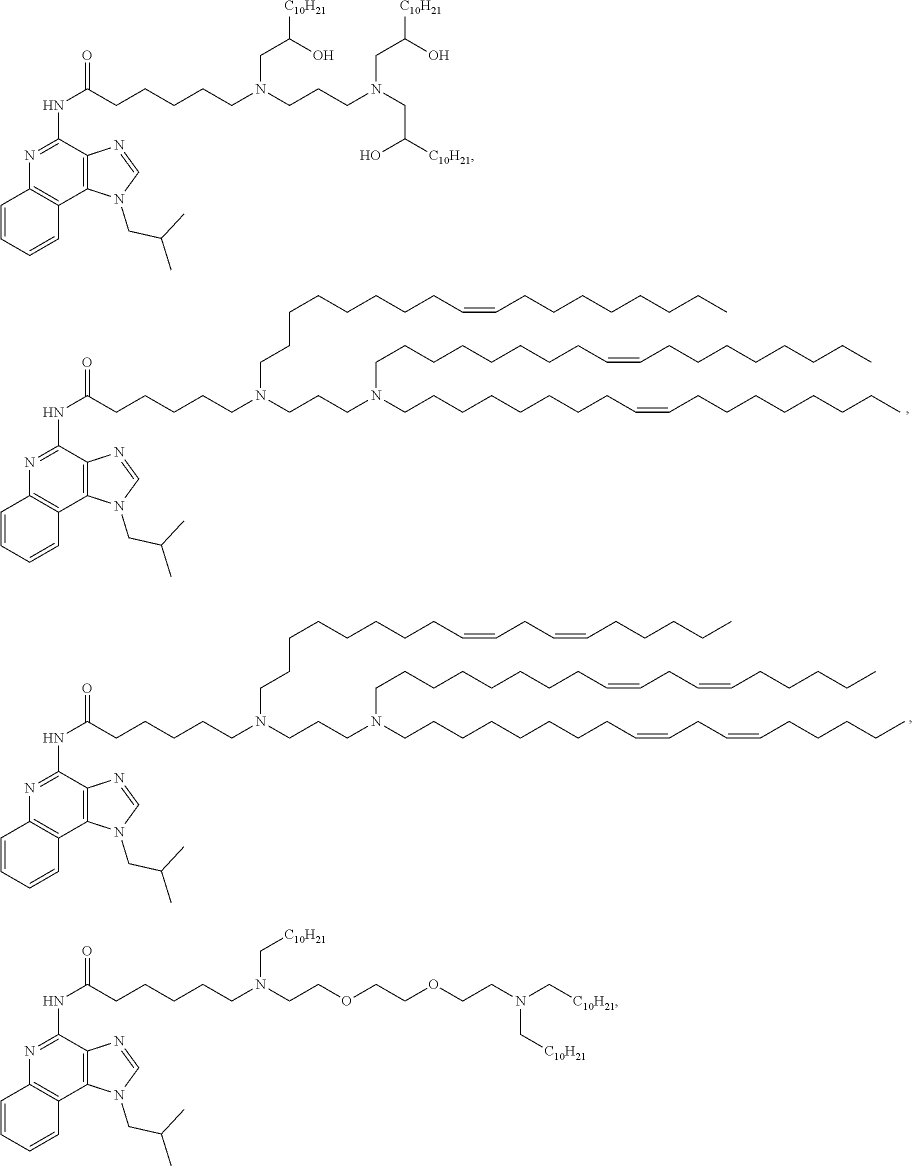

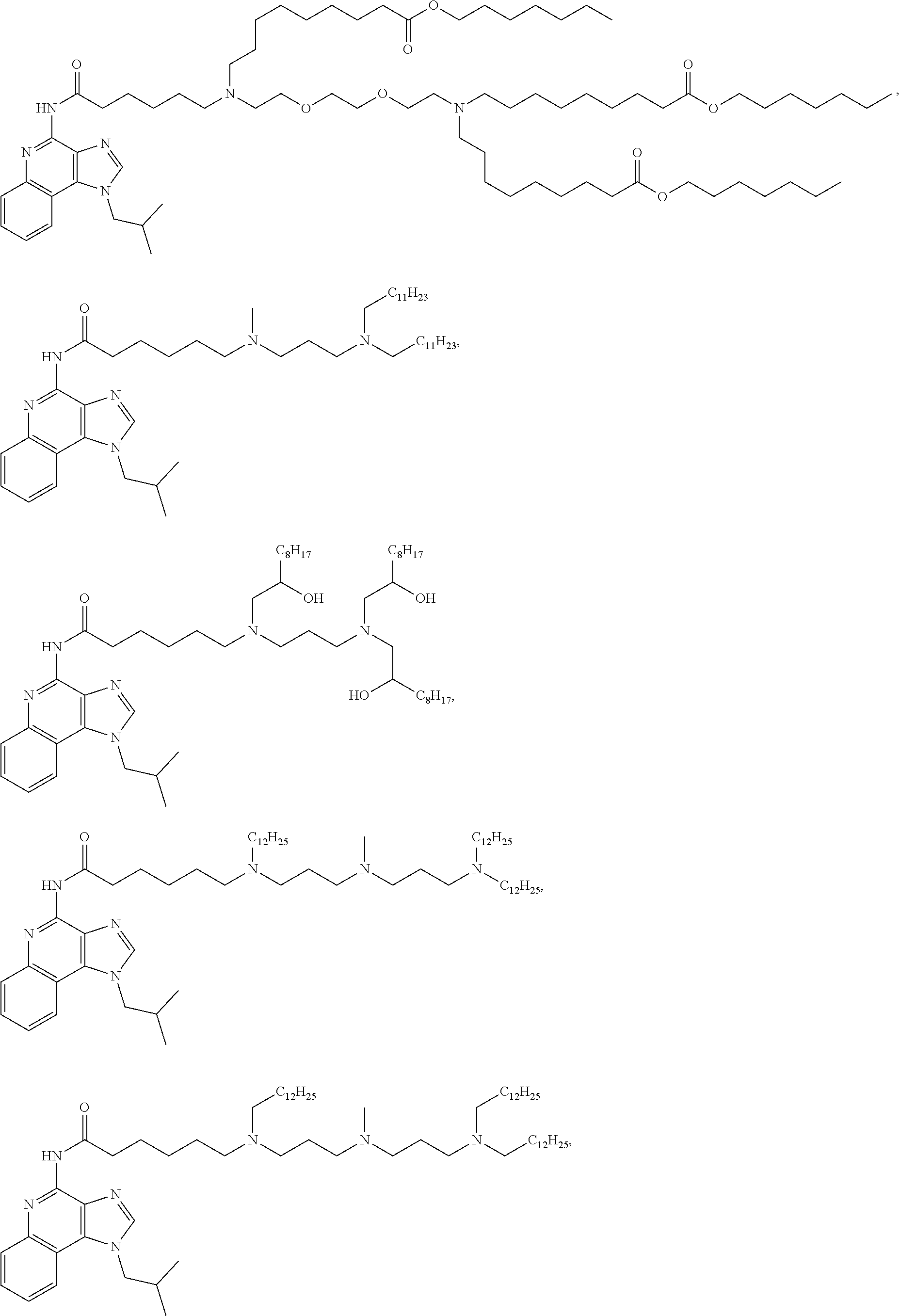

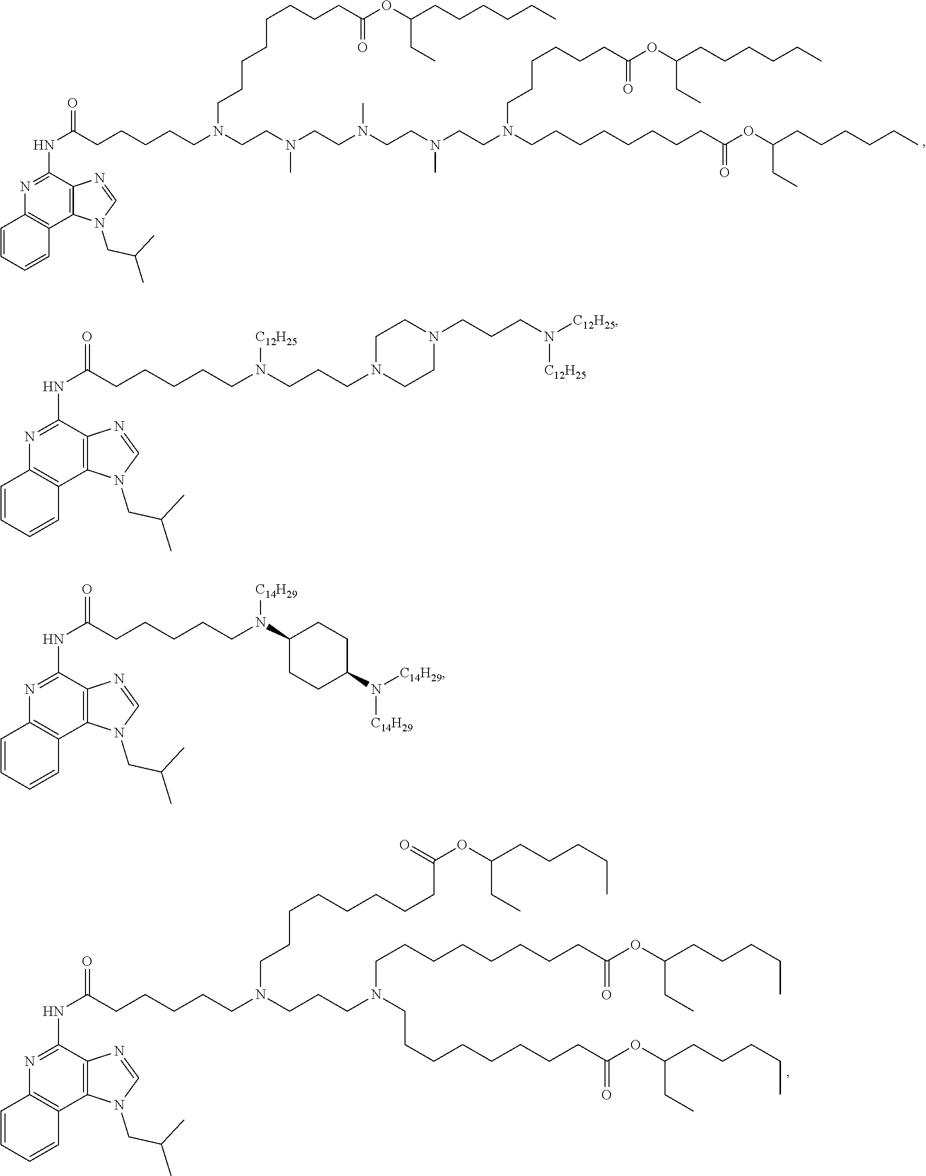

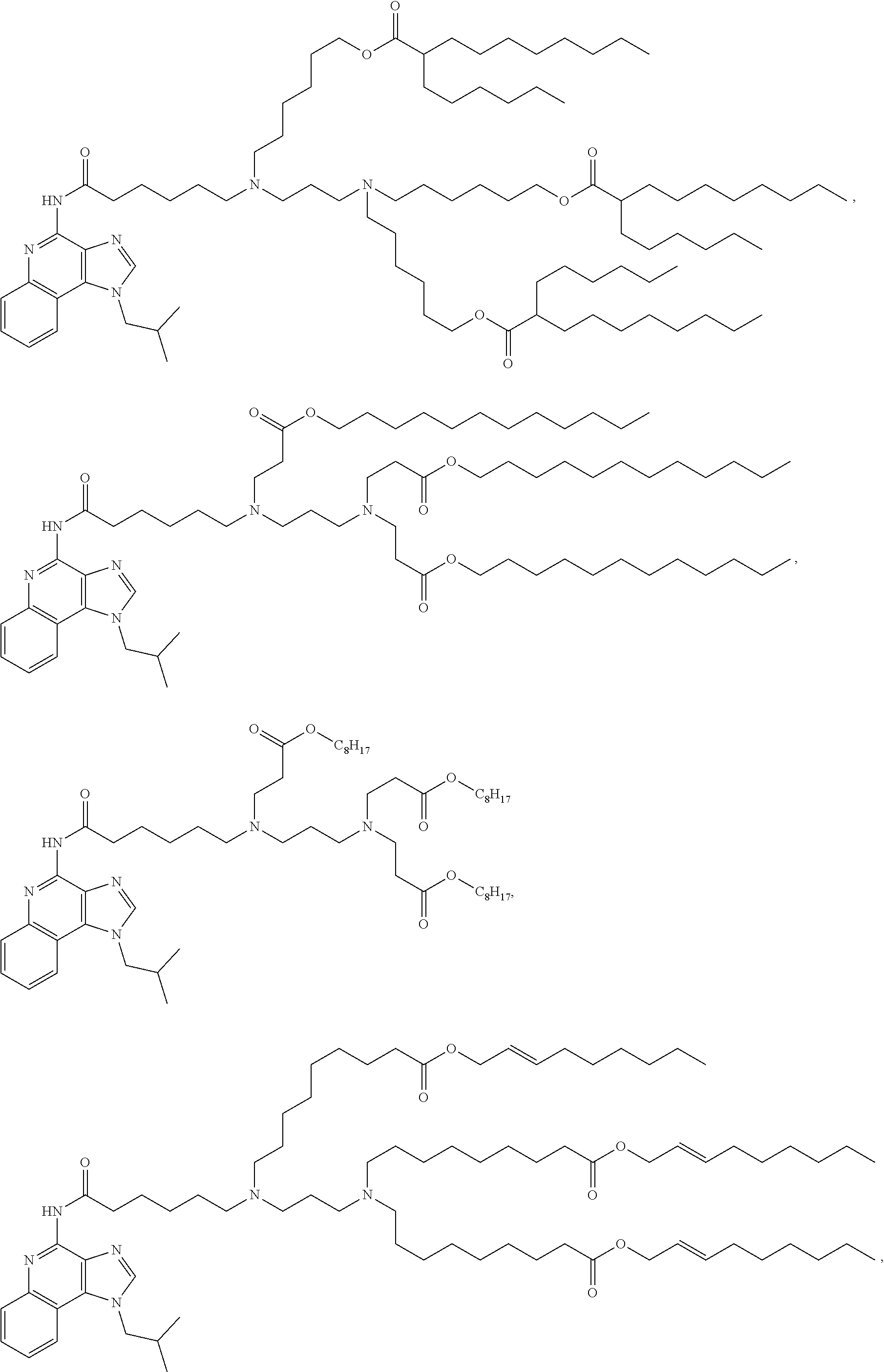

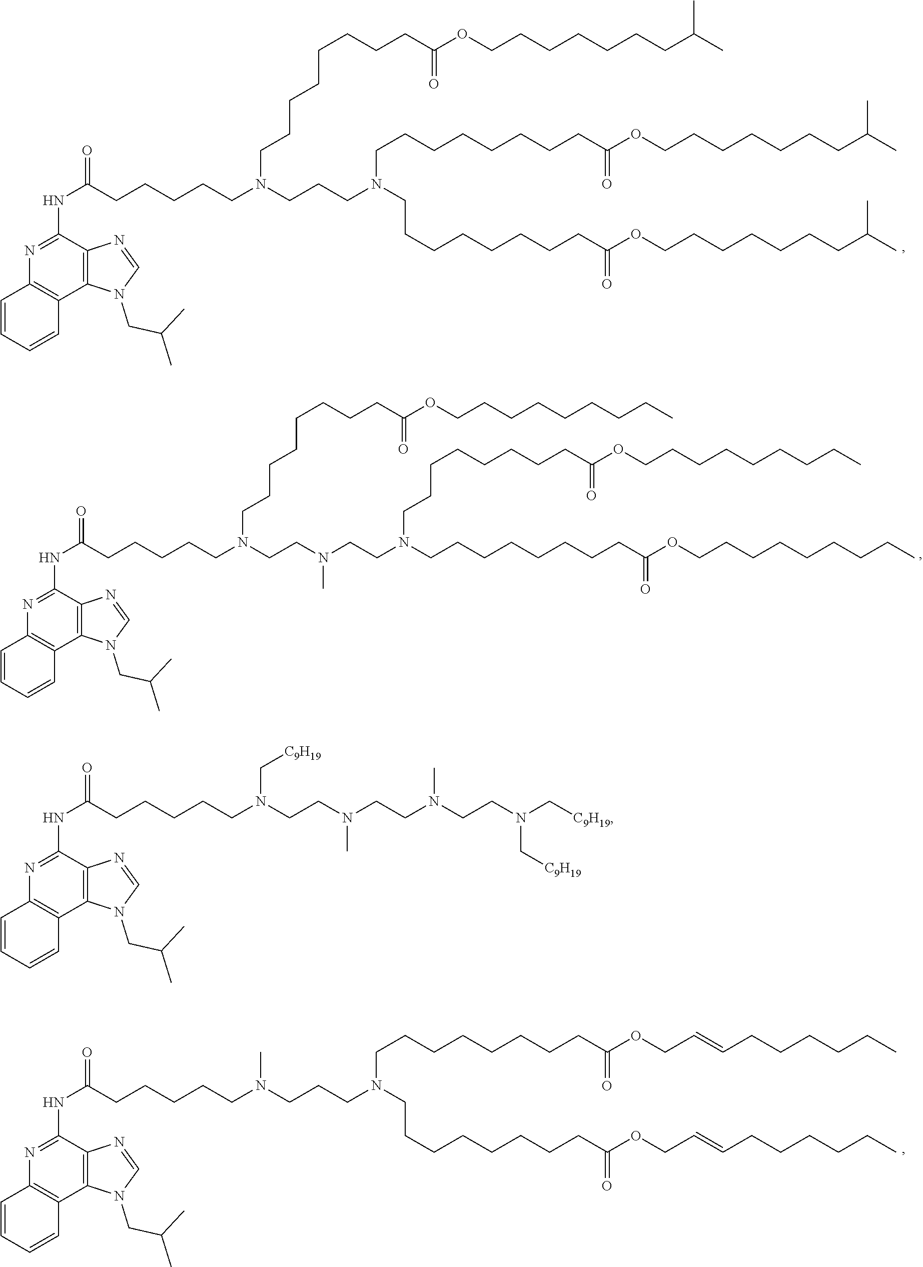

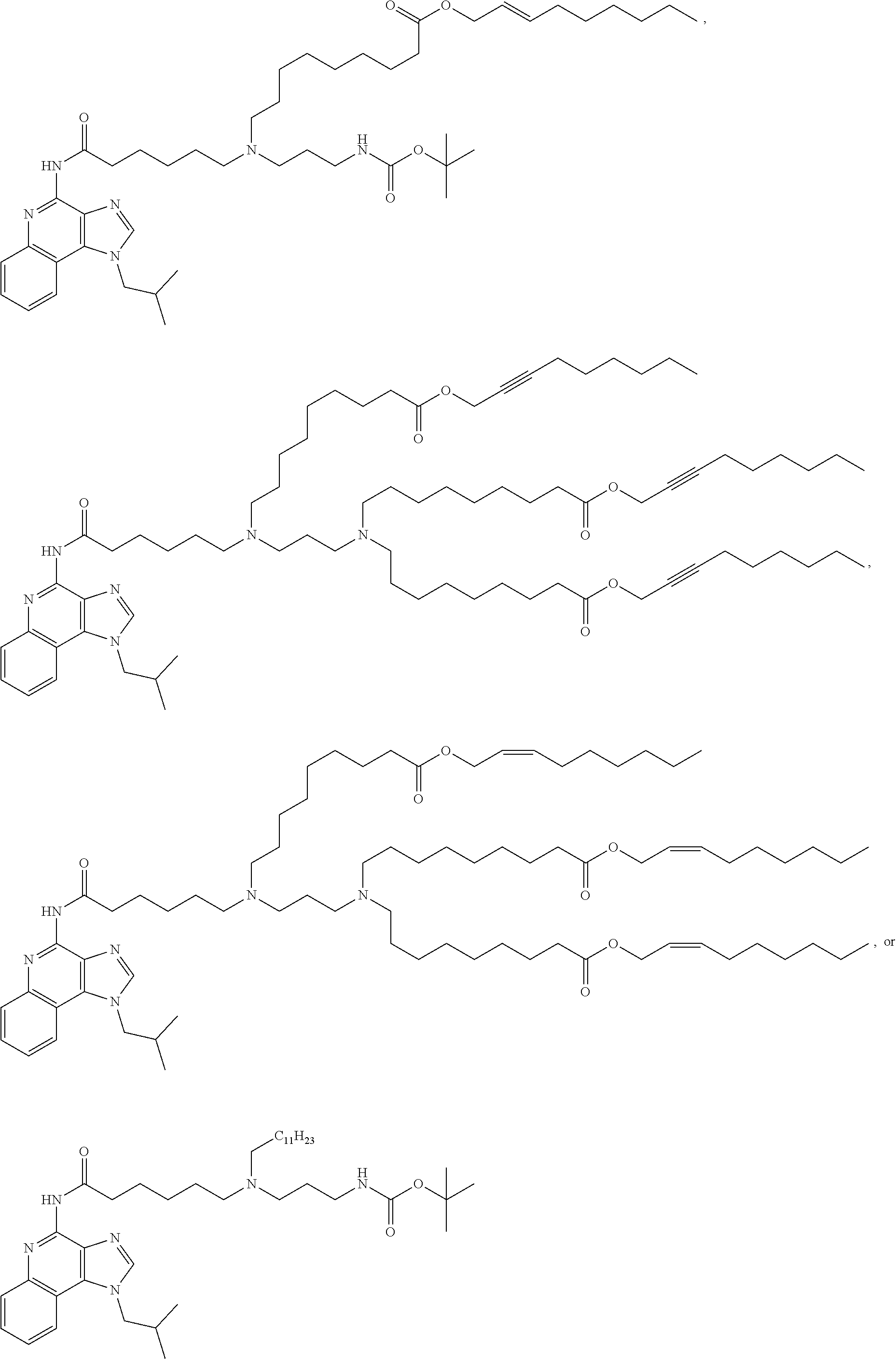

or a salt thereof.

In some embodiments, the compound is selected from the following:

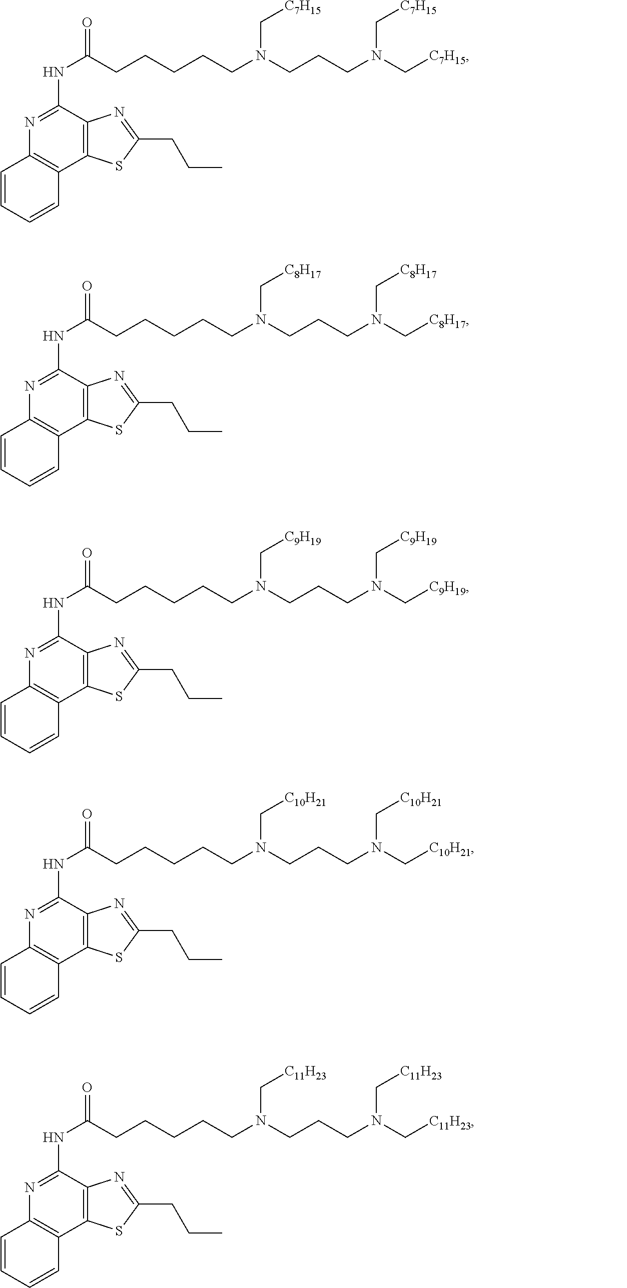

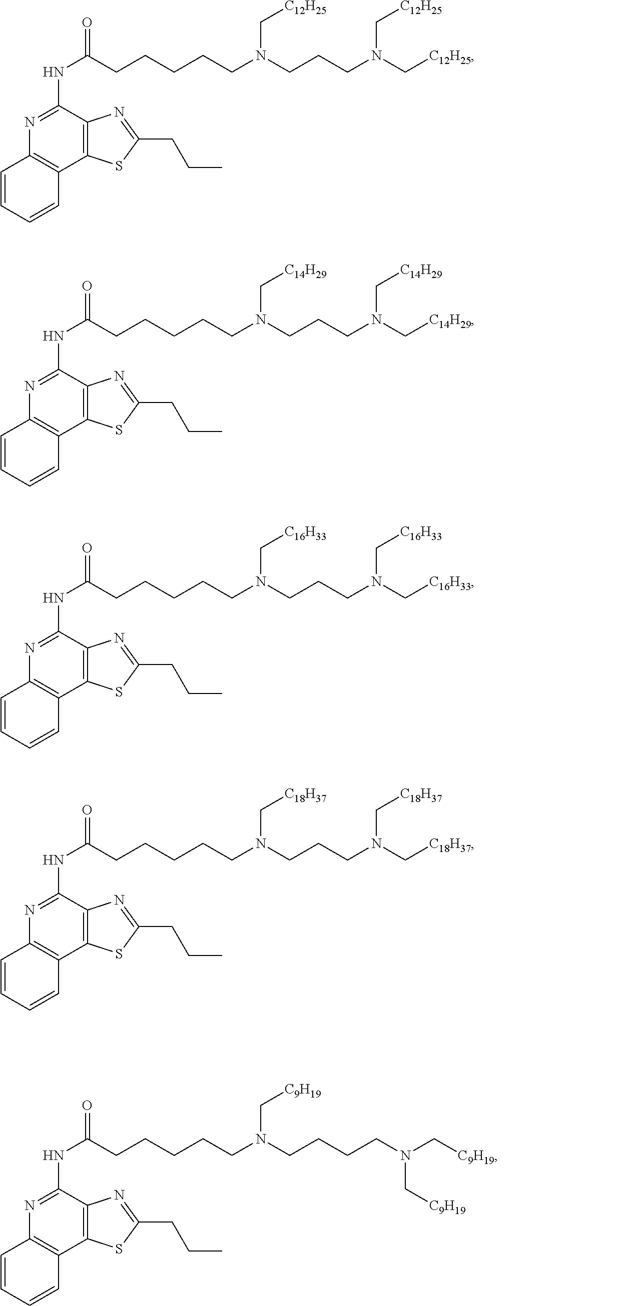

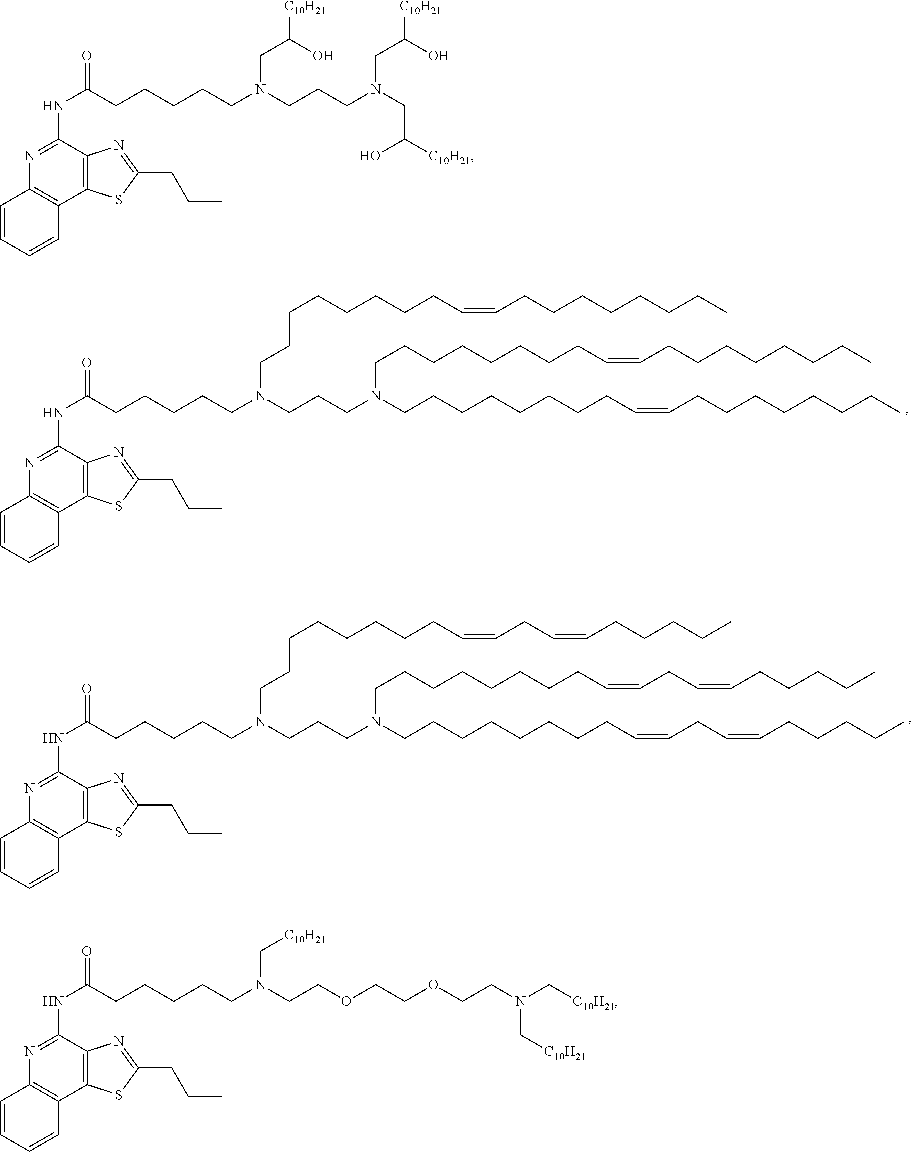

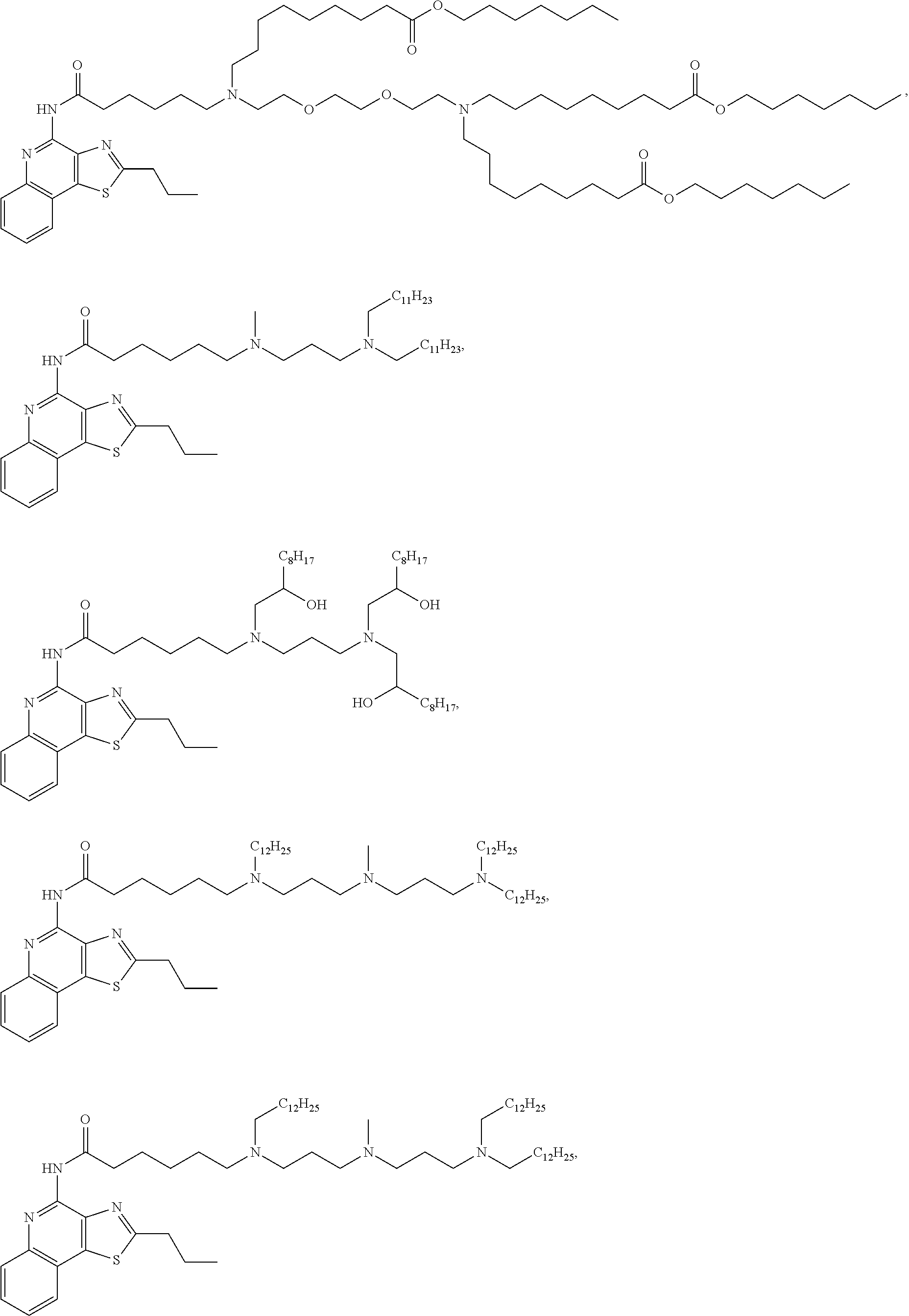

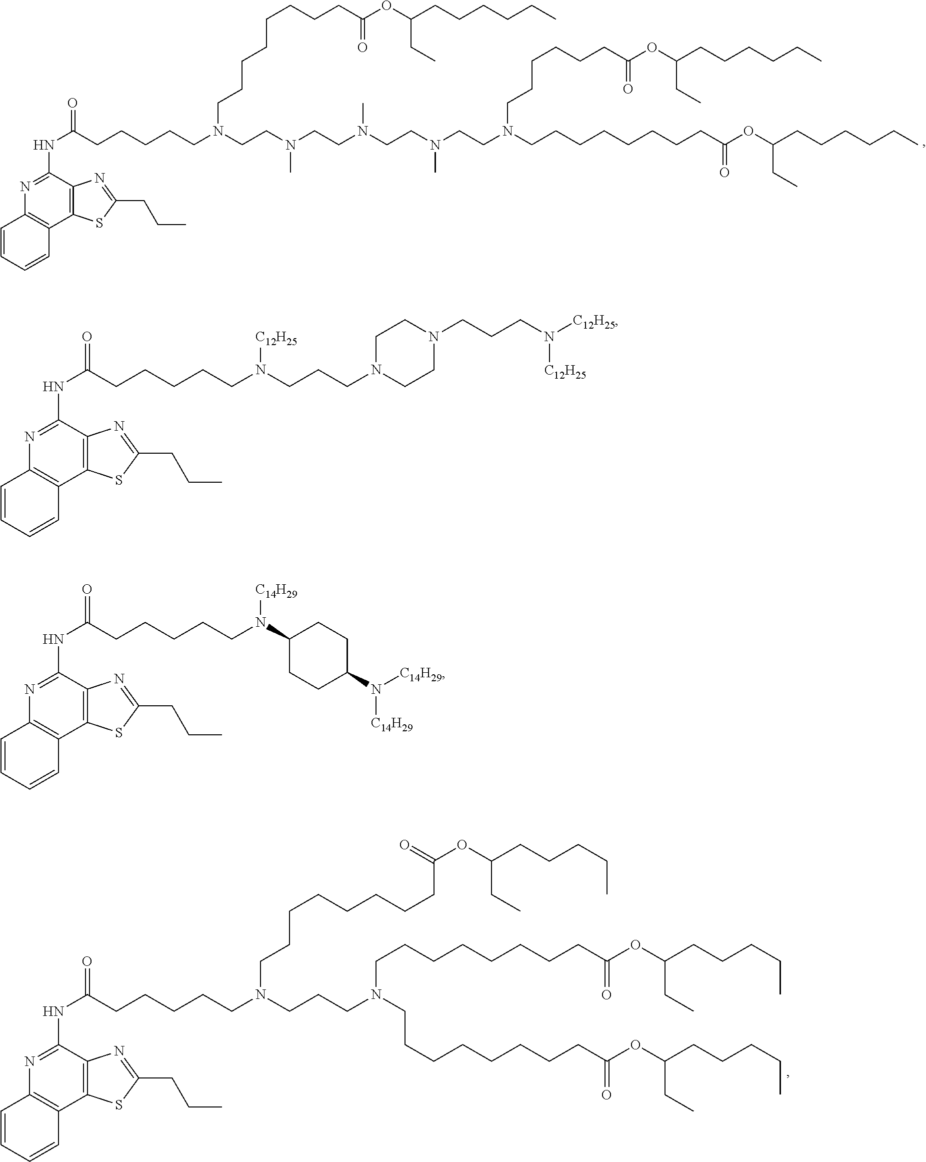

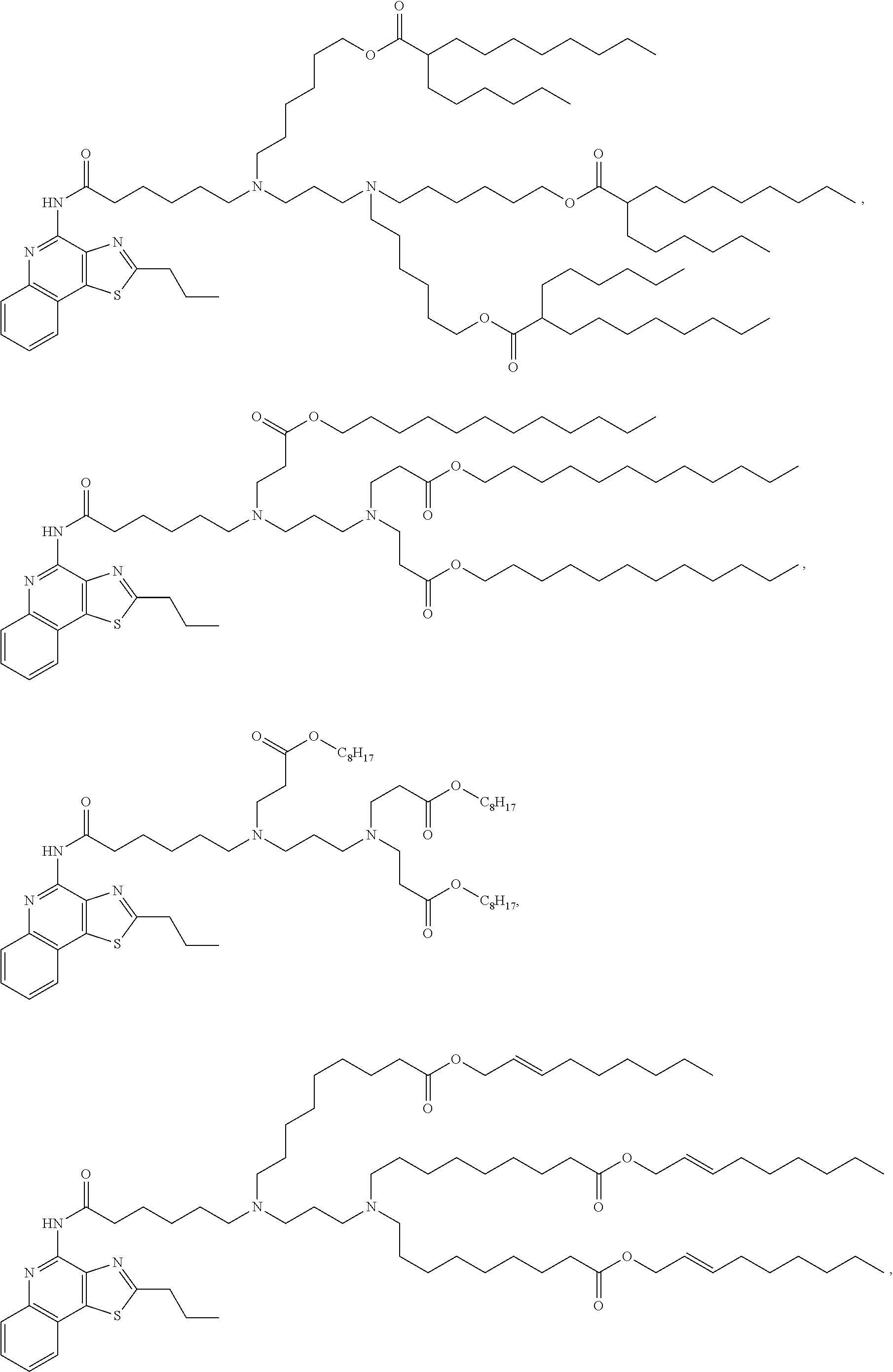

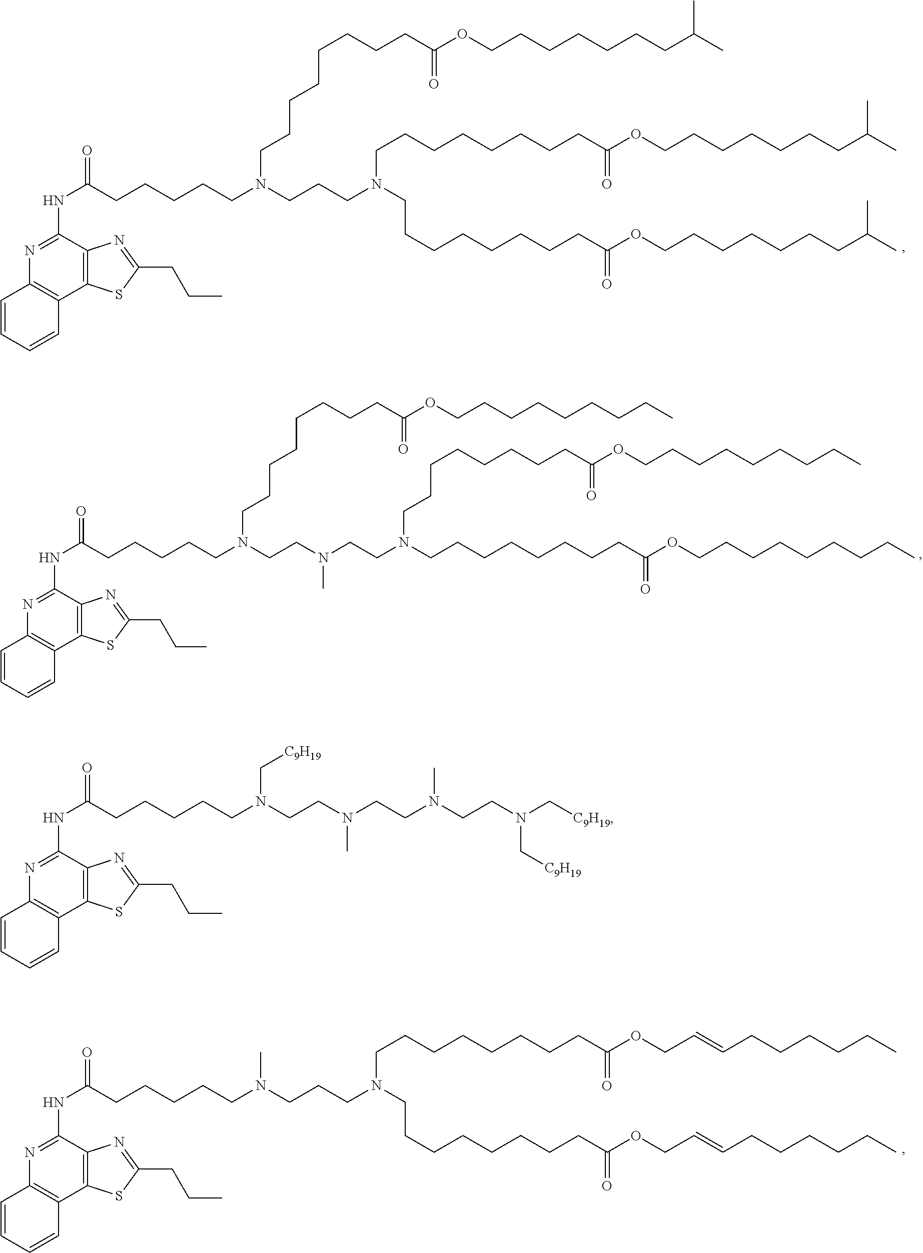

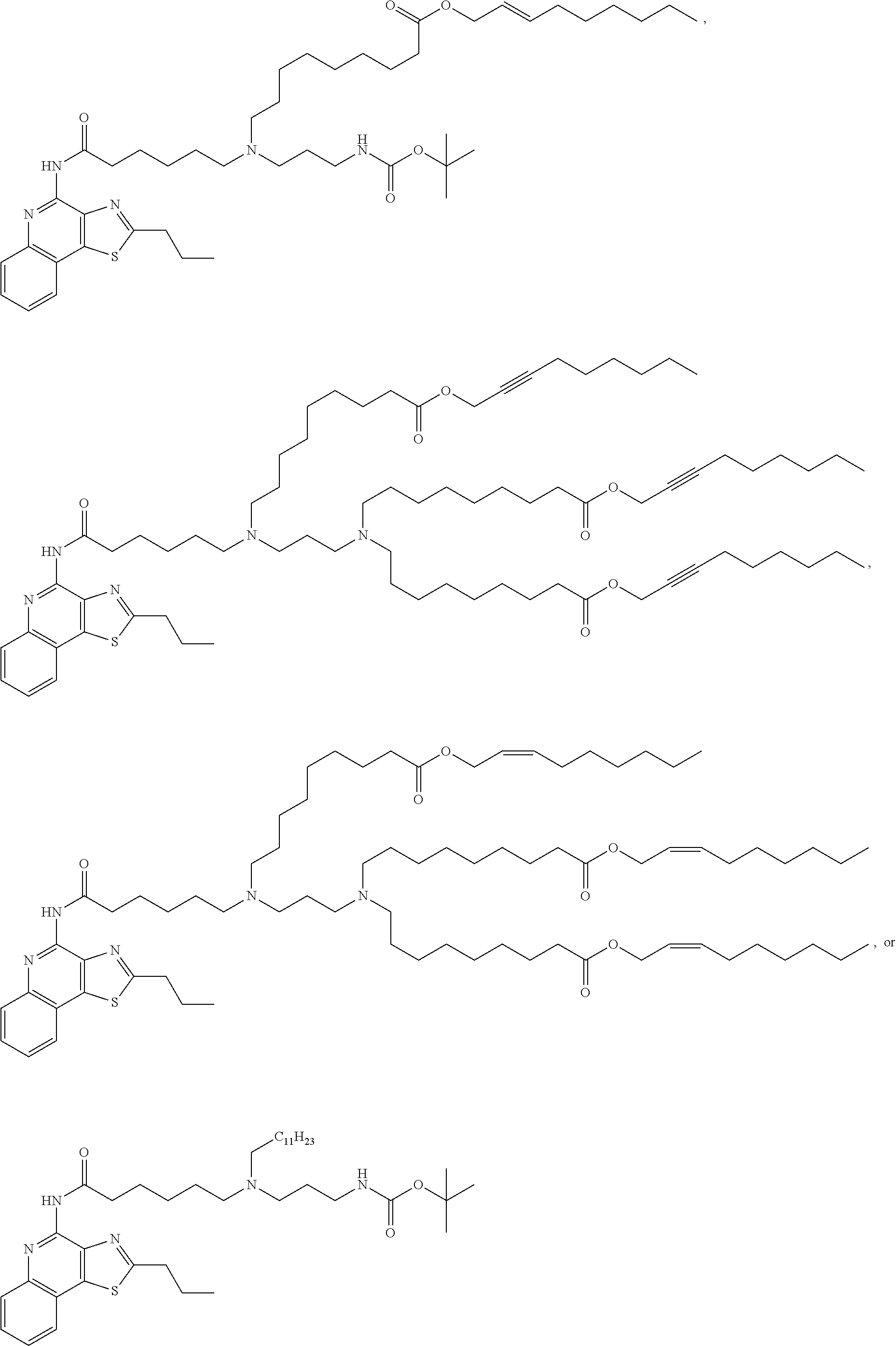

or a salt thereof.

In some embodiments, the compound is selected from the following:

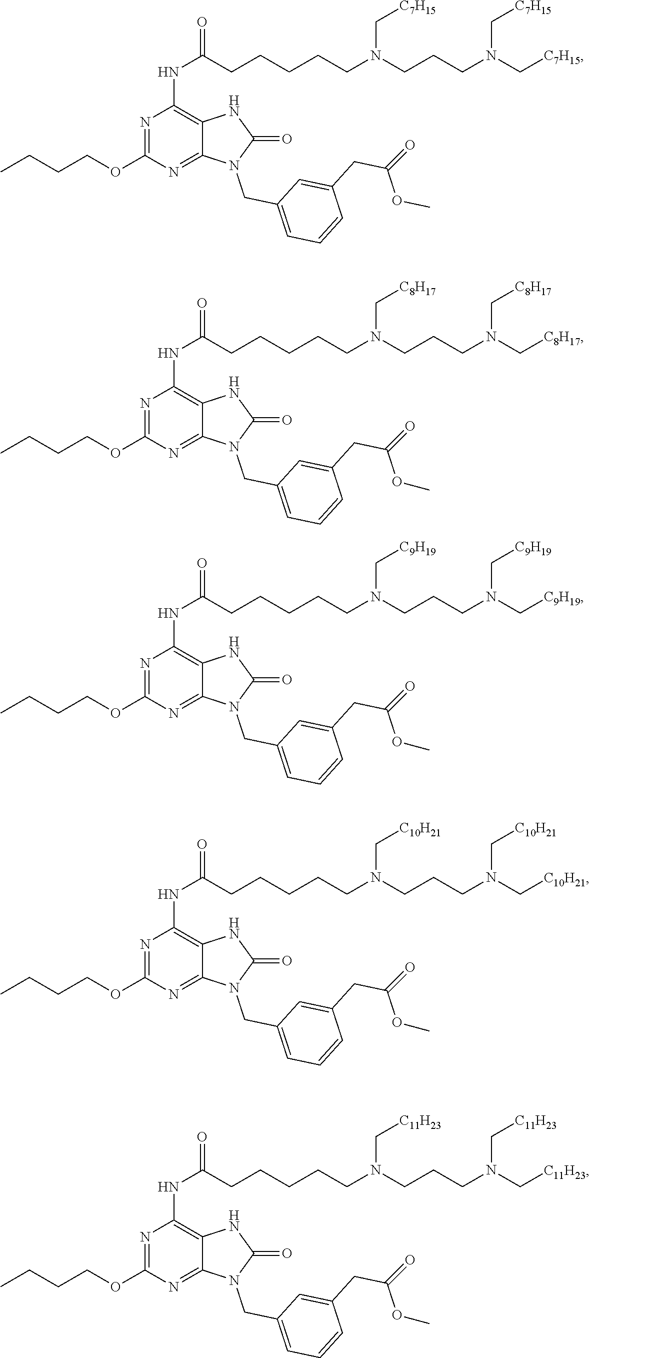

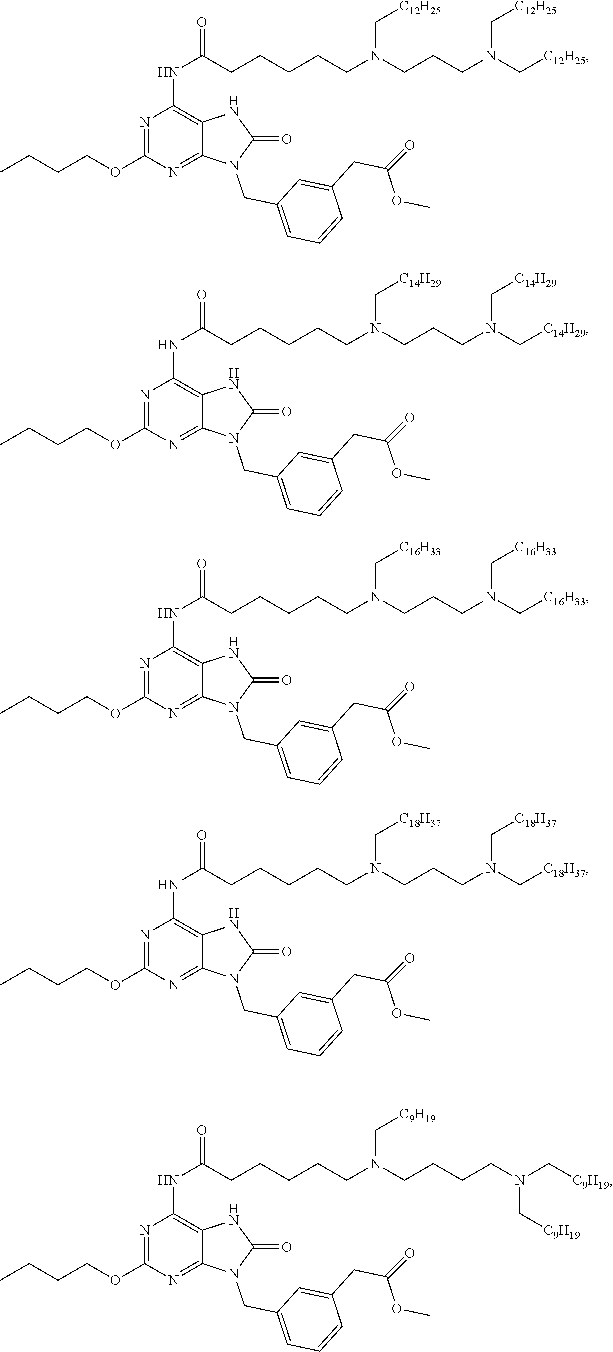

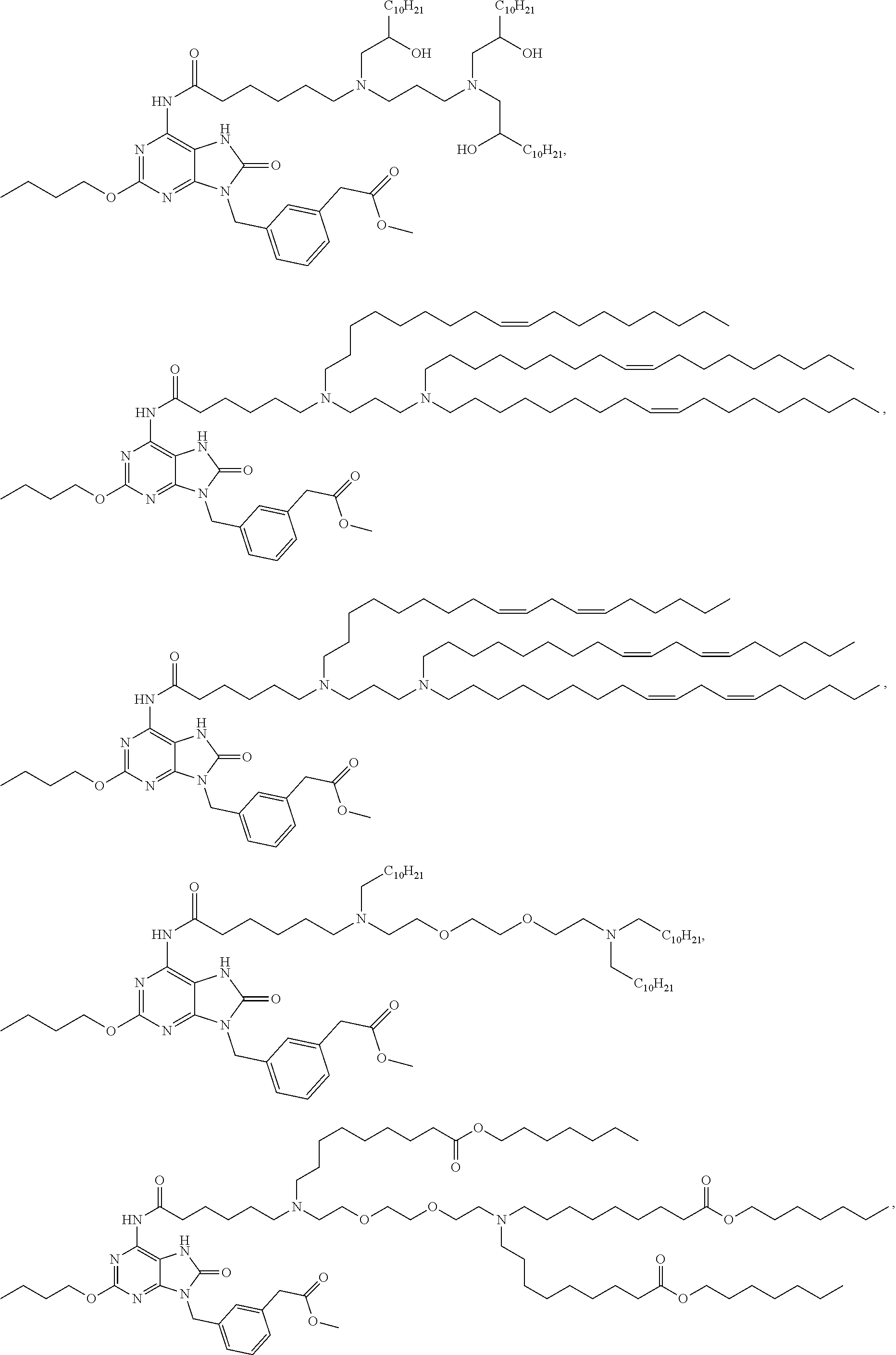

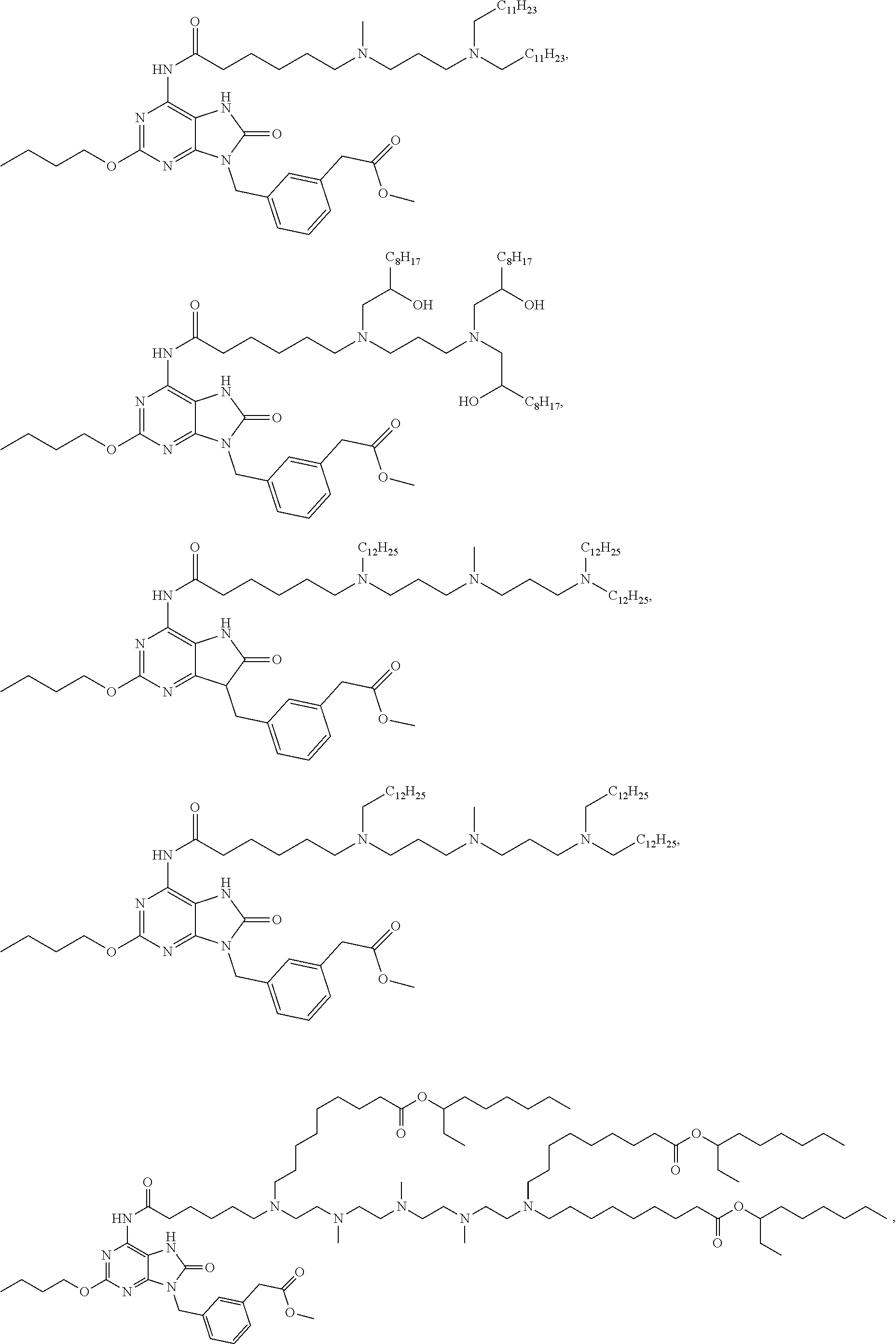

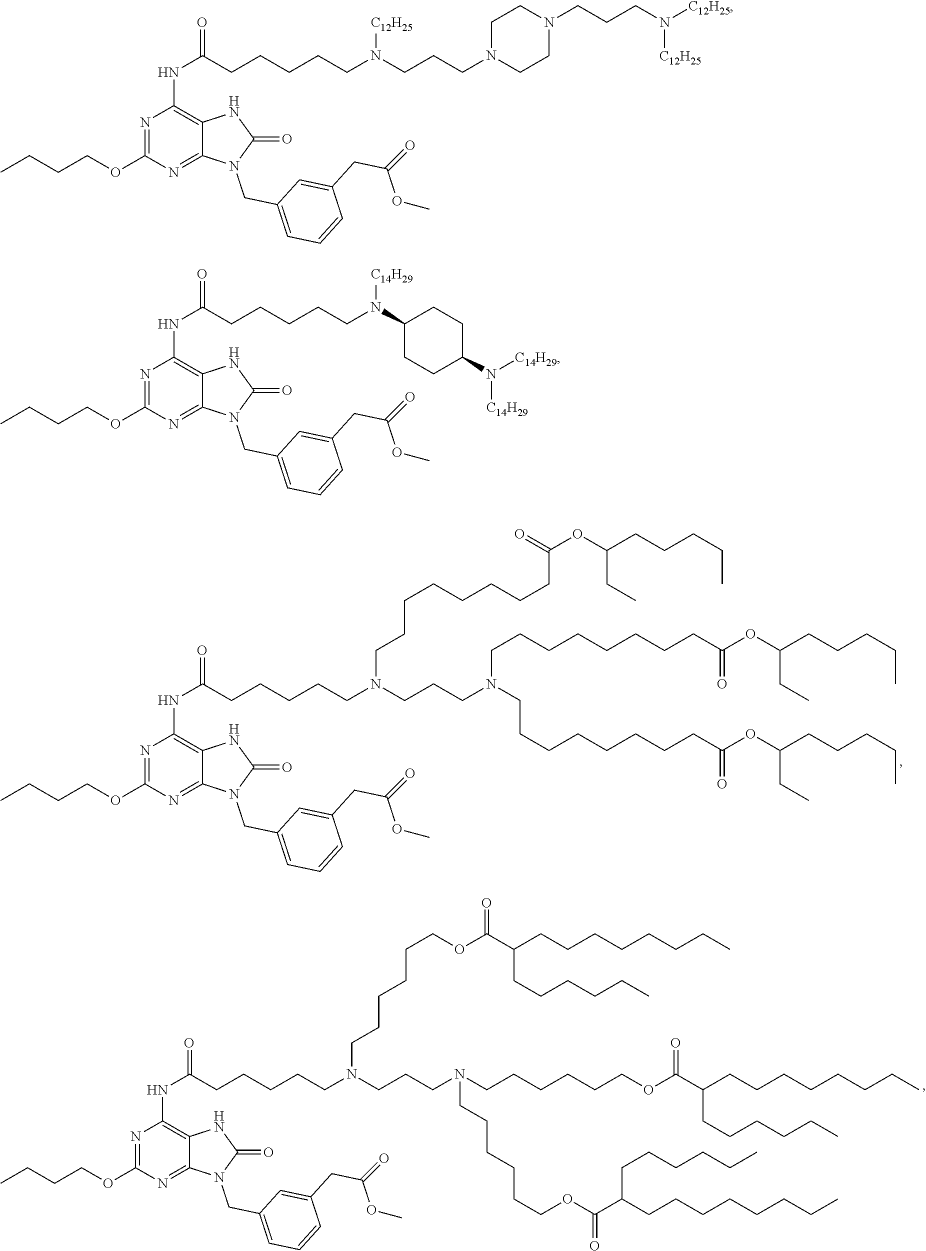

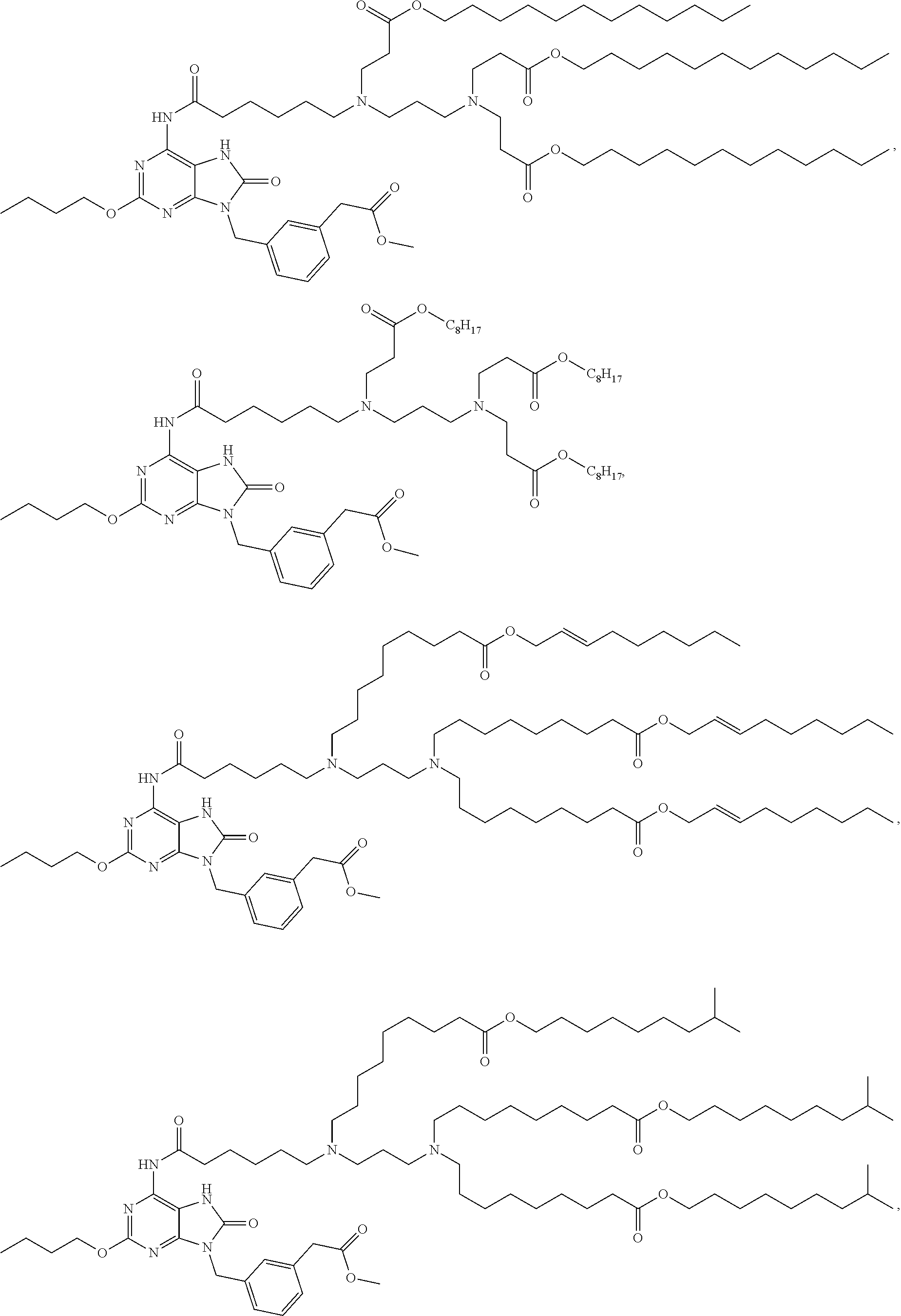

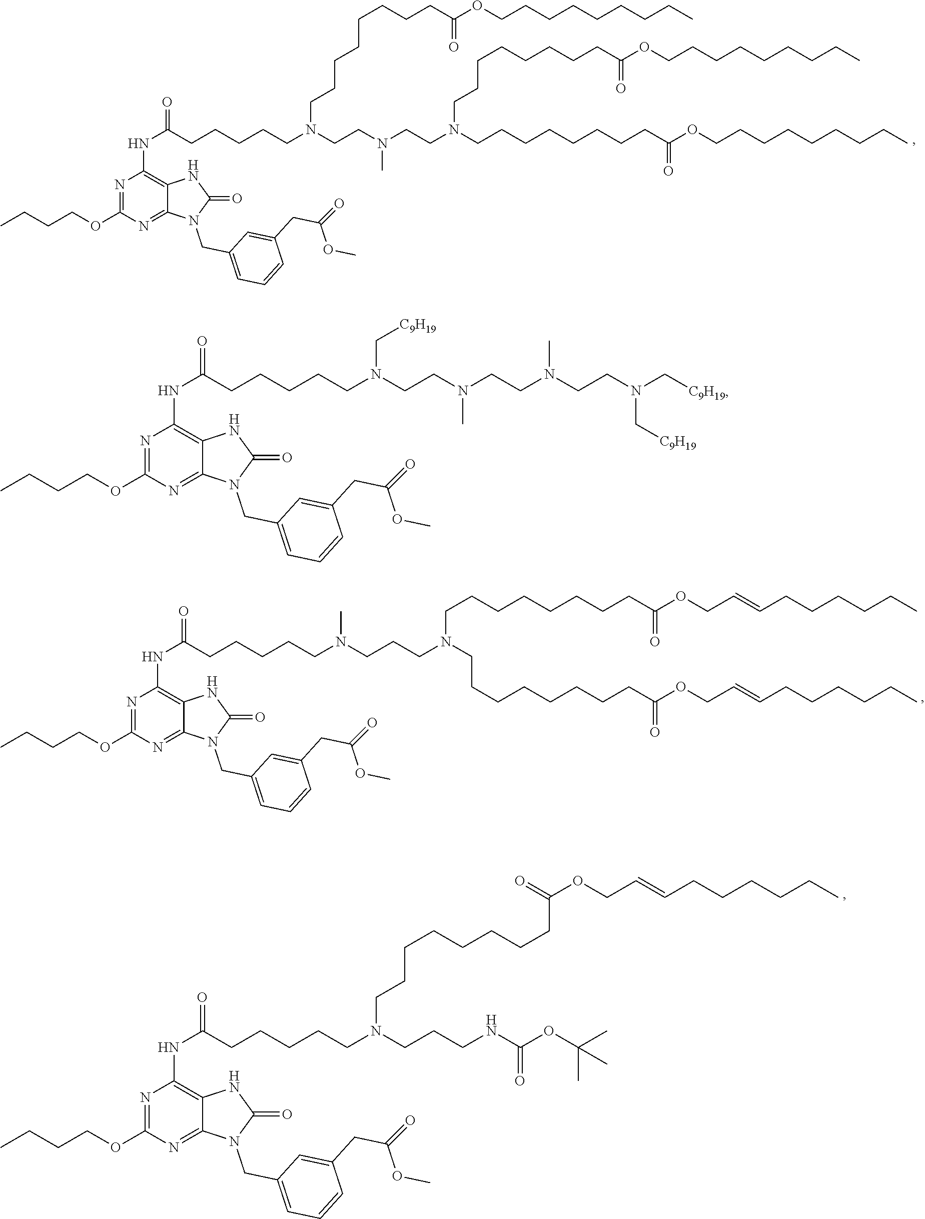

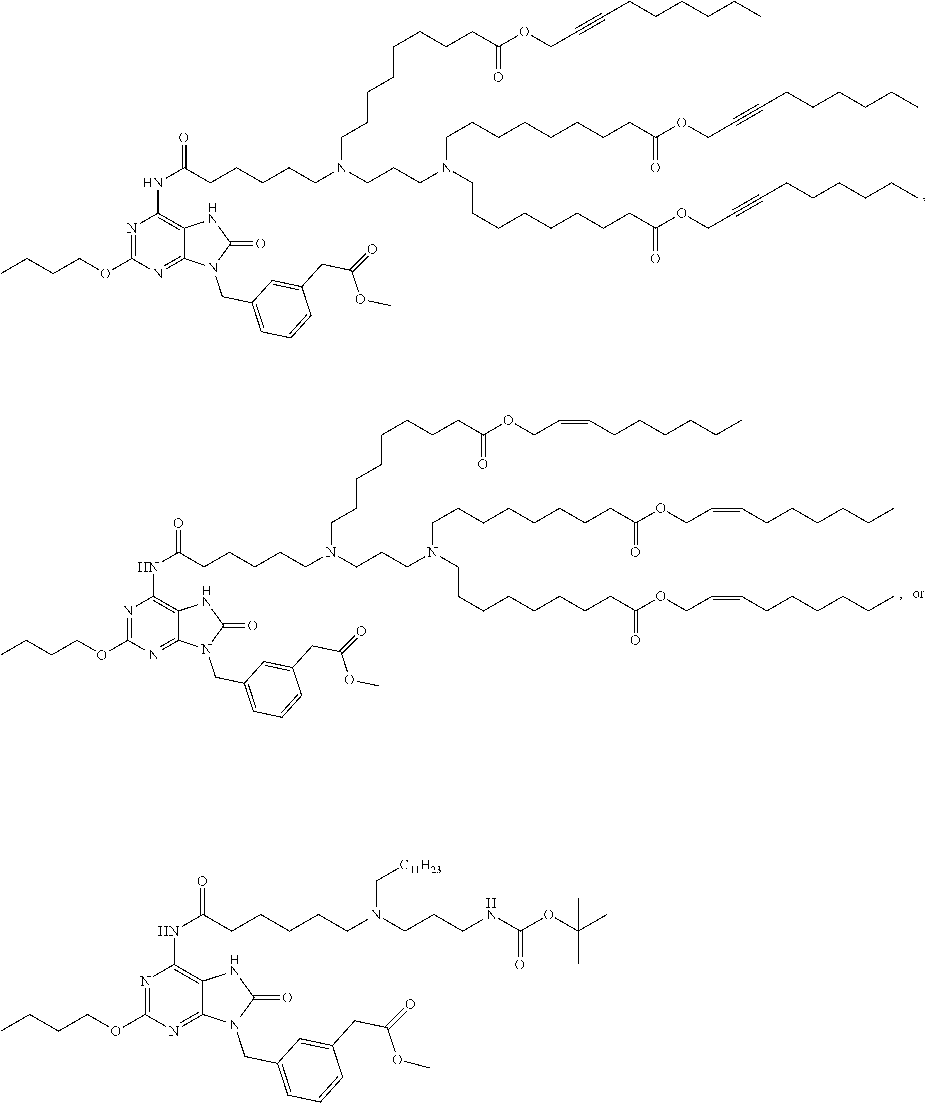

or a salt thereof.

In some embodiments, the compound is selected from the following:

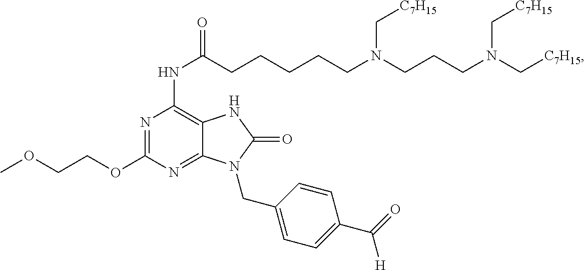

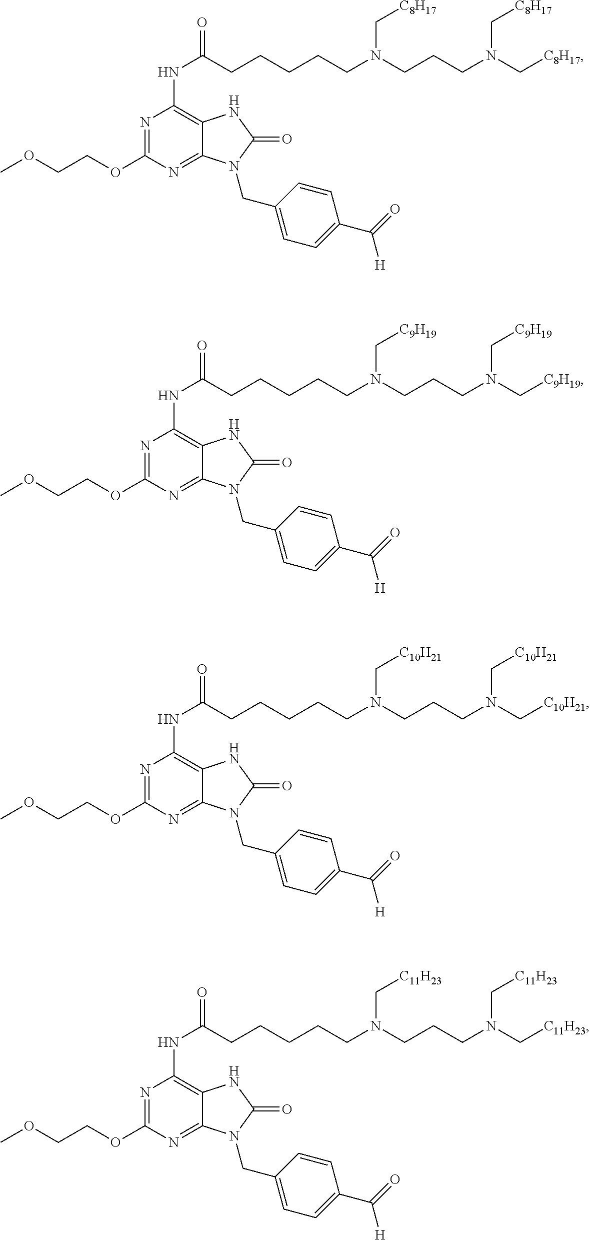

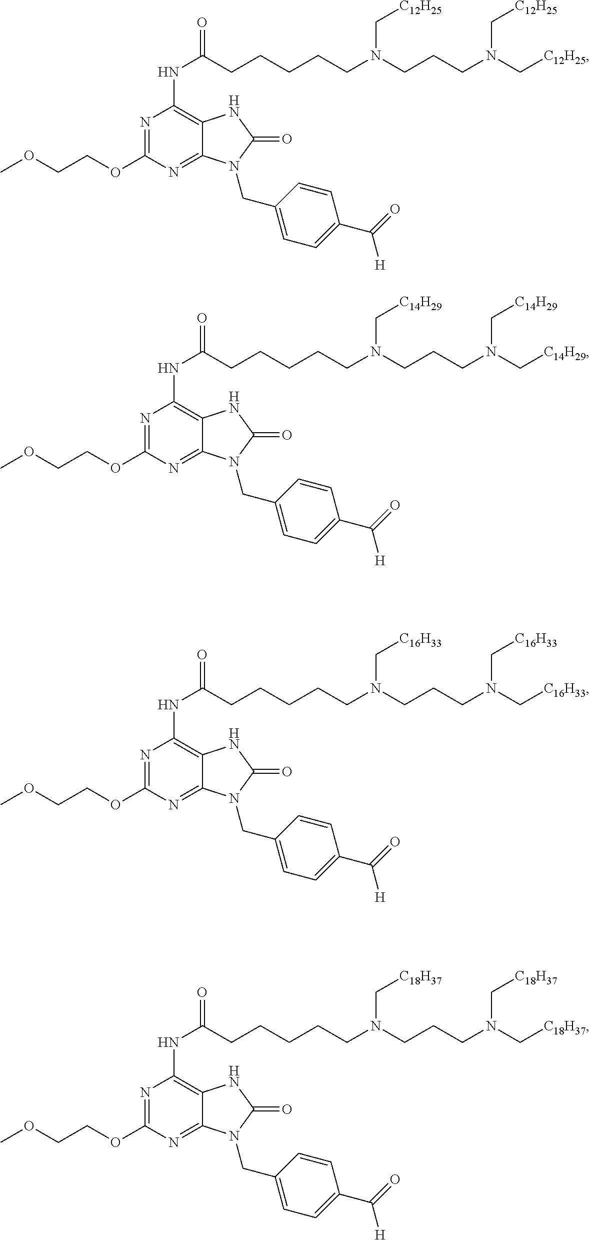

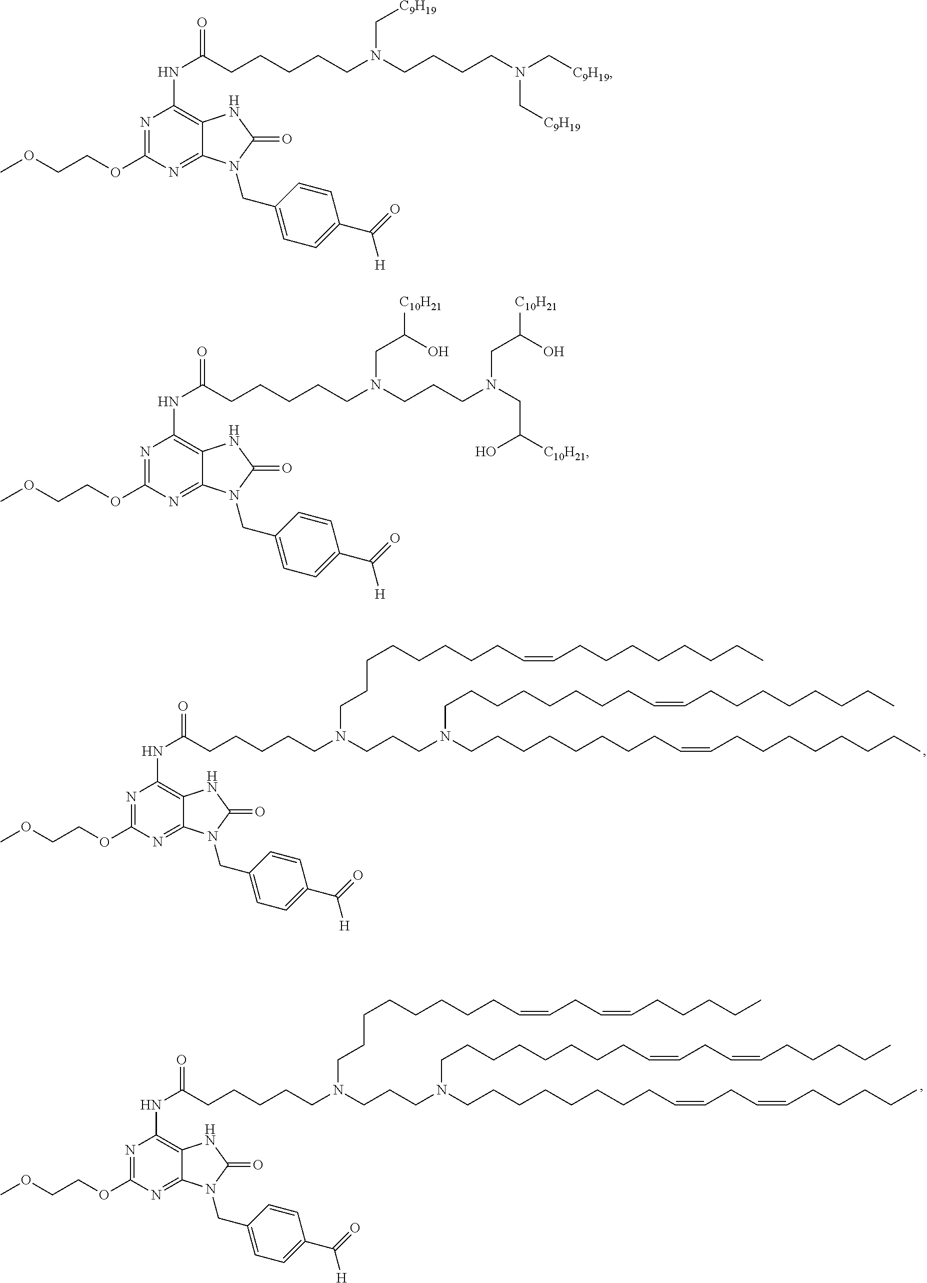

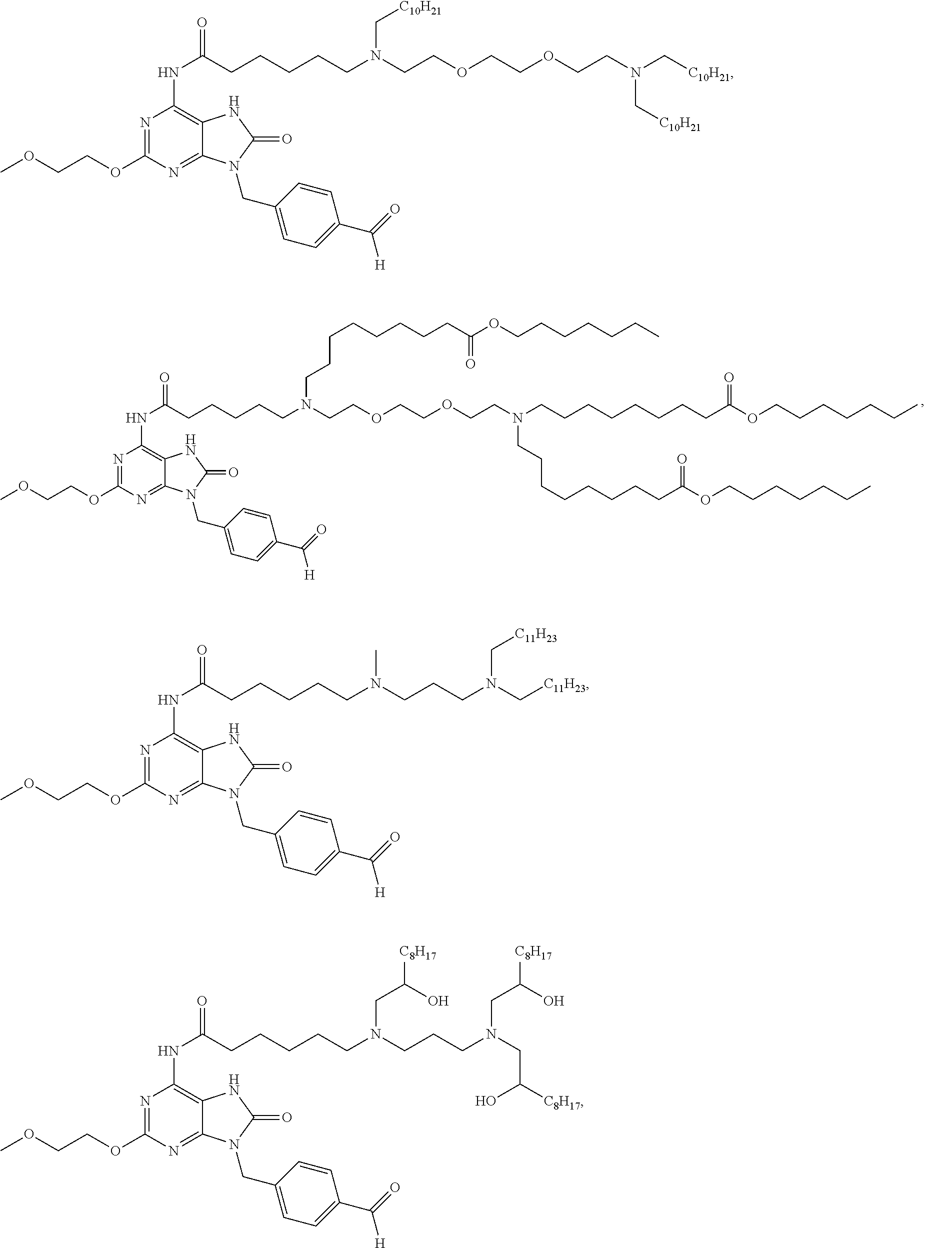

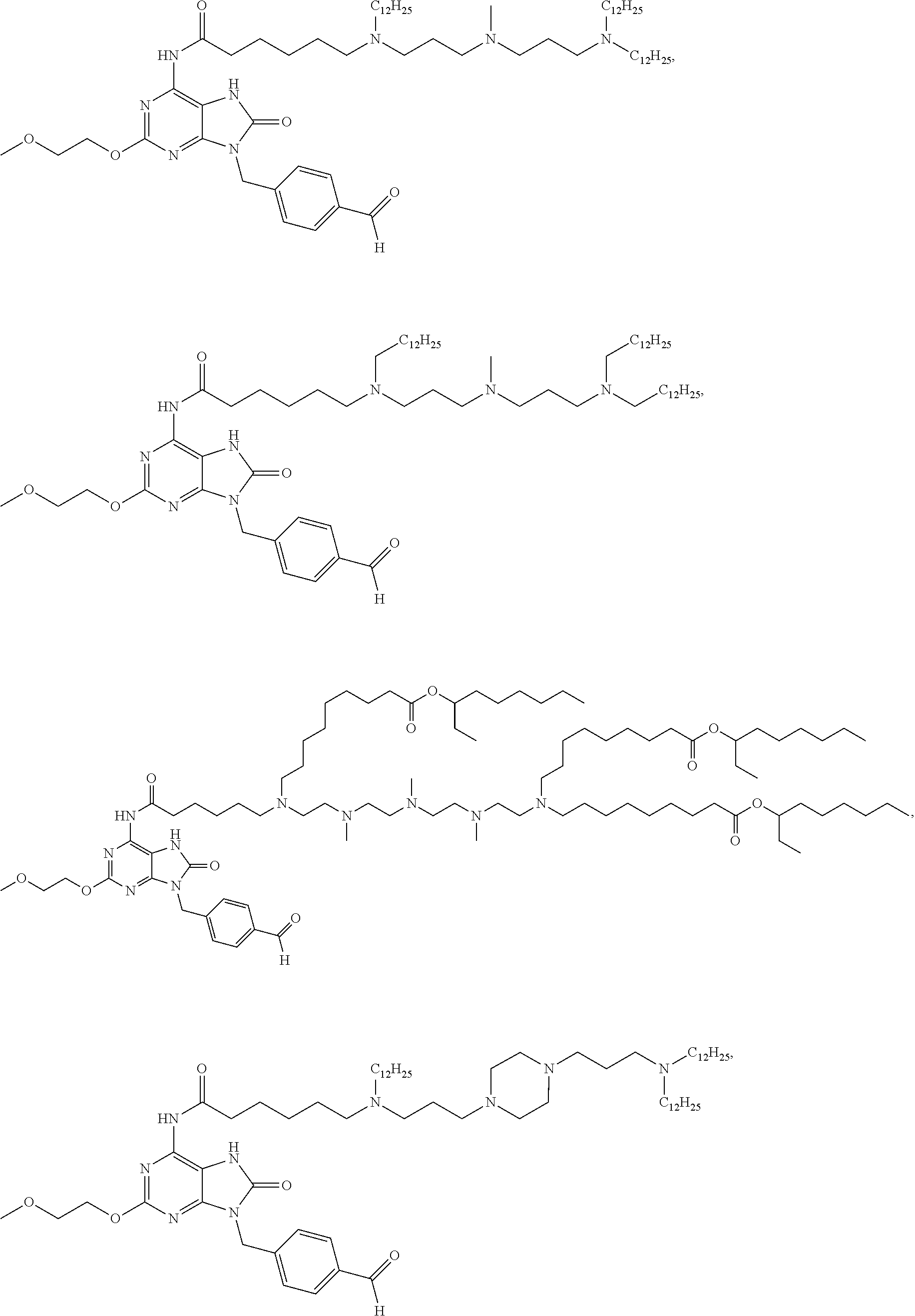

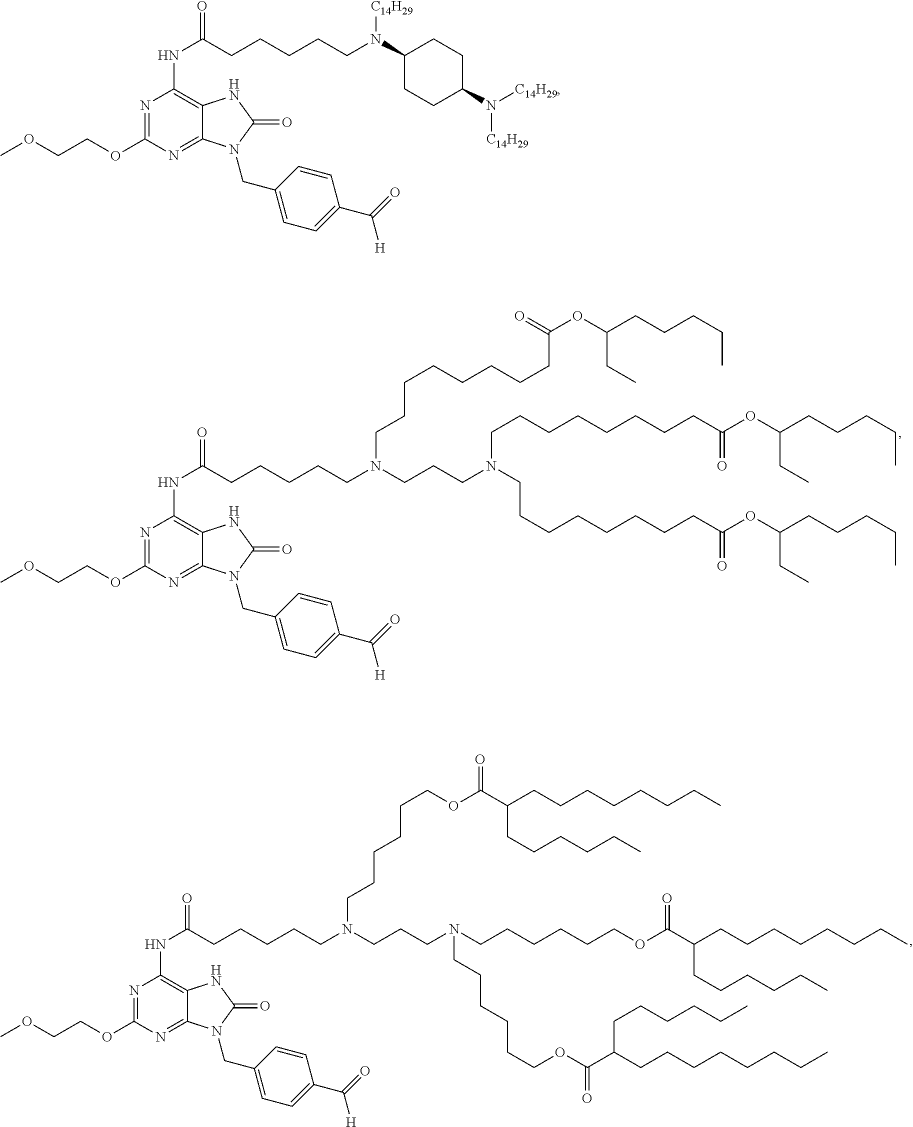

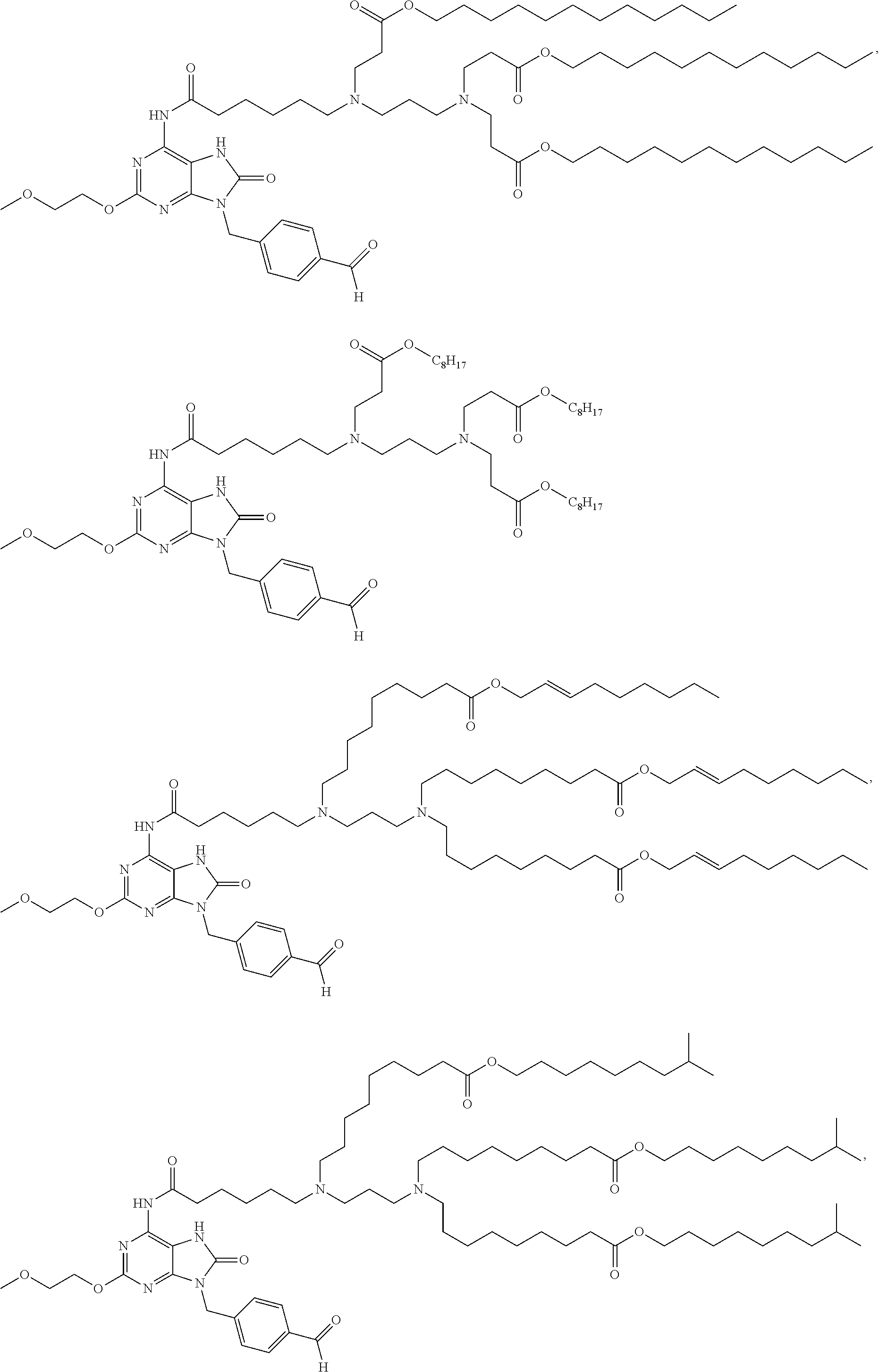

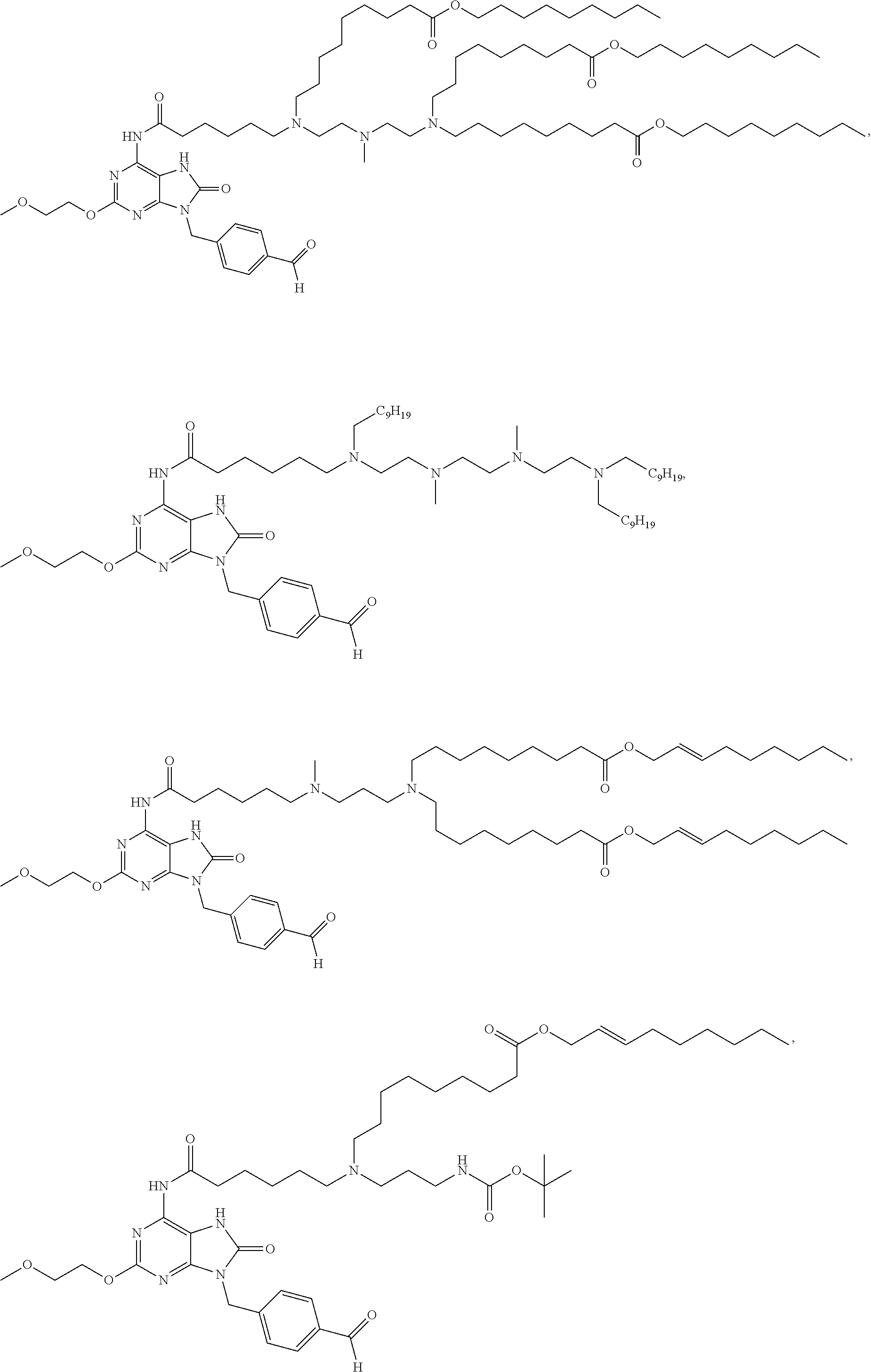

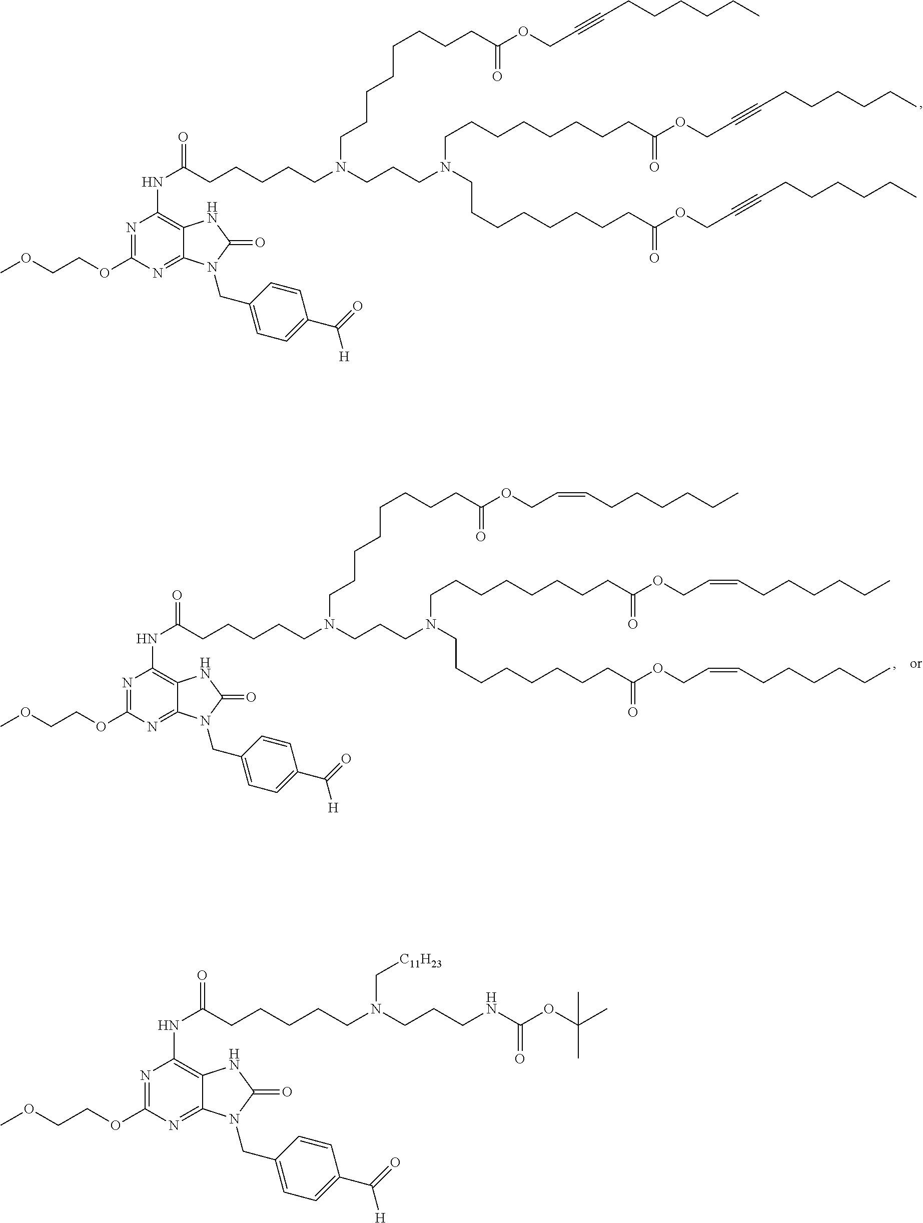

or a salt thereof.

In some embodiments, the compound is selected from the following:

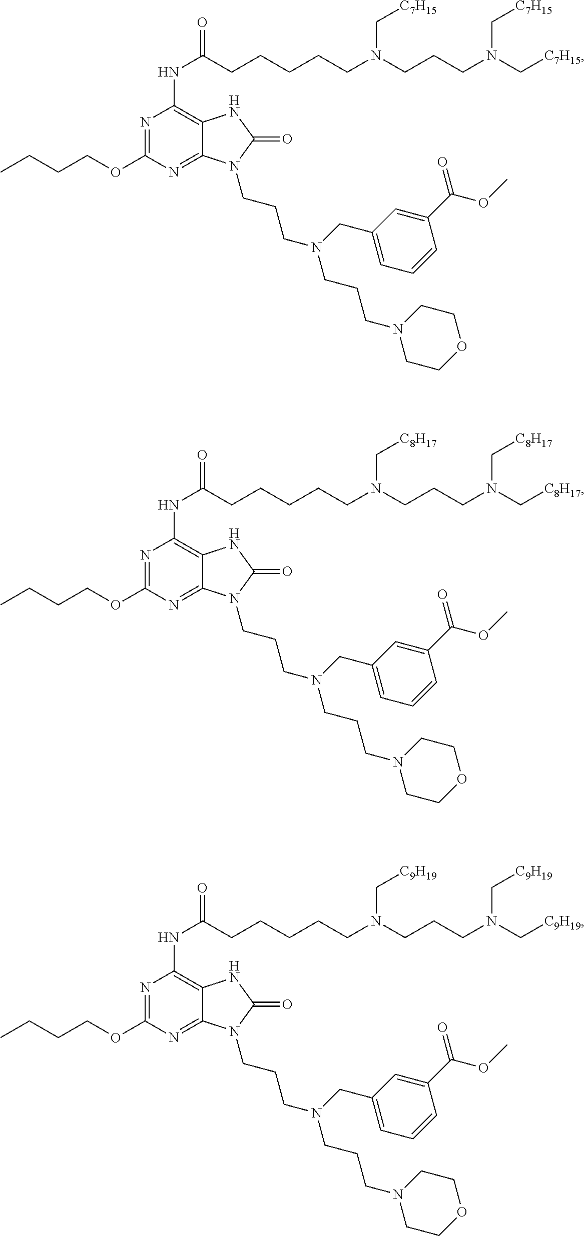

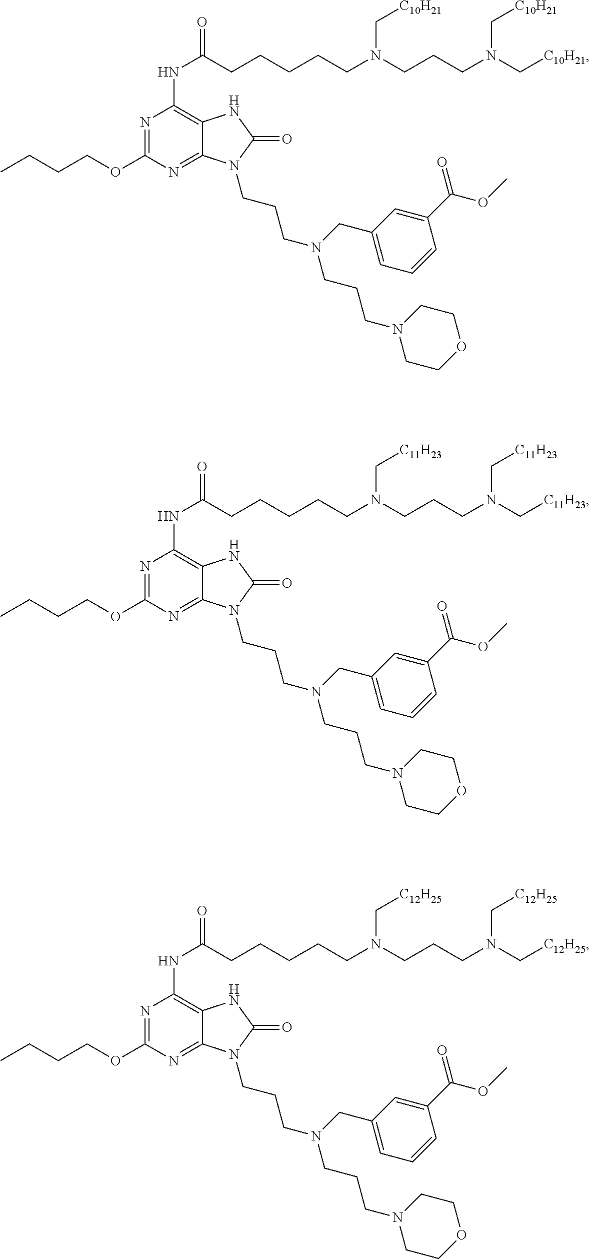

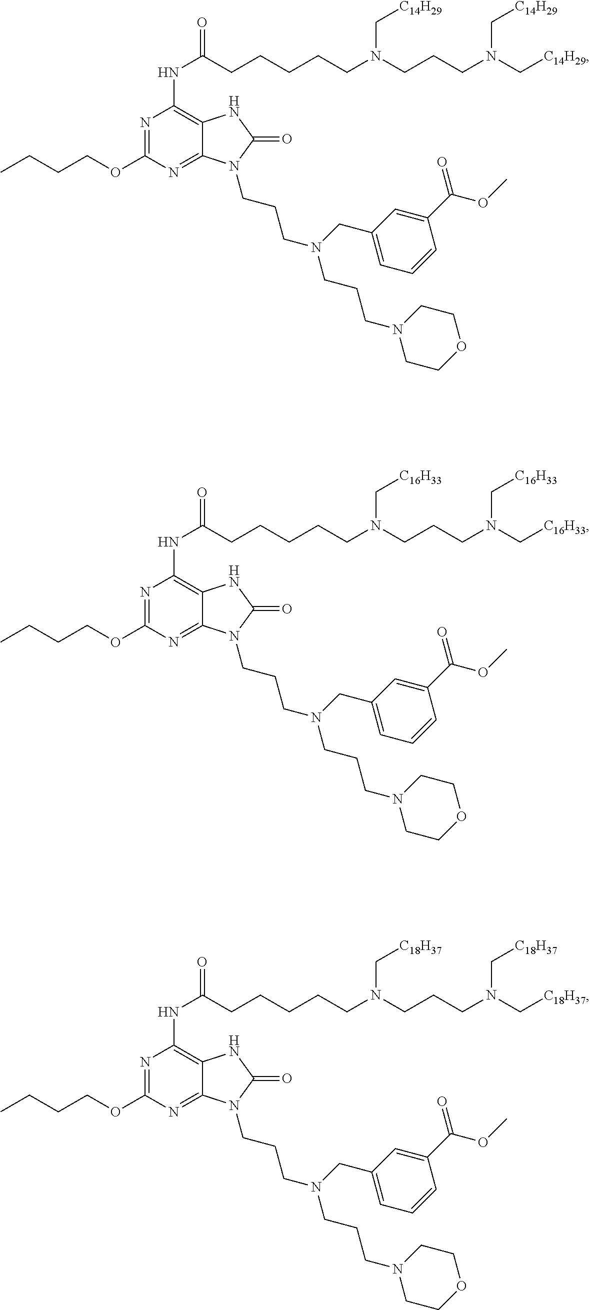

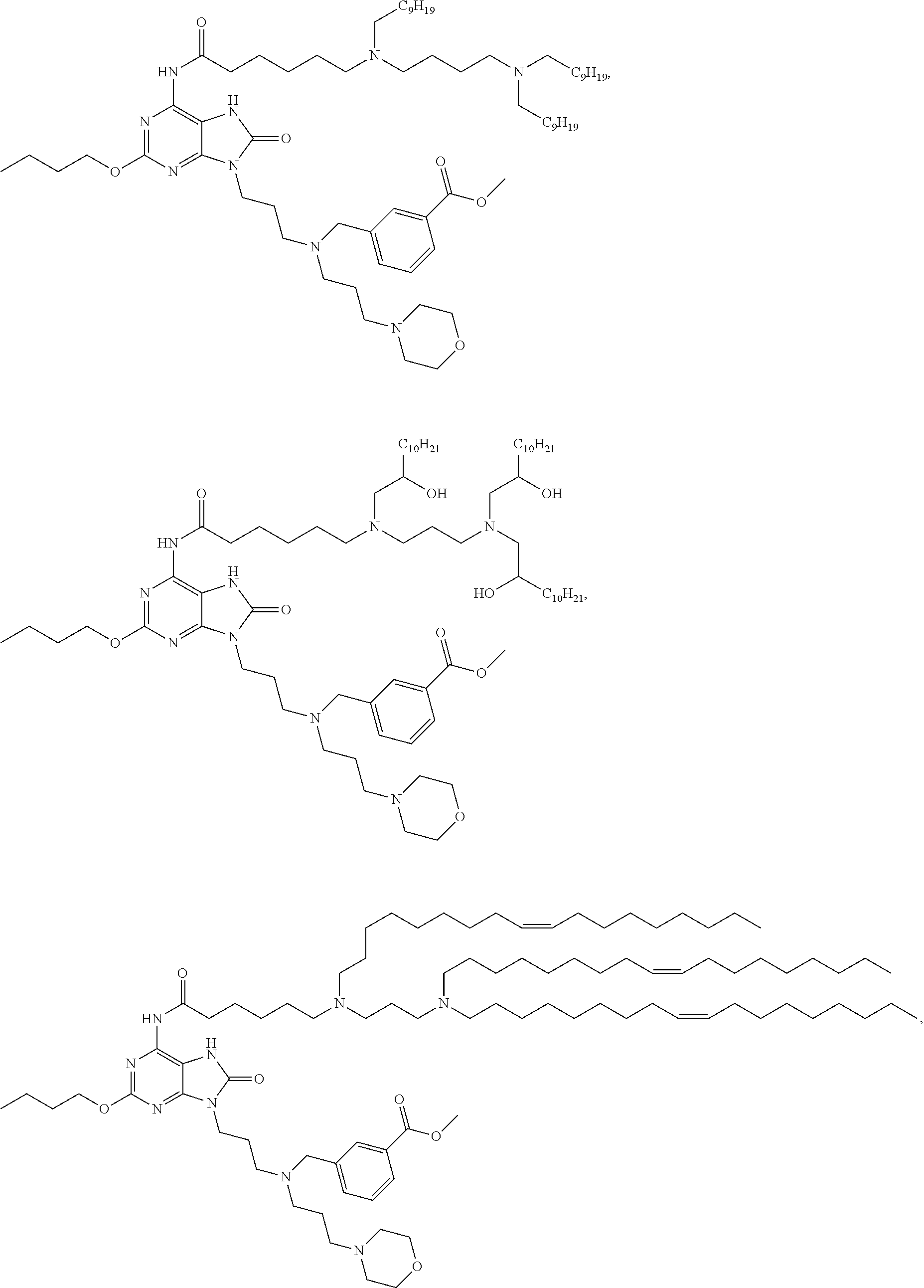

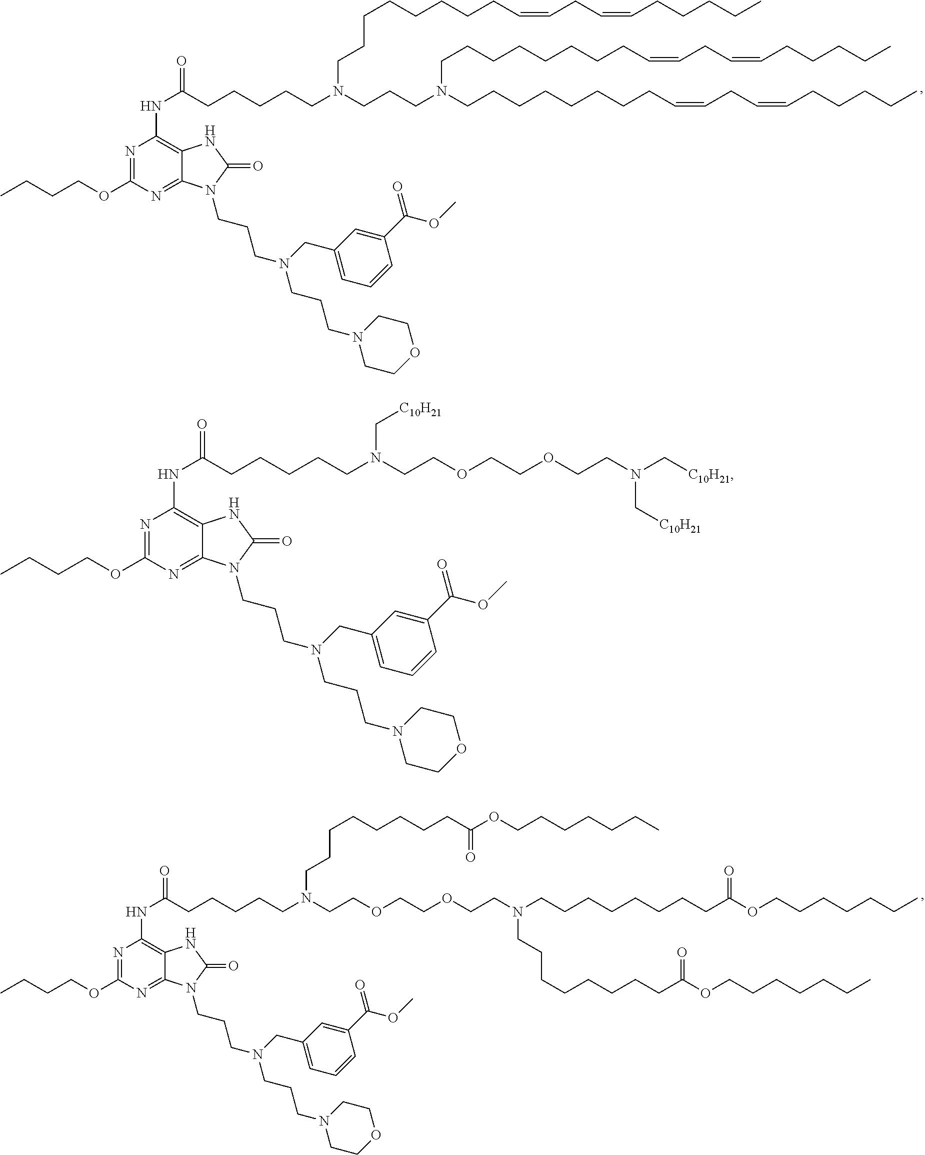

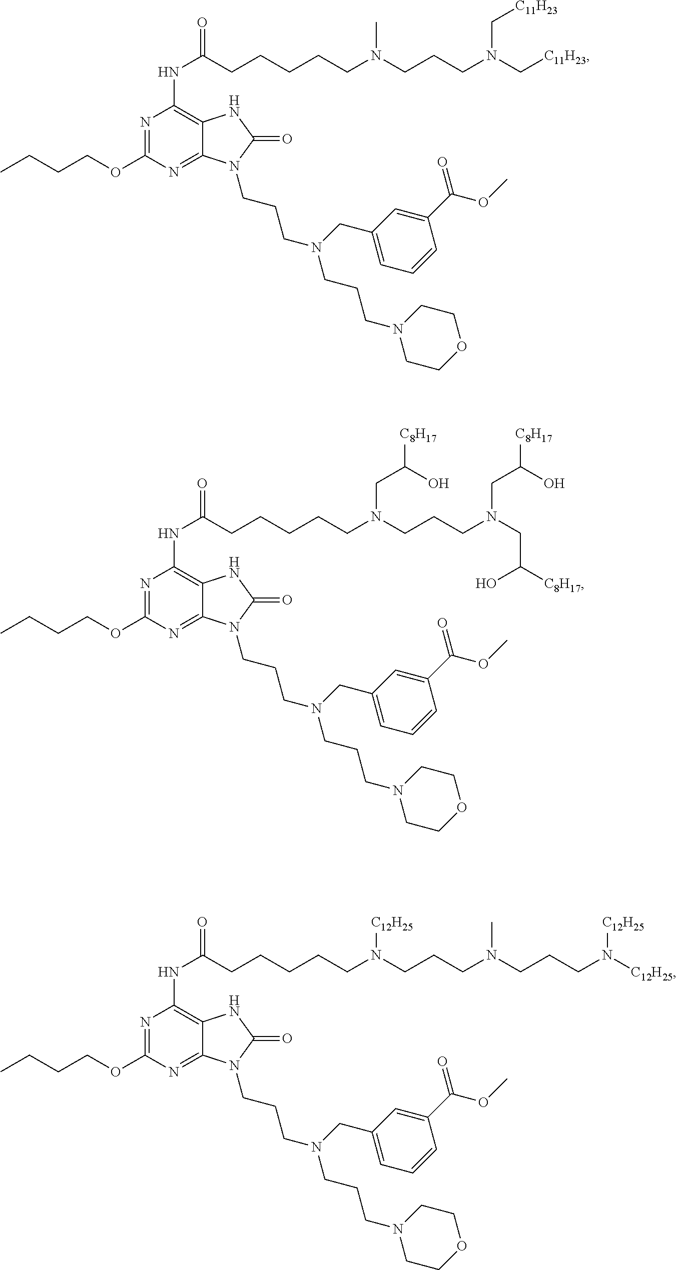

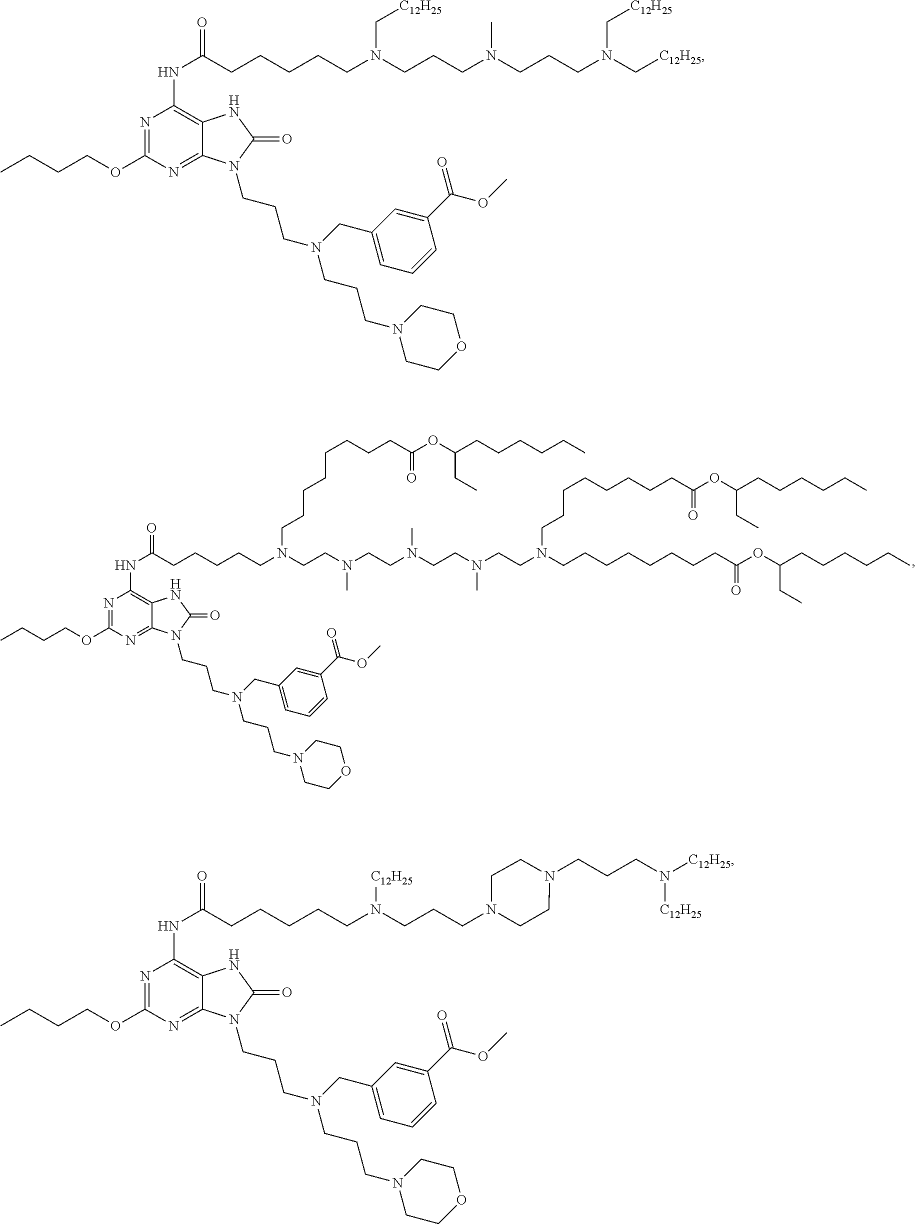

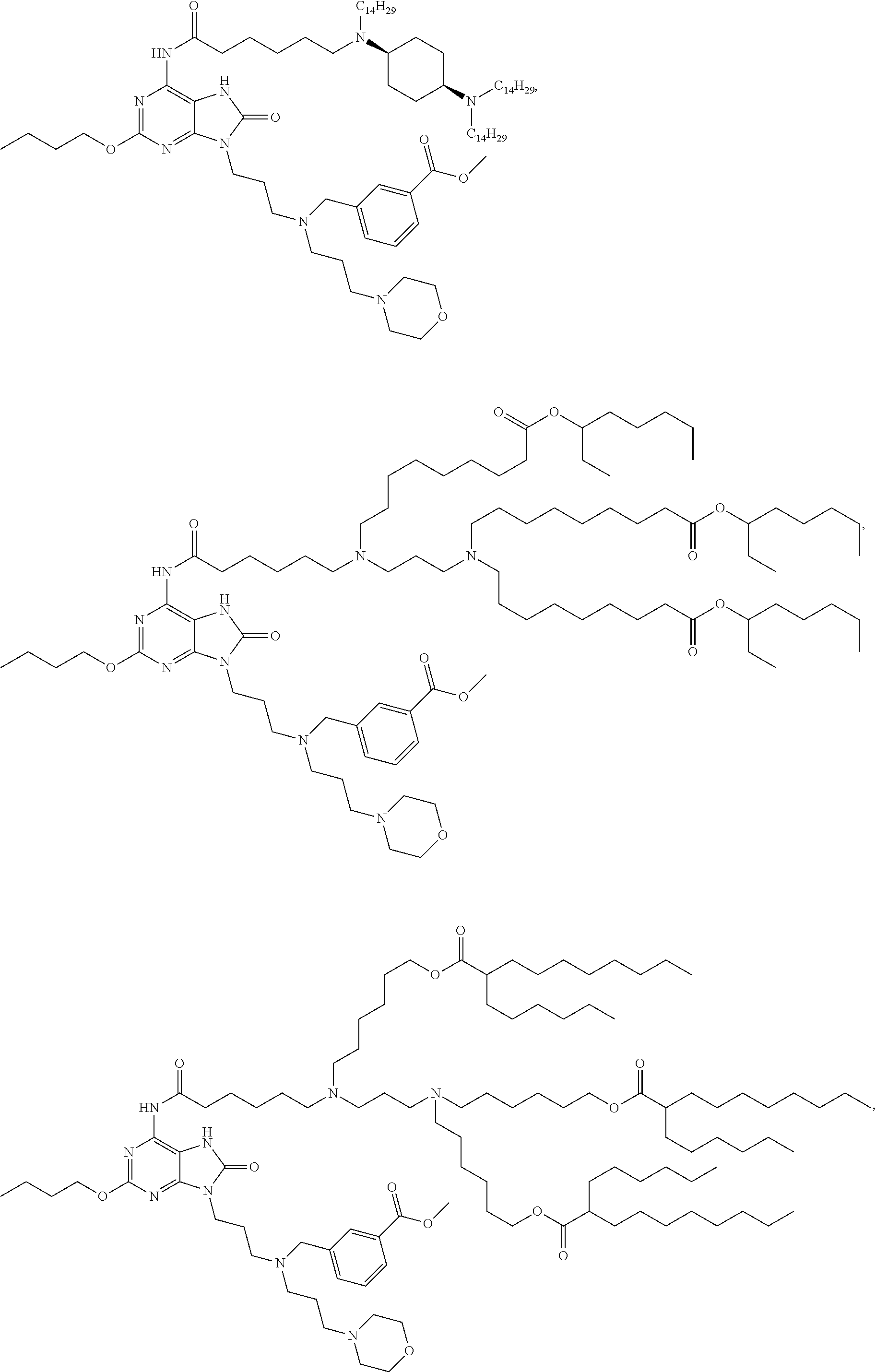

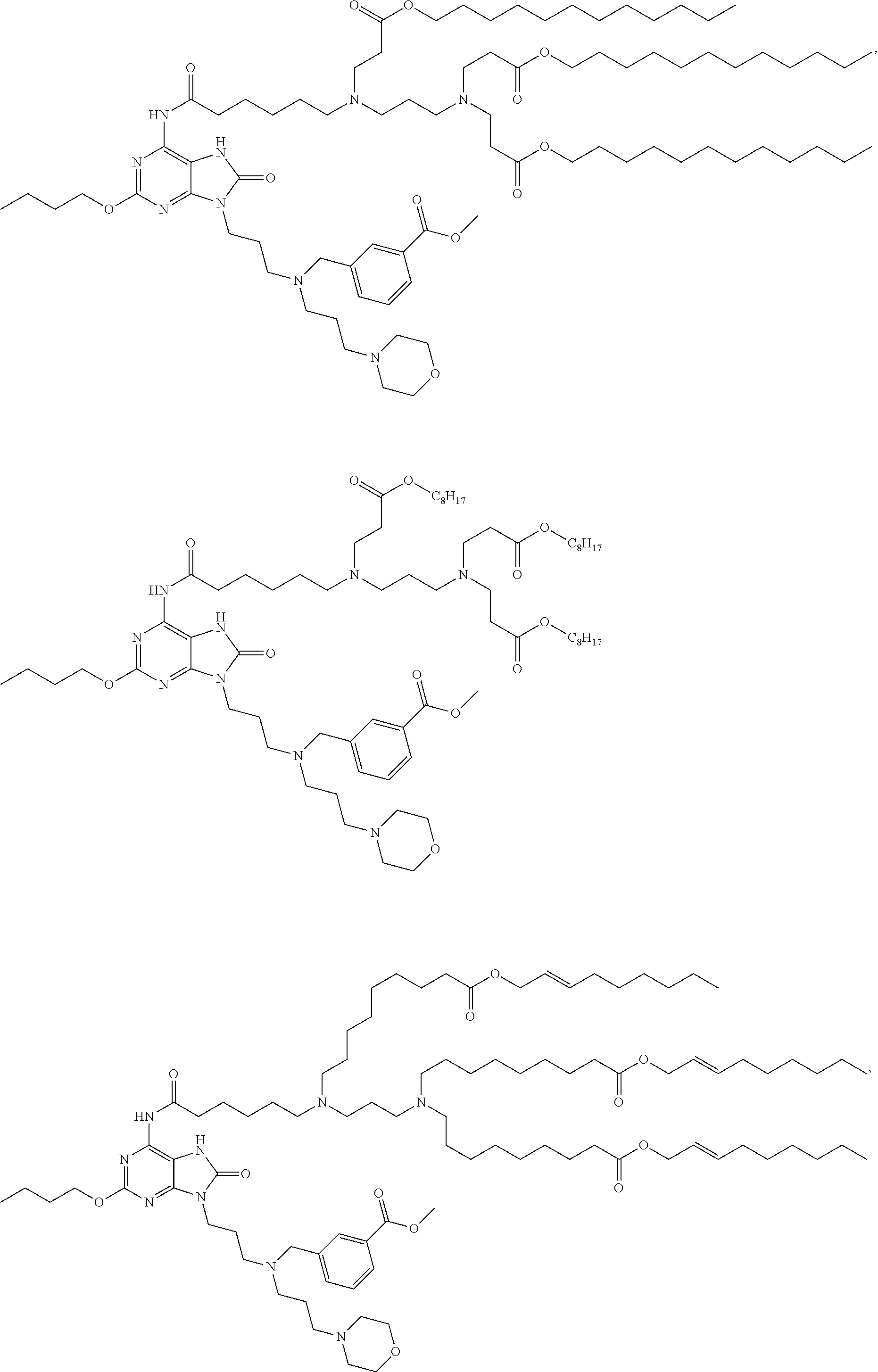

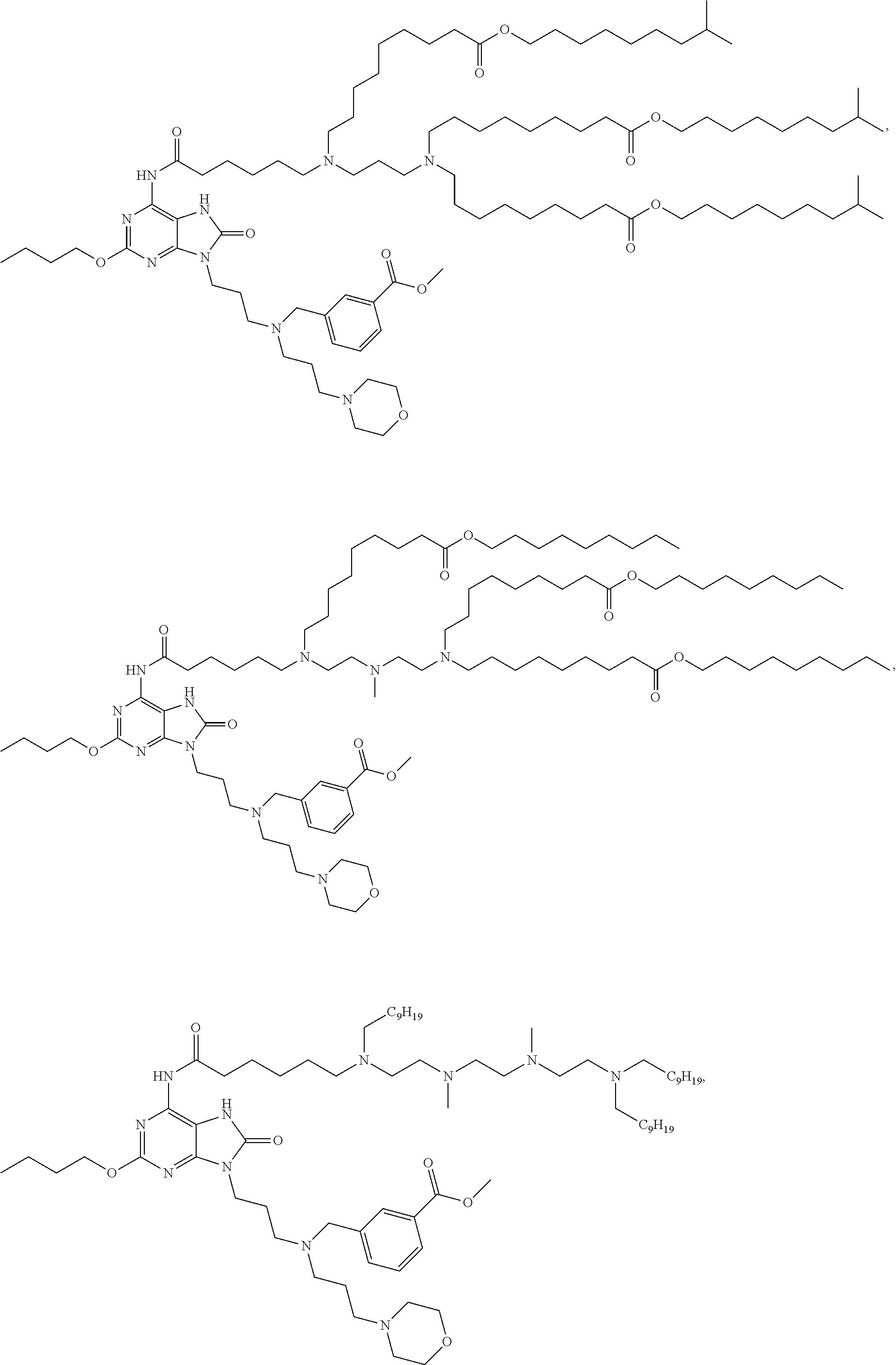

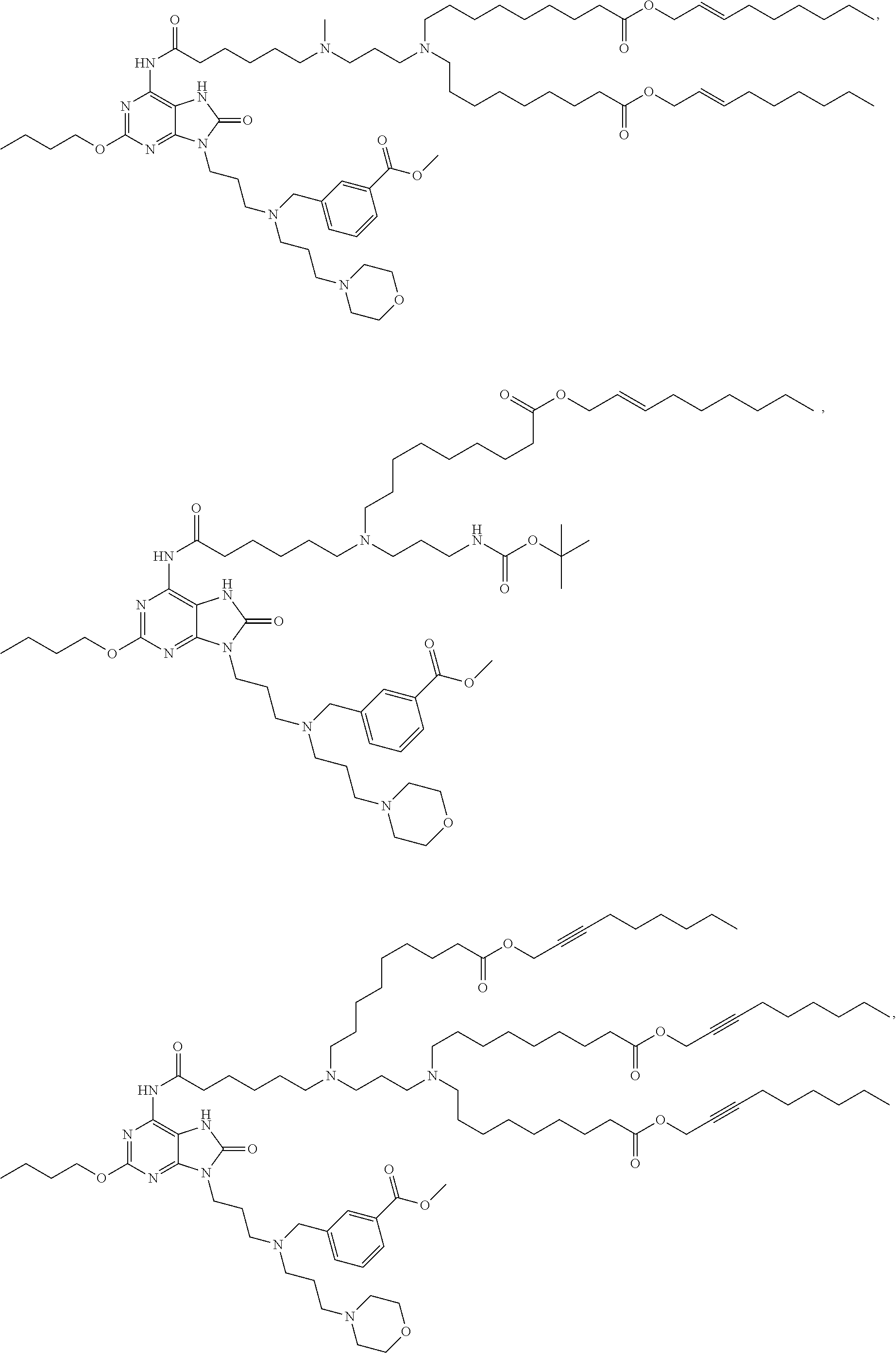

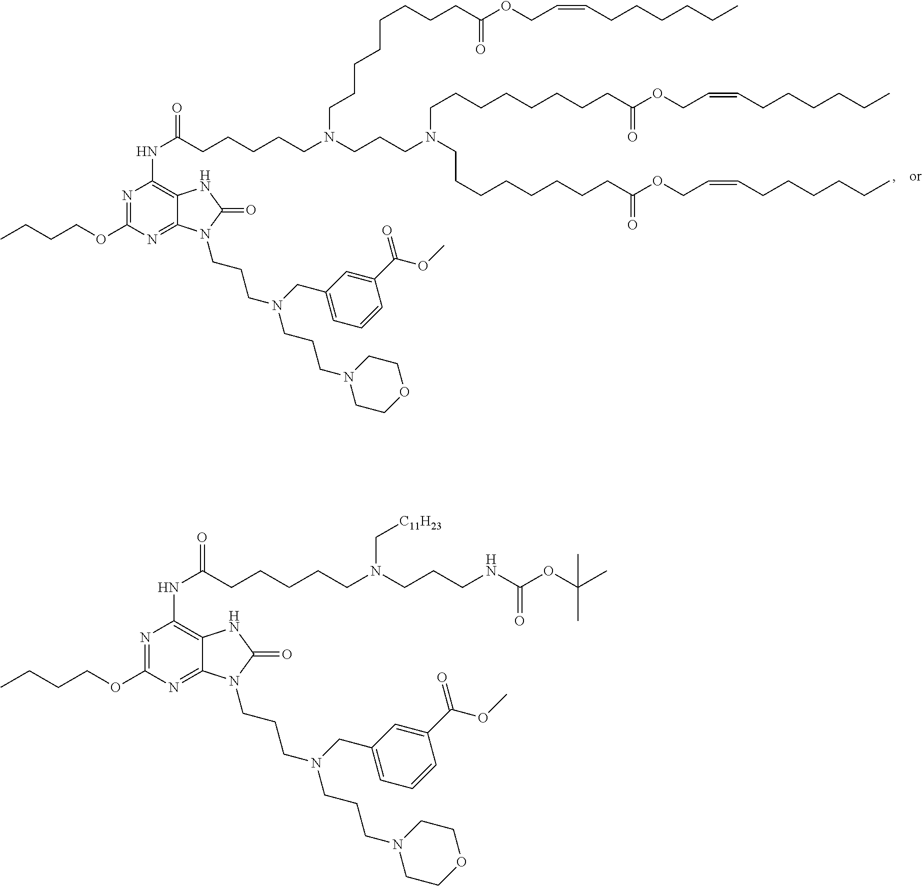

or a salt thereof.

In some embodiments, the compound is selected from the following:

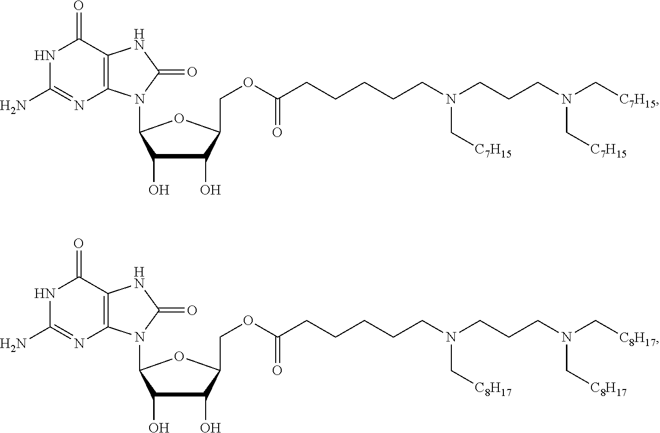

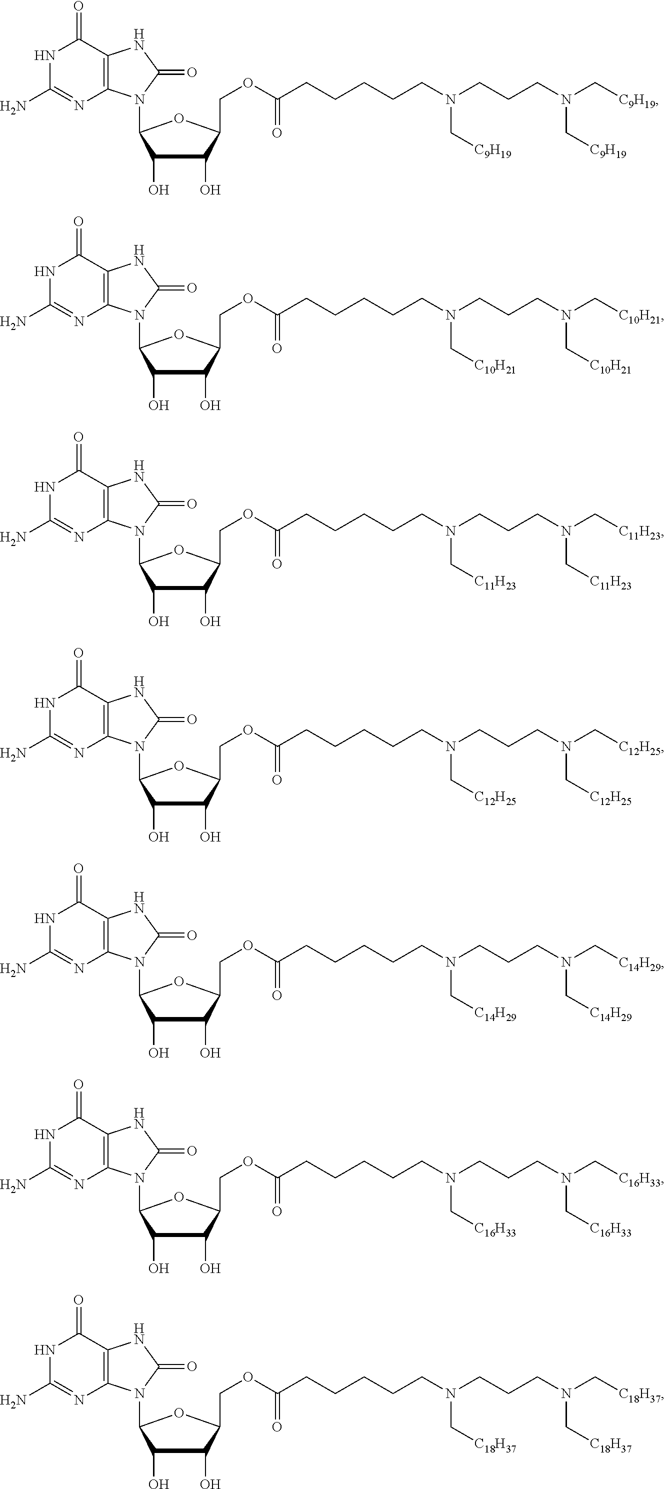

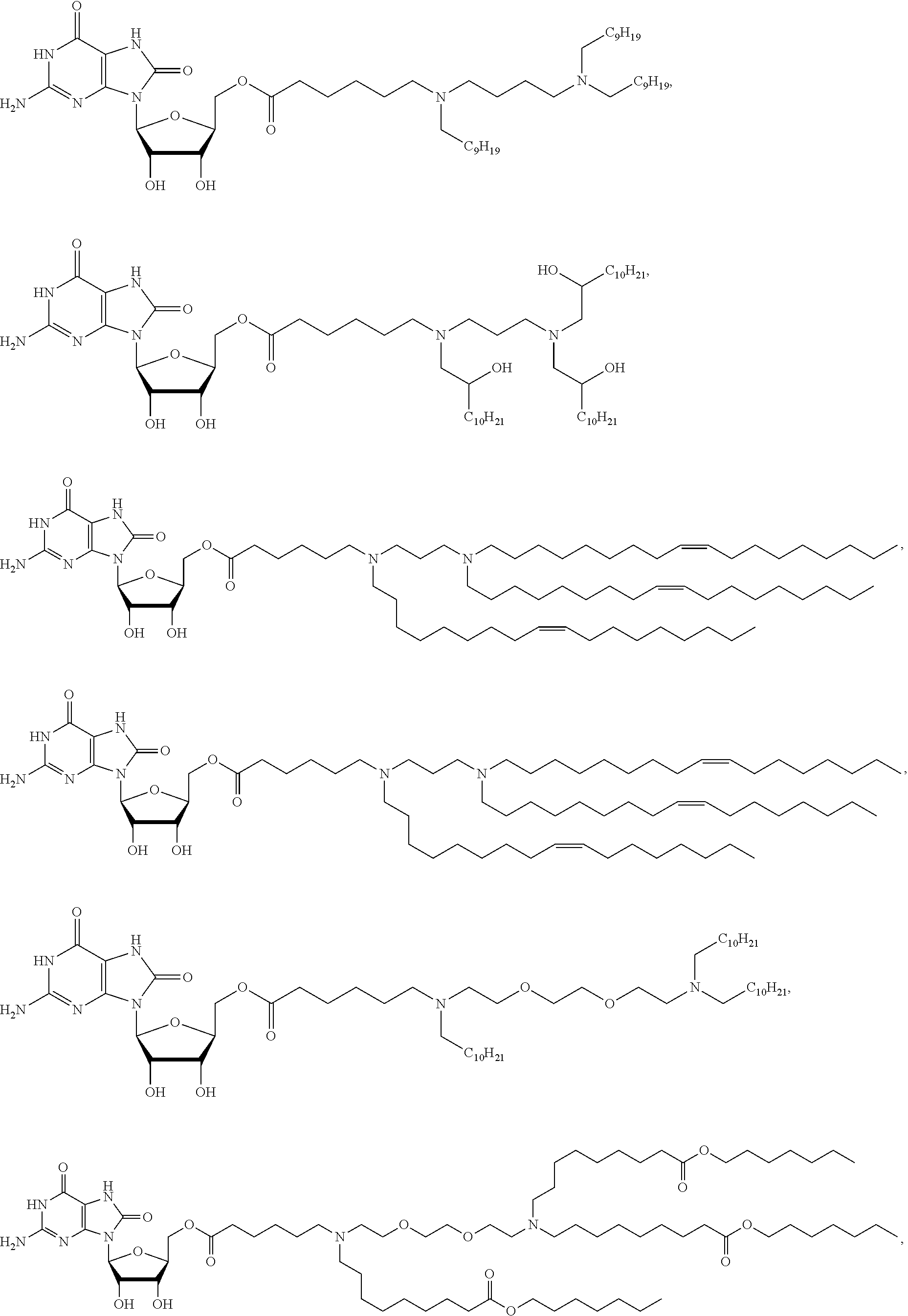

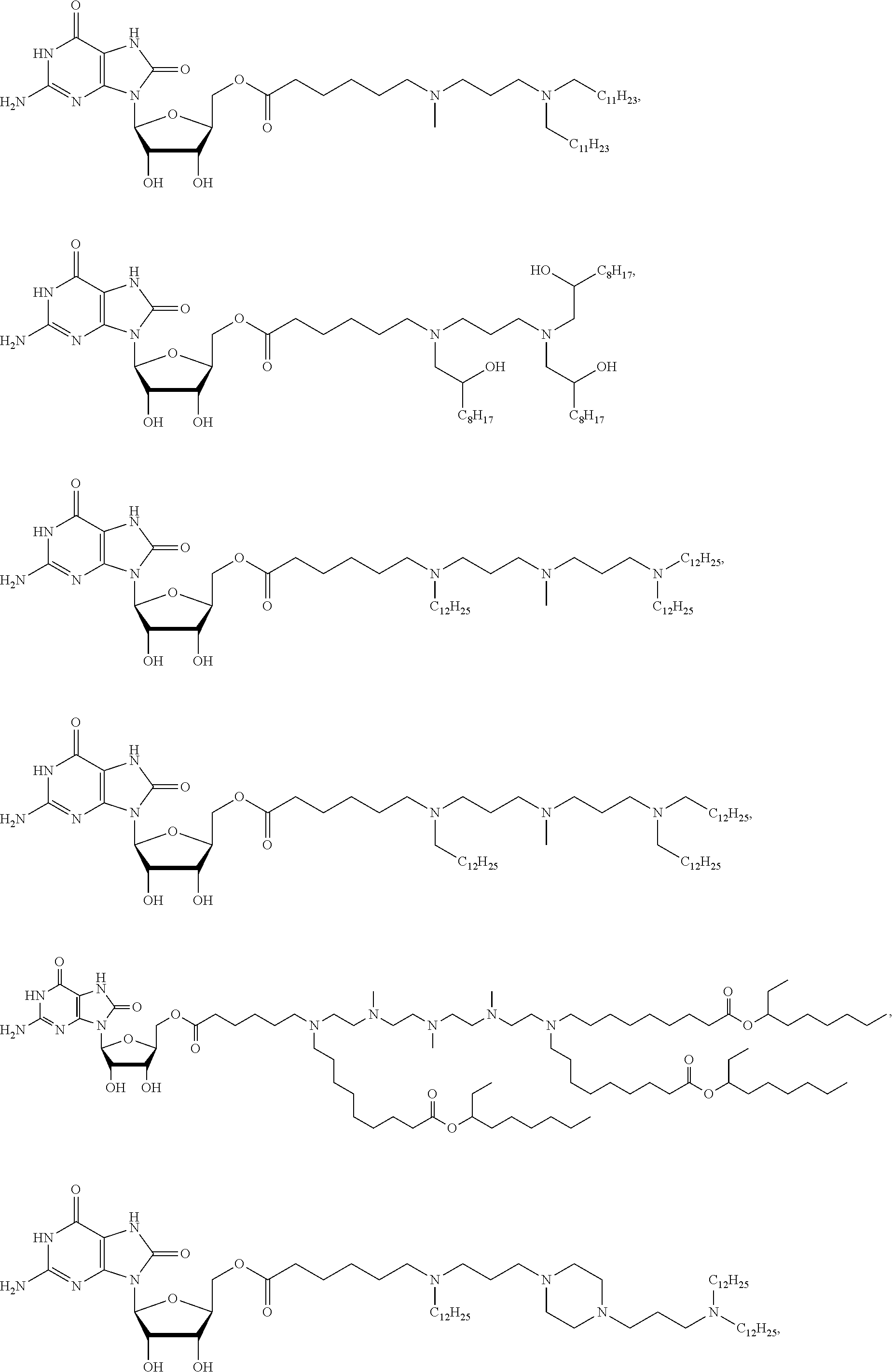

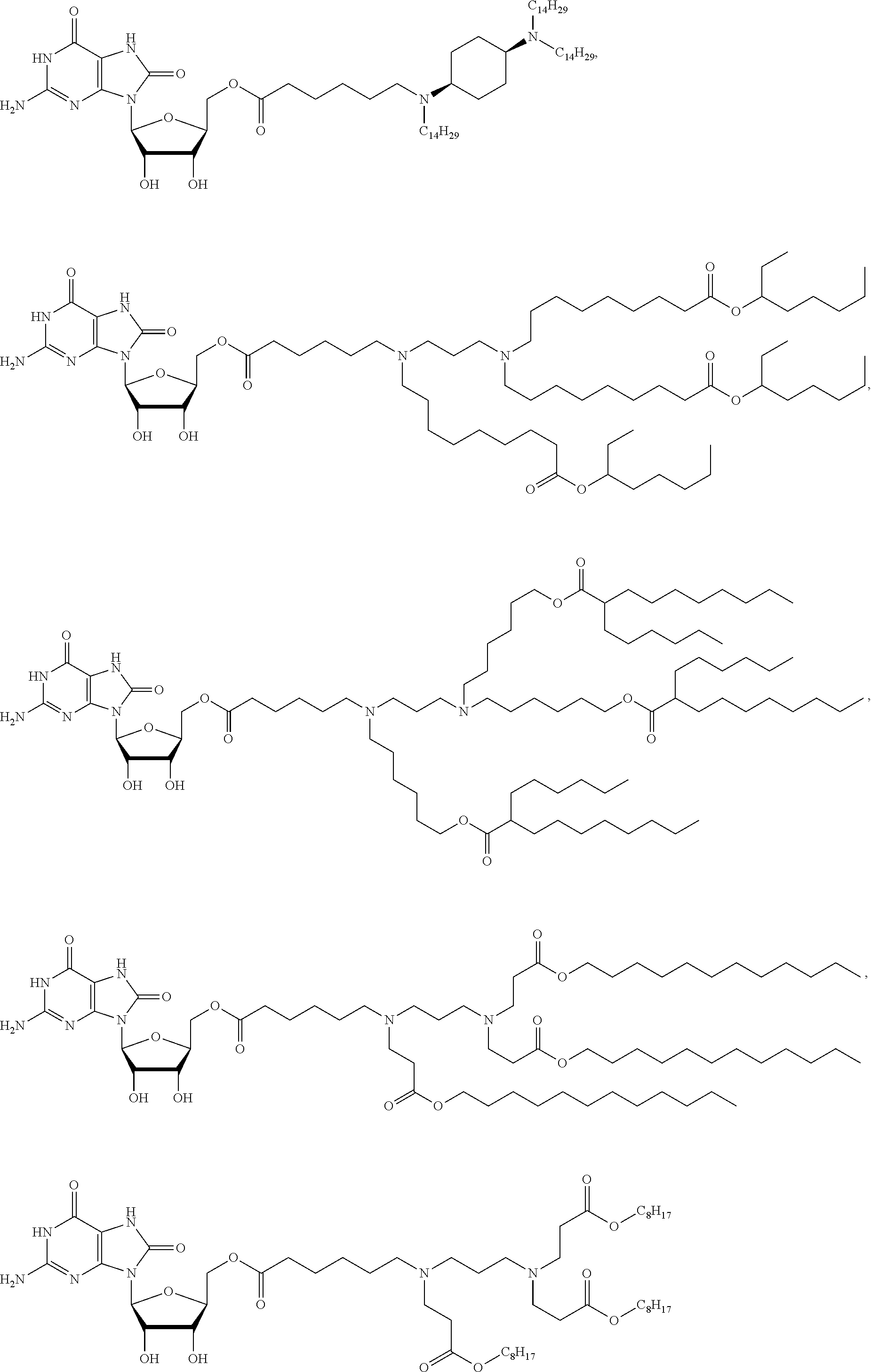

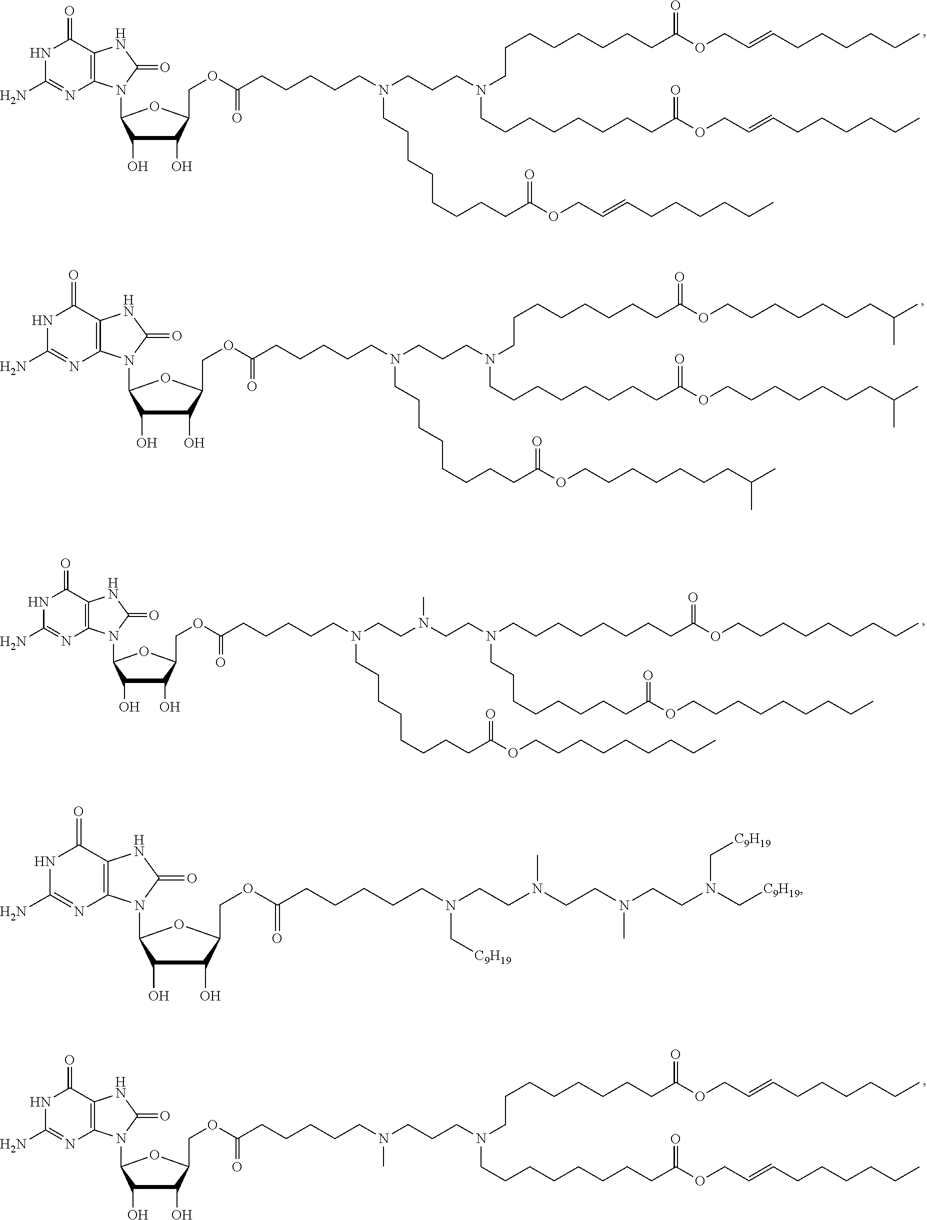

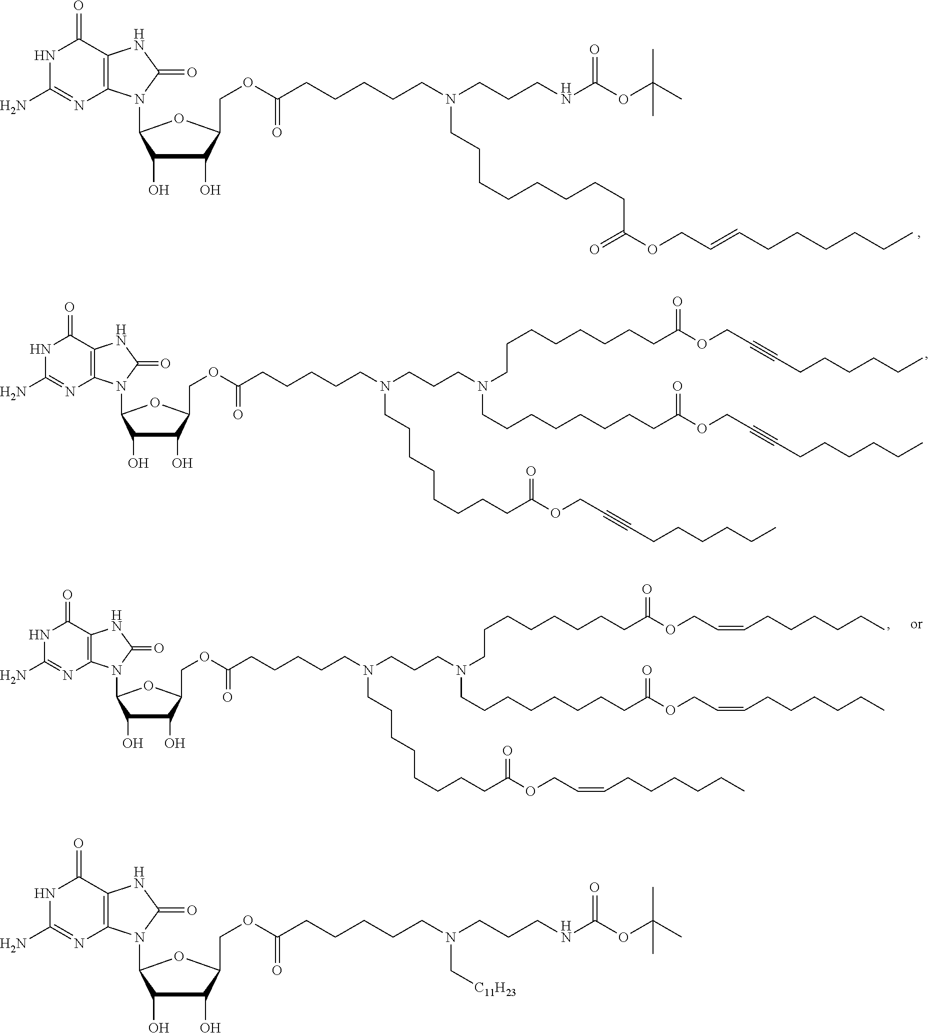

or a salt thereof.

In another aspect, disclosed herein is a composition comprising:

a compound of Formula A, I, II, III, IV, V, VI, VII, VIII, IX, or X; and

an agent.

In some embodiments, the agent is a nucleic acid. In some embodiments, the agent is a polynucleotide. In some embodiments, the agent is an RNA. In some embodiments, the agent is an mRNA.

### Nanoparticles

In one aspect, the disclosure provides a nanoparticle comprising:

a compound of Formula A, I, II, III, IV, V, VI, VII, VIII, IX, or X;

a non-cationic lipid;

a polyethylene glycol-lipid; and

a sterol.

In one aspect, the disclosure provides a nanoparticle comprising: a compound of Formula A; a non-cationic lipid; a polyethylene glycol-lipid; and a sterol.

In one aspect, the disclosure provides a nanoparticle comprising: a compound of Formula I; a non-cationic lipid; a polyethylene glycol-lipid; and a sterol.

In one aspect, the disclosure provides a nanoparticle comprising: a compound of Formula II; a non-cationic lipid; a polyethylene glycol-lipid; and a sterol.

In one aspect, the disclosure provides a nanoparticle comprising: a compound of Formula III; a non-cationic lipid; a polyethylene glycol-lipid; and a sterol.

In one aspect, the disclosure provides a nanoparticle comprising: a compound of Formula IV; a non-cationic lipid; a polyethylene glycol-lipid; and a sterol.

In one aspect, the disclosure provides a nanoparticle comprising: a compound of Formula V; a non-cationic lipid; a polyethylene glycol-lipid; and a sterol.

In one aspect, the disclosure provides a nanoparticle comprising: a compound of Formula VI; a non-cationic lipid; a polyethylene glycol-lipid; and a sterol.

In one aspect, the disclosure provides a nanoparticle comprising: a compound of Formula VII; a non-cationic lipid; a polyethylene glycol-lipid; and a sterol.

In one aspect, the disclosure provides a nanoparticle comprising: a compound of Formula VIII; a non-cationic lipid; a polyethylene glycol-lipid; and a sterol.

In one aspect, the disclosure provides a nanoparticle comprising: a compound of Formula IX; a non-cationic lipid; a polyethylene glycol-lipid; and a sterol.

In one aspect, the disclosure provides a nanoparticle comprising: a compound of Formula X; a non-cationic lipid; a polyethylene glycol-lipid; and a sterol.

The various compounds of Formula A, I, II, III, IV, V, VI, VII, VIII, IX, or X are described in the Compounds section above. In some embodiments, the nanoparticle comprises a compound of Formula A, I, II, III, IV, V, VI, VII, VIII, IX, or X in a molar ratio of about 10% to about 40%. In some embodiments, the nanoparticle comprises a compound of Formula A, I, II, III, IV, V, VI, VII, VIII, IX, or X in a molar ratio of about 10%, about 15%, about 20%, about 25%, about 30%, about 35%, or about 40%. In one embodiment, the nanoparticle comprises a compound of Formula A, I, II, III, IV, V, VI, VII, VIII, IX, or X in a molar ratio of about 20%.

In some embodiments, the nanoparticle comprises a non-cationic lipid. In some embodiments, the non-cationic lipid interacts with the lipids as a helper lipid. In some embodiments, the non-cationic lipid can include, but is not limited to, 1,2-dioleoyl-sn-glycero-3-phosphoethanolamine (DOPE), 1-palmitoyl-2-oleoyl-sn-glycero-3-phosphoethanolamine (POPE), 1,2-distearoyl-sn-glycero-3-phosphocholine (DSPC), 1-stearoyl-2-oleoyl-sn-glycero-3-phosphoethanolamine (SOPE), DPPC (1,2-dipalmitoyl-sn-glycero-3-phosphocholine), 1,2-dioleyl-sn-glycero-3-phosphotidylcholine (DOPC), 1,2-dipalmitoyl-sn-glycero phosphoethanolamine (DPPE), 1,2-dimyristoyl-sn-glycero-3-phosphoethanolamine (DMPE), 1,2-dioleoyl-5/7-glycero-3-phospho-(1′-rac-glycerol) (DOPG), or combinations thereof. In one embodiment, the non-cationic lipid is 1,2-dioleoyl-sn-glycero-3-phosphoethanolamine (DOPE). In one embodiment, the non-cationic lipid is 1-palmitoyl-2-oleoyl-sn-glycero-3-phosphoethanolamine (POPE), In one embodiment, the non-cationic lipid is 1,2-distearoyl-sn-glycero-3-phosphocholine (DSPC). In one embodiment, the non-cationic lipid is 1-stearoyl-2-oleoyl-sn-glycero-3-phosphoethanolamine (SOPE). While several non-cationic lipids are described here, additional non-cationic lipids can be used in combination with the compounds disclosed herein.

In some embodiments, the nanoparticle comprises a non-cationic lipid in a molar ratio of about 10% to about 40%. In some embodiments, the nanoparticle comprises a non-cationic lipid in a molar ratio of about 10%, about 15%, about 20%, about 25%, about 30%, about 35%, or about 40%. In one embodiment, the nanoparticle comprises a non-cationic lipid in a molar ratio of about 30%.

In some embodiments, the nanoparticle includes a polyethylene glycol-lipid (PEG-lipid). PEG-lipid is incorporated to form a hydrophilic outer layer and stabilize the particles. Nonlimiting examples of polyethylene glycol-lipids include PEG-modified lipids such as PEG-modified phosphatidylethanolamines, PEG-modified phosphatidic acids, PEG-modified ceramides, PEG-modified dialkylamines, PEG-modified diacylglycerols, and PEG-modified dialkylglycerols. Representative polyethylene glycol-lipids include DMG-PEG, DLPE-PEGs, DMPE-PEGs, DPPC-PEGs, and DSPE-PEGs. In one embodiment, the polyethylene glycol-lipid is 1,2-dimyristoyl-sn-glycerol, methoxypolyethylene glycol (DMG-PEG). In one embodiment, the polyethylene glycol-lipid is 1,2-dimyristoyl-sn-glycerol, methoxypolyethylene glycol-2000 (DMG-PEG2000). DMG-PEGXXXX means 1,2-dimyristoyl-sn-glycerol, methoxypolyethylene glycol-XXXX, wherein XXXX signifies the molecular weight of the polyethylene glycol moiety, e.g. DMG-PEG2000 or DMG-PEG5000.

In some embodiments, the nanoparticle comprises a polyethylene glycol-lipid in a molar ratio of about 0% to about 5%. In some embodiments, the nanoparticle comprises a polyethylene glycol-lipid in a molar ratio of about 0%, about 0.25%, about 0.5%, about 0.75%, about 1%, about 1.5%, about 2%, about 3%, about 4%, or about 5%. In one embodiment, the nanoparticle comprises a polyethylene glycol-lipid in a molar ratio of about 0.75%.

In some embodiments, the nanoparticle includes a sterol. Sterols are well known to those skilled in the art and generally refers to those compounds having a perhydrocyclopentanophenanthrene ring system and having one or more OH substituents. Examples of sterols include, but are not limited to, cholesterol, campesterol, ergosterol, sitosterol, and the like.

In some embodiments, the sterol is selected from a cholesterol-based lipid. In some embodiments, the one or more cholesterol-based lipids are selected from cholesterol, PEGylated cholesterol, DC-Choi (N,N-dimethyl-N-ethylcarboxamidocholesterol), 1,4-bis(3-N-oleylamino-propyl)piperazine, or combinations thereof.

The sterol can be used to tune the particle permeability and fluidity base on its function in cell membranes. In one embodiment, the sterol is cholesterol.

In some embodiments, the nanoparticle comprises a sterol in a molar ratio of about 25% to about 50%. In some embodiments, the nanoparticle comprises a sterol in a molar ratio of about 25%, about 30%, about 35%, about 40%, about 45%, or about 50%. In one embodiment, the nanoparticle comprises a sterol in a molar ratio of about 40%.

In one embodiment, the disclosure provides a nanoparticle comprising:
a compound of Formula A, I, II, III, IV, V, VI, VII, VIII, IX, or X;
1,2-dioleoyl-sn-glycero-3-phosphoethanolamine (DOPE);
1,2-dimyristoyl-sn-glycerol, methoxypolyethylene glycol (DMG-PEG2000); and cholesterol.

In one embodiment, the disclosure provides a nanoparticle comprising:
a compound of Formula A, I, II, III, IV, V, VI, VII, VIII, IX, or X;
1-palmitoyl-2-oleoyl-sn-glycero-3-phosphoethanolamine (POPE);
1,2-dimyristoyl-sn-glycerol, methoxypolyethylene glycol (DMG-PEG2000); and cholesterol.

In one embodiment, the disclosure provides a nanoparticle comprising:
a compound of Formula A, I, II, III, IV, V, VI, VII, VIII, IX, or X;
1,2-distearoyl-sn-glycero-3-phosphocholine (DSPC);
1,2-dimyristoyl-sn-glycerol, methoxypolyethylene glycol (DMG-PEG2000); and cholesterol.

In one embodiment, the nanoparticle further comprises an agent. In one embodiment, the nanoparticle further comprises a therapeutic agent. In one embodiment, the nanoparticle further comprises a diagnostic agent.

The agents delivered into cells can be a polynucleotide. Polynucleotides or oligonucleotides that can be introduced according to the methods herein include DNA, cDNA, and RNA sequences of all types. For example, the polynucleotide can be double stranded DNA, single-stranded DNA, complexed DNA, encapsulated DNA, naked RNA, encapsulated RNA, messenger RNA (mRNA), tRNA, short interfering RNA (siRNA), double stranded RNA (dsRNA), micro-RNA (miRNA), antisense RNA (asRNA) and combinations thereof. The polynucleotides can also be DNA constructs, such as expression vectors, expression vectors encoding a desired gene product (e.g., a gene product homologous or heterologous to the subject into which it is to be introduced), and the like. In one embodiment, the agent is an mRNA.

### Compositions

Compositions, as described herein, comprising an active compound and an excipient of some sort may be useful in a variety of medical and non-medical applications. For example, pharmaceutical compositions comprising an active compound and an excipient may be useful in the delivery of an effective amount of an agent to a subject in need thereof. Nutraceutical compositions comprising an active compound and an excipient may be useful in the delivery of an effective amount of a nutraceutical, e.g., a dietary supplement, to a subject in need thereof. Cosmetic compositions comprising an active compound and an excipient may be formulated as a cream, ointment, balm, paste, film, or liquid, etc., and may be useful in the application of make-up, hair products, and materials useful for personal hygiene, etc. Compositions comprising an active compound and an excipient may be useful for non-medical applications, e.g., such as an emulsion or emulsifier, useful, for example, as a food component, for extinguishing fires, for disinfecting surfaces, for oil cleanup, etc.

In certain embodiments, the composition further comprises an agent, as described herein. For example, in certain embodiments, the agent is a small molecule, organometallic compound, nucleic acid, protein, peptide, polynucleotide, metal, targeting agent, an isotopically labeled chemical compound, drug, vaccine, immunological agent, or an agent useful in bioprocessing. In certain embodiments, the agent is a polynucleotide. In certain embodiments, the polynucleotide is DNA or RNA. In certain embodiments, the RNA is RNAi, dsRNA, siRNA, shRNA, miRNA, or antisense RNA. In certain embodiments, the polynucleotide and the one or more active compounds are not covalently attached.

In one aspect, the disclosure provides a composition comprising:

a compound of Formula A, I, II, III, IV, V, VI, VII, VIII, IX, or X; and

an agent.

In one aspect, the disclosure provides a composition comprising:

a nanoparticle, comprising a compound of Formula A, I, II, III, IV, V, VI, VII, VIII, IX, or X; and

an agent.

In another aspect, disclosed herein is a composition comprising:

a nanoparticle, comprising a compound of Formula A, I, II, III, IV, V, VI, VII, VIII, IX, or X; and an agent,

wherein the agent comprises an mRNA encoding at least one antigenic polypeptide or an immunogenic fragment thereof capable of inducing an immune response to the antigenic polypeptide.

In some embodiments, the mRNA encoding at least one antigenic polypeptide or an immunogenic fragment thereof capable of inducing an immune response to the antigenic polypeptide is encapsulated by the nanoparticle.

In some aspects, disclosed herein is a pharmaceutical composition comprising a pharmaceutically acceptable carrier and a nanoparticle comprising an mRNA at least one antigenic polypeptide or an immunogenic fragment thereof capable of inducing an immune response to the antigenic polypeptide.

In another aspect, disclosed herein is a composition comprising:

a nanoparticle, comprising a compound of Formula A, I, II, III, IV, V, VI, VII, VIII, IX, or X; and an agent,

wherein the agent comprises an mRNA encoding the co-stimulatory molecule; and

an antibody, a ligand, or an antigen binding fragment thereof that specifically binds a co-stimulatory molecule.

In some embodiments, the mRNA encoding the co-stimulatory molecule is encapsulated by the nanoparticle.

In some aspects, disclosed herein is a pharmaceutical composition comprising a pharmaceutically acceptable carrier and an effective amount of an antibody, a ligand, or an antigen binding fragment thereof that specifically binds a co-stimulatory molecule and a nanoparticle comprising an mRNA encoding the co-stimulatory molecule.

In some embodiments, the composition further comprises an immunotherapeutic agent. In some embodiments, the immunotherapeutic agent is selected from an anti-CD40 antibody, anti-PDL1 antibody, an anti-PD1 antibody, an anti-CTLA4 antibody, or a combination thereof.

In some aspects, disclosed herein is a pharmaceutical composition comprising a pharmaceutically acceptable carrier and an effective amount of an antibody, a ligand, or an antigen binding fragment thereof that specifically binds a co-stimulatory molecule and a nanoparticle comprising an mRNA encoding the co-stimulatory molecule.

In some aspects, disclosed herein is a pharmaceutical composition comprising a pharmaceutically acceptable carrier and an effective amount of an antibody or antigen binding fragment thereof that specifically binds a co-stimulatory molecule and a nanoparticle comprising an mRNA encoding the co-stimulatory molecule.

### Agents

Agents to be delivered by the compounds, compositions, and systems described herein may be therapeutic, diagnostic, or prophylactic agents. Any chemical compound to be administered to a subject may be delivered using the particles or nanoparticles described herein. The agent may be an organic molecule (e.g., a therapeutic agent, a drug), inorganic molecule, nucleic acid, protein, amino acid, peptide, polypeptide, polynucleotide, targeting agent, isotopically labeled organic or inorganic molecule, vaccine, immunological agent, etc.

In certain embodiments, the agents are organic molecules with pharmaceutical activity, e.g., a drug. In certain embodiments, the drug is an antibiotic, anti-viral agent, anesthetic, steroidal agent, anti-inflammatory agent, anti-neoplastic agent, anti-cancer agent, antigen, vaccine, antibody, decongestant, antihypertensive, sedative, birth control agent, progestational agent, anti-cholinergic, analgesic, anti-depressant, anti-psychotic, f3-adrenergic blocking agent, diuretic, cardiovascular active agent, vasoactive agent, non-steroidal anti-inflammatory agent, nutritional agent, etc.

In certain embodiments of the present disclosure, the agent to be delivered may be a mixture of agents.

Diagnostic agents include gases; metals; commercially available imaging agents used in positron emissions tomography (PET), computer assisted tomography (CAT), single photon emission computerized tomography, x-ray, fluoroscopy, and magnetic resonance imaging (MRI); and contrast agents. Examples of suitable materials for use as contrast agents in MM include gadolinium chelates, as well as iron, magnesium, manganese, copper, and chromium. Examples of materials useful for CAT and x-ray imaging include iodine-based materials.

Therapeutic and prophylactic agents include, but are not limited to, antibiotics, nutritional supplements, and vaccines. Vaccines may comprise isolated proteins or peptides, inactivated organisms and viruses, dead organisms and viruses, genetically altered organisms or viruses, cell extracts, and RNA encoding at least one antigenic polypeptide or an immunogenic fragment thereof (e.g., an immunogenic fragment capable of inducing an immune response to the antigenic polypeptide). Therapeutic and prophylactic agents may be combined with interleukins, interferon, cytokines, and adjuvants such as cholera toxin, alum, Freund's adjuvant, etc. Prophylactic agents include antigens of such bacterial organisms as Streptococccus pneumoniae, Haemophilus influenzae, Staphylococcus aureus, Streptococcus pyrogenes, Corynebacterium diphtheria, Listeria monocytogenes, Bacillus anthracis, Clostridium tetani, Clostridium botulinum, Clostridium perfringens, Neisseria meningitidis, Neisseria gonorrhoeae, Streptococcus mutans, Pseudomonas aeruginosa, Salmonella typhi, Haemophilus parainfluenzae, Bordetella pertussis, Francisella tularensis, Yersinia pestis, Vibrio cholerae, Legionella pneumophila, Mycobacterium tuberculosis, Mycobacterium leprae, Treponema pallidum, Leptospirosis interrogans, Borrelia burgdorferi, Camphylobacter jejuni, and the like; antigens of such viruses as smallpox, influenza A and B, respiratory syncytial virus, parainfluenza, measles, HIV, varicella-zoster, herpes simplex 1 and 2, cytomegalovirus, Epstein-Barr virus, rotavirus, rhinovirus, adenovirus, papillomavirus, poliovirus, mumps, rabies, rubella, coxsackieviruses, equine encephalitis, Japanese encephalitis, yellow fever, Rift Valley fever, hepatitis A, B, C, D, and E virus, and the like; antigens of fungal, protozoan, and parasitic organisms such as Cryptococcus neoformans, Histoplasma capsulatum, Candida albicans, Candida tropicalis, Nocardia asteroides, Rickettsia ricketsii, Rickettsia typhi, Mycoplasma pneumoniae, Chlamydial psittaci,

Chlamydial trachomatis, Plasmodium falciparum, Trypanosoma brucei, Entamoeba histolytica, Toxoplasma gondii, Trichomonas vaginalis, Schistosoma mansoni, and the like. These antigens may be in the form of whole killed organisms, peptides, proteins, glycoproteins, carbohydrates, or combinations thereof.

In some aspects, the agent is a ribonucleic acid (RNA) (e.g., mRNA) polynucleotide having an open reading frame encoding at least one (e.g., at least 2, 3, 4 or 5) antigenic polypeptide or an immunogenic fragment thereof (e.g., an immunogenic fragment capable of inducing an immune response to the antigenic polypeptide).

In some embodiments, the RNA (e.g., mRNA) maybe used to induce a balanced immune response against respiratory viruses. The term “respiratory viruses” refers herein to viruses causing respiratory diseases. For example, negative-sense, single-stranded RNA virus of the family Paramyxoviridae such as human Metapneumovirus (hMPV), human parainfluenza viruses (hPIV) types 1, 2, and 3 (hPIV1, hPIV2 and hPIV3, respectively), RSV, and Measles virus (MeV). Another example of respiratory viruses are coronaviruses. Coronaviruses are enveloped viruses with a positive-sense single-stranded RNA genome and with a nucleocapsid of helical symmetry. Coronaviruses are species of virus belonging to the subfamily Coronavirinae in the family Coronaviridae, in the order Nidovirales. Representative examples of betacoronaviruses include, but are not limited to an embecovirus 1 (e.g., Betacoronavirus 1, Human coronavirus OC43, China Rattus coronavirus HKU24, Human coronavirus HKU1, Murine coronavirus), a hibecovirus (e.g., Bat Hp-betacoronavirus Zhejiang2013), a merbecovirus (e.g., Hedgehog coronavirus 1, Middle East respiratory syndrome-related coronavirus (MERS-CoV), Pipistrellus bat coronavirus HKUS, Tylonycteris bat coronavirus HKU4), a nobecovirus (e.g., Rousettus bat coronavirus GCCDCl, Rousettus bat coronavirus HKU9), a sarbecovirus (e.g., severe acute respiratory syndrome coronavirus (SARS-CoV), severe acute respiratory syndrome coronavirus 2 (SARS-CoV-2).

Representative examples of gammacoronaviruses include, but are not limited to, a cegacovirus (e.g., Beluga whale coronavirus SQ1) and an Igacovirus (e.g., Avian coronavirus (IBV)).

Representative examples of deltacoronaviruses include, but are not limited to, an andecovirus (e.g., Wigeon coronavirus HKU20), a buldecovirus (e.g., Bulbul coronavirus HKU11, Porcine coronavirus HKU15 (PorCoV HKU15), Munia coronavirus HKU13, White-eye coronavirus HKU16), a herdecovirus (e.g., Night heron coronavirus HKU19), and a moordecovirus (e.g., Common moorhen coronavirus HKU21).

In some embodiments, the coronavirus is a human coronavirus. Representative examples of human coronaviruses include, but are not limited to, human coronavirus 229E (HCoV-229E), human coronavirus 0C43 (HCoV-OC43), human coronavirus HKU1 (HCoV-HKU1), Human coronavirus NL63 (HCoV-NL63), severe acute respiratory syndrome coronavirus (SARS-CoV), severe acute respiratory syndrome coronavirus 2 (SARS-CoV-2), and Middle East respiratory syndrome-related coronavirus (MERS-CoV).

In some embodiments, the RNA (e.g., mRNA) polynucleotide has an open reading frame encoding at least one (e.g., at least 2, 3, 4 or 5) hMPV, PIV, RSV, MeV, or a BetaCoV (e.g., MERS-CoV, SARS-CoV, SARS-CoV2, HCoV-OC43, HCoV-229E, HCoV-NL63, HCoV-NL, HCoV-NH, HCoV-HKU1) antigenic polypeptide, or any combination of two or more of the antigenic polypeptides. Herein, use of the term “antigenic polypeptide” encompasses immunogenic fragments of the antigenic polypeptide (an immunogenic fragment that induces (or is capable of inducing) an immune response to hMPV, PIV, RSV, MeV, or a BetaCoV), unless otherwise stated.

In some embodiments, the agent is an RNA (e.g., mRNA) vaccine that can induce a balanced immune response against hMPV, PIV, RSV, MeV, and/or BetaCoV (e.g., MERS-CoV, SARS-CoV, SARS-CoV2, HCoV-OC43, HCoV-229E, HCoV-NL63, HCoV-NL, HCoV-NH and/or HCoV-HKU1), or any combination of two or more of the foregoing viruses, comprising both cellular and humoral immunity, without risking the possibility of insertional mutagenesis, for example.

In some aspects, the agent is an mRNA encoding a co-stimulatory molecule.

In some embodiments, the co-stimulatory molecule is selected from ICOS, CD28, CD27, HVEM, LIGHT, CD40L, 4-1BB, OX40, DR3, GITR, CD30, SLAM, CD2, CD226, Galectin9, TIM1, LFA1, B7-H2, B7-1, B7-2, CD70, LIGHT, HVEM, CD40, 4-1BBL, OX40L, TL1A, GITRL, CD30L, SLAM, CD48, CD58, CD155, CD112, CD80, CD86, ICOSL, TIM4, ICAM1, or LFA3.

In some embodiments, the co-stimulatory molecule comprises OX40. In some embodiments, the co-stimulatory molecule comprises 4-1BB (CD137). In some embodiments, the co-stimulatory molecule comprises CD30. In some embodiments, the co-stimulatory molecule comprises CD2. In some embodiments, the co-stimulatory molecule comprises B7-H2. In some embodiments, the co-stimulatory molecule comprises B7-1. In some embodiments, the co-stimulatory molecule comprises B7-2. In some embodiments, the co-stimulatory molecule comprises CD70. In some embodiments, the co-stimulatory molecule comprises CD40. In some embodiments, the co-stimulatory molecule comprises 4-1BBL. In some embodiments, the co-stimulatory molecule comprises OX40L.

The sequences for the co-stimulatory molecules include, for example (for human sequences): ICOS (NCBI Reference Sequence: NM 012092.3), CD28 (NCBI Reference Sequence: NM 006139.4), CD27 (NCBI Reference Sequence: NM 001242.4), HVEM (NCBI Reference Sequence: NM 003820.3), LIGHT (NCBI Reference Sequence: NM 003807.4), CD40L (NCBI Reference Sequence: NM 000074.2), 4-1BB (NCBI Reference Sequence: NM 001561.5), OX40 (NCBI Reference Sequence: NM 003327.4), DR3 (NCBI Reference Sequence: NM 148965.1), GITR (NCBI Reference Sequence: NM 004195.3), CD30 (GenBank: M83554.1), SLAM (NCBI Reference Sequence: NM 003037.4), CD2 (NCBI Reference Sequence: NM 001328609.1), CD226 (NCBI Reference Sequence: NM 006566.3), Galectin-9 (GenBank: AB040130.2), TIM1 (GenBank: U02082.1), B7-H2 (NCBI Reference Sequence: NM 015259.5), B7-1 (NCBI Reference Sequence: NM 005191.4), B7-2 (NCBI Reference Sequence: NM 175862.5), CD70 (NCBI Reference Sequence: NM 001252.5), CD40 (NCBI Reference Sequence: NM 001250.5), 4-1BBL (NCBI Reference Sequence: NM 003811.4), OX40L (NCBI Reference Sequence: NM 003326.5), TL1A (NCBI Reference Sequence: NM 005118.4), GITRL (GenBank: AY358868.1), CD30L (NCBI Reference Sequence: NM 001244.3), SLAM (GenBank: U33017.1), CD48 (NCBI Reference Sequence: NM 001778.4), CD58 (NCBI Reference Sequence: NM 001779.3), CD155 (NCBI Reference Sequence: NM 006505.5), CD112 (NCBI Reference Sequence: NM 001042724.2), TIM3 (GenBank: AF450242.1), TIM4 (NCBI Reference Sequence: NM 138379.3), ICAM1 (NCBI Reference Sequence: NM 000201.3).

In some embodiments, the antibody or antigen binding fragment thereof that specifically binds a co-stimulatory molecule is BMS 986178. In some embodiments, the antibody or antigen binding fragment thereof that specifically binds a co-stimulatory molecule is GSK3174998. In some embodiments, the antibody or antigen binding fragment thereof that specifically binds a co-stimulatory molecule is PF-04518600. In some embodiments, the antibody or antigen binding fragment thereof that specifically binds a co-stimulatory molecule is MOXR0916. In some embodiments, the antibody or antigen binding fragment thereof that specifically binds a co-stimulatory molecule is PF-04518600. In some embodiments, the antibody or antigen binding fragment thereof that specifically binds a co-stimulatory molecule is MEDI6383. In some embodiments, the antibody or antigen binding fragment thereof that specifically binds a co-stimulatory molecule is MEDI0562. In some embodiments, the antibody or antigen binding fragment thereof that specifically binds a co-stimulatory molecule is INCAGN01949. In some embodiments, the antibody or antigen binding fragment thereof that specifically binds a co-stimulatory molecule is InVivoPlus anti-mouse OX40 (clone OX-86) (Company: BioXcell, Catalog: BP0031).

Additional antibodies or antigen binding fragments thereof that specifically bind a co-stimulatory molecule can include, for example: for mouse, InVivoPlus anti-mouse 4-1BB (CD137) (clone LOB12.3) (Company: BioXcell, Catalog: BP0169), InVivoPlus anti-mouse CD40 (clone FGK4.5/FGK45) (Company: BioXcell, Catalog: BP0016-2); for human, anti-human OX40, BMS 986178, GSK3174998, PF-04518600, MOXR0916, PF-04518600, MEDI6383, MEDI0562, INCAGN01949; anti-human 4-1BB, Utomilumab, Urelumab; anti-human CD40, CP-870893, APX005M, ADC-1013, JNJ-64457107, SEA-CD40, R07009789. In some embodiments, the mRNA encoding the co-stimulatory molecule comprises a heterologous 5′ untranslated region (5′UTR). In some embodiments, the mRNA encoding the co-stimulatory molecule comprises a heterologous 3′ untranslated region (3′UTR).

In some embodiments, the nucleic acids (for example, the mRNA encoding the co-stimulatory molecule) disclosed herein comprise at least one chemically modified nucleotide. In some embodiments, the at least one chemically modified nucleotide comprises a chemically modified nucleobase, a chemically modified ribose, a chemically modified phosphodiester linkage, or a combination thereof.

In one embodiment, the at least one chemically modified nucleotide is a chemically modified nucleobase.

In one embodiment, the chemically modified nucleobase is selected from 5-formylcytidine (5fC), 5-methylcytidine (5meC), 5-methoxycytidine (5moC), 5-hydroxycytidine (5hoC), 5-hydroxymethylcytidine (5hmC), 5-formyluridine (5fU), 5-methyluridine (5-meU), 5-methoxyuridine (5moU), 5-carboxymethylesteruridine (5camU), pseudouridine (Ψ), N¹-methylpseudouridine (me¹Ψ), N⁶-methyladenosine (me⁶A), or thienoguanosine (^{th}G).

In some embodiments, the chemically modified nucleobase is 5-methoxyuridine (5moU). In some embodiments, the chemically modified nucleobase is pseudouridine (Ψ). In some embodiments, the chemically modified nucleobase is N′-methylpseudouridine (me¹Ψ).

The structures of these modified nucleobases are shown below:

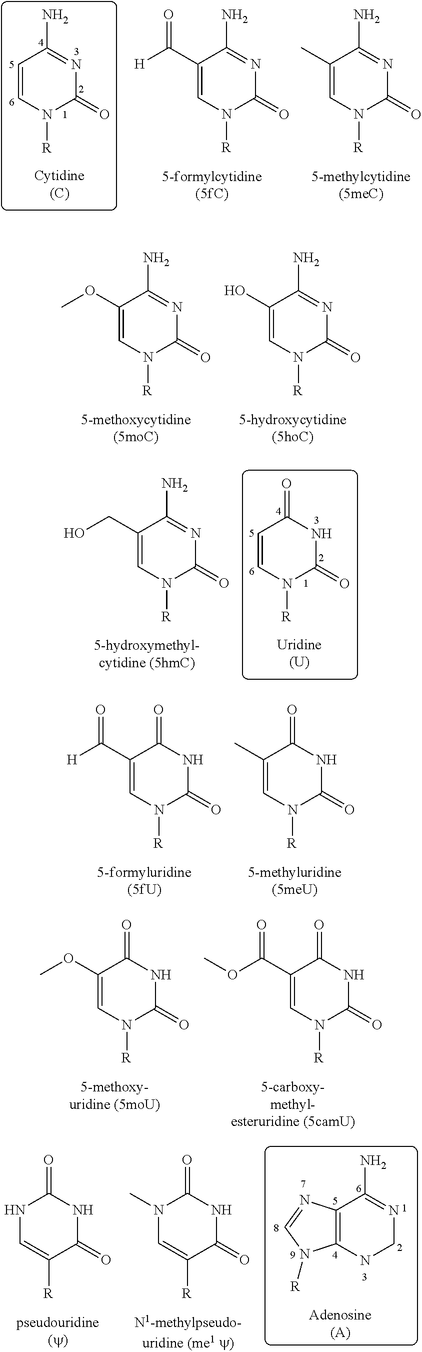

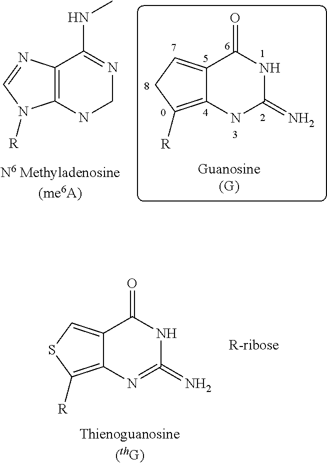

In one embodiment, the at least one chemically modified nucleotide is a chemically modified ribose.

In one embodiment, the chemically modified ribose is selected from 2′-O-methyl (2′-O-Me), 2′-Fluoro (2′-F), 2′-deoxy-2′-fluoro-beta-D-arabino-nucleic acid (2′F-ANA), 4′-S, 4′-SFANA, 2′-azido, UNA, 2′-O-methoxy-ethyl (2′-O-ME), 2′-O-Allyl, 2′-O-Ethylamine, 2′-O-Cyanoethyl, Locked nucleic acid (LAN), Methylene-cLAN, N-MeO-amino BNA, or N-MeO-aminooxy BNA. In one embodiment, the chemically modified ribose is 2′-O-methyl (2′-O-Me). In one embodiment, the chemically modified ribose is 2′-Fluoro (2′-F).

The structures of these modified riboses are shown below:

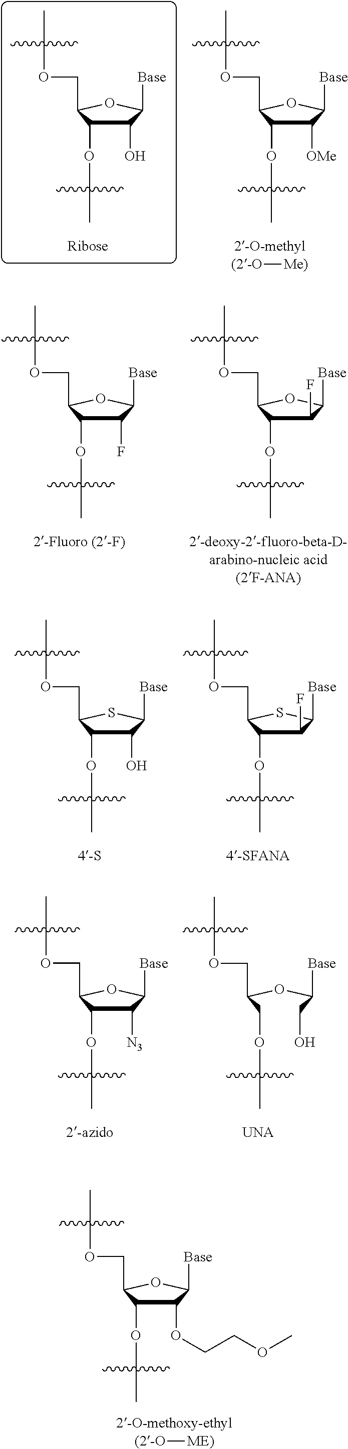

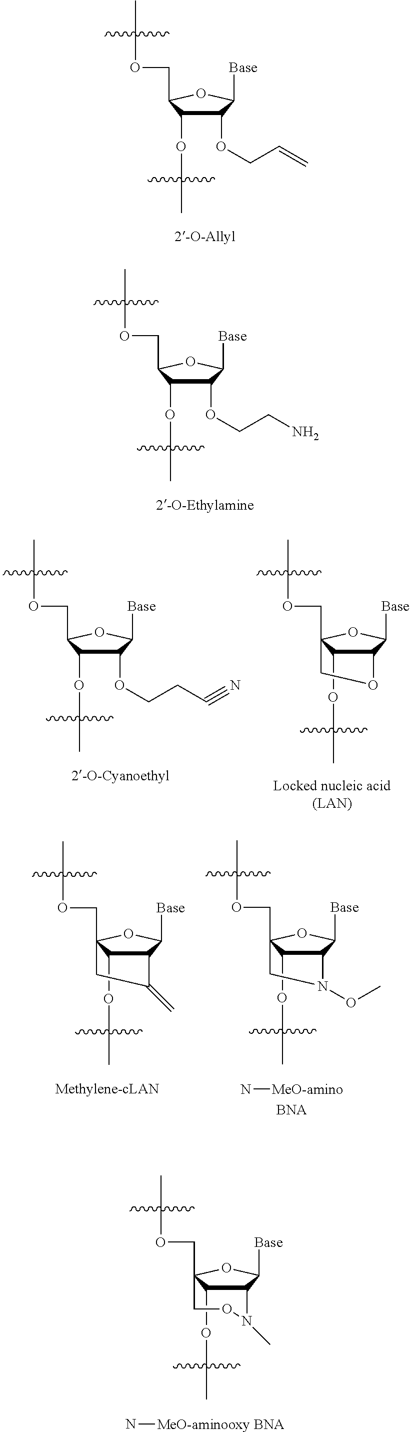

In one embodiment, the at least one chemically modified nucleotide is a chemically modified phosphodiester linkage.

In one embodiment, the chemically modified phosphodiester linkage is selected from phosphorothioate (PS), boranophosphate, phosphodithioate (PS2), 3′,5′-amide, N3′-phosphoramidate (NP), Phosphodiester (PO), or 2′,5′-phosphodiester (2′,5′-PO). In one embodiment, the chemically modified phosphodiester linkage is phosphorothioate.

The structures of these modified phosphodiester linkages are shown below:

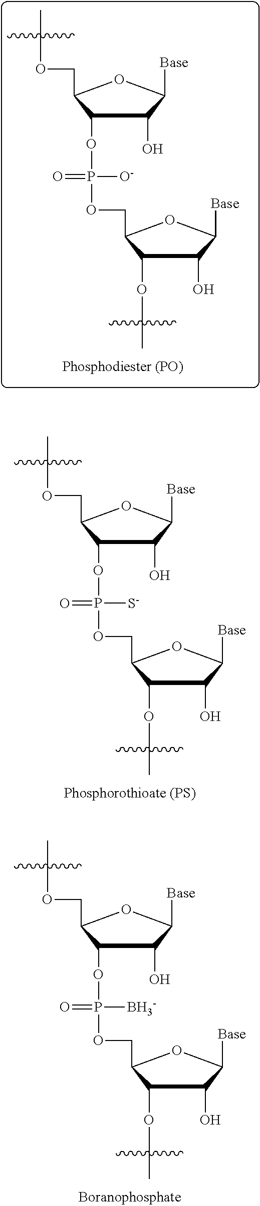

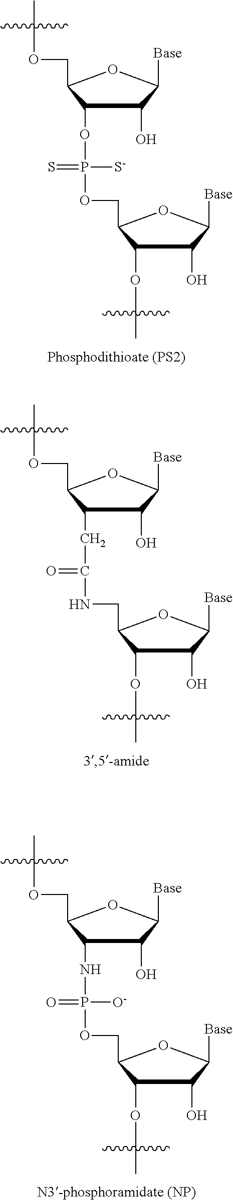

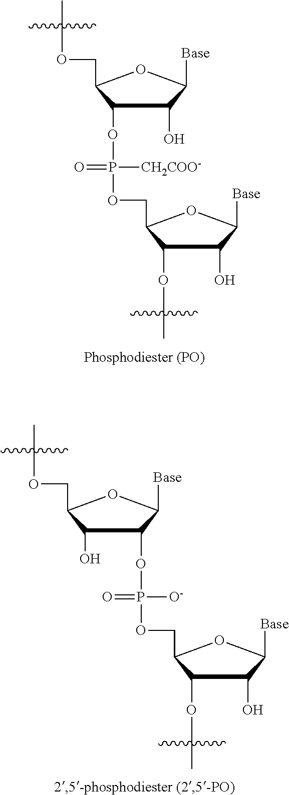

In some embodiments, the co-stimulatory molecule is selected from ICOS, CD28, CD27, HVEM, LIGHT, CD40L, 4-1BB, OX40, DR3, GITR, CD30, SLAM, CD2, CD226, Galectin9, TIM1, LFA1, B7-H2, B7-1, B7-2, CD70, LIGHT, HVEM, CD40, 4-1BBL, OX40L, TL1A, GITRL, CD30L, SLAM, CD48, CD58, CD155, CD112, CD80, CD86, ICOSL, TIM3, TIM4, ICAM1, or LFA3. In some embodiments, the co-stimulatory molecule comprises OX40. In some embodiments, the co-stimulatory molecule comprises 4-1BB (CD137).

In some embodiments, the mRNA encoding the co-stimulatory molecule is isolated. In some embodiments, the mRNA encoding the co-stimulatory molecule is recombinant. In some embodiments, the antibody or antigen binding fragment thereof is isolated. In some embodiments, the antibody or antigen binding fragment thereof is recombinant. In some embodiments, the antibody is a monoclonal antibody.

In some embodiments, the co-stimulatory molecule is ICOS. In some embodiments, the co-stimulatory molecule is CD28. In some embodiments, the co-stimulatory molecule is CD27. In some embodiments, the co-stimulatory molecule is HVEM. In some embodiments, the co-stimulatory molecule is LIGHT. In some embodiments, the co-stimulatory molecule is CD40L. In some embodiments, the co-stimulatory molecule is 4-1BB. In some embodiments, the co-stimulatory molecule is DR3. In some embodiments, the co-stimulatory molecule is GITR. In some embodiments, the co-stimulatory molecule is CD30. In some embodiments, the co-stimulatory molecule is SLAM. In some embodiments, the co-stimulatory molecule is CD2. In some embodiments, the co-stimulatory molecule is CD226. In some embodiments, the co-stimulatory molecule is Galectin9. In some embodiments, the co-stimulatory molecule is TIM1. In some embodiments, the co-stimulatory molecule is LFA1. In some embodiments, the co-stimulatory molecule is B7-H2. In some embodiments, the co-stimulatory molecule is B7-1. In some embodiments, the co-stimulatory molecule is B7-2. In some embodiments, the co-stimulatory molecule is CD70. In some embodiments, the co-stimulatory molecule is LIGHT. In some embodiments, the co-stimulatory molecule is HVEM. In some embodiments, the co-stimulatory molecule is CD40. In some embodiments, the co-stimulatory molecule is 4-1BBL. In some embodiments, the co-stimulatory molecule is OX40L. In some embodiments, the co-stimulatory molecule is TL1A. In some embodiments, the co-stimulatory molecule is GITRL. In some embodiments, the co-stimulatory molecule is CD30L. In some embodiments, the co-stimulatory molecule is SLAM. In some embodiments, the co-stimulatory molecule is CD48. In some embodiments, the co-stimulatory molecule is CD58. In some embodiments, the co-stimulatory molecule is CD155. In some embodiments, the co-stimulatory molecule is CD112. In some embodiments, the co-stimulatory molecule is CD80. In some embodiments, the co-stimulatory molecule is CD86. In some embodiments, the co-stimulatory molecule is ICOSL. In some embodiments, the co-stimulatory molecule is TIM3. In some embodiments, the co-stimulatory molecule is TIM4. In some embodiments, the co-stimulatory molecule is ICAM1. In some embodiments, the co-stimulatory molecule is LFA3.

In some embodiments, the co-stimulatory molecule is CD40. In some embodiments, the CD40 co-stimulatory molecule comprises the mRNA sequence SEQ ID NO: 23. In some embodiments, the CD40 co-stimulatory molecule comprises a nucleic acid sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 23, or a variant or a fragment thereof.

In some embodiments, the co-stimulatory molecule is OX40. In some embodiments, the OX40 co-stimulatory molecule comprises the mRNA sequence SEQ ID NO: 1. In some embodiments, the OX40 co-stimulatory molecule comprises the mRNA sequence SEQ ID NO: 2. In some embodiments, the OX40 co-stimulatory molecule comprises the mRNA sequence SEQ ID NO: 5. In some embodiments, the co-stimulatory molecule is OX40. In some embodiments, the OX40 co-stimulatory molecule comprises the mRNA sequence SEQ ID NO: 6.

In some embodiments, the OX40 co-stimulatory molecule comprises a nucleic acid sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 1, or a variant or a fragment thereof. In some embodiments, the OX40 co-stimulatory molecule comprises a nucleic acid sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 2, or a variant or a fragment thereof. In some embodiments, the OX40 co-stimulatory molecule comprises a nucleic acid sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 5, or a variant or a fragment thereof. In some embodiments, the OX40 co-stimulatory molecule comprises a nucleic acid sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 6, or a variant or a fragment thereof.

In some embodiments, the co-stimulatory molecule is encoded by a nucleic acid sequence at least 60% (for example, at least 60%, at least 65%, at least 70%, at least 75%, at least 80%, at least 85%, at least 90%, at least 95%, at least 96%, at least 97%, at least 98%, at least 99%) identical to a sequence of a co-stimulatory molecule selected from ICOS, CD28, CD27, HVEM, LIGHT, CD40L, 4-1BB, OX40, DR3, GITR, CD30, SLAM, CD2, CD226, Galectin9, TIM1, LFA1, B7-H2, B7-1, B7-2, CD70, LIGHT, HVEM, CD40, 4-1BBL, OX40L, TL1A, GITRL, CD30L, SLAM, CD48, CD58, CD155, CD112, CD80, CD86, ICOSL, TIM4, ICAM1, LFA3, or a variant or a fragment thereof.

In some embodiments, the mRNA encoding the co-stimulatory molecule comprises a modified 5′ untranslated region (5′UTR). In some embodiments, the mRNA encoding the co-stimulatory molecule comprises a modified 3′ untranslated region (3′UTR). For example, a modified sequence could include insertions, deletions, or nucleotide substitutions.

In some embodiments, the mRNA encoding the co-stimulatory molecule comprises a heterologous 5′ untranslated region (5′UTR) comprising the mRNA sequence SEQ ID NO: 3. In some embodiments, the mRNA encoding the co-stimulatory molecule comprises a heterologous 3′ untranslated region (3′UTR) comprising the mRNA sequence SEQ ID NO: 4. In some embodiments, the mRNA encoding the co-stimulatory molecule comprises a heterologous 5′ untranslated region (5′UTR) comprising a nucleic acid sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 3, or a variant or a fragment thereof. In some embodiments, the mRNA encoding the co-stimulatory molecule comprises a heterologous 3′ untranslated region (3′UTR) comprising a nucleic acid sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 4, or a variant or a fragment thereof.

### Methods

In one aspect, provided herein is a method for the delivery of an agent (for example, a polynucleotide) into a cell comprising;

introducing into the cell a composition comprising;

a nanoparticle, comprising;
a compound of Formula A, I, II, III, IV, V, VI, VII, VIII, IX, or X;
a non-cationic lipid;
a polyethylene glycol-lipid;
a sterol; and

an agent.

In one aspect, disclosed herein is a method for the delivery of an agent into a cell comprising;

introducing into the cell a composition comprising;

a nanoparticle comprising;
a compound of Formula A:

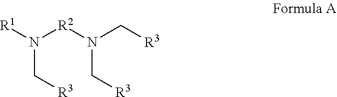

or a salt thereof, wherein:
R¹ is

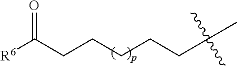

wherein p is an integer from 1 to 20;

R⁶ is a toll-like receptor (TLR) agonist or a derivative thereof;

R² is alkyl, cycloalkyl, heterocycloalkyl, alkylheterocycloalkyl, amide, amine, alkylamide, ether, alkylether,

each R³ is independently selected from alkyl, alkenyl, alkynyl, ester, or alkylester;
a non-cationic lipid;
a polyethylene glycol-lipid;
a sterol; and

an agent.

In some embodiments, R⁶ is

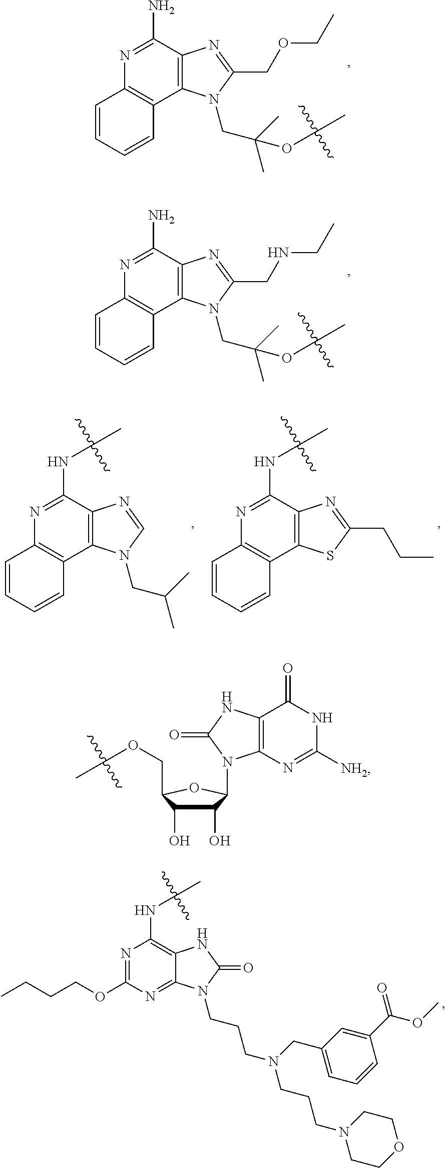

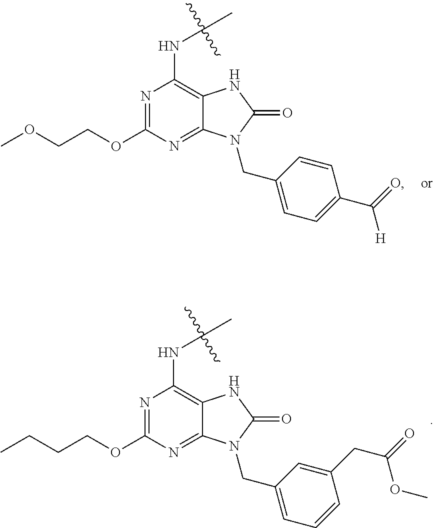 [SMILES: *Nc1nc(OCCOC)nc2c1[nH]c(=O)n2Cc1ccc(C=O)cc1 ; *Nc1nc(OCCCC)nc2c1[nH]c(=O)n2Cc1cccc(CC(=O)OC)c1]

In some embodiments, R⁶ is

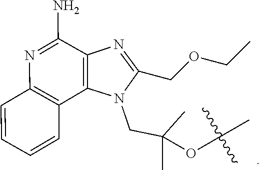 [SMILES: *OC(C)(C)Cn1c(COCC)nc2c(N)nc3ccccc3c21]

In some embodiments, R¹ is

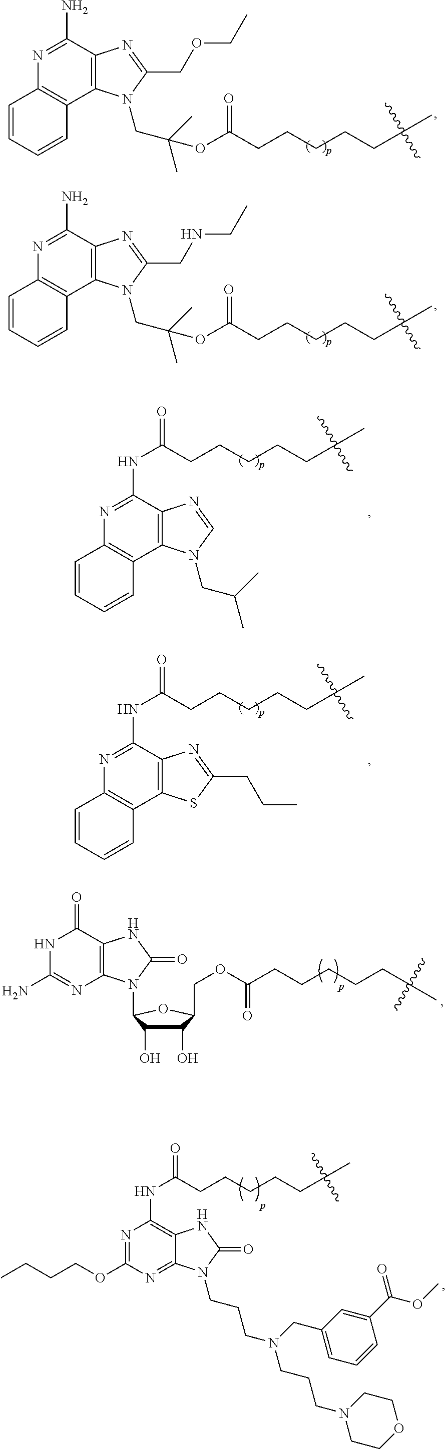

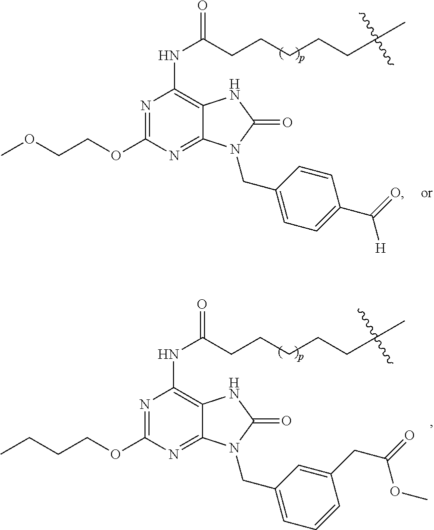

wherein p is an integer from 1 to 20.

In some embodiments, R¹ is

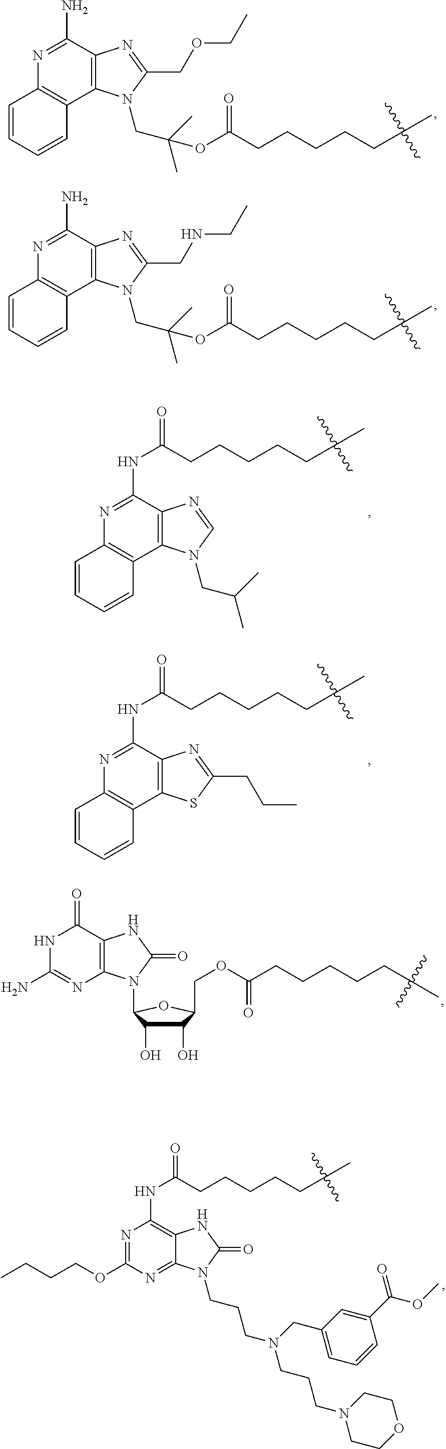

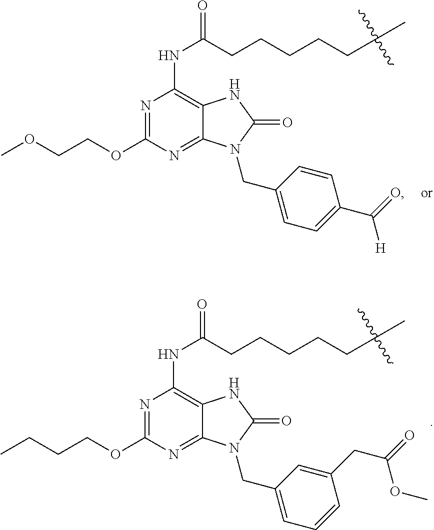 [SMILES: *CCCCCC(=O)Nc1nc(OCCOC)nc2c1[nH]c(=O)n2Cc1ccc(C=O)cc1 ; *CCCCCC(=O)Nc1nc(OCCCC)nc2c1[nH]c(=O)n2Cc1cccc(CC(=O)OC)c1]

In some embodiments, R¹ is

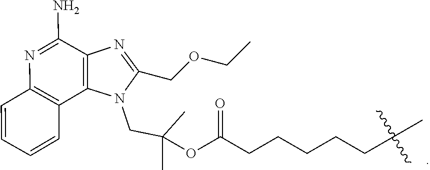 [SMILES: *CCCCCC(=O)OC(C)(C)Cn1c(COCC)nc2c(N)nc3ccccc3c21]

In some embodiments, R² is

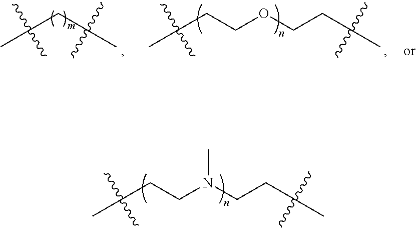

wherein m is an integer from 1 to 20,
wherein n is an integer from 1 to 3.

In one aspect, disclosed herein is a method for the delivery of an agent into a cell comprising;
introducing into the cell a composition comprising;
a nanoparticle comprising;
   a compound of Formula I, II, III, IV, V, VI, VII, VIII, IX, or X:

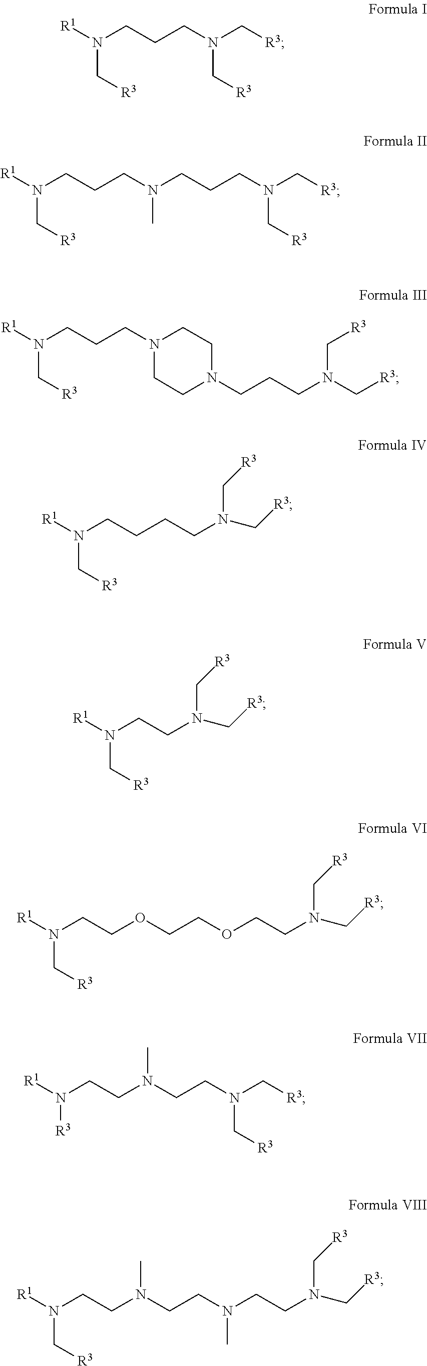

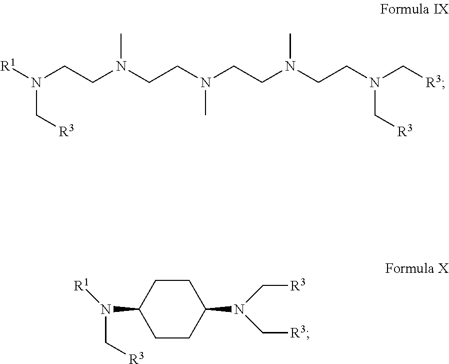

or a salt thereof, wherein:
R¹ is

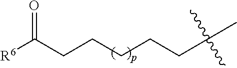

wherein p is an integer from 1 to 20;
R⁶ is a toll-like receptor (TLR) agonist or a derivative thereof; and
each R³ is independently selected from alkyl, alkenyl, alkynyl, ester, or alkylester;
   a non-cationic lipid;
   a polyethylene glycol-lipid;
   a sterol; and

an agent.

In some embodiments, R⁶ is

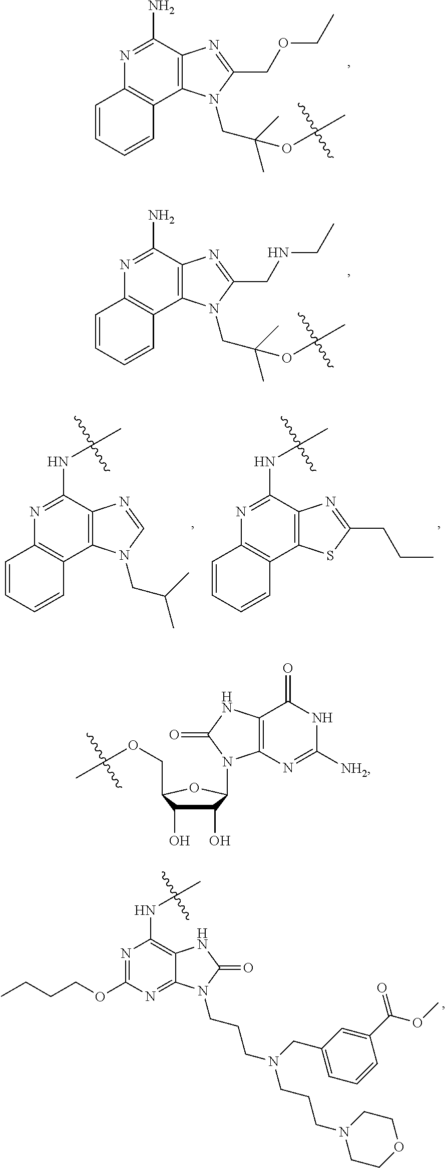

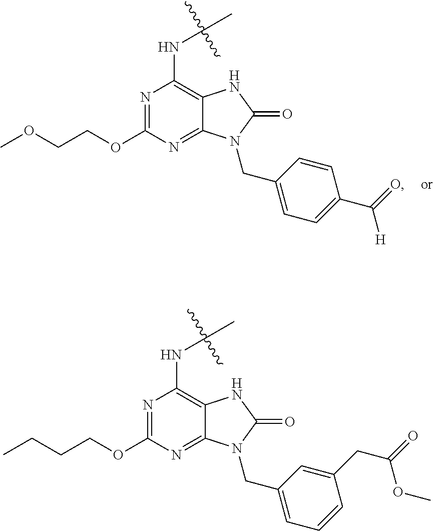 [SMILES: *Nc1nc(OCCOC)nc2c1[nH]c(=O)n2Cc1ccc(C=O)cc1 ; *Nc1nc(OCCCC)nc2c1[nH]c(=O)n2Cc1cccc(CC(=O)OC)c1]

In some embodiments, R⁶ is

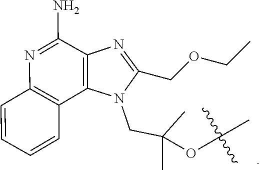 [SMILES: *OC(C)(C)Cn1c(COCC)nc2c(N)nc3ccccc3c21]

In some embodiments. R¹ is

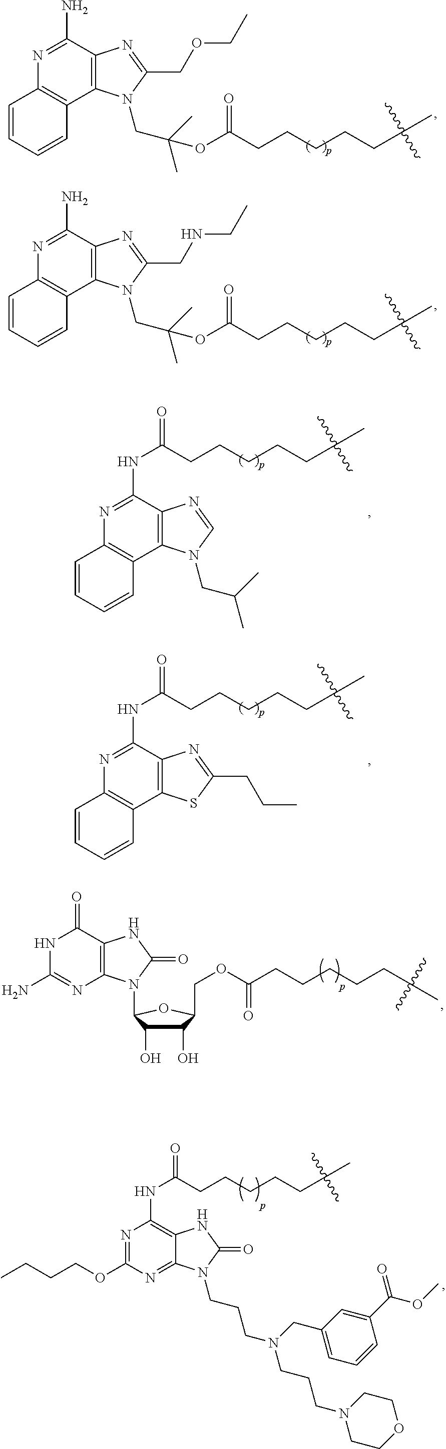

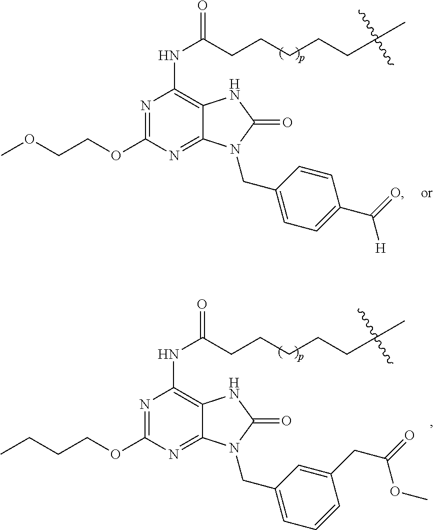

wherein p is an integer from 1 to 20.

In some embodiments, R¹ is

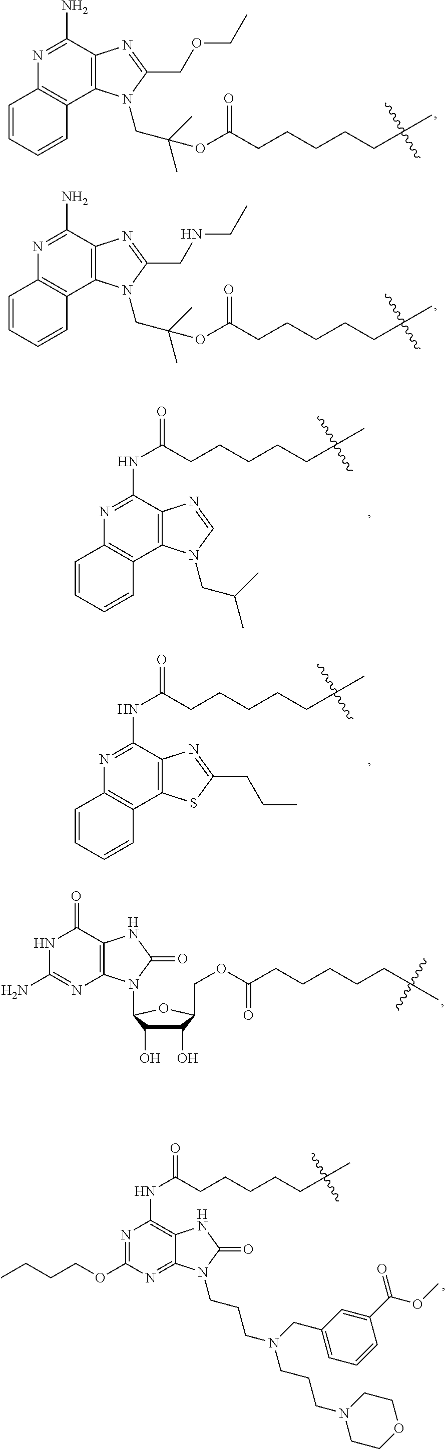

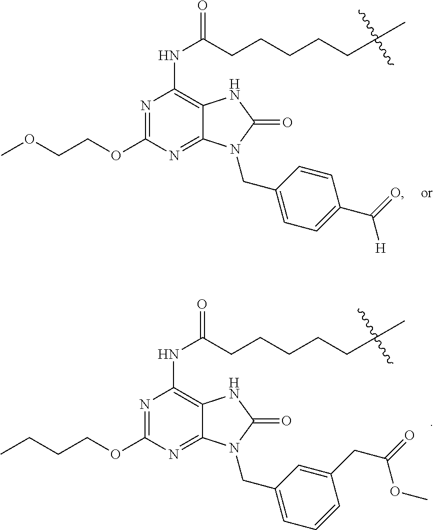 [SMILES: *CCCCCC(=O)Nc1nc(OCCOC)nc2c1[nH]c(=O)n2Cc1ccc(C=O)cc1 ; *CCCCCC(=O)Nc1nc(OCCCC)nc2c1[nH]c(=O)n2Cc1cccc(CC(=O)OC)c1]

In some embodiments, R¹ is

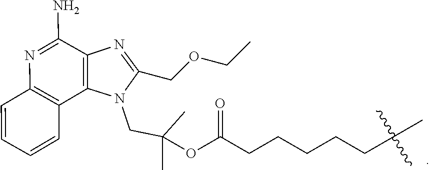 [SMILES: *CCCCCC(=O)OC(C)(C)Cn1c(COCC)nc2c(N)nc3ccccc3c21]

In some embodiments, each R³ is independently selected from alkyl, alkenyl, alkynyl, ester, or alkylester. In some embodiments, each R³ is independently selected from alkyl, alkenyl, or alkylester. In some embodiments, each R³ is independently selected from C₇₋₁₇alkyl, C₇₋₂₀alkenyl, or C₁₋₁₀alkylester, wherein the C₁₋₁₀alkylester is substituted with an alkyl or alkenyl group.

In some embodiments, each R³ is independently selected from an alkyl. In some embodiments, each R³ is independently selected from a C₇₋₁₇alkyl. In some embodiments, each R³ is independently selected from a C₉₋₁₂alkyl. In some embodiments, each R³ is independently selected from a C₉alkyl. In some embodiments, each R³ is independently selected from a C₁₀alkyl. In some embodiments, each R³ is independently selected from a C₁₁alkyl. In some embodiments, each R³ is independently selected from a C₁₀alkyl.

In some embodiments, each R³ is independently selected from an alkenyl. In some embodiments, each R³ is independently selected from a C₇₋₂₀alkenyl. In some embodiments, each R³ is independently selected from a C₉alkenyl.

In some embodiments, each R³ is independently selected from an alkylester. In some embodiments, each R³ is independently selected from an alkylester, wherein the alkylester is substituted with an alkyl or alkenyl group. In some embodiments, each R³ is independently selected from a C₁₋₁₀alkylester, wherein the C₁₋₁₀alkylester is substituted with an alkyl or alkenyl group. In some embodiments, each R³ is independently selected from a C₁₋₁₀alkylester, wherein the C₁₋₁₀alkylester is substituted with C₆₋₈alkyl group. In some embodiments, each R³ is independently selected from a C₁₋₁₀alkylester, wherein the C₁₋₁₀alkylester is substituted with C₆₋₁₀alkenyl group.

In some embodiments, each R³ is independently selected from a linear or branched saturated alkyl chain comprising 7 to 17 carbons. In some embodiments, each R³ is independently selected from

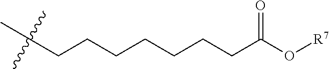

wherein each R⁷ is independently selected from a linear or branched saturated alkyl chain comprising 4 to 9 carbons.

In some embodiments, at least one R³ is C₇-C₁₇ alkyl. In some embodiments, at least one R³ is C₇₋₂₀alkenyl. In some embodiments, at least one R³ is C₁₋₁₀alkylester. In some embodiments, at least one R³ is C₁₋₁₀alkylester substituted with an alkyl or alkenyl group.

In some embodiments, at least one R³ is a branched alkyl. In some embodiments, at least one R³ is an unbranched alkyl. In some embodiments, at least one R³ is a branched alkenyl. In some embodiments, at least one R³ is an unbranched alkenyl.

In some embodiments, R³ are independently selected from: C₇ alkyl, C₈ alkyl, C₉ alkyl, C₁₀ alkyl, C₁₁ alkyl, C₁₂ alkyl, C₁₃ alkyl, C₁₄ alkyl, C₁₅ alkyl, C₁₆ alkyl, C₁₇ alkyl;

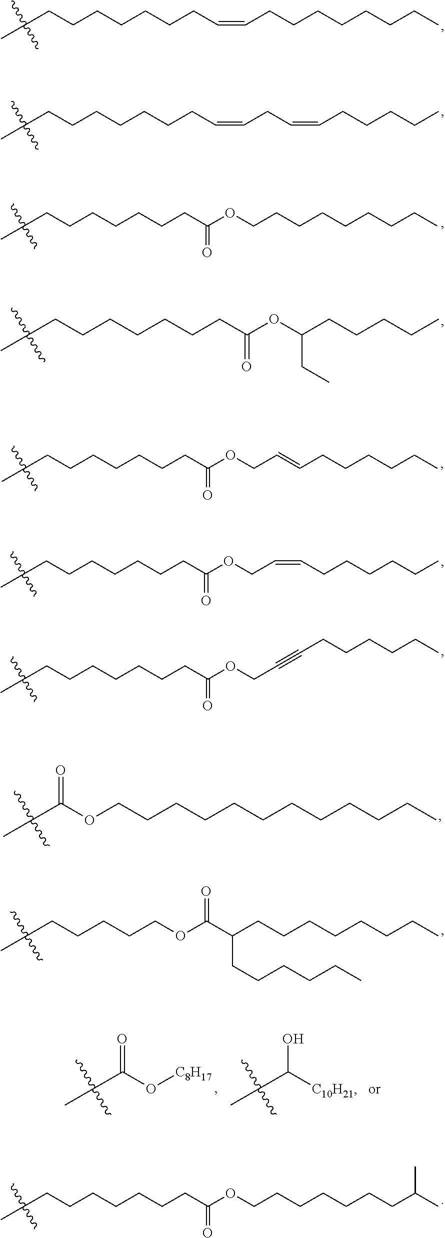

In some embodiments, a nanoparticle comprising any compound as described in the Compounds section above, is used in the methods herein, for delivery of an agent into a cell.

In some embodiments, the agent is a polynucleotide. In some embodiments, the agent is an RNA. In some embodiments, the agent is an mRNA. In some embodiments, the agent is a therapeutic agent, diagnostic agent, or prophylactic agent.

In some embodiments, provided herein are methods for the delivery of polynucleotides. In some embodiments, provided herein are methods for the delivery of polynucleotides (for example, mRNA) to correct a mutation in a genome. For example, mRNAs can be delivered to correct mutations that cause hemophilia (due to mutations in the genes encoding Factor VIII (F8; hemophilia A) or Factor IX (F9; hemoglobin B). In some embodiments, provided herein are methods for the delivery of polynucleotides. In some embodiments, provided herein are methods for the delivery of polynucleotides (for example, mRNA) to provide expression of the mRNA (and translation to produce a protein) in a cell. In some embodiments, provided herein are methods for the delivery of polynucleotides (for example, mRNA) to induce an immune response in a subject. In some embodiments, the RNA (e.g., mRNA) polynucleotide having an open reading frame encoding at least one (e.g., at least 2, 3, 4 or 5) hMPV, PIV, RSV, MeV, and/or a BetaCoV (e.g., MERS-CoV, SARS-CoV, SARS-CoV2, HCoV-OC43, HCoV-229E, HCoV-NL63, HCoV-NL, HCoV-NH, HCoV-HKU1) antigenic polypeptide, or any combination of two or more of the antigenic polypeptides.

In some aspects, disclosed herein is a method of stimulating a T cell comprising administering to a subject an effective amount of a composition comprising: an antibody, a ligand, or an antigen binding fragment thereof that specifically binds a co-stimulatory molecule; and a nanoparticle comprising an mRNA encoding the co-stimulatory molecule.

In some embodiments, the subject is a mammal. In some embodiments, the mammal is a human.

In some aspects, disclosed herein is a method of treating a cancer comprising administering to a subject in need thereof an effective amount of an antibody, a ligand, or an antigen binding fragment thereof that specifically binds a co-stimulatory molecule and a nanoparticle comprising an mRNA encoding the co-stimulatory molecule.

In some embodiments, the cancer comprises colorectal cancer or melanoma. In some embodiments, the compositions herein are used to treat both local and metastatic tumors.

In some embodiments, the subject is a mammal. In some embodiments, the mammal is a human.

In some embodiments, the method further comprises administering an additional therapeutic agent. In some embodiments, the additional therapeutic agent comprises an additional immunotherapeutic agent. In some embodiments, the immunotherapeutic agent is selected from an anti-CD40 antibody, an anti-PDL1 antibody, an anti-PD1 antibody, an anti-CTLA4 antibody, or a combination thereof.

In some aspects, disclosed herein is a method of treating a cancer comprising administering to a subject in need thereof an effective amount of an antibody, a ligand, or an antigen binding fragment thereof that specifically binds a co-stimulatory molecule and a nanoparticle comprising an mRNA encoding the co-stimulatory molecule.

In some aspects, disclosed herein is a method of treating a cancer comprising administering to a subject in need thereof an effective amount of an antibody or antigen binding fragment thereof that specifically binds a co-stimulatory molecule and a nanoparticle comprising an mRNA encoding the co-stimulatory molecule.

In some embodiments, the cancer comprises melanoma, colorectal cancer, lung cancer, colon cancer, or lymphoma. In some embodiments, the cancer comprises colorectal cancer or melanoma. In some embodiments, the cancer is colorectal cancer. In some embodiments, the cancer is melanoma. In some embodiments, the composition herein are used to treat both local and metastatic tumors.

In some embodiments, the compositions and methods described herein are useful for treating or preventing metastasis or recurrence of a cancer. In some embodiments, the compositions and methods described herein are useful for the prevention of recurrence of excised solid tumors. In some embodiments, the compositions and methods described herein are useful for the prevention of metastasis of excised solid tumors.

In one aspect, the methods described herein are used to treat cancer, for example, melanoma, lung cancer (including lung adenocarcinoma, basal cell carcinoma, squamous cell carcinoma, large cell carcinoma, bronchioloalveolar carcinoma, bronchogenic carcinoma, non-small-cell carcinoma, small cell carcinoma, mesothelioma); breast cancer (including ductal carcinoma, lobular carcinoma, inflammatory breast cancer, clear cell carcinoma, mucinous carcinoma, serosal cavities breast carcinoma); colorectal cancer (colon cancer, rectal cancer, colorectal adenocarcinoma); anal cancer; pancreatic cancer (including pancreatic adenocarcinoma, islet cell carcinoma, neuroendocrine tumors); prostate cancer; prostate adenocarcinoma; ovarian carcinoma (ovarian epithelial carcinoma or surface epithelial-stromal tumor including serous tumor, endometrioid tumor and mucinous cystadenocarcinoma, sex-cord-stromal tumor); liver and bile duct carcinoma (including hepatocellular carcinoma, cholangiocarcinoma, hemangioma); esophageal carcinoma (including esophageal adenocarcinoma and squamous cell carcinoma); oral and oropharyngeal squamous cell carcinoma; salivary gland adenoid cystic carcinoma; bladder cancer; bladder carcinoma; carcinoma of the uterus (including endometrial adenocarcinoma, ocular, uterine papillary serous carcinoma, uterine clear-cell carcinoma, uterine sarcomas, leiomyosarcomas, mixed mullerian tumors); glioma, glioblastoma, medulloblastoma, and other tumors of the brain; kidney cancers (including renal cell carcinoma, clear cell carcinoma, Wilm's tumor); cancer of the head and neck (including squamous cell carcinomas); cancer of the stomach (gastric cancers, stomach adenocarcinoma, gastrointestinal stromal tumor); testicular cancer; germ cell tumor; neuroendocrine tumor; cervical cancer; carcinoids of the gastrointestinal tract, breast, and other organs; signet ring cell carcinoma; mesenchymal tumors including sarcomas, fibrosarcomas, haemangioma, angiomatosis, haemangiopericytoma, pseudoangiomatous stromal hyperplasia, myofibroblastoma, fibromatosis, inflammatory myofibroblastic tumor, lipoma, angiolipoma, granular cell tumor, neurofibroma, schwannoma, angiosarcoma, liposarcoma, rhabdomyosarcoma, osteosarcoma, leiomyoma, leiomysarcoma, skin, including melanoma, cervical, retinoblastoma, head and neck cancer, pancreatic, brain, thyroid, testicular, renal, bladder, soft tissue, adenal gland, urethra, cancers of the penis, myxosarcoma, chondrosarcoma, osteosarcoma, chordoma, malignant fibrous histiocytoma, lymphangiosarcoma, mesothelioma, squamous cell carcinoma; epidermoid carcinoma, malignant skin adnexal tumors, adenocarcinoma, hepatoma, hepatocellular carcinoma, renal cell carcinoma, hypernephroma, cholangiocarcinoma, transitional cell carcinoma, choriocarcinoma, seminoma, embryonal cell carcinoma, glioma anaplastic; glioblastoma multiforme, neuroblastoma, medulloblastoma, malignant meningioma, malignant schwannoma, neurofibrosarcoma, parathyroid carcinoma, medullary carcinoma of thyroid, bronchial carcinoid, pheochromocytoma, Islet cell carcinoma, malignant carcinoid, malignant paraganglioma, melanoma, Merkel cell neoplasm, cystosarcoma phylloide, salivary cancers, thymic carcinomas, and cancers of the vagina among others.

In some embodiments, the compositions and methods described herein are useful in treating or preventing a cancer. In some cases, the cancer is a circulating cancer cell (circulating tumor cell). In some cases, the cancer is a metastatic cancer cell.

In some embodiments, the subject is a mammal. In some embodiments, the mammal is a human.

In some embodiments, the antibody or antigen binding fragment thereof and the nanoparticle are administered by intramuscularly injection or systematically.

In some embodiments, the method further comprises administering an additional therapeutic agent. In some embodiments, the additional therapeutic agent comprises an additional immunotherapeutic agent. In some embodiments, the immunotherapeutic agent is selected from an anti-CD40 antibody, an anti-PDL1 antibody, an anti-PD1 antibody, an anti-CTLA4 antibody, or a combination thereof.

In one embodiment, the immunotherapeutic agent is an anti-PDL1 antibody. In one embodiment, the anti-PDL1 antibody is selected from atezolizumab, durvalumab, or avelumab. In one embodiment, the anti-PDL1 antibody is atezolizumab (MPDL3280A)(Roche). In one embodiment, the anti-PDL1 antibody is durvalumab (MEDI4736). In one embodiment, the anti-PDL1 antibody is avelumab (MS0010718C).

In one embodiment, the immunotherapeutic agent is a programmed death protein 1 (PD-1) inhibitor or programmed death protein ligand 1 or 2 inhibitor. PD-1 inhibitors are known in the art, and include, for example, nivolumab (BMS), pembrolizumab (Merck), pidilizumab (CureTech/Teva), AMP-244 (Amplimmune/GSK), BMS-936559 (BMS), and MEDI4736 (Roche/Genentech).

In one embodiment, the immunotherapeutic agent is an anti-PD1 antibody. In one embodiment, the anti-PD1 antibody is nivolumab. In one embodiment, the anti-PD1 antibody is pembrolizumab.

In one embodiment, the immunotherapeutic agent is an anti-CTLA4 antibody. In one embodiment, the anti-CTLA4 antibody is ipilimumab.

In some embodiments, the additional therapeutic agent is an anti-neoplastic agent. For example, the anti-neoplastic agent can be selected from the group consisting of Abiraterone Acetate, Abitrexate (Methotrexate), Abraxane (Paclitaxel Albumin-stabilized Nanoparticle Formulation), ABVD, ABVE, ABVE-PC, AC, AC-T, Adcetris (Brentuximab Vedotin), ADE, Ado-Trastuzumab Emtansine, Adriamycin (Doxorubicin Hydrochloride), Adrucil (Fluorouracil), Afatinib Dimaleate, Afinitor (Everolimus), Akynzeo (Netupitant and Palonosetron Hydrochloride), Aldara (Imiquimod), Aldesleukin, Alemtuzumab, Alimta (Pemetrexed Disodium), Aloxi (Palonosetron Hydrochloride), Ambochlorin (Chlorambucil), Amboclorin (Chlorambucil), Aminolevulinic Acid, Anastrozole, Aprepitant, Aredia (Pamidronate Disodium), Arimidex (Anastrozole), Aromasin (Exemestane), Arranon (Nelarabine), Arsenic Trioxide, Arzerra (Ofatumumab), Asparaginase Erwinia chrysanthemi, Avastin (Bevacizumab), Axitinib, Azacitidine, BEACOPP, Becenum (Carmustine), Beleodaq (Belinostat), Belinostat, Bendamustine Hydrochloride, BEP, Bevacizumab, Bexarotene, Bexxar (Tositumomab and Iodine I 131 Tositumomab), Bicalutamide, BiCNU (Carmustine), Bleomycin, Blinatumomab, Blincyto (Blinatumomab), Bortezomib, Bosulif (Bosutinib), Bosutinib, Brentuximab Vedotin, Busulfan, Busulfex (Busulfan), Cabazitaxel, Cabozantinib-S-Malate, CAF, Campath (Alemtuzumab), Camptosar (Irinotecan Hydrochloride), Capecitabine, CAPDX, Carboplatin, CARBOPLATIN-TAXOL, Carfilzomib, Carmubris (Carmustine), Carmustine, Carmustine Implant, Casodex (Bicalutamide), CeeNU (Lomustine), Ceritinib, Cerubidine (Daunorubicin Hydrochloride), Cervarix (Recombinant HPV Bivalent Vaccine), Cetuximab, Chlorambucil, CHLORAMBUCIL-PREDNISONE, CHOP, Cisplatin, Clafen (Cyclophosphamide), Clofarabine, Clofarex (Clofarabine), Clolar (Clofarabine), CMF, Cometriq (Cabozantinib-S-Malate), COPP, COPP-ABV, Cosmegen (Dactinomycin), Crizotinib, CVP, Cyclophosphamide, Cyfos (Ifosfamide), Cyramza (Ramucirumab), Cytarabine, Cytarabine, Liposomal, Cytosar-U (Cytarabine), Cytoxan (Cyclophosphamide), Dabrafenib, Dacarbazine, Dacogen (Decitabine), Dactinomycin, Dasatinib, Daunorubicin Hydrochloride, Decitabine, Degarelix, Denileukin Diftitox, Denosumab, DepoCyt (Liposomal Cytarabine), DepoFoam (Liposomal Cytarabine), Dexrazoxane Hydrochloride, Dinutuximab, Docetaxel, Doxil (Doxorubicin Hydrochloride Liposome), Doxorubicin Hydrochloride, Doxorubicin Hydrochloride Liposome, Dox-SL (Doxorubicin Hydrochloride Liposome), DTIC-Dome (Dacarbazine), Efudex (Fluorouracil), Elitek (Rasburicase), Ellence (Epirubicin Hydrochloride), Eloxatin (Oxaliplatin), Eltrombopag Olamine, Emend (Aprepitant), Enzalutamide, Epirubicin Hydrochloride, EPOCH, Erbitux (Cetuximab), Eribulin Mesylate, Erivedge (Vismodegib), Erlotinib Hydrochloride, Erwinaze (Asparaginase Erwinia chrysanthemi), Etopophos (Etoposide Phosphate), Etoposide, Etoposide Phosphate, Evacet (Doxorubicin Hydrochloride Liposome), Everolimus, Evista (Raloxifene Hydrochloride), Exemestane, Fareston (Toremifene), Farydak (Panobinostat), Faslodex (Fulvestrant), FEC, Femara (Letrozole), Filgrastim, Fludara (Fludarabine Phosphate), Fludarabine Phosphate, Fluoroplex (Fluorouracil), Fluorouracil, Folex (Methotrexate), Folex PFS (Methotrexate), FOLFIRI, FOLFIRI-BEVACIZUMAB, FOLFIRI-CETUXIMAB, FOLFIRINOX, FOLFOX, Folotyn (Pralatrexate), FU-LV, Fulvestrant, Gardasil (Recombinant HPV Quadrivalent Vaccine), Gardasil 9 (Recombinant HPV Nonavalent Vaccine), Gazyva (Obinutuzumab), Gefitinib, Gemcitabine Hydrochloride, GEMCITABINE-CISPLATIN, GEMCITABINE-OXALIPLATIN, Gemtuzumab Ozogamicin, Gemzar (Gemcitabine Hydrochloride), Gilotrif (Afatinib Dimaleate), Gleevec (Imatinib Mesylate), Gliadel (Carmustine Implant), Gliadel wafer (Carmustine Implant), Glucarpidase, Goserelin Acetate, Halaven (Eribulin Mesylate), Herceptin (Trastuzumab), HPV Bivalent Vaccine, Recombinant, HPV Nonavalent Vaccine, Recombinant, HPV Quadrivalent Vaccine, Recombinant, Hycamtin (Topotecan Hydrochloride), Hyper-CVAD, Ibrance (Palbociclib), Ibritumomab Tiuxetan, Ibrutinib, ICE, Iclusig (Ponatinib Hydrochloride), Idamycin (Idarubicin Hydrochloride), Idarubicin Hydrochloride, Idelalisib, Ifex (Ifosfamide), Ifosfamide, Ifosfamidum (Ifosfamide), Imatinib Mesylate, Imbruvica (Ibrutinib), Imiquimod, Inlyta (Axitinib), Interferon Alfa-2b, Recombinant, Intron A (Recombinant Interferon Alfa-2b), Iodine I 131 Tositumomab and Tositumomab, Ipilimumab, Iressa (Gefitinib), Irinotecan Hydrochloride, Istodax (Romidepsin), Ixabepilone, Ixempra (Ixabepilone), Jakafi (Ruxolitinib Phosphate), Jevtana (Cabazitaxel), Kadcyla (Ado-Trastuzumab Emtansine), Keoxifene (Raloxifene Hydrochloride), Kepivance (Palifermin), Keytruda (Pembrolizumab), Kyprolis (Carfilzomib), Lanreotide Acetate, Lapatinib Ditosylate, Lenalidomide, Lenvatinib Mesylate, Lenvima (Lenvatinib Mesylate), Letrozole, Leucovorin Calcium, Leukeran (Chlorambucil), Leuprolide Acetate, Levulan (Aminolevulinic Acid), Linfolizin (Chlorambucil), LipoDox (Doxorubicin Hydrochloride Liposome), Liposomal Cytarabine, Lomustine, Lupron (Leuprolide Acetate), Lupron Depot (Leuprolide Acetate), Lupron Depot-Ped (Leuprolide Acetate), Lupron Depot-3 Month (Leuprolide Acetate), Lupron Depot-4 Month (Leuprolide Acetate), Lynparza (Olaparib), Marqibo (Vincristine Sulfate Liposome), Matulane (Procarbazine Hydrochloride), Mechlorethamine Hydrochloride, Megace (Megestrol Acetate), Megestrol Acetate, Mekinist (Trametinib), Mercaptopurine, Mesna, Mesnex (Mesna), Methazolastone (Temozolomide), Methotrexate, Methotrexate LPF (Methotrexate), Mexate (Methotrexate), Mexate-AQ (Methotrexate), Mitomycin C, Mitoxantrone Hydrochloride, Mitozytrex (Mitomycin C), MOPP, Mozobil (Plerixafor), Mustargen (Mechlorethamine Hydrochloride), Mutamycin (Mitomycin C), Myleran (Busulfan), Mylosar (Azacitidine), Mylotarg (Gemtuzumab Ozogamicin), Nanoparticle Paclitaxel (Paclitaxel Albumin-stabilized Nanoparticle Formulation), Navelbine (Vinorelbine Tartrate), Nelarabine, Neosar (Cyclophosphamide), Netupitant and Palonosetron Hydrochloride, Neupogen (Filgrastim), Nexavar (Sorafenib Tosylate), Nilotinib, Nivolumab, Nolvadex (Tamoxifen Citrate), Nplate (Romiplostim), Obinutuzumab, Odomzo (Sonidegib), OEPA, Ofatumumab, OFF, Olaparib, Omacetaxine Mepesuccinate, Oncaspar (Pegaspargase), Ondansetron Hydrochloride, Ontak (Denileukin Diftitox), Opdivo (Nivolumab), OPPA, Oxaliplatin, Paclitaxel, Paclitaxel Albumin-stabilized Nanoparticle Formulation, PAD, Palbociclib, Palifermin, Palonosetron Hydrochloride, Palonosetron Hydrochloride and Netupitant, Pamidronate Disodium, Panitumumab, Panobinostat, Paraplat (Carboplatin), Paraplatin (Carboplatin), Pazopanib Hydrochloride, Pegaspargase, Peginterferon Alfa-2b, PEG-Intron (Peginterferon Alfa-2b), Pembrolizumab, Pemetrexed Disodium, Perj eta (Pertuzumab), Pertuzumab, Platinol (Cisplatin), Platinol-AQ (Cisplatin), Plerixafor, Pomalidomide, Pomalyst (Pomalidomide), Ponatinib Hydrochloride, Pralatrexate, Prednisone, Procarbazine Hydrochloride, Proleukin (Aldesleukin), Prolia (Denosumab), Promacta (Eltrombopag Olamine), Provenge (Sipuleucel-T), Purinethol (Mercaptopurine), Purixan (Mercaptopurine), Radium 223 Dichloride, Raloxifene Hydrochloride, Ramucirumab, Rasburicase, R-CHOP, R-CVP, Recombinant Human Papillomavirus (HPV) Bivalent Vaccine, Recombinant Human Papillomavirus (HPV) Nonavalent Vaccine, Recombinant Human Papillomavirus (HPV) Quadrivalent Vaccine, Recombinant Interferon Alfa-2b, Regorafenib, R-EPOCH, Revlimid (Lenalidomide), Rheumatrex (Methotrexate), Rituxan (Rituximab), Rituximab, Romidepsin, Romiplostim, Rubidomycin (Daunorubicin Hydrochloride), Ruxolitinib Phosphate, Sclerosol Intrapleural Aerosol (Talc), Siltuximab, Sipuleucel-T, Somatuline Depot (Lanreotide Acetate), Sonidegib, Sorafenib Tosylate, Sprycel (Dasatinib), STANFORD V, Sterile Talc Powder (Talc), Steritalc (Talc), Stivarga (Regorafenib), Sunitinib Malate, Sutent (Sunitinib Malate), Sylatron (Peginterferon Alfa-2b), Sylvant (Siltuximab), Synovir (Thalidomide), Synribo (Omacetaxine Mepesuccinate), TAC, Tafinlar (Dabrafenib), Talc, Tamoxifen Citrate, Tarabine PFS (Cytarabine), Tarceva (Erlotinib Hydrochloride), Targretin (Bexarotene), Tasigna (Nilotinib), Taxol (Paclitaxel), Taxotere (Docetaxel), Temodar (Temozolomide), Temozolomide, Temsirolimus, Thalidomide, Thalomid (Thalidomide), Thiotepa, Toposar (Etoposide), Topotecan Hydrochloride, Toremifene, Torisel (Temsirolimus), Tositumomab and Iodine I 131 Tositumomab, Totect (Dexrazoxane Hydrochloride), TPF, Trametinib, Trastuzumab, Treanda (Bendamustine Hydrochloride), Trisenox (Arsenic Trioxide), Tykerb (Lapatinib Ditosylate), Unituxin (Dinutuximab), Vandetanib, VAMP, Vectibix (Panitumumab), VeIP, Velban (Vinblastine Sulfate), Velcade (Bortezomib), Velsar (Vinblastine Sulfate), Vemurafenib, VePesid (Etoposide), Viadur (Leuprolide Acetate), Vidaza (Azacitidine), Vinblastine Sulfate, Vincasar PFS (Vincristine Sulfate), Vincristine Sulfate, Vincristine Sulfate Liposome, Vinorelbine Tartrate, VIP, Vismodegib, Voraxaze (Glucarpidase), Vorinostat, Votrient (Pazopanib Hydrochloride), Wellcovorin (Leucovorin Calcium), Xalkori (Crizotinib), Xeloda (Capecitabine), XELIRI, XELOX, Xgeva (Denosumab), Xofigo (Radium 223 Dichloride), Xtandi (Enzalutamide), Yervoy (Ipilimumab), Zaltrap (Ziv-Aflibercept), Zelboraf (Vemurafenib), Zevalin (Ibritumomab Tiuxetan), Zinecard (Dexrazoxane Hydrochloride), Ziv-Aflibercept, Zofran (Ondansetron Hydrochloride), Zoladex (Goserelin Acetate), Zoledronic Acid, Zolinza (Vorinostat), Zometa (Zoledronic Acid), Zydelig (Idelalisib), Zykadia (Ceritinib), and Zytiga (Abiraterone Acetate).

In some aspects, disclosed herein is a method of stimulating a T cell comprising administering to a subject an effective amount of a composition comprising: an antibody, a ligand, or an antigen binding fragment thereof that specifically binds a co-stimulatory molecule; and a nanoparticle comprising an mRNA encoding the co-stimulatory molecule.

In some aspects, disclosed herein is a method of stimulating a T cell comprising administering to a subject an effective amount of a composition comprising: an antibody or antigen binding fragment thereof that specifically binds a co-stimulatory molecule; and a nanoparticle comprising an mRNA encoding the co-stimulatory molecule.

In some embodiments, the antigen binding fragment that specifically binds a co-stimulatory molecule comprises an CD40 ligand or a functional fragment thereof that binds to CD40. In some embodiments, the CD40 ligand is encoded by a nucleic acid sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO:24.

In some embodiments, the antigen binding fragment that specifically binds a co-stimulatory molecule comprises an OX40 ligand or a functional fragment thereof that binds to OX 40. In some embodiments, the OX 40 ligand is encoded by a nucleic acid sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 13 or 14.

In some embodiments, the antigen binding fragment that specifically binds a co-stimulatory molecule comprises an ICOS ligand or a functional fragment thereof that binds to ICOS. In some embodiments, the ICOS ligand is encoded by a nucleic acid sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 15 or 16.

In some embodiments, the antigen binding fragment that specifically binds a co-stimulatory molecule comprises a CD137 ligand or a functional fragment thereof that binds to CD137. In some embodiments, the CD137 ligand is encoded by a nucleic acid sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 19 or 20. In some embodiments, the subject is a mammal. In some embodiments, the mammal is a human. In some embodiments, the T-cells comprise CD4+ T-cells, CD8+ T-cells, or combinations thereof. In some embodiments, the T-cells comprise CD8+ T-cells. CD8+ T-cells are also referred to as cytotoxic T-cells and can function to kill specifically recognized cells (e.g., tumor cells).

In some embodiments, the antibody or antigen binding fragment thereof that specifically binds a co-stimulatory molecule and the nanoparticle comprising an mRNA encoding the co-stimulatory molecule are administered concurrently (simultaneously or immediately thereafter). In some embodiments, the antibody or antigen binding fragment thereof that specifically binds a co-stimulatory molecule and the nanoparticle comprising an mRNA encoding the co-stimulatory molecule are administered sequentially.

Also disclosed herein are methods of treating a disease or a condition such as an inflammation disorder (including an autoimmune disease) or lymphoid proliferative diseases, comprising administering to a subject in need thereof an effective amount of an antibody, a ligand, or an antigen binding fragment thereof that specifically binds a co-stimulatory molecule and a nanoparticle comprising an mRNA encoding the co-stimulatory molecule.

Further disclosed herein are methods of treating a disease or a condition such as an inflammation disorder (including an autoimmune disease) or lymphoid proliferative diseases, comprising administering to a subject in need thereof an effective amount of an antibody or antigen binding fragment thereof that specifically binds a co-stimulatory molecule and a nanoparticle comprising an mRNA encoding the co-stimulatory molecule.

In one embodiment, provided herein is a method of treating an inflammation disorder, including autoimmune diseases in a subject. The method comprises administering to said subject a therapeutically effective amount of a compound, a combination of compounds, or a composition provided herein, or a pharmaceutically acceptable form thereof, or a pharmaceutical composition as provided herein. Examples of autoimmune diseases include but are not limited to acute disseminated encephalomyelitis (ADEM), Addison's disease, antiphospholipid antibody syndrome (APS), aplastic anemia, autoimmune hepatitis, autoimmune skin disease, coeliac disease, Crohn's disease, Diabetes mellitus (type 1), Goodpasture's syndrome, Graves' disease, Guillain-Barré syndrome (GBS), Hashimoto's disease, lupus erythematosus, multiple sclerosis, myasthenia gravis, opsoclonus myoclonus syndrome (OMS), optic neuritis, Ord's thyroiditis, oemphigus, polyarthritis, primary biliary cirrhosis, psoriasis, rheumatoid arthritis, Reiter's syndrome, Takayasu's arteritis, temporal arteritis (also known as “giant cell arteritis”), warm autoimmune hemolytic anemia, Wegener's granulomatosis, alopecia universalis (e.g., inflammatory alopecia), Chagas disease, chronic fatigue syndrome, dysautonomia, endometriosis, hidradenitis suppurativa, interstitial cystitis, neuromyotonia, sarcoidosis, scleroderma, ulcerative colitis, vitiligo, and vulvodynia. Other disorders include bone-resorption disorders and thrombosis.

Inflammation takes on many forms and includes, but is not limited to, acute, adhesive, atrophic, catarrhal, chronic, cirrhotic, diffuse, disseminated, exudative, fibrinous, fibrosing, focal, granulomatous, hyperplastic, hypertrophic, interstitial, metastatic, necrotic, obliterative, parenchymatous, plastic, productive, proliferous, pseudomembranous, purulent, sclerosing, seroplastic, serous, simple, specific, subacute, suppurative, toxic, traumatic, and/or ulcerative inflammation.

Exemplary inflammatory conditions include, but are not limited to, inflammation associated with acne, anemia (e.g., aplastic anemia, haemolytic autoimmune anaemia), asthma, arteritis (e.g., polyarteritis, temporal arteritis, periarteritis nodosa, Takayasu's arteritis), arthritis (e.g., crystalline arthritis, osteoarthritis, psoriatic arthritis, gout flare, gouty arthritis, reactive arthritis, rheumatoid arthritis and Reiter's arthritis), ankylosing spondylitis, amylosis, amyotrophic lateral sclerosis, autoimmune diseases, allergies or allergic reactions, atherosclerosis, bronchitis, bursitis, chronic prostatitis, conjunctivitis, Chagas disease, chronic obstructive pulmonary disease, cermatomyositis, diverticulitis, diabetes (e.g., type I diabetes mellitus, type 2 diabetes mellitus), a skin condition (e.g., psoriasis, eczema, burns, dermatitis, pruritus (itch)), endometriosis, Guillain-Barre syndrome, infection, ischaemic heart disease, Kawasaki disease, glomerulonephritis, gingivitis, hypersensitivity, headaches (e.g., migraine headaches, tension headaches), ileus (e.g., postoperative ileus and ileus during sepsis), idiopathic thrombocytopenic purpura, interstitial cystitis (painful bladder syndrome), gastrointestinal disorder (e.g., selected from peptic ulcers, regional enteritis, diverticulitis, gastrointestinal bleeding, eosinophilic gastrointestinal disorders (e.g., eosinophilic esophagitis, eosinophilic gastritis, eosinophilic gastroenteritis, eosinophilic colitis), gastritis, diarrhea, gastroesophageal reflux disease (GORD, or its synonym GERD), inflammatory bowel disease (IBD) (e.g., Crohn's disease, ulcerative colitis, collagenous colitis, lymphocytic colitis, ischaemic colitis, diversion colitis, Behcet's syndrome, indeterminate colitis) and inflammatory bowel syndrome (IBS)), lupus, multiple sclerosis, morphea, myeasthenia gravis, myocardial ischemia, nephrotic syndrome, pemphigus vulgaris, pernicious aneaemia, peptic ulcers, polymyositis, primary biliary cirrhosis, neuroinflammation associated with brain disorders (e.g., Parkinson's disease, Huntington's disease, and Alzheimer's disease), prostatitis, chronic inflammation associated with cranial radiation injury, pelvic inflammatory disease, polymyalgia rheumatic, reperfusion injury, regional enteritis, rheumatic fever, systemic lupus erythematosus, scleroderma, scierodoma, sarcoidosis, spondyloarthopathies, Sjogren's syndrome, thyroiditis, transplantation rejection, tendonitis, trauma or injury (e.g., frostbite, chemical irritants, toxins, scarring, burns, physical injury), vasculitis, vitiligo and Wegener's granulomatosis. In certain embodiments, the inflammatory disorder is selected from arthritis (e.g., rheumatoid arthritis), inflammatory bowel disease, inflammatory bowel syndrome, asthma, psoriasis, endometriosis, interstitial cystitis and prostatistis. In certain embodiments, the inflammatory condition is an acute inflammatory condition (e.g., for example, inflammation resulting from infection). In certain embodiments, the inflammatory condition is a chronic inflammatory condition (e.g., conditions resulting from asthma, arthritis and inflammatory bowel disease). The compounds can also be useful in treating inflammation associated with trauma and non-inflammatory myalgia.

Immune disorders, such as auto-immune disorders include, but are not limited to, arthritis (including rheumatoid arthritis, spondyloarthopathies, gouty arthritis, degenerative joint diseases such as osteoarthritis, systemic lupus erythematosus, Sjogren's syndrome, ankylosing spondylitis, undifferentiated spondylitis, Behcet's disease, haemolytic autoimmune anaemias, multiple sclerosis, amyotrophic lateral sclerosis, amylosis, acute painful shoulder, psoriatic, and juvenile arthritis), asthma, atherosclerosis, osteoporosis, bronchitis, tendonitis, bursitis, skin condition (e.g., psoriasis, eczema, burns, dermatitis, pruritus (itch)), enuresis, eosinophilic disease, gastrointestinal disorder (e.g., selected from peptic ulcers, regional enteritis, diverticulitis, gastrointestinal bleeding, eosinophilic gastrointestinal disorders (e.g., eosinophilic esophagitis, eosinophilic gastritis, eosinophilic gastroenteritis, eosinophilic colitis), gastritis, diarrhea, gastroesophageal reflux disease (GORD, or its synonym GERD), inflammatory bowel disease (IBD) (e.g., Crohn's disease, ulcerative colitis, collagenous colitis, lymphocytic colitis, ischaemic colitis, diversion colitis, Behcet's syndrome, indeterminate colitis) and inflammatory bowel syndrome (IBS)), relapsing polychondritis (e.g., atrophic polychondritis and systemic polychondromalacia), and disorders ameliorated by a gastroprokinetic agent (e.g., ileus, postoperative ileus and ileus during sepsis; gastroesophageal reflux disease (GORD, or its synonym GERD); eosinophilic esophagitis, gastroparesis such as diabetic gastroparesis; food intolerances and food allergies and other functional bowel disorders, such as non-ulcerative dyspepsia (NUD) and non-cardiac chest pain (NCCP, including costo-chondritis)).

### EXAMPLES

The following examples are set forth below to illustrate the compounds, compositions, methods, and results according to the disclosed subject matter. These examples are not intended to be inclusive of all aspects of the subject matter disclosed herein, but rather to illustrate representative methods and results. These examples are not intended to exclude equivalents and variations of the present invention which are apparent to one skilled in the art.

### Example 1: Nanomaterials for Nucleic Acid Delivery

Efficient delivery of mRNA is a key step and challenge for the application of mRNA therapeutics. Despite promising data from ongoing clinical trials, the clinical use of mRNA requires the discovery and development of more efficient delivery systems.

Disclosed herein are nanomaterials for gene therapy and drug delivery applications. These compounds were designed and synthesized with toll-like receptor (TLR) agonist heads and tunable lipid tails.

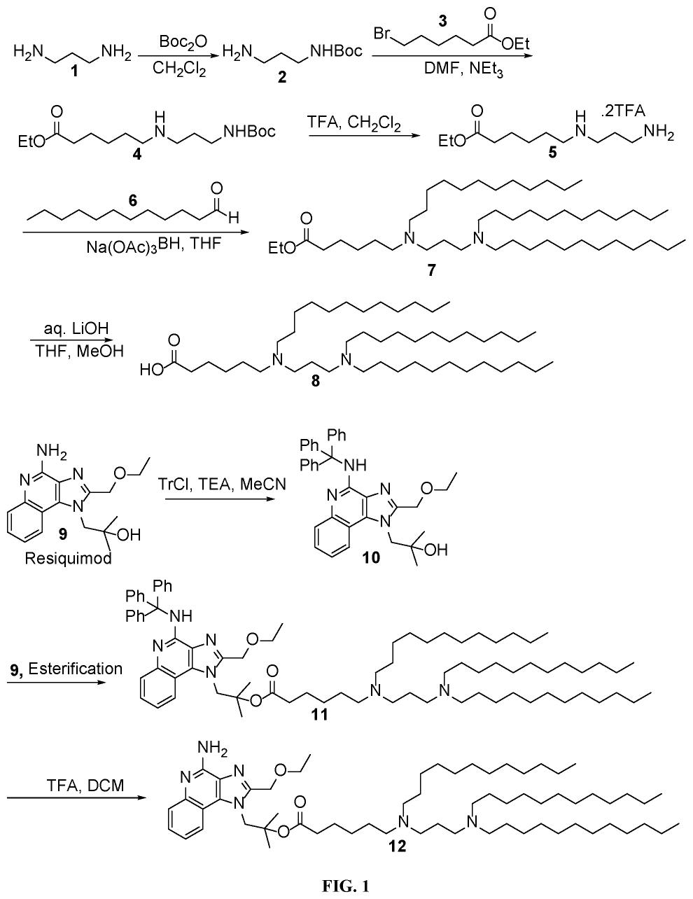

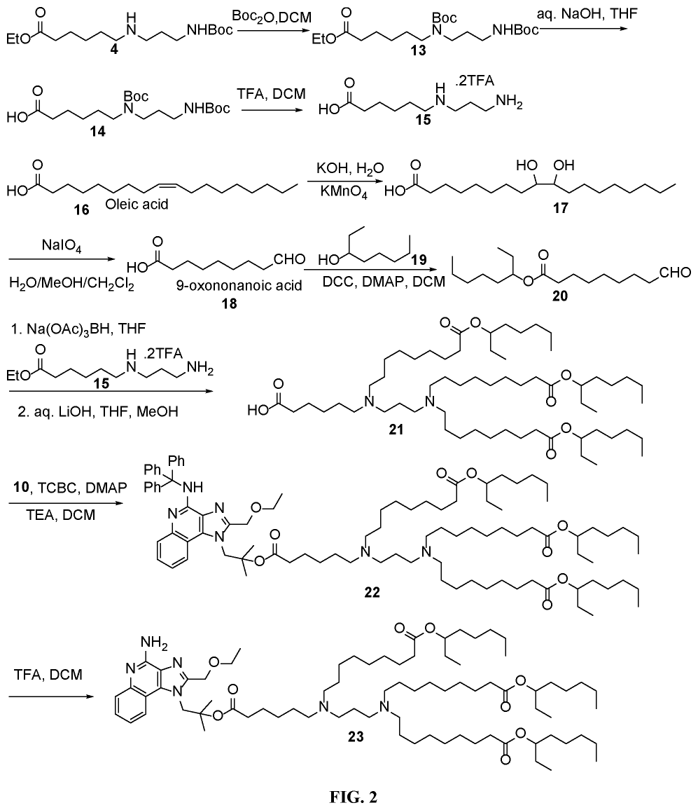

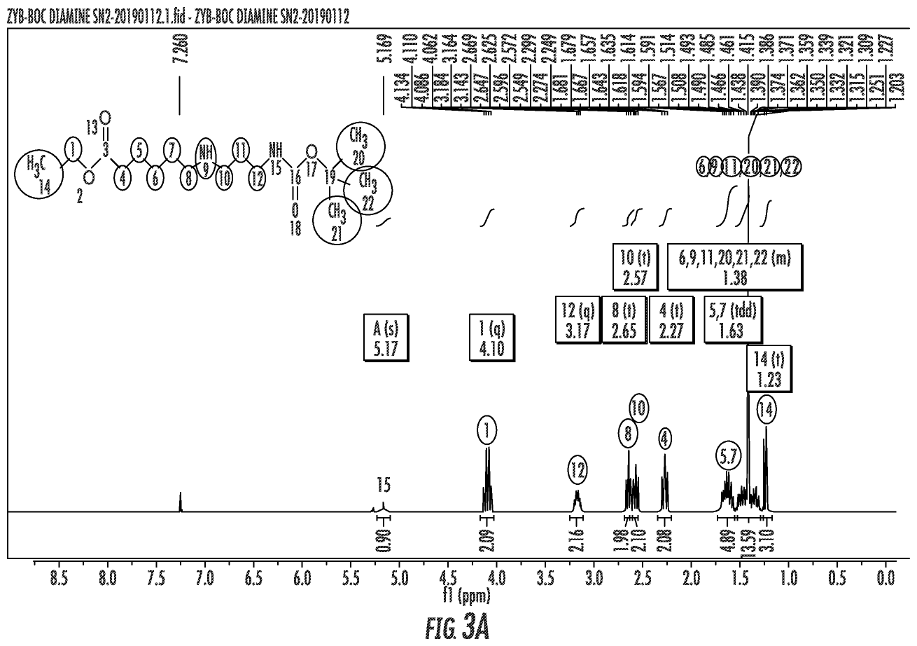

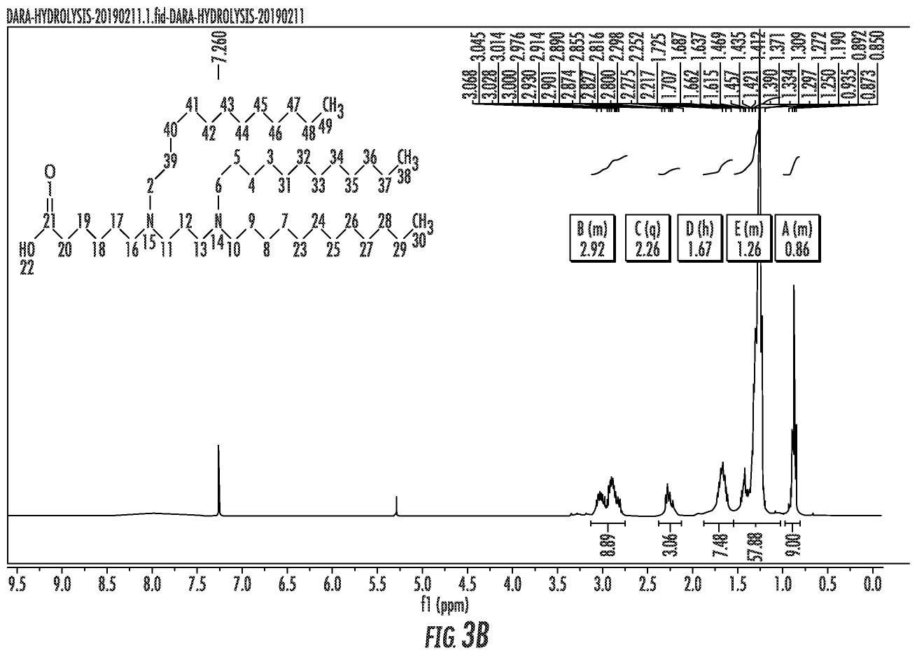

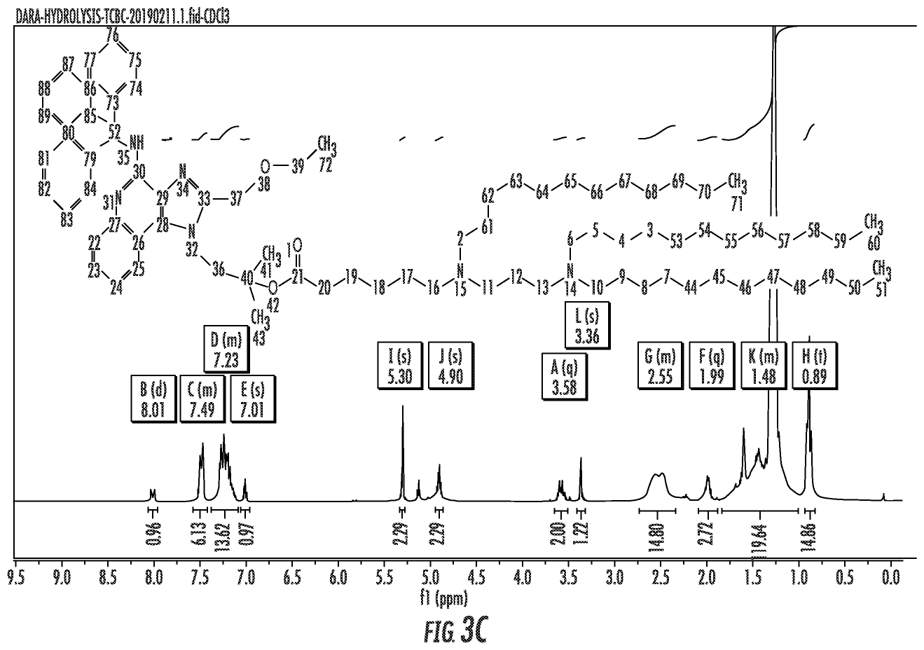

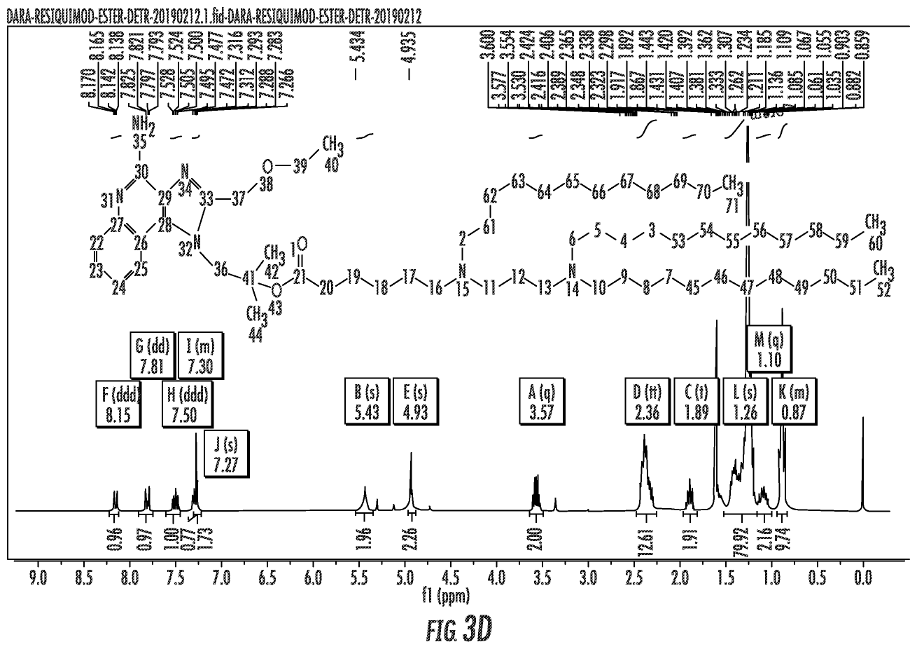

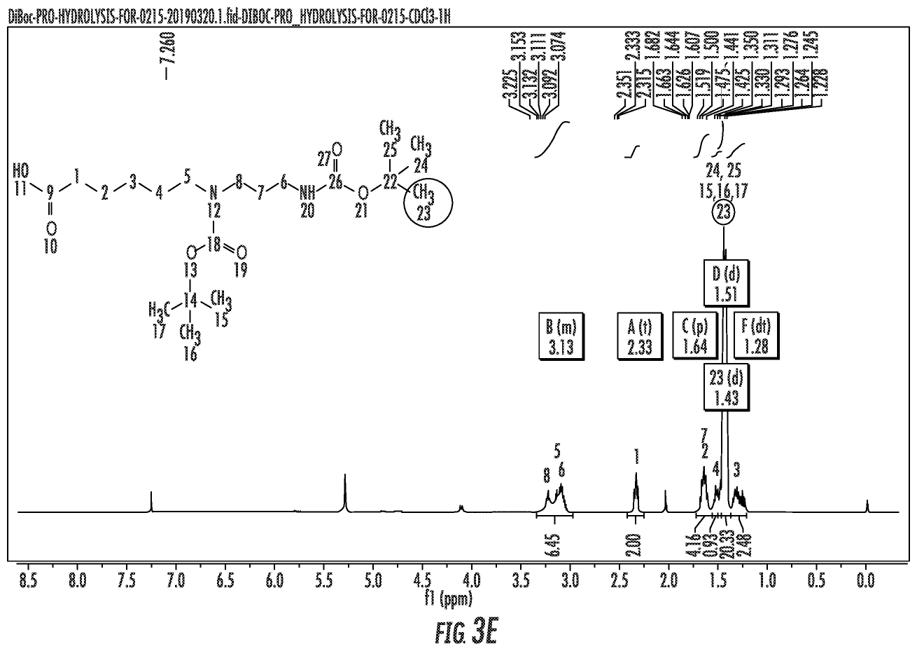

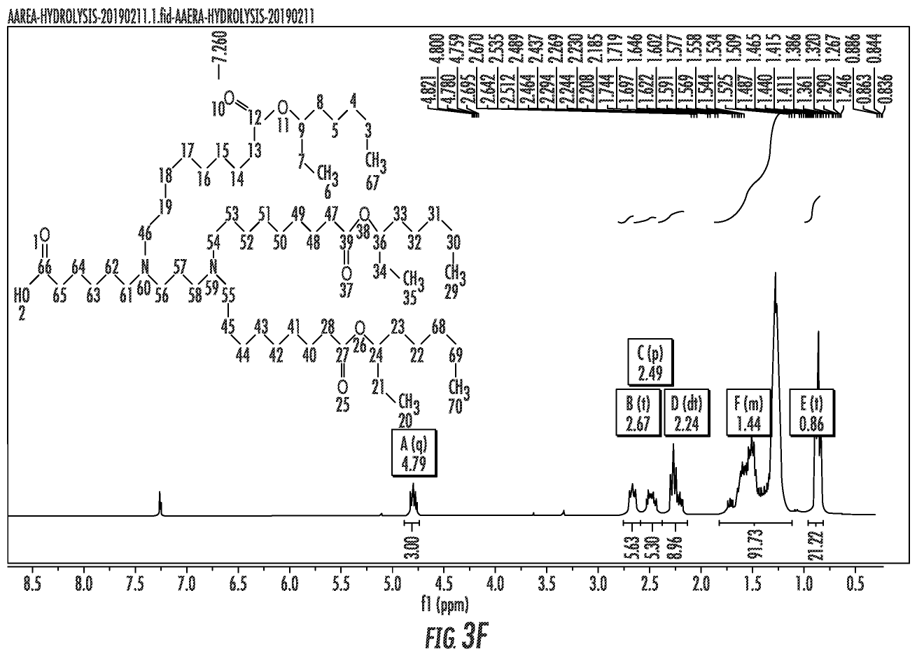

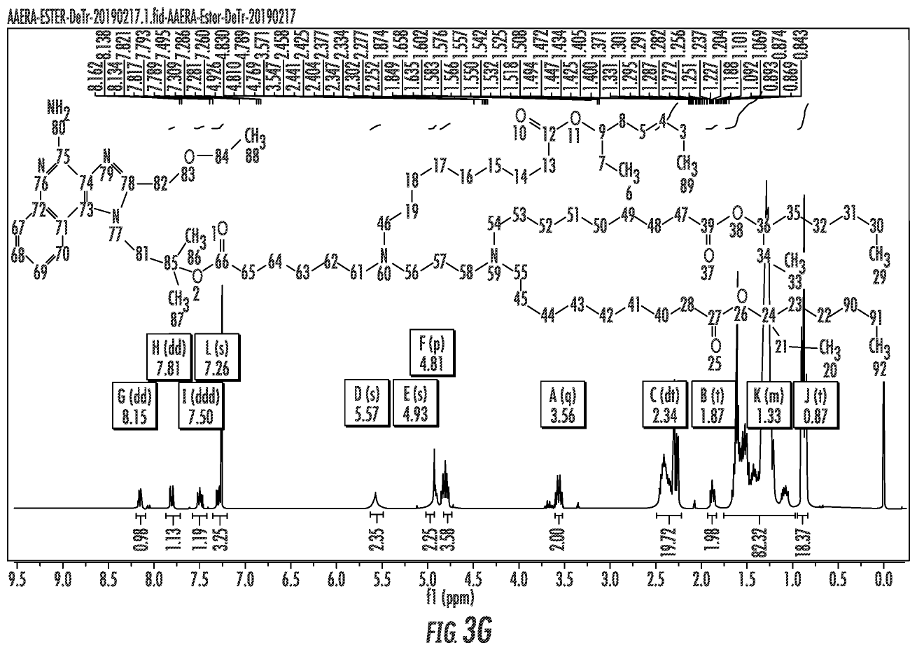

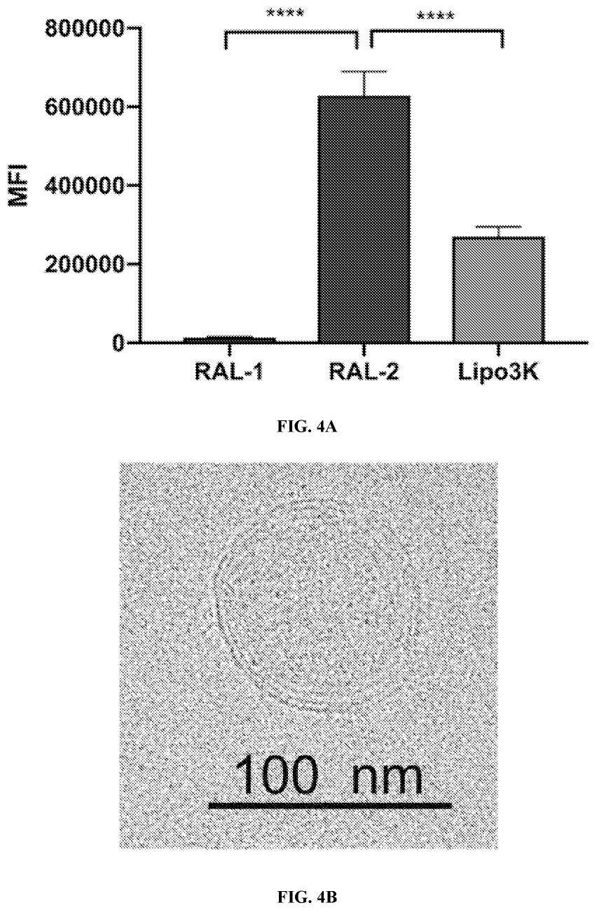

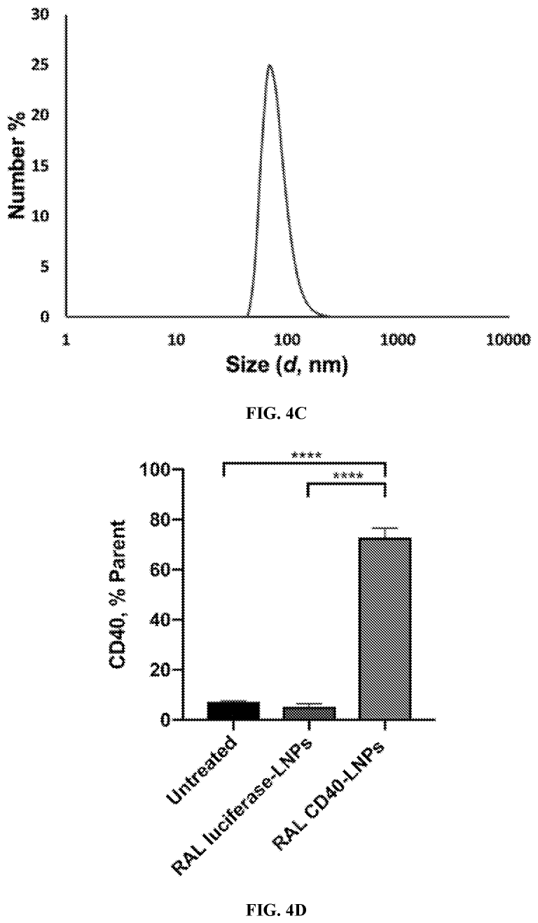

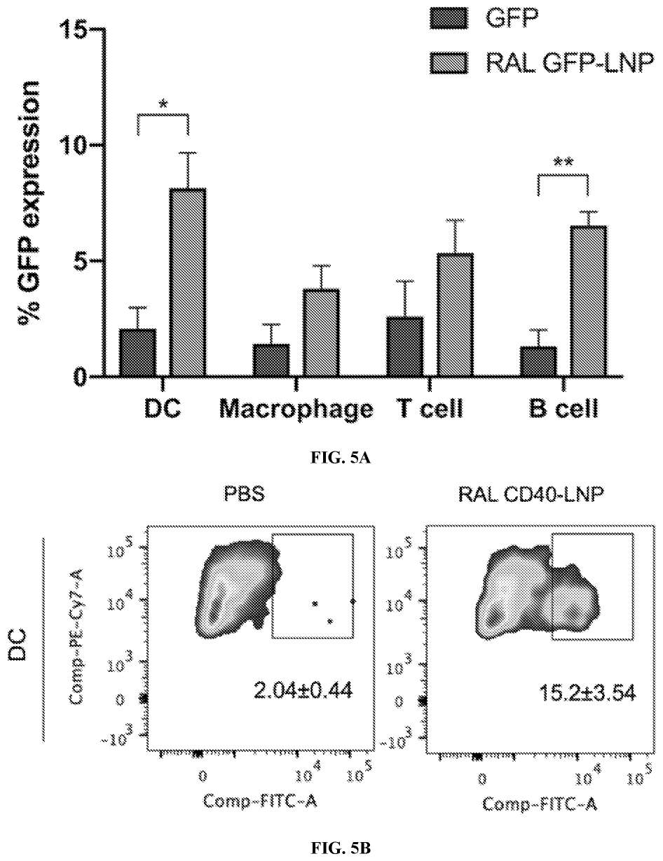

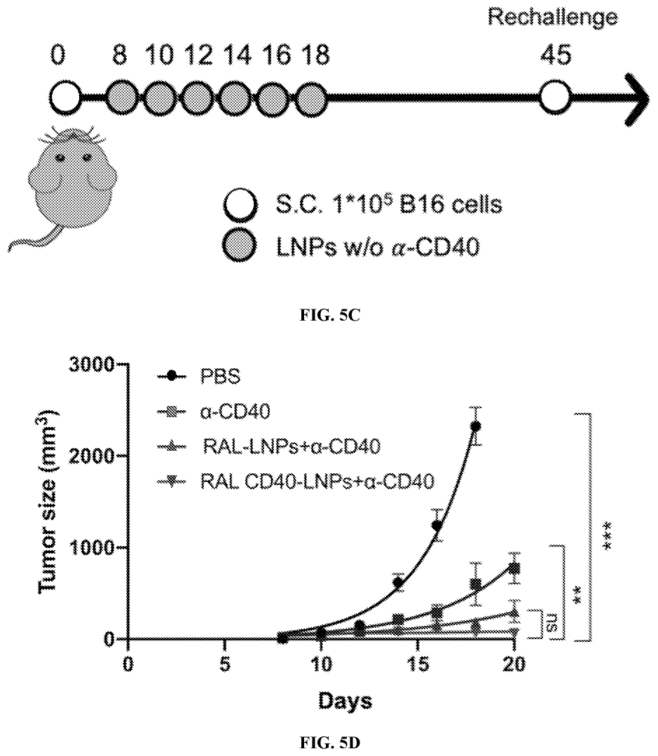

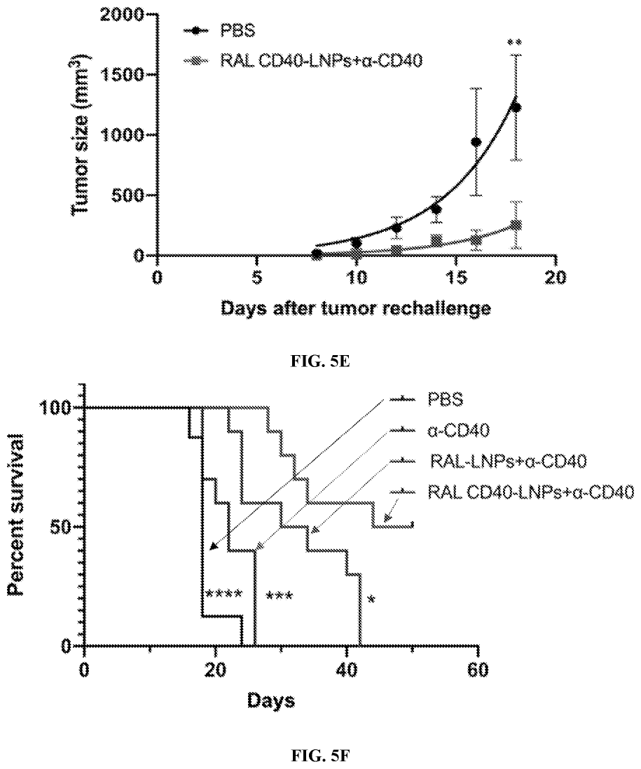

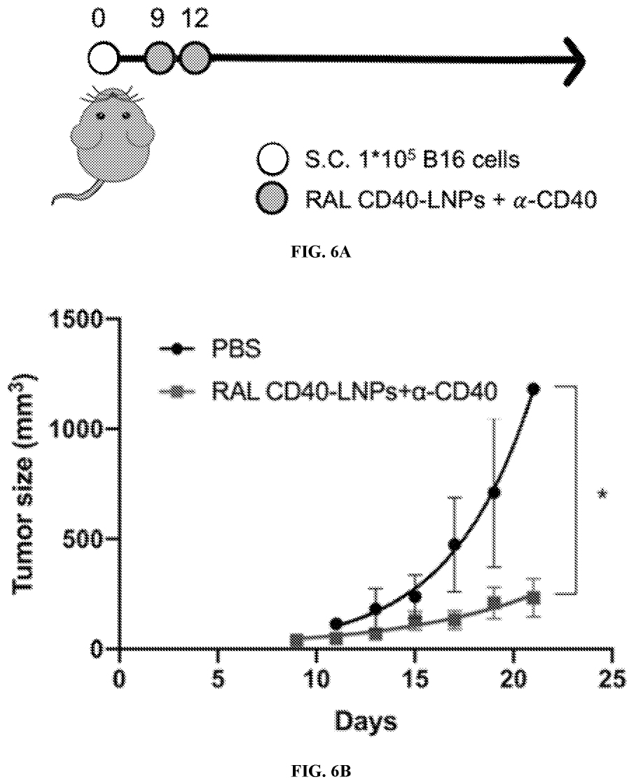

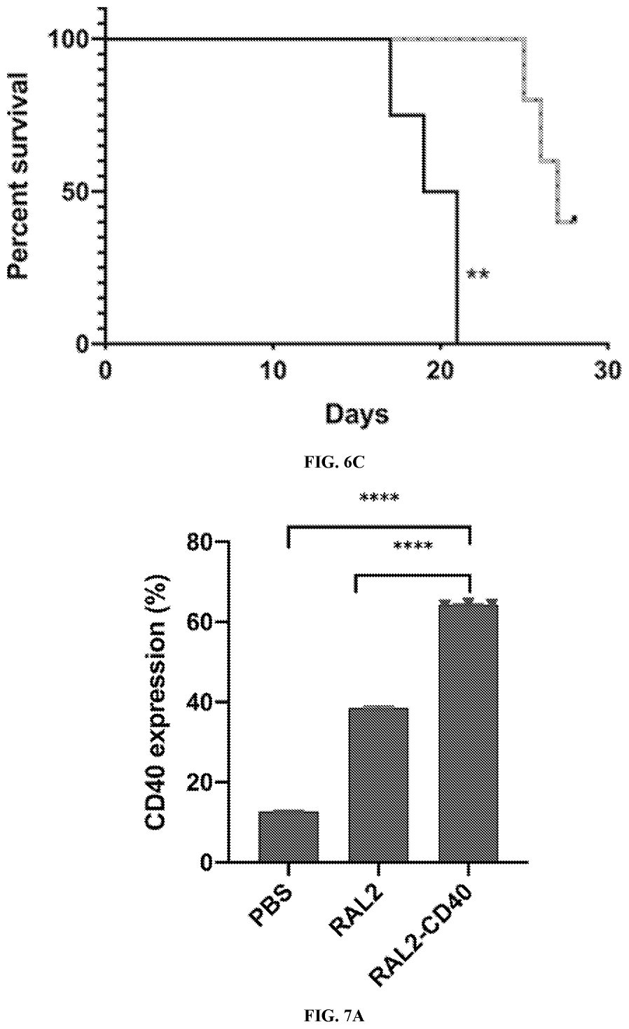

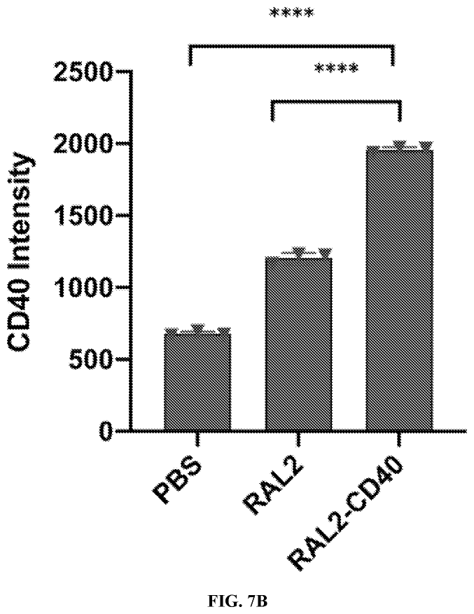

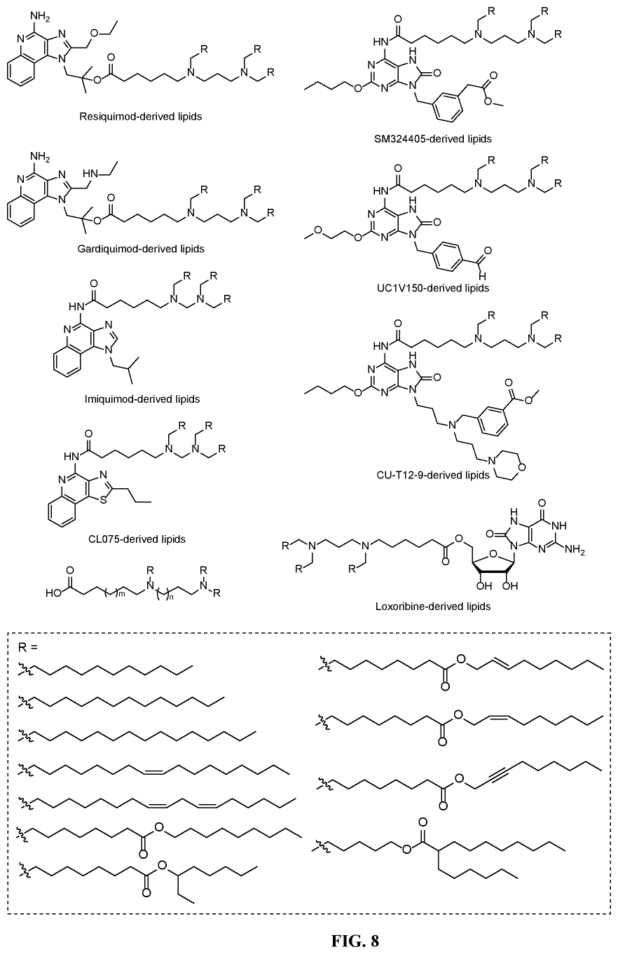

These nanomaterials are composed of three parts, including the toll-like receptor (TLR) agonist heads, the amino cores and lipid tails. FIGS. 1 and 2 show the synthetic routes to these materials.

The synthetic route of Compound 12 is shown in FIG. 1.

Experimental Procedure:

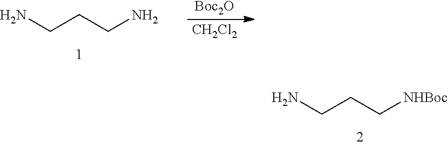 [SMILES: "Boc2O" ; NCCCN ; "CH2Cl2" ; CC(C)(C)OC(=O)NCCCN]

To a solution of 1 (8.64 mL) in 100 mL of DCM was added a solution of Boc₂O (4.3 g) in 50 mL of DCM dropwise at 40° C. over 3 h. The clear solution became white and cloudy, white precipitate was generated. The mixture was stirred for an additional 1 h, then the obtained mixture was washed with 50 mL of 1 M aq. NaHCO₃ solution. The aqueous phase was extracted with 50 mL of DCM for three more times. The organic phase was combined and washed with 20 mL of 1 M NaHCO₃, and dried over anhydrous Na₂SO₄. The solution was filtered and the solvent was removed under reduced pressure. The residue light yellow solid (3.0 g) was used directly without further purification in the following step.

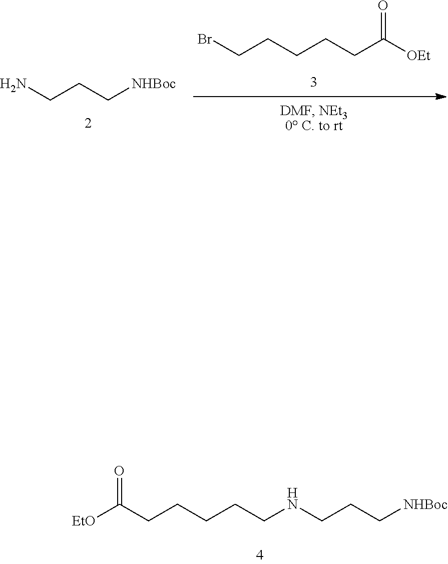 [SMILES: CCOC(=O)CCCCCBr ; CC(C)(C)OC(=O)NCCCN ; "DMF, NEt30° C. to rt" ; CCOC(=O)CCCCCNCCCNC(=O)OC(C)(C)C]

To a solution of 2 (3.0 g) and triethylamine (2.4 mL) in DMF (10 mL) was slowly added a solution of 3 (2.4 g) in DMF (10 mL) at 0° C. The resulting mixture was stirred for 48 h. Then the mixture was diluted with 100 mL of EA, then the organic phase was washed with 50 mL of brine for two times. The aqueous phase was extracted with EA (30 mL×2 times). The organic phase was combined and dried over anhydrous Na₂SO₄. The solution was filtered and the solvent was removed under reduced pressure. The residue yellow oil was purified via silica gel chromatography, 2.25 g of 4 was obtained.

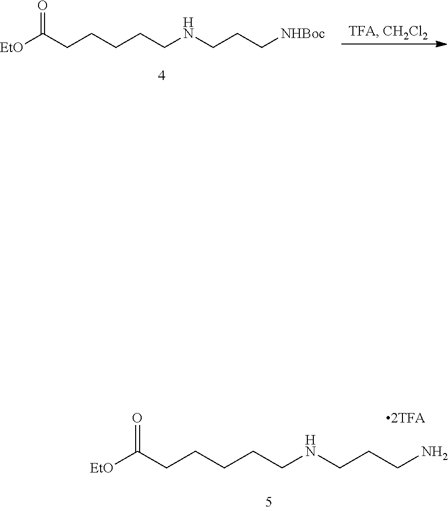

TFA (10 mL) was added dropwise to a solution of 4 (2.25 g) in DCM (30 mL) at 0° C. over 3 min. The resulting mixture was stirred atroom temperaturefor 1 h. The solvent and excess TFA was removed under reduced pressure. The residue was dissolved in methanol (5 mL) and concentrated. The residue oil was used in the next step directly without further purification.

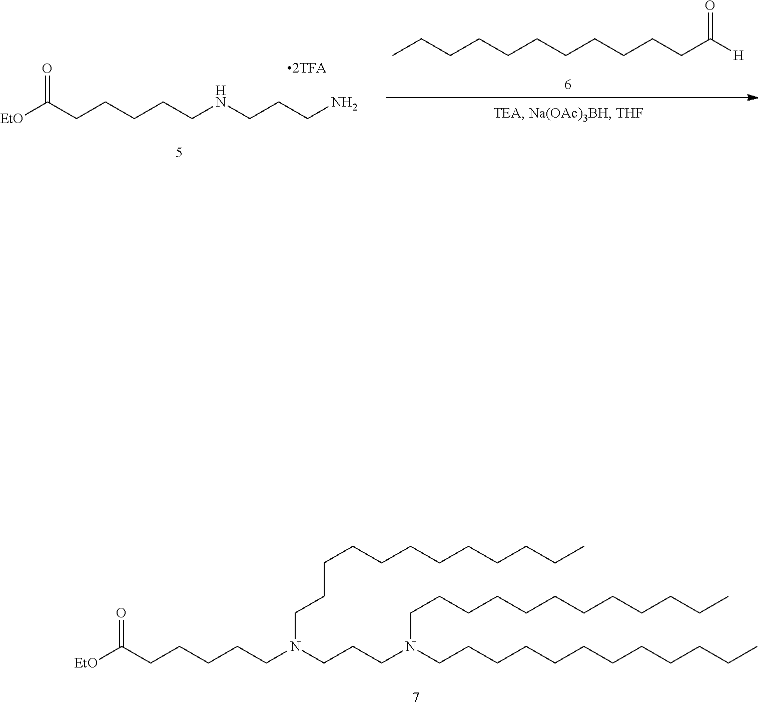 [SMILES: CCCCCCCCCCCC=O ; CCOC(=O)CCCCCNCCCN ; "TEA, Na(OAc)3BH, THF" ; CCCCCCCCCCCCN(CCCCCCCCCCCC)CCCN(CCCCCCCCCCCC)CCCCCC(=O)OCC]

To a solution of 5 (616 mg, 1.39 mmol) in THF (10 mL) was added TEA (2.6 eq., d=0.728, 0.50 mL) at room temperature, the mixture was kept stirring for 1 h. Then Dodecanal (1.02 g, 5.6 eq.) was added and stirred for 30 min. Na(OAc)₃BH (1.76 g, 8.4 mmol) was added to above solution, and the resulting mixture was stirred for 12 h. Aq. NaHCO₃ solution (50 mL) was added to quench the reaction, and the pH value was adjusted to about 10. The aq. phase was extracted with EA (60 mL×2 times), the organic phase was combined and dried over anhydrous Na₂SO₄. The solution was filtered and the solvent was removed under reduced pressure. The residue oil was used in the next step without further purification.

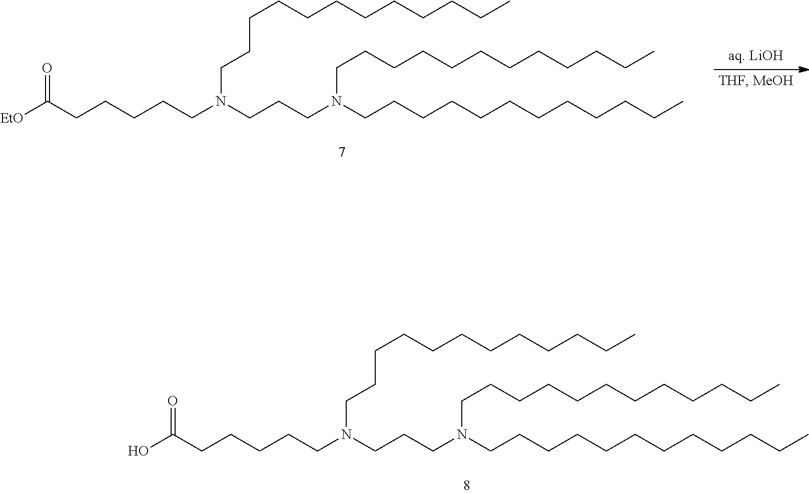

To a solution of 7 in THF (6 mL) and MeOH (2 mL) was added 1 N aq. NaOH solution (3 mL), the mixture was refluxed for 12 h. Aq. NH₄C₁ solution was added to quench the reaction, and the pH value was adjusted to about 10. The aq. phase was extracted with DCM (20 mL x 3 times), the organic phase was combined and dried over anhydrous Na₂SO₄. The solution was filtered and the solvent was removed under reduced pressure. The residue was purified via CombiFlash system. 560 mg acid was obtained, 53.8% for 2 steps.

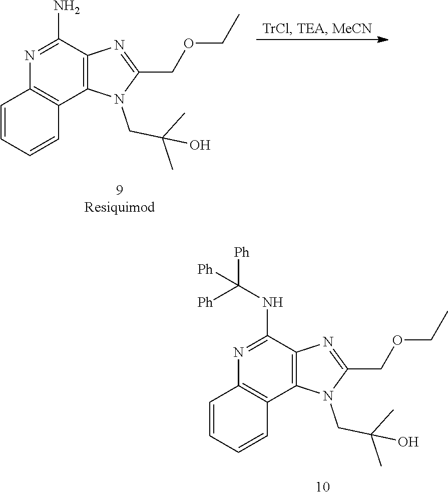

To a solution of 9 (100 mg, 0.318 mmol) and TEA (0.115 mL) in DCM was added Trityl chloride (106.4 mg) at 0° C. Then the mixture was allowed to warm to room temperature and stirred overnight. DCM was removed under reduced pressure. Then cold MeCN was added to the residue, 10 precipitated and was isolated by filtration at 0° C., washed with cold MeCN. The filtrate was further purified via silica gel chromatography. 165 mg desired product was obtained as a white solid.

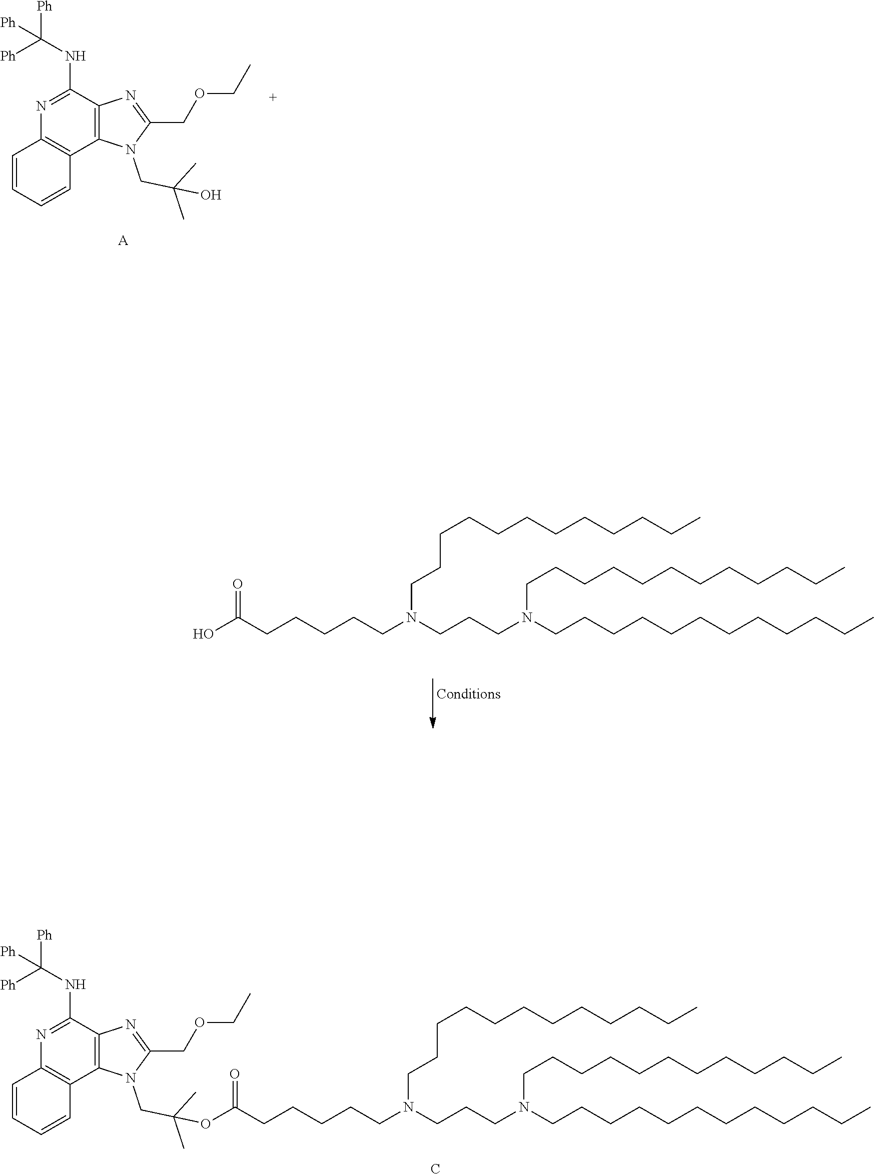

| Entry | Conditions | Results |
|---|---|---|
| 1 | DCC, DMAP, DCM, rt to 50° C. | No desired product |
| 2 | EDC, DMAP, DCM, rt to 50° C. | No desired product |
| 3 | CDI, DCM, rt to 40° C. | No desired product |
| 4 | TCBC, TEA, DMAP, DCM, rt to 40° C. | Trace |
| 5 | TCBC, TEA, DMAP, THF, rt to 40° C. | No reaction |
| 6 | TCBC, TEA, DMAP, THF/Toluene | Trace |
| | (1:2), rt to 40° C. | |
| 7 | TCBC, TEA, DMAP, Toluene, rt to 40° | Yield 50% |
| | C. | |

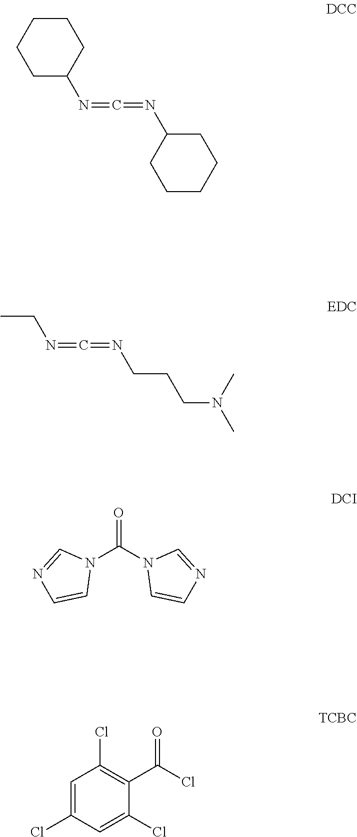

Investigation of Reaction Conditions

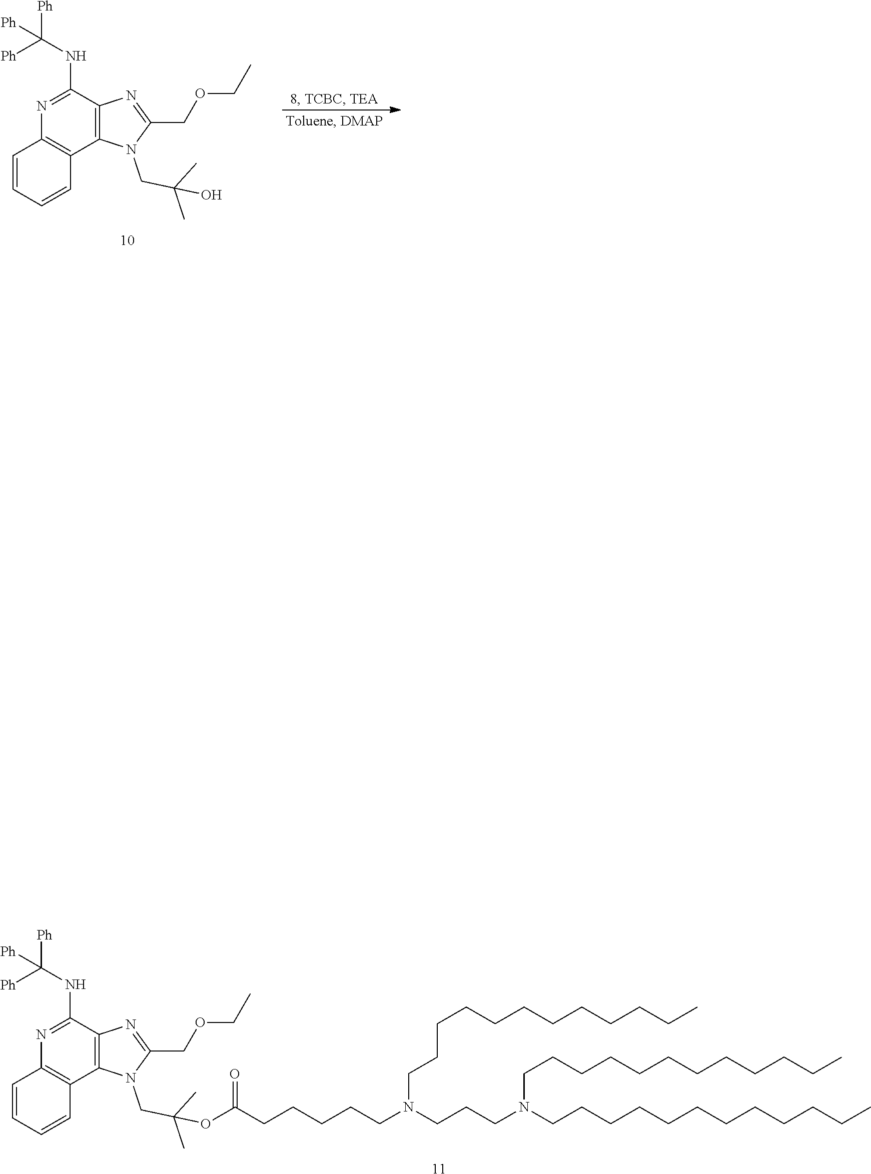

To a solution of 8 (44.8 mg, 0.129 mmol) and TEA (0.027 mL) in 1 mL of Toluene was added 2,4,6-Trichlorobenzoyl Chloride (21.8 mg), and stirred at room temperature for 1 hour. The solution was then allowed to warm to 40° C. and stirred for 2 h. Then a solution of 10 (36 mg) and DMAP (15.7 mg) in 1 mL of anhydrous Toluene was added to the above mixture, the obtained cloudy mixture was stirred for 12 hours at 40° C. Then the reaction mixture was diluted with 20 mL of water and extracted with DCM (15 mL* 3 times). The organic phase dried over anhydrous Na₂SO₄. The solution was filtered and the solvent was removed under reduced pressure. The residue was purified via CombiFlash system 28 mg of 11 was obtained, yield 35%. (5 mg A was recovered via silica gel chromatography.)

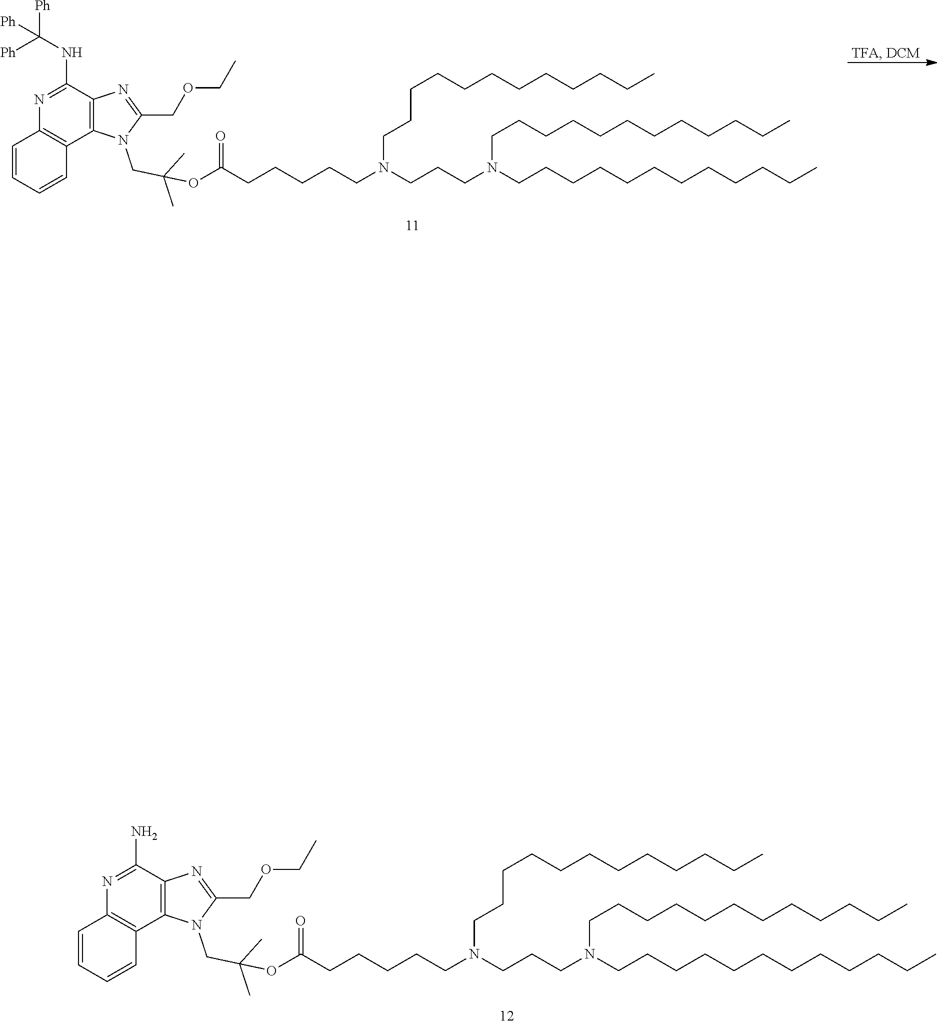

To a solution of 11 (28 mg, 0.0227 mmol) in 2 mL of DCM was added Trifluoroacetic acid (0.5 mL) dropwise at 0° C. The solution was then allowed to warm to room temperature and stirred for 2 h. The reaction was quenched by addition of saturated aqueous NaHCO₃ solution, and extracted with DCM (15 mL* 3 times). The organic phase dried over anhydrous Na₂SO₄. The solution was filtered and the solvent was removed under reduced pressure. The residue was purified via CombiFlash system. 12 mg desired product was obtained, yield 53%. Synthetic route of Compound 23 is shown in FIG. 2.

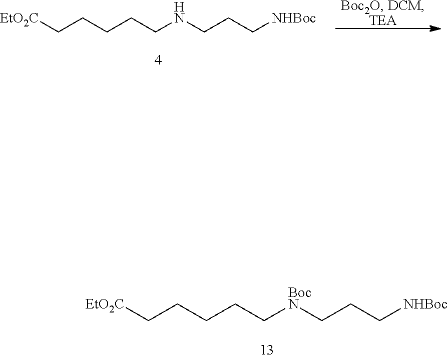 [SMILES: "Boc2O, DCM,TEA" ; CCOC(=O)CCCCCNCCCNC(=O)OC(C)(C)C ; CCOC(=O)CCCCCN(CCCNC(=O)OC(C)(C)C)C(=O)OC(C)(C)C]

4 (475 mg, 1.5 mg) was dissolved in CH₂Cl₂ (8 mL) which was followed by the addition of Boc₂O (50 mg, 0.22 mmol) and Et₃N (26 mg, 0.22 mmol) at room temperature and the mixture was stirred for 2 h. After completion of the reaction, water was added into it, organic phase was separated, aqueous layer was extracted with CH₂Cl₂ (3×20 mL). Organic phases were combined, washed with brine, dried over anhyd. Na₂SO₄ and concentrated to get crude product, which was used directly in the next step.

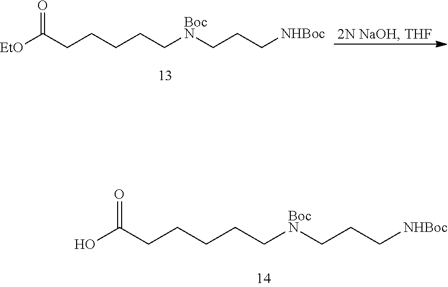

To a solution of 13 (1.5 mmol) in THF (6 mL) and EtOH (1.5 mL) was added 2 N aq. NaOH solution (3 ml) at r.t. The reaction mixture was stirred 3 hours at room temperature. The pH of reaction mixture was adjusted to 2 with 1 N HCl aq. solution, extracted with DCM (3×25 mL), the organic phase was combined and washed with 60 mL of 1 M NaHCO₃, and dried over anhydrous Na₂SO₄. The solution was filtered and the solvent was removed under reduced pressure. The residue oil was used directly without further purification in the following step.

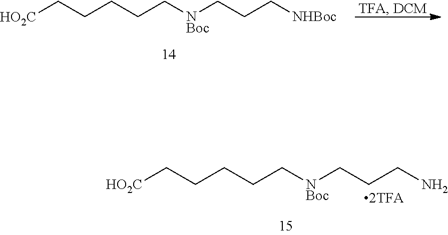

To a suspension of compound 14 (1.5 mmol) in CH₂Cl₂ (7 mL) was added trifluoroacetic acid (2.3 mL). The mixture was allowed to warm to room temperature, stirred at room temperature for 1 hour and monitored with thin layer chromatography (TLC). Upon completion of the reaction, the solvent was evaporated and the residue was dissolved in MeOH and concentrated, the residue solid was used in the next step without further purification.

Compound 17 was synthesis according to Synthetic Communications, 44: 1149-1154, 2014.

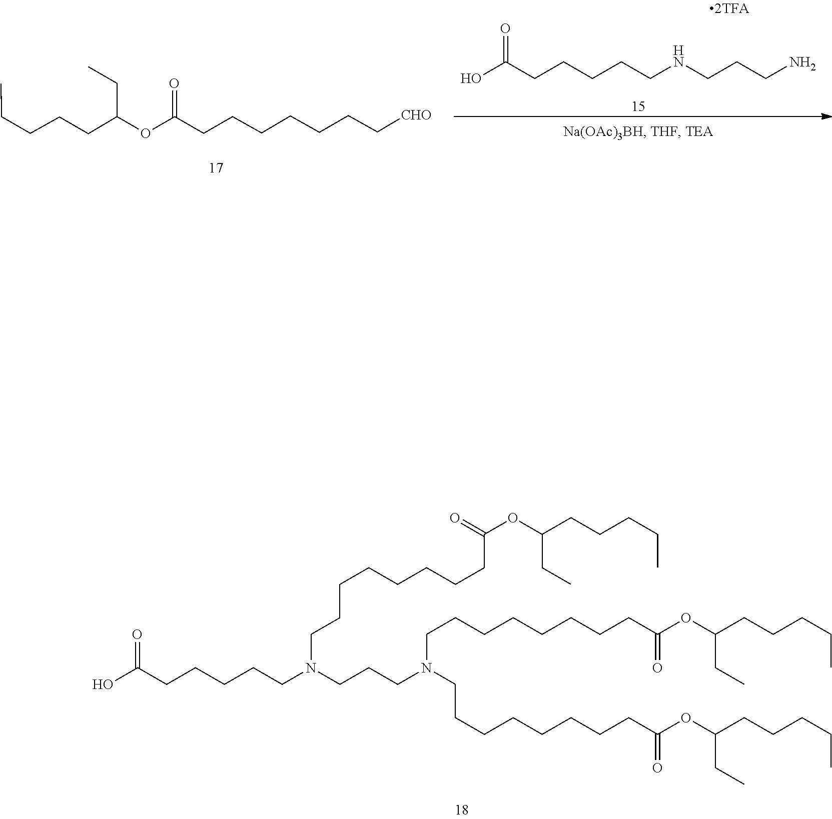

To a solution of 15 (0.6 mmol) in THF (10 mL) was added TEA (0.22 mL) and the mixture was kept stirring for 30 min. Then 17 (0.72 g) was added and stirred for another 30 min. Na(OAc)₃BH (763 mg) was added to above solution, and the resulting mixture was stirred for 12 h. The reaction was quenched with 30 mL of water. The aq. phase was extracted with EA (60 ml×2 times), the organic phase was combined and dried over anhydrous Na₂SO₄. The solution was filtered and the solvent was removed under reduced pressure. The residue was purified via CombiFlash system. 420 mg of C was obtained.

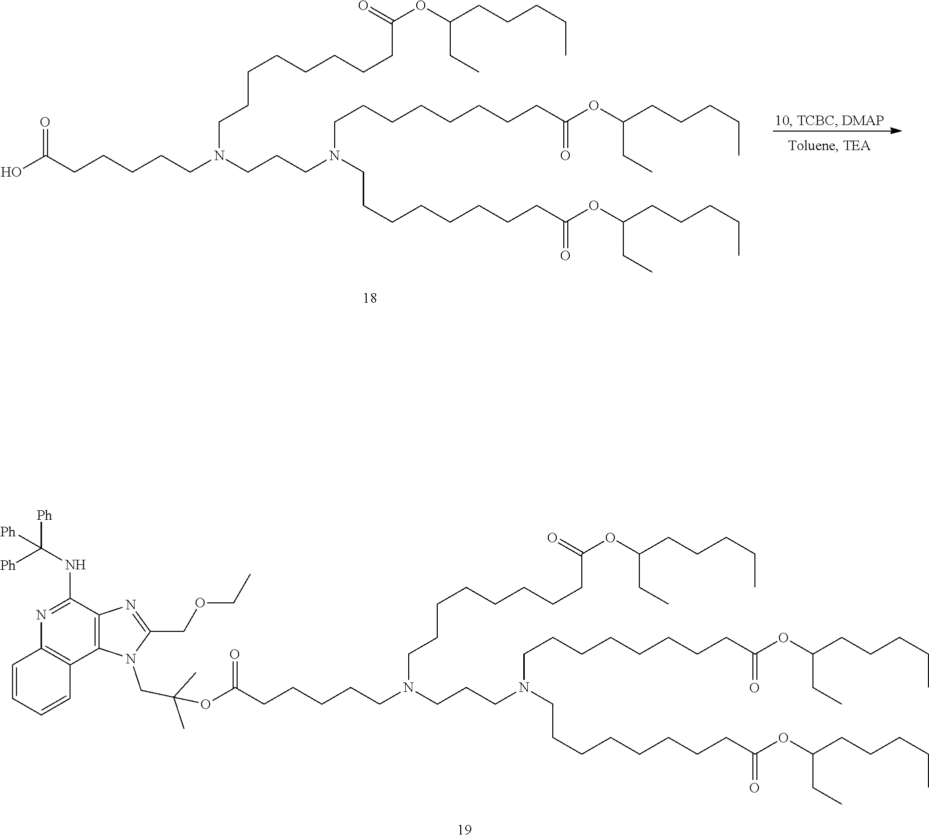 [SMILES: CCCCCC(CC)OC(=O)CCCCCCCCN(CCCCCCCCC(=O)OC(CC)CCCCC)CCCN(CCCCCCCCC(=O)OC(CC)CCCCC)CCCCCC(=O)O ; "10, TCBC, DMAP" ; "Toluene, TEA" ; CCCCCC(CC)OC(=O)CCCCCCCCN(CCCCCCCCC(=O)OC(CC)CCCCC)CCCN(CCCCCCCCC(=O)OC(CC)CCCCC)CCCCCC(=O)OC(C)(C)Cn1c(COCC)nc2c(NC(c3ccccc3)(c3ccccc3)c3ccccc3)nc3ccccc3c21]

### Materials:

A (F.W. 556.7, 1.0 eq, 180 mg, 0.323 mmol), 2,4,6-Trichlorobenzoyl Chloride (F.W.=225.5, 1.5 eq., 141 mg), NEt₃ (F.W.=101.2, 3.0 eq., 0.72.8 g/mL, 174 μL), DMAP (F.W.=122.2, 2.0 eq., 78.9 mg), and B (F.W. 993.6, 1.1 eq., 440 mg).

To a solution of 21 (180 mg, 0.323 mmol) and TEA (0.174 mL) in 2 mL of Toluene was added 2,4,6-Trichlorobenzoyl Chloride (141 mg), and stirred at room temperature for 1 hour. The solution was then allowed to warm to 40° C. and stirred for 2 hours. Then a solution of 10 (180 mg) and DMAP (78.9 mg) in 2 mL of anhydrous Toluene was added to the above mixture, the obtained cloudy mixture was stirred for 12 hours at 40° C. Then the reaction mixture was diluted with 20 mL of water and extracted with DCM (15 mL* 3 times). The organic phase dried over anhydrous Na₂SO₄. The solution was filtered and the solvent was removed under reduced pressure. The reaction mixture was diluted with 20 mL of water and extracted with DCM (15 mL* 3 times). The organic phase dried over anhydrous Na₂SO₄. The solution was filtered and the solvent was removed under reduced pressure. The residue was purified via CombiFlash system, 250 mg of 22 was obtained, 50.5% yield.

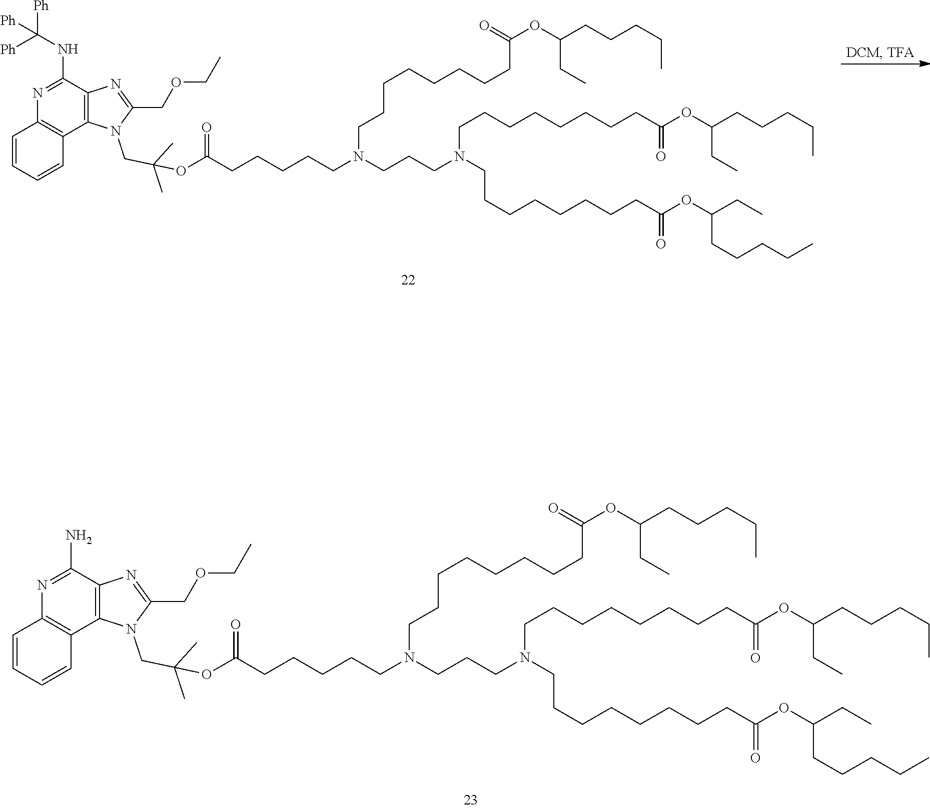

To a solution of 22 (250 mg, 0.163 mmol) in 6 mL of DCM was added Trifluoroacetic acid (2 mL) dropwise at 0° C. The solution was then allowed to warmed to room temperature and stirred for 2 hours. The reaction was quenched by addition of saturated aqueous NaHCO₃ solution (30 mL), and extracted with DCM (30 mL* 3 times). The organic phase dried over anhydrous Na₂SO₄. The solution was filtered and the solvent was removed under reduced pressure. The residue was purified via CombiFlash system. 173 mg of compound 23 was obtained, yield 82%.

SEQUENCES

RNA sequences
Human OX40 mRNA (With 5′UTR and 3′UTR)(SEQ ID NO: 1)
GGGAAAAGUAGAAAGAAAGAAAGAAGAGAAAAUAAAGACAAAGAGCCA
CCAUGUGCGUGGGAGCACGGAGACUGGGAAGGGGACCUUGCGCCGCCCUGCUG
CUGCUGGGCCUGGGCCUGUCCACCGUGACAGGCCUGCACUGCGUGGGCGACAC
CUACCCUUCUAACGAUAGGUGCUGUCACGAGUGUCGCCCAGGCAAUGGCAUGG
UGUCCAGGUGCUCCCGCUCUCAGAACACCGUGUGCCGGCCUUGUGGCCCAGGC
UUCUAUAAUGACGUGGUGAGCUCCAAGCCCUGCAAGCCUUGUACAUGGUGCAA
CCUGCGGAGCGGCUCCGAGAGAAAGCAGCUGUGCACCGCCACACAGGAUACCG
UGUGCCGGUGUAGAGCCGGCACACAGCCACUGGACUCUUACAAGCCAGGAGUG
GAUUGUGCACCUUGCCCACCUGGCCACUUUAGCCCAGGCGACAACCAGGCCUG
UAAGCCCUGGACCAAUUGCACACUGGCAGGCAAGCACACCCUGCAGCCAGCAU
CUAAUUCUAGCGAUGCCAUCUGCGAGGACAGAGAUCCACCAGCAACCCAGCCU
CAGGAGACACAGGGACCUCCAGCCAGGCCAAUCACCGUGCAGCCAACAGAGGC
AUGGCCUCGGACCUCUCAGGGACCAAGCACAAGACCCGUGGAGGUGCCUGGAG
GAAGGGCAGUGGCAGCUAUCUUGGGGCUCGGGUUGGUACUGGGACUGCUUGG
CCCACUUGCUAUCUUGCUGGCUCUGUAUCUGCUGAGGCGCGACCAGCGCCUGC
CCCCUGAUGCACACAAGCCACCAGGAGGAGGAAGCUUCCGGACCCCAAUCCAG
GAGGAGCAGGCAGACGCACACUCCACACUGGCCAAGAUCUGAUUGUGUAUGCG
UUAAUAAAAAGAAGGAACUCGUA
Mouse OX40 mRNA (With 5′UTR and 3′UTR)(SEQ ID NO: 2)
GGGAAAAGUAGAAAGAAAGAAAGAAGAGAAAAUAAAGACAAAGAGCCA
CCAUGUAUGUGUGGGUUCAGCAGCCCACAGCCCUUCUGCUGCUGGGACUCACA
CUUGGAGUUACAGCAAGGCGGCUCAACUGUGUUAAACAUACCUACCCCAGUGG
UCACAAGUGCUGUCGUGAGUGCCAGCCAGGCCAUGGUAUGGUGAGCCGCUGUG
AUCAUACCAGGGAUACUCUAUGUCAUCCGUGUGAGACUGGCUUCUACAAUGA
AGCUGUCAAUUAUGAUACCUGCAAGCAGUGUACACAGUGCAACCAUCGAAGU
GGAAGUGAACUCAAGCAGAAUUGCACACCUACUCAGGAUACUGUCUGCAGAU
GUAGACCAGGCACCCAACCUCGGCAGGACAGCGGCUACAAGCUUGGAGUUGAC
UGUGUUCCCUGCCCUCCUGGCCACUUUUCUCCAGGCAACAACCAGGCCUGCAA
GCCCUGGACCAAUUGUACCUUAUCUGGAAAGCAGACCCGCCACCCAGCCAGUG
ACAGCUUGGACGCAGUCUGUGAGGACAGAAGCCUCCUGGCCACACUGCUCUGG
GAGACCCAGCGCCCUACAUUCAGGCCAACCACUGUCCAAUCCACCACAGUCUG
GCCCAGGACUUCUGAGUUGCCCUCUCCACCCACCUUGGUGACUCCUGAGGGCC
CUGCAUUUGCUGUUCUCCUAGGCCUGGGCCUGGGCCUGCUGGCUCCCUUGACU
GUCCUGCUGGCCUUGUACCUGCUCCGGAAGGCUUGGAGAUUGCCUAACACUCC
CAAACCUUGUUGGGGAAACAGCUUCAGGACCCCGAUCCAGGAGGAACACACAG
ACGCACACUUUACUCUGGCCAAGAUCUGAUUGUGUAUGCGUUAAUAAAAAGA
AGGAACUCGUA
HETEROLOGOUS 5′UTR/3′UTR
5′UTR:
GGGAAAAGUAGAAAGAAAGAAAGAAGAGAAAAUAAAGACAAAGAGCCACC
(SEQ ID NO: 3)
3′UTR: UUGUGUAUGCGUUAAUAAAAAGAAGGAACUCGUA (SEQ ID NO: 4)
Human OX40 coding sequence (SEQ ID NO: 5)
AUGUGCGUGGGAGCACGGAGACUGGGAAGGGGACCUUGCGCCGCCCUG
CUGCUGCUGGGCCUGGGCCUGUCCACCGUGACAGGCCUGCACUGCGUGGGCGA
CACCUACCCUUCUAACGAUAGGUGCUGUCACGAGUGUCGCCCAGGCAAUGGCA
UGGUGUCCAGGUGCUCCCGCUCUCAGAACACCGUGUGCCGGCCUUGUGGCCCA
GGCUUCUAUAAUGACGUGGUGAGCUCCAAGCCCUGCAAGCCUUGUACAUGGUG
CAACCUGCGGAGCGGCUCCGAGAGAAAGCAGCUGUGCACCGCCACACAGGAUA
CCGUGUGCCGGUGUAGAGCCGGCACACAGCCACUGGACUCUUACAAGCCAGGA
GUGGAUUGUGCACCUUGCCCACCUGGCCACUUUAGCCCAGGCGACAACCAGGC
CUGUAAGCCCUGGACCAAUUGCACACUGGCAGGCAAGCACACCCUGCAGCCAG
CAUCUAAUUCUAGCGAUGCCAUCUGCGAGGACAGAGAUCCACCAGCAACCCAG
CCUCAGGAGACACAGGGACCUCCAGCCAGGCCAAUCACCGUGCAGCCAACAGA
GGCAUGGCCUCGGACCUCUCAGGGACCAAGCACAAGACCCGUGGAGGUGCCUG
GAGGAAGGGCAGUGGCAGCUAUCUUGGGGCUCGGGUUGGUACUGGGACUGCU
UGGCCCACUUGCUAUCUUGCUGGCUCUGUAUCUGCUGAGGCGCGACCAGCGCC
UGCCCCCUGAUGCACACAAGCCACCAGGAGGAGGAAGCUUCCGGACCCCAAUC
CAGGAGGAGCAGGCAGACGCACACUCCACACUGGCCAAGAUCUGA
Mouse OX40 coding sequence (SEQ ID NO: 6)
AUGUAUGUGUGGGUUCAGCAGCCCACAGCCCUUCUGCUGCUGGGACUC
ACACUUGGAGUUACAGCAAGGCGGCUCAACUGUGUUAAACAUACCUACCCCAG
UGGUCACAAGUGCUGUCGUGAGUGCCAGCCAGGCCAUGGUAUGGUGAGCCGCU
GUGAUCAUACCAGGGAUACUCUAUGUCAUCCGUGUGAGACUGGCUUCUACAA
UGAAGCUGUCAAUUAUGAUACCUGCAAGCAGUGUACACAGUGCAACCAUCGA
AGUGGAAGUGAACUCAAGCAGAAUUGCACACCUACUCAGGAUACUGUCUGCA
GAUGUAGACCAGGCACCCAACCUCGGCAGGACAGCGGCUACAAGCUUGGAGUU
GACUGUGUUCCCUGCCCUCCUGGCCACUUUUCUCCAGGCAACAACCAGGCCUG
CAAGCCCUGGACCAAUUGUACCUUAUCUGGAAAGCAGACCCGCCACCCAGCCA
GUGACAGCUUGGACGCAGUCUGUGAGGACAGAAGCCUCCUGGCCACACUGCUC
UGGGAGACCCAGCGCCCUACAUUCAGGCCAACCACUGUCCAAUCCACCACAGU
CUGGCCCAGGACUUCUGAGUUGCCCUCUCCACCCACCUUGGUGACUCCUGAGG
GCCCUGCAUUUGCUGUUCUCCUAGGCCUGGGCCUGGGCCUGCUGGCUCCCUUG
ACUGUCCUGCUGGCCUUGUACCUGCUCCGGAAGGCUUGGAGAUUGCCUAACAC
UCCCAAACCUUGUUGGGGAAACAGCUUCAGGACCCCGAUCCAGGAGGAACACA
CAGACGCACACUUUACUCUGGCCAAGAUCUGA
DNA sequences
Human OX40 mRNA (With 5′UTR and 3′UTR)(SEQ ID NO: 7)
GGGAAAAGTAGAAAGAAAGAAAGAAGAGAAAATAAAGACAAAGAGCCA
CCATGTGCGTGGGAGCACGGAGACTGGGAAGGGGACCTTGCGCCGCCCTGCTGC
TGCTGGGCCTGGGCCTGTCCACCGTGACAGGCCTGCACTGCGTGGGCGACACCTA
CCCTTCTAACGATAGGTGCTGTCACGAGTGTCGCCCAGGCAATGGCATGGTGTCC
AGGTGCTCCCGCTCTCAGAACACCGTGTGCCGGCCTTGTGGCCCAGGCTTCTATA
ATGACGTGGTGAGCTCCAAGCCCTGCAAGCCTTGTACATGGTGCAACCTGCGGA
GCGGCTCCGAGAGAAAGCAGCTGTGCACCGCCACACAGGATACCGTGTGCCGGT
GTAGAGCCGGCACACAGCCACTGGACTCTTACAAGCCAGGAGTGGATTGTGCAC
CTTGCCCACCTGGCCACTTTAGCCCAGGCGACAACCAGGCCTGTAAGCCCTGGAC
CAATTGCACACTGGCAGGCAAGCACACCCTGCAGCCAGCATCTAATTCTAGCGA
TGCCATCTGCGAGGACAGAGATCCACCAGCAACCCAGCCTCAGGAGACACAGGG
ACCTCCAGCCAGGCCAATCACCGTGCAGCCAACAGAGGCATGGCCTCGGACCTC
TCAGGGACCAAGCACAAGACCCGTGGAGGTGCCTGGAGGAAGGGCAGTGGCAG
CTATCTTGGGGCTCGGGTTGGTACTGGGACTGCTTGGCCCACTTGCTATCTTGCTG
GCTCTGTATCTGCTGAGGCGCGACCAGCGCCTGCCCCCTGATGCACACAAGCCAC
CAGGAGGAGGAAGCTTCCGGACCCCAATCCAGGAGGAGCAGGCAGACGCACAC
TCCACACTGGCCAAGATCTGATTGTGTATGCGTTAATAAAAAGAAGGAACTCGT
A
Mouse OX40 mRNA (With 5′UTR and 3′UTR)(SEQ ID NO: 8)
GGGAAAAGTAGAAAGAAAGAAAGAAGAGAAAATAAAGACAAAGAGCCA
CCATGTATGTGTGGGTTCAGCAGCCCACAGCCCTTCTGCTGCTGGGACTCACACT
TGGAGTTACAGCAAGGCGGCTCAACTGTGTTAAACATACCTACCCCAGTGGTCAC
AAGTGCTGTCGTGAGTGCCAGCCAGGCCATGGTATGGTGAGCCGCTGTGATCAT
ACCAGGGATACTCTATGTCATCCGTGTGAGACTGGCTTCTACAATGAAGCTGTCA
ATTATGATACCTGCAAGCAGTGTACACAGTGCAACCATCGAAGTGGAAGTGAAC
TCAAGCAGAATTGCACACCTACTCAGGATACTGTCTGCAGATGTAGACCAGGCA
CCCAACCTCGGCAGGACAGCGGCTACAAGCTTGGAGTTGACTGTGTTCCCTGCCC
TCCTGGCCACTTTTCTCCAGGCAACAACCAGGCCTGCAAGCCCTGGACCAATTGT
ACCTTATCTGGAAAGCAGACCCGCCACCCAGCCAGTGACAGCTTGGACGCAGTC
TGTGAGGACAGAAGCCTCCTGGCCACACTGCTCTGGGAGACCCAGCGCCCTACA
TTCAGGCCAACCACTGTCCAATCCACCACAGTCTGGCCCAGGACTTCTGAGTTGC
CCTCTCCACCCACCTTGGTGACTCCTGAGGGCCCTGCATTTGCTGTTCTCCTAGGC
CTGGGCCTGGGCCTGCTGGCTCCCTTGACTGTCCTGCTGGCCTTGTACCTGCTCCG
GAAGGCTTGGAGATTGCCTAACACTCCCAAACCTTGTTGGGGAAACAGCTTCAG
GACCCCGATCCAGGAGGAACACACAGACGCACACTTTACTCTGGCCAAGATCTG
ATTGTGTATGCGTTAATAAAAAGAAGGAACTCGTA
HETEROLOGOUS 5′UTR/3′UTR
5′UTR:
GGGAAAAGTAGAAAGAAAGAAAGAAGAGAAAATAAAGACAAAGAGCCACC
(SEQ ID NO: 9)
3′UTR: TTGTGTATGCGTTAATAAAAAGAAGGAACTCGTA (SEQ ID NO: 10)
Human OX40 coding sequence (SEQ ID NO: 11)
ATGTGCGTGGGAGCACGGAGACTGGGAAGGGGACCTTGCGCCGCCCTGCT
GCTGCTGGGCCTGGGCCTGTCCACCGTGACAGGCCTGCACTGCGTGGGCGACAC
CTACCCTTCTAACGATAGGTGCTGTCACGAGTGTCGCCCAGGCAATGGCATGGTG
TCCAGGTGCTCCCGCTCTCAGAACACCGTGTGCCGGCCTTGTGGCCCAGGCTTCT
ATAATGACGTGGTGAGCTCCAAGCCCTGCAAGCCTTGTACATGGTGCAACCTGCG
GAGCGGCTCCGAGAGAAAGCAGCTGTGCACCGCCACACAGGATACCGTGTGCCG
GTGTAGAGCCGGCACACAGCCACTGGACTCTTACAAGCCAGGAGTGGATTGTGC
ACCTTGCCCACCTGGCCACTTTAGCCCAGGCGACAACCAGGCCTGTAAGCCCTGG
ACCAATTGCACACTGGCAGGCAAGCACACCCTGCAGCCAGCATCTAATTCTAGC
GATGCCATCTGCGAGGACAGAGATCCACCAGCAACCCAGCCTCAGGAGACACAG
GGACCTCCAGCCAGGCCAATCACCGTGCAGCCAACAGAGGCATGGCCTCGGACC
TCTCAGGGACCAAGCACAAGACCCGTGGAGGTGCCTGGAGGAAGGGCAGTGGC
AGCTATCTTGGGGCTCGGGTTGGTACTGGGACTGCTTGGCCCACTTGCTATCTTG
CTGGCTCTGTATCTGCTGAGGCGCGACCAGCGCCTGCCCCCTGATGCACACAAGC
CACCAGGAGGAGGAAGCTTCCGGACCCCAATCCAGGAGGAGCAGGCAGACGCA
CACTCCACACTGGCCAAGATCTGA
Mouse OX40 coding sequence (SEQ ID NO: 12)
ATGTATGTGTGGGTTCAGCAGCCCACAGCCCTTCTGCTGCTGGGACTCACA
CTTGGAGTTACAGCAAGGCGGCTCAACTGTGTTAAACATACCTACCCCAGTGGTC
ACAAGTGCTGTCGTGAGTGCCAGCCAGGCCATGGTATGGTGAGCCGCTGTGATC
ATACCAGGGATACTCTATGTCATCCGTGTGAGACTGGCTTCTACAATGAAGCTGT
CAATTATGATACCTGCAAGCAGTGTACACAGTGCAACCATCGAAGTGGAAGTGA
ACTCAAGCAGAATTGCACACCTACTCAGGATACTGTCTGCAGATGTAGACCAGG
CACCCAACCTCGGCAGGACAGCGGCTACAAGCTTGGAGTTGACTGTGTTCCCTGC
CCTCCTGGCCACTTTTCTCCAGGCAACAACCAGGCCTGCAAGCCCTGGACCAATT
GTACCTTATCTGGAAAGCAGACCCGCCACCCAGCCAGTGACAGCTTGGACGCAG
TCTGTGAGGACAGAAGCCTCCTGGCCACACTGCTCTGGGAGACCCAGCGCCCTA
CATTCAGGCCAACCACTGTCCAATCCACCACAGTCTGGCCCAGGACTTCTGAGTT
GCCCTCTCCACCCACCTTGGTGACTCCTGAGGGCCCTGCATTTGCTGTTCTCCTAG
GCCTGGGCCTGGGCCTGCTGGCTCCCTTGACTGTCCTGCTGGCCTTGTACCTGCTC
CGGAAGGCTTGGAGATTGCCTAACACTCCCAAACCTTGTTGGGGAAACAGCTTC
AGGACCCCGATCCAGGAGGAACACACAGACGCACACTTTACTCTGGCCAAGATC
TGA
Coding sequence of mouse OX40 ligand (SEQ ID NO: 13).
ATGGAAGGGGAAGGGGTTCAACCCCTGGATGAGAATCTGGAAAACGGAT
CAAGGCCAAGATTCAAGTGGAAGAAGACGCTAAGGCTGGTGGTCTCTGGGATCA
AGGGAGCAGGGATGCTTCTGTGCTTCATCTATGTCTGCCTGCAACTCTCTTCCTCT
CCGGCAAAGGACCCTCCAATCCAAAGACTCAGAGGAGCAGTTACCAGATGTGAG
GATGGGCAACTATTCATCAGCTCATACAAGAATGAGTATCAAACTATGGAGGTG
CAGAACAATTCGGTTGTCATCAAGTGCGATGGGCTTTATATCATCTACCTGAAGG
GCTCCTTTTTCCAGGAGGTCAAGATTGACCTTCATTTCCGGGAGGATCATAATCC
CATCTCTATTCCAATGCTGAACGATGGTCGAAGGATTGTCTTCACTGTGGTGGCC
TCTTTGGCTTTCAAAGATAAAGTTTACCTGACTGTAAATGCTCCTGATACTCTCTG
CGAACACCTCCAGATAAATGATGGGGAGCTGATTGTTGTCCAGCTAACGCCTGG
ATACTGTGCTCCTGAAGGATCTTACCACAGCACTGTGAACCAAGTACCACTGTGA
Coding sequence of human OX40 ligand (SEQ ID NO: 14)
ATGGAAAGGGTCCAACCCCTGGAAGAGAATGTGGGAAATGCAGCCAGGC
CAAGATTCGAGAGGAACAAGCTATTGCTGGTGGCCTCTGTAATTCAGGGACTGG
GGCTGCTCCTGTGCTTCACCTACATCTGCCTGCACTTCTCTGCTCTTCAGGTATCA
CATCGGTATCCTCGAATTCAAAGTATCAAAGTACAATTTACCGAATATAAGAAG
GAGAAAGGTTTCATCCTCACTTCCCAAAAGGAGGATGAAATCATGAAGGTGCAG
AACAACTCAGTCATCATCAACTGTGATGGGTTTTATCTCATCTCCCTGAAGGGCT
ACTTCTCCCAGGAAGTCAACATTAGCCTTCATTACCAGAAGGATGAGGAGCCCCT
CTTCCAACTGAAGAAGGTCAGGTCTGTCAACTCCTTGATGGTGGCCTCTCTGACT
TACAAAGACAAAGTCTACTTGAATGTGACCACTGACAATACCTCCCTGGATGACT
TCCATGTGAATGGCGGAGAACTGATTCTTATCCATCAAAATCCTGGTGAATTCTG
TGTCCTTTGA
Coding sequence of mouse ICOS (SEQ ID NO: 15)
ATGAA GCCGTACTTCTGCCGTGTCT TTGTCTTCTG CTTCCTAATC
AGACTTTTAA CAGGAGAAAT CAATGGCTCGGCCGATCATA GGATGTTTTC
ATTTCACAAT GGAGGTGTAC AGATTTCTTG TAAATACCCTGAGACTGTCC
AGCAGTTAAA AATGCGATTG TTCAGAGAGA GAGAAGTCCT
CTGCGAACTCACCAAGACCA AGGGAAGCGG AAATGCGGTG TCCATCAAGA
ATCCAATGCT CTGTCTATATCATCTGTCAA ACAACAGCGT CTCTTTTTTC
CTAAACAACC CAGACAGCTC CCAGGGAAGCTATTACTTCT GCAGCCTGTC
CATTTTTGAC CCACCTCCTT TTCAAGAAAG GAACCTTAGTGGAGGATATT
TGCATATTTA
TGAATCCCAGCTCTGCTGCCAGCTGAAGCTCTGGCTACCCGTAGGGTGTGCAGCT
TTCGT TGTGGTACTC CTTTTTGGAT GCATACTTAT CATCTGGTTTTCAAAAAAGA
AATACGGATCCAGTGTGCATGACCCTAATAGTGAATACATGTTCATGGCGGCAGT
CAACA CAAACAAAAA GTCTAGACTT GCAGGTGTGA CCTCATAA
Coding sequence of human ICOS (SEQ ID NO: 16)
ATG AAGTCAGGCC TCTGGTATTT CTTTCTCTTC TGCTTGCGCA
TTAAAGTTTTAACAGGAGAA ATCAATGGTT CTGCCAATTA TGAGATGTTT
ATATTTCACA ACGGAGGTGT ACAAATTTTA TGCAAATATC CTGACATTGT
CCAGCAATTT AAAATGCAGT TGCTGAAAGGGGGGCAAATA CTCTGCGATC
TCACTAAGAC AAAAGGAAGT GGAAACACAG TGTCCATTAA GAGTCTGAAA
TTCTGCCATT CTCAGTTATC CAACAACAGT GTCTCTTTTT TTCTATACAA
CTTGGACCAT TCTCATGCCA ACTATTACTT CTGCAACCTA TCAATTTTTG
ATCCTCCTCCTTTTAAAGTA ACTCTTACAG GAGGATATTT GCATATTTAT
GAATCACAAC TTTGTTGCCAGCTGAAGTTC TGGTTACCCA TAGGATGTGC
AGCCTTTGTT GTAGTCTGCA TTTTGGGATGCATACTTATT TGTTGGCTTA
CAAAAAAGAA GTATTCATCC AGTGTGCACG ACCCTAACGGTGAATACATG
TTCATGAGAG CAGTGAACAC AGCCAAAAAA TCTAGACTCA CAGATGTGAC
CCTATAA
Coding sequence of mouse CD137 (4-1BB)(SEQ ID NO: 17)
ATGGGAAAC AACTGTTACA ACGTGGTGGT CATTGTGCTG
CTGCTAGTGGGCTGTGAGAA GGTGGGAGCC GTGCAGAACT CCTGTGATAA
CTGTCAGCCT GGTACTTTCTGCAGAAAATA CAATCCAGTC TGCAAGAGCT
GCCCTCCAAG TACCTTCTCC AGCATAGGTGGACAGCCGAA CTGTAACATC
TGCAGAGTGT GTGCAGGCTA TTTCAGGTTC AAGAAGTTTTGCTCCTCTAC
CCACAACGCG GAGTGTGAGT GCATTGAAGG ATTCCATTGC
TTGGGGCCACAGTGCACCAG ATGTGAAAAG GACTGCAGGC CTGGCCAGGA
GCTAACGAAG CAGGGTTGCAAAACCTGTAG CTTGGGAACA TTTAATGACC
AGAACGGTAC TGGCGTCTGT CGACCCTGGACGAACTGCTC TCTAGACGGA
AGGTCTGTGC TTAAGACCGG GACCACGGAG AAGGACGTGGTGTGTGGACC
CCCTGTGGTG AGCTTCTCTC CCAGTACCAC CATTTCTGTG
ACTCCAGAGGGAGGACCAGG AGGGCACTCC TTGCAGGTCC TTACCTTGTT
CCTGGCGCTG ACATCGGCTTTGCTGCTGGC CCTGATCTTC ATTACTCTCC
TGTTCTCTGT GCTCAAATGG ATCAGGAAAA
AATTCCCCCA CATATTCAAG CAACCATTTA AGAAGACCAC
TGGAGCAGCT CAAGAGGAAGATGCTTGTAG CTGCCGATGT CCACAGGAAG
AAGAAGGAGG AGGAGGAGGC TATGAGCTGTGA
Coding sequence of human CD137 (4-1BB)(SEQ ID NO: 18)
ATGGGAAAC AGCTGTTACA ACATAGTAGC CACTCTGTTGCTGGTCCTCA
ACTTTGAGAGGACAAGATCATTGCAGGATCCTTGTAGTAACTGCCCAGCTGGTAC
ATTCTGTGATAATAACAGGAATCAGATTTGCAGTCCCTGTCCTCCAAATAGTTTC
TCCAGCGCAGGTGGACAAAGGACCTGTGACATATGCAGGCAGTGTAAAGGTGTT
TTCAGGACCAGGAAGGAGTGTTCCTCCACCAGCAATGCAGAGTGTGACTGCACT
CCAGGGTTTCACTGCCTGGGGGCAGGATGCAGCATGTGTGAACAGGATTGTAAA
CAAGGTCAAGAACTGACAAAAAAAGGTTGTAAAGACTGTTGCTTTGGGACATTT
AACGATCAGAAACGTGGCATCTGTCGACCCTGGACAAACTGTTCTTTGGATGGA
AAGTCTGTGCTTGTGAATGGGACGAAGGAGAGGGACGTGGTCTGTGGACCATCT
CCAGCCGACCTCTCTCCGGGAGCATCCTCTGTGACCCCGCCTGCCCCTGCGAGAG
AGCCAGGACACTCTCCGCAGATCATCTCCTTCTTTCTTGCGCTGACGTCGACTGC
GTTGCTCTTCCTGCTGTTCTTCCTCACGCTCCGTTTCTCTGTTGTTAAACGGGGCA
GAAAGAAACTCCTGTATATATTCAAACAAC
CATTTATGAGACCAGTACAAACTACTCAAGAGGAAGATGGCTGTAGCTGC
CGATTTCCAG AAGAAGAAGA AGGAGGATGTGAACTGTGA
Coding sequence of mouse CD137 ligand (4-1BBL)(SEQ ID NO: 19)
ATGGACCAGCACACACTTGATGTGGAGGATACCGCGGATGCCAGACATCC
AGCAGGTACTTCGTGCCCCTCGGATGCGGCGCTCCTCAGAGATACCGGGCTCCTC
GCGGACGCTGCGCTCCTCTCAGATACTGTGCGCCCCACAAATGCCGCGCTCCCCA
CGGATGCTGCCTACCCTGCGGTTAATGTTCGGGATCGCGAGGCCGCGTGGCCGCC
TGCACTGAACTTCTGTTCCCGCCACCCAAAGCTCTATGGCCTAGTCGCTTTGGTTT
TGCTGCTTCTGATCGCCGCCTGTGTTCCTATCTTCACCCGCACCGAGCCTCGGCCA
GCGCTCACAATCACCACCTCGCCCAACCTGGGTACCCGAGAGAATAATGCAGAC
CAGGTCACCCCTGTTTCCCACATTGGCTGCCCCAACACTACACAACAGGGCTCTC
CTGTGTTCGCCAAGCTACTGGCTAAAAACCAAGCATCGTTGTGCAATACAACTCT
GAACTGGCACAGCCAAGATGGAGCTGGGAGCTCATACCTATCTCAAGGTCTGAG
GTACGAAGAAGACAAAAAGGAGTTGGTGGTAGACAGTCCCGGGCTCTACTACGT
ATTTTTGGAACTGAAGCTCAGTCCAACATTCACAAACACAGGCCACAAGGTGCA
GGGCTGGGTCTCTCTTGTTTTGCAAGCAAAGCCTCAGGTAGATGACTTTGACAAC
TTGGCCCTGACAGTGGAACTGTTCCCTTGCTCCATGGAGAACAAGTTAGTGGACC
GTTCCTGGAGTCAACTGTTGCTCCTGAAGGCTGGCCACCGCCTCAGTGTGGGTCT
GAGGGCTTATCTGCATGGAGCCCAGGATGCATACAGAGACTGGGAGCTGTCTTA
TCCCAACACCACCAGCTTTGGACTCTTTCTTGTGAAACCCGACAACCCATGGGAA
TGA
Coding sequence of human CD137 ligand (4-1BBL)(SEQ ID NO: 20)
ATGGAATACGCCTCTGACGCTTCACTGGACCCCGAAGCCCCGTGGCCTCC
CGCGCCCCGCGCTCGCGCCTGCCGCGTACTGCCTTGGGCCCTGGTCGCGGGGCTG
CTGCTGCTGCTGCTGCTCGCTGCCGCCTGCGCCGTCTTCCTCGCCTGCCCCTGGGC
CGTGTCCGGGGCTCGCGCCTCGCCCGGCTCCGCGGCCAGCCCGAGACTCCGCGA
GGGTCCCGAGCTTTCGCCCGACGATCCCGCCGGCCTCTTGGACCTGCGGCAGGGC
ATGTTTGCGCAGCTGGTGGCCCAAAATGTTCTGCTGATCGATGGGCCCCTGAGCT
GGTACAGTGACCCAGGCCTGGCAGGCGTGTCCCTGACGGGGGGCCTGAGCTACA
AAGAGGACACGAAGGAGCTGGTGGTGGCCAAGGCTGGAGTCTACTATGTCTTCT
TTCAACTAGAGCTGCGGCGCGTGGTGGCCGGCGAGGGCTCAGGCTCCGTTTCACT
TGCGCTGCACCTGCAGCCACTGCGCTCTGCTGCTGGGGCCGCCGCCCTGGCTTTG
ACCGTGGACCTGCCACCCGCCTCCTCCGAGGCTCGGAACTCGGCCTTCGGTTTCC
AGGGCCGCTTGCTGCACCTGAGTGCCGGCCAGCGCCTGGGCGTCCATCTTCACAC
TGAGGCCAGGGCACGCCATGCCTGGCAGCTTACCCAGGGCGCCACAGTCTTGGG
ACTCTTCCGGGTGACCCCCGAAATCCCAGCCGGACTCCCTTCACCGAGGTCGGAA
TAA
Coding sequence of mouse GITR (SEQ ID NO: 21)
ATGGGGGCATGGGCCATGCTGTATGGAGTCTCGATGCTCTGTGTGCTGGA
CCTAGGTCAGCCGAGTGTAGTTGAGGAGCCTGGCTGTGGCCCTGGCAAGGTTCA
GAACGGAAGTGGCAACAACACTCGCTGCTGCAGCCTGTATGCTCCAGGCAAGGA
GGACTGTCCAAAAGAAAGGTGCATATGTGTCACACCTGAGTACCACTGTGGAGA
CCCTCAGTGCAAGATCTGCAAGCACTACCCCTGCCAACCAGGCCAGAGGGTGGA
GTCTCAAGGGGATATTGTGTTTGGCTTCCGGTGTGTTGCCTGTGCCATGGGCACC
TTCTCCGCAGGTCGTGACGGTCACTGCAGACTTTGGACCAACTGTTCTCAGTTTG
GATTTCTCACCATGTTCCCTGGGAACAAGACCCACAATGCTGTGTGCATCCCGGA
GCCACTGCCCACTGAGCAATACGGCCATTTGACTGTCATCTTCCTGGTCATGGCT
GCATGCATTTTCTTCCTAACCACAGTCCAGCTCGGCCTGCACATATGGCAGCTGA
GGAGGCAACACATGTGTCCTCGAGAGACCCAGCCATTCGCGGAGGTGCAGTTGT
CAGCTGAGGATGCTTGCAGCTTCCAGTTCCCTGAGGAGGAACGCGGGGAGCAGA
CAGAAGAAAAGTGTCATCTGGGGGGTCGGTGGCCAT GA
Coding sequence of human GITR (SEQ ID NO: 22)
ATGGCACAG CACGGGGCGA TGGGCGCGTT TCGGGCCCTG TGCGGCCTGG
CGCTGCTGTG CGCGCTCAGC CTGGGTCAGC GCCCCACCGG GGGTCCCGGG
TGCGGCCCTG GGCGCCTCCT GCTTGGGACG GGAACGGACG CGCGCTGCTG
CCGGGTTCAC ACGACGCGCT GCTGCCGCGA TTACCCGGGC GAGGAGTGCT
GTTCCGAGTG GGACTGCATGTGTGTCCAGC CTGAATTCCA CTGCGGAGAC
CCTTGCTGCA CGACCTGCCG GCACCACCCTTGTCCCCCAG GCCAGGGGGT
ACAGTCCCAG GGGAAATTCA GTTTTGGCTT CCAGTGTATCGACTGTGCCT
CGGGGACCTT CTCCGGGGGC CACGAAGGCC ACTGCAAACC TTGGACAGAC
TGCACCCAGT TCGGGTTTCT CACTGTGTTC CCTGGGAACA AGACCCACAA
CGCTGTGTGCGTCCCAGGGT CCCCGCCGGC AGAGCCGCTT GGGTGGCTGA
CCGTCGTCCT CCTGGCCGTGGCCGCCTGCG TCCTCCTCCT GACCTCGGCC
CAGCTTGGAC TGCACATCTG GCAGCTGAGG AGTCAGTGCA TGTGGCCCCG
AGAGACCCAG CTGCTGCTGG AGGTGCCGCC GTCGACCGAA GACGCCAGAA
GCTGCCAGTT CCCCGAGGAA GAGCGGGGCG AGCGATCGGC
AGAGGAGAAGGGGCGGCTGG GAGACCTGTG GGTGTGA
CD40 coding sequence (SEQ ID NO: 23)
ATGGTGTCTTTGCCTCGGCTGTGCGCGCTATGGGGCTGCTTGTTGACAGCG
GTCCATCTAGGGCAGTGTGTTACGTGCAGTGACAAACAGTACCTCCACGATGGCC
AGTGCTGTGATTTGTGCCAGCCAGGAAGCCGACTGACAAGCCACTGCACAGCTC
TTGAGAAGACCCAATGCCACCCATGTGACTCAGGCGAATTCTCAGCCCAGTGGA
ACAGGGAGATTCGCTGTCACCAGCACAGACACTGTGAACCCAATCAAGGGCTTC
GGGTTAAGAAGGAGGGCACCGCAGAATCAGACACTGTCTGTACCTGTAAGGAAG
GACAACACTGCACCAGCAAGGATTGCGAGGCATGTGCTCAGCACACGCCCTGTA
TCCCTGGCTTTGGAGTTATGGAGATGGCCACTGAGACCACTGATACCGTCTGTCA
TCCCTGCCCAGTCGGCTTCTTCTCCAATCAGTCATCACTTTTCGAAAAGTGTTATC
CCTGGACAAGCTGTGAGGATAAGAACTTGGAGGTCCTACAGAAAGGAACGAGTC
AGACTAATGTCATCTGTGGTTTAAAGTCCCGGATGCGAGCCCTGCTGGTCATTCC
TGTCGTGATGGGCATCCTCATCACCATTTTCGGGGTGTTTCTCTATATCAAAAAG
GTGGTCAAGAAACCAAAGGATAATGAGATCTTACCCCCTGCGGCTCGACGGCAA
GATCCCCAGGAGATGGAAGATTATCCCGGTCATAACACCGCTGCTCCAGTGCAG
GAGACACTGCACGGGTGTCAGCCTGTCACACAGGAGGATGGTAAAGAGAGTCGC
ATCTCAGTGCAGGAGCGGCAGGTGACAGACAGCATAGCCTTGAGGCCCCTGGTC
TAA
CD40 ligand (CD40L) coding sequence (SEQ ID NO: 24)
ATGATAGAAACATACAGCCAACCTTCCCCCAGATCCGTGGCAACTGGACT
TCCAGCGAGCATGAAGATTTTTATGTATTTACTTACTGTTTTCCTTATCACCCAAA
TGATTGGATCTGTGCTTTTTGCTGTGTATCTTCATAGAAGATTGGATAAGGTCGA
AGAGGAAGTAAACCTTCATGAAGATTTTGTATTCATAAAAAAGCTAAAGAGATG
CAACAAAGGAGAAGGATCTTTATCCTTGCTGAACTGTGAGGAGATGAGAAGGCA
ATTTGAAGACCTTGTCAAGGATATAACGTTAAACAAAGAAGAGAAAAAAGAAAA
CAGCTTTGAAATGCAAAGAGGTGATGAGGATCCTCAAATTGCAGCACACGTTGT
AAGCGAAGCCAACAGTAATGCAGCATCCGTTCTACAGTGGGCCAAGAAAGGATA
TTATACCATGAAAAGCAACTTGGTAATGCTTGAAAATGGGAAACAGCTGACGGT
TAAAAGAGAAGGACTCTATTATGTCTACACTCAAGTCACCTTCTGCTCTAATCGG
GAGCCTTCGAGTCAACGCCCATTCATCGTCGGCCTCTGGCTGAAGCCCAGCAGTG
GATCTGAGAGAATCTTACTCAAGGCGGCAAATACCCACAGTTCCTCCCAGCTTTG
CGAGCAGCAGTCTGTTCACTTGGGCGGAGTGTTTGAATTACAAGCTGGTGCTTCT
GTGTTTGTCAACGTGACTGAAGCAAGCCAAGTGATCCACAGAGTTGGCTTCTCAT
CTTTTGGCTTACTCAAACTCTGA

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will appreciate that numerous changes and modifications can be made to the preferred embodiments of the invention and that such changes and modifications can be made without departing from the spirit of the invention. It is, therefore, intended that the appended claims cover all such equivalent variations as fall within the true spirit and scope of the invention.

1. A compound of Formula A: 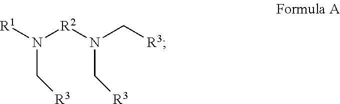
or a salt thereof, wherein:
R¹ is 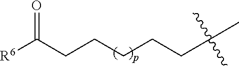
wherein p is an integer from 1 to 20.
R⁶ is a toll-like receptor (TLR) agonist or a derivative thereof;
R² is alkyl, cycloalkyl, heterocycloalkyl, alkylheterocycloalkyl, amine, amide, alkylamide, ether, or alkylether; and
each R³ is independently selected from alkyl, alkenyl, alkynyl, ester, or alkylester.

2. The compound of claim 1, wherein R⁶ comprises:

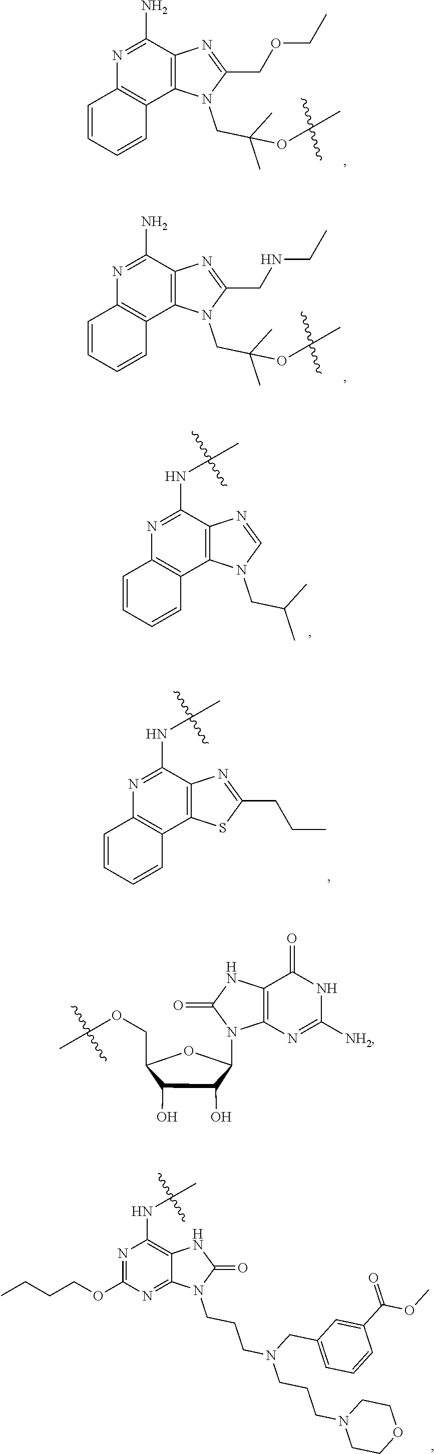

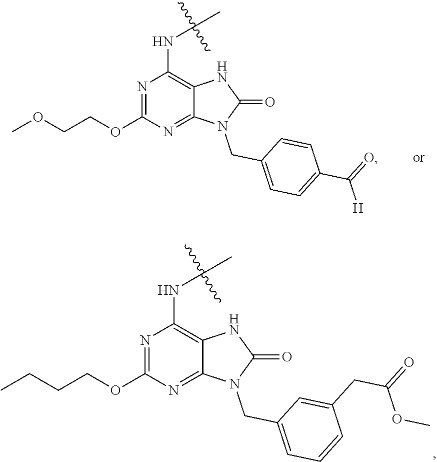 [SMILES: *Nc1nc(OCCOC)nc2c1[nH]c(=O)n2Cc1ccc(C=O)cc1 ; *Nc1nc(OCCCC)nc2c1[nH]c(=O)n2Cc1cccc(CC(=O)OC)c1]

3. The compound of claim 1, wherein R¹ comprises: 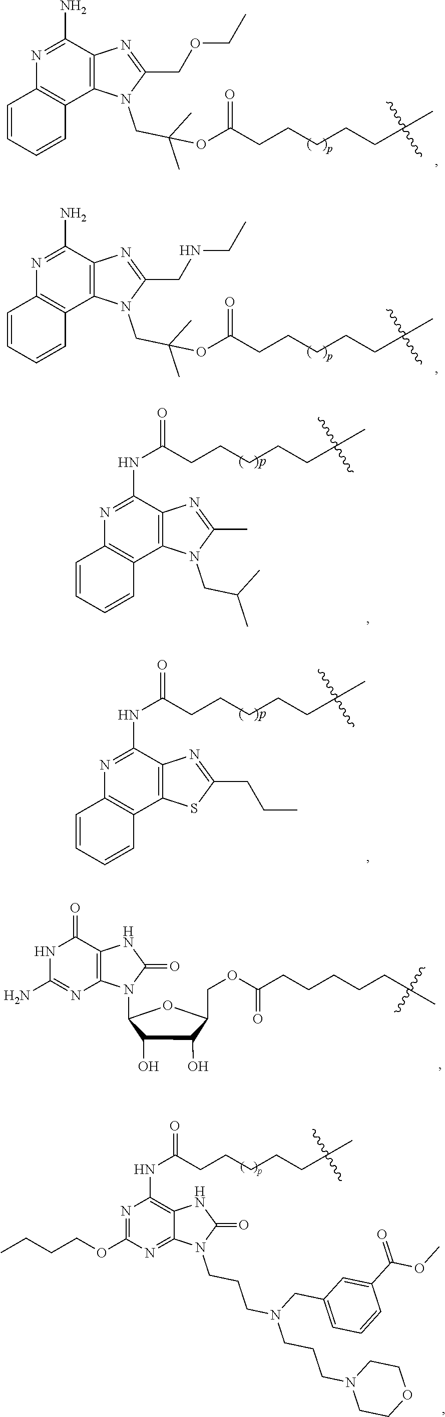 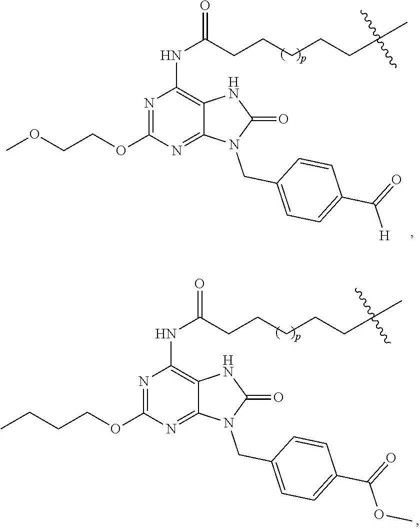
wherein p is an integer from 1 to 20.

4. The compound of claim 1, wherein R¹ comprises:

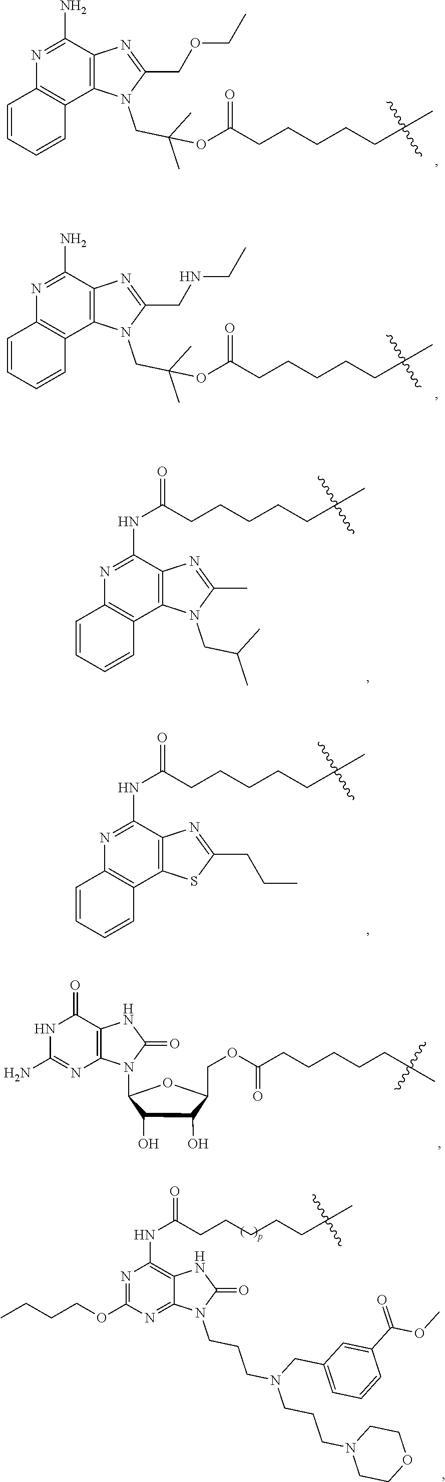

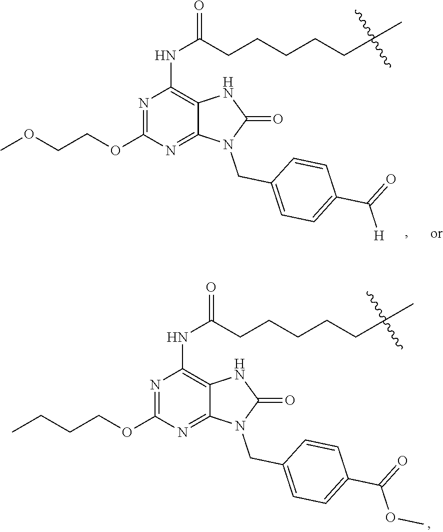 [SMILES: *CCCCCC(=O)Nc1nc(OCCOC)nc2c1[nH]c(=O)n2Cc1ccc(C=O)cc1 ; *CCCCCC(=O)Nc1nc(OCCCC)nc2c1[nH]c(=O)n2Cc1ccc(C(=O)OC)cc1]

5. The compound of claim 1, wherein R¹ is

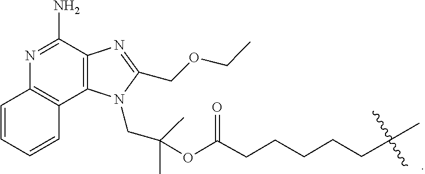 [SMILES: *CCCCCC(=O)OC(C)(C)Cn1c(COCC)nc2c(N)nc3ccccc3c21]

6. The compound of claim 1, wherein R² comprises: 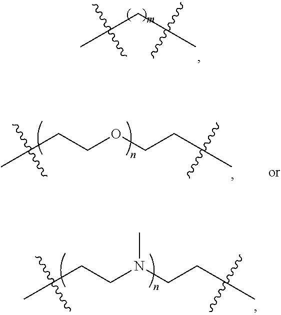
wherein m is an integer from 1 to 20,
wherein n is an integer from 1 to 3.

7. The compound of claim 1, wherein the compound has the formula: 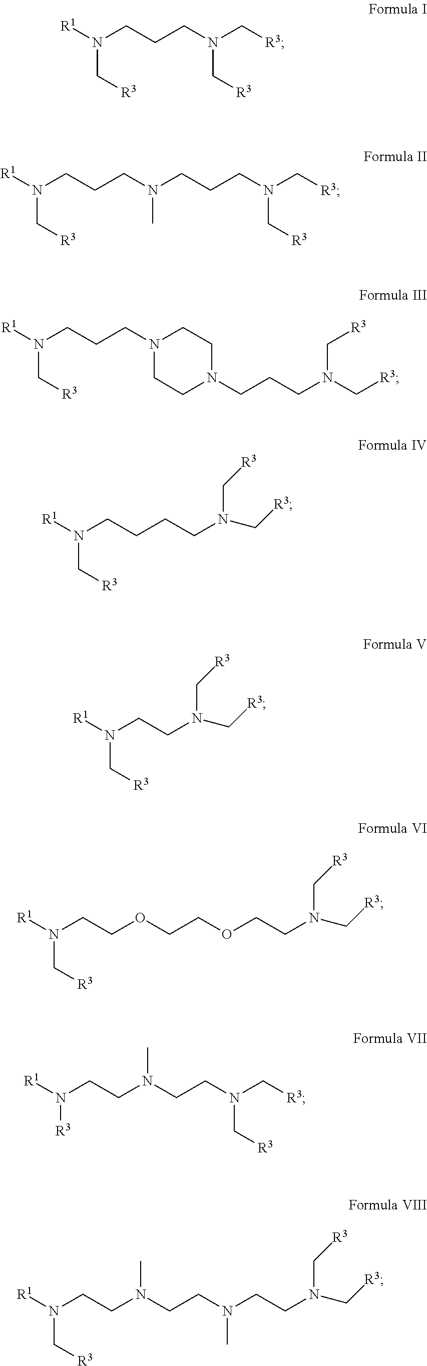 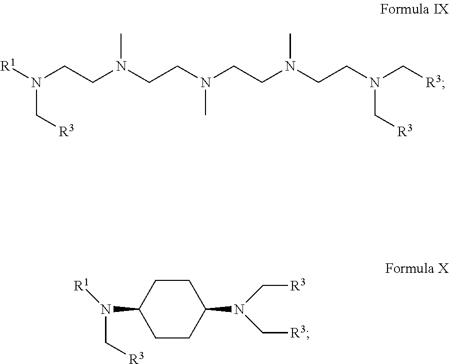
or a salt thereof, wherein:
R¹ is 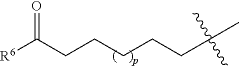
wherein p is an integer from 1 to 20.
R⁶ is a toll-like receptor (TLR) agonist or a derivative thereof; and
each R³ is independently selected from alkyl, alkenyl, alkynyl, ester, or alkylester.

8. The compound of claim 1, having Formula I: 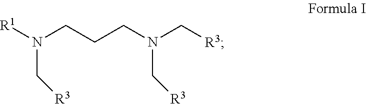
or a salt thereof, wherein:
R¹ is 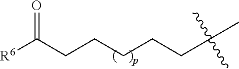
wherein p is an integer from 1 to 20.
R⁶ is a toll-like receptor (TLR) agonist or a derivative thereof; and
each R³ is independently selected from alkyl, alkenyl, alkynyl, ester, or alkylester.

9. The compound of claim 8, wherein R⁶ comprises:

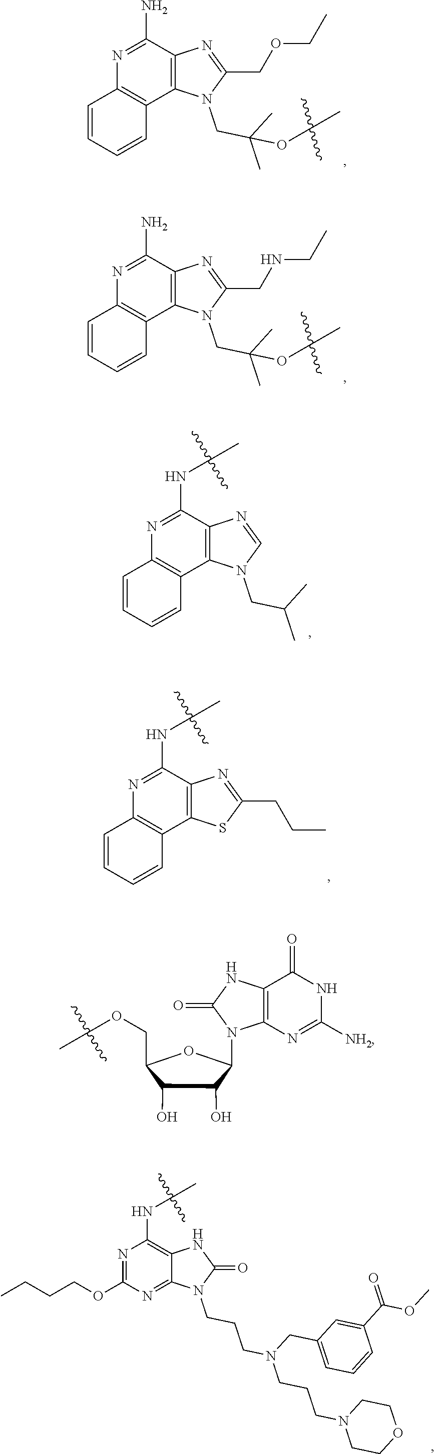

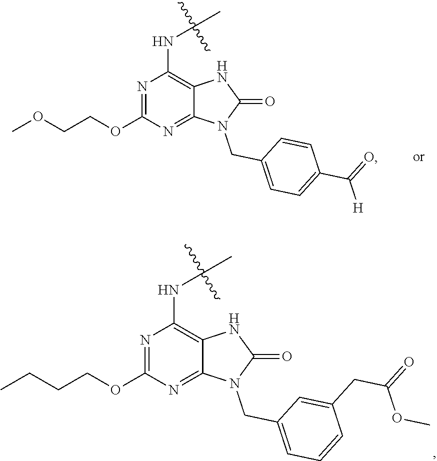 [SMILES: *Nc1nc(OCCOC)nc2c1[nH]c(=O)n2Cc1ccc(C=O)cc1 ; *Nc1nc(OCCCC)nc2c1[nH]c(=O)n2Cc1cccc(CC(=O)OC)c1]

10. The compound of claim 8, wherein R¹ comprises: 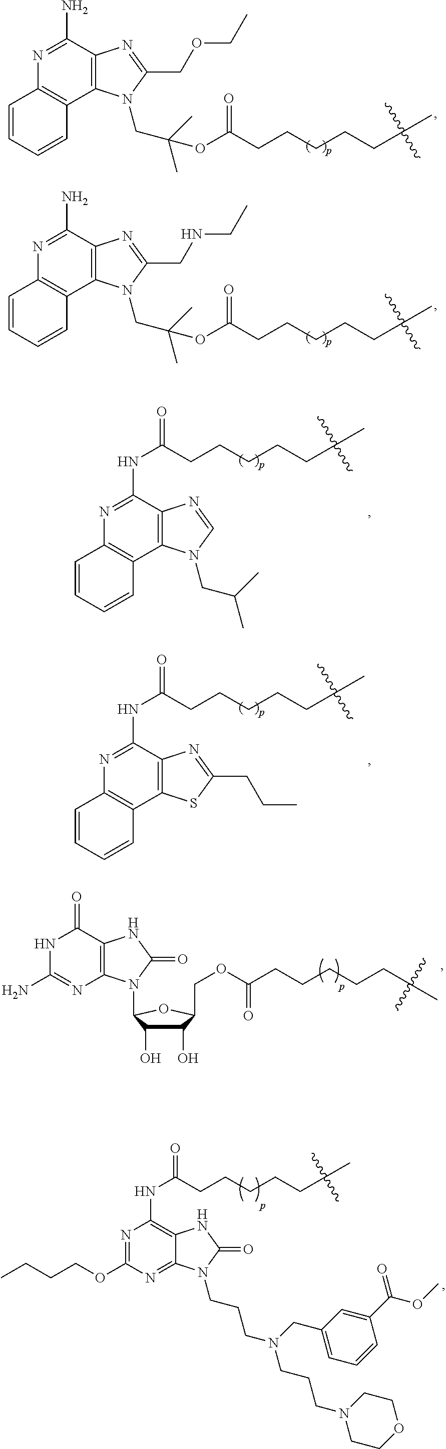 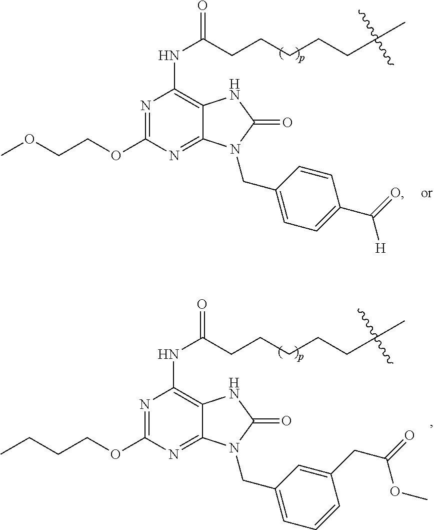
wherein p is an integer from 1 to 20.

11. The compound of claim 8, wherein R¹ comprises:

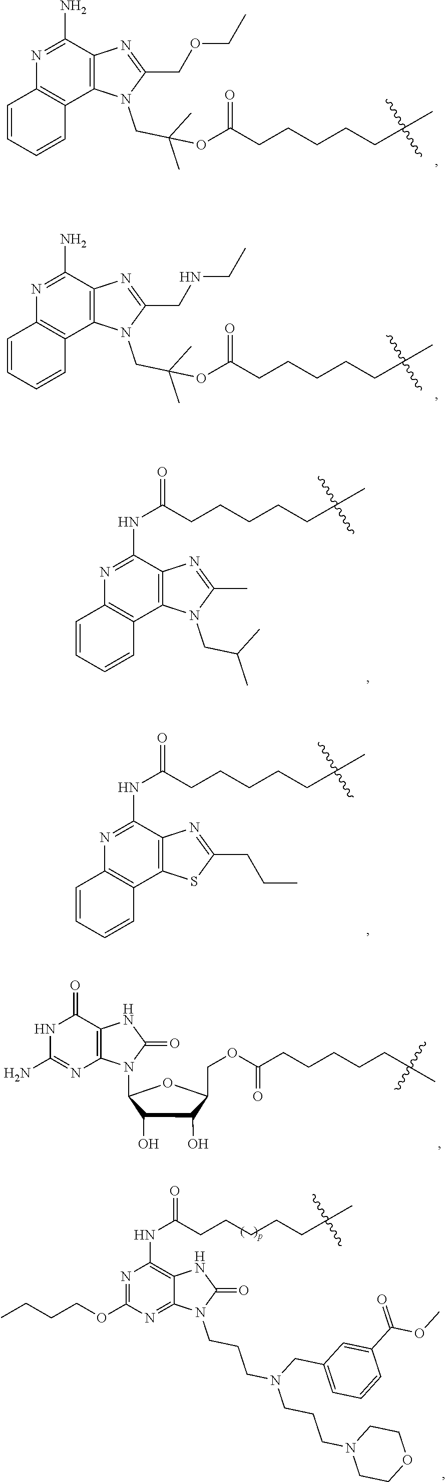

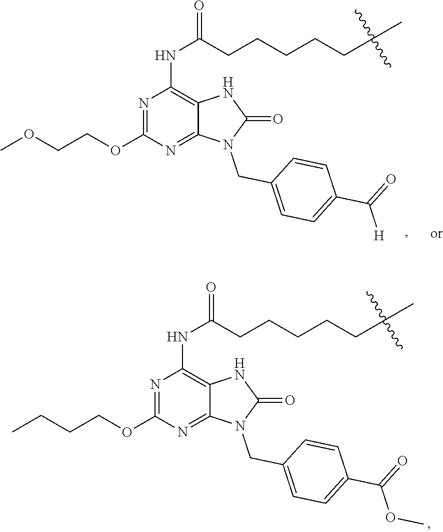 [SMILES: *CCCCCC(=O)Nc1nc(OCCOC)nc2c1[nH]c(=O)n2Cc1ccc(C=O)cc1 ; *CCCCCC(=O)Nc1nc(OCCCC)nc2c1[nH]c(=O)n2Cc1ccc(C(=O)OC)cc1]

12. The compound of claim 7, wherein R¹ is

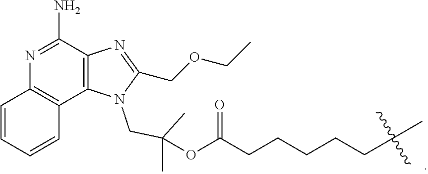 [SMILES: *CCCCCC(=O)OC(C)(C)Cn1c(COCC)nc2c(N)nc3ccccc3c21]

13. The compound of claim 1, wherein the compound is selected from: 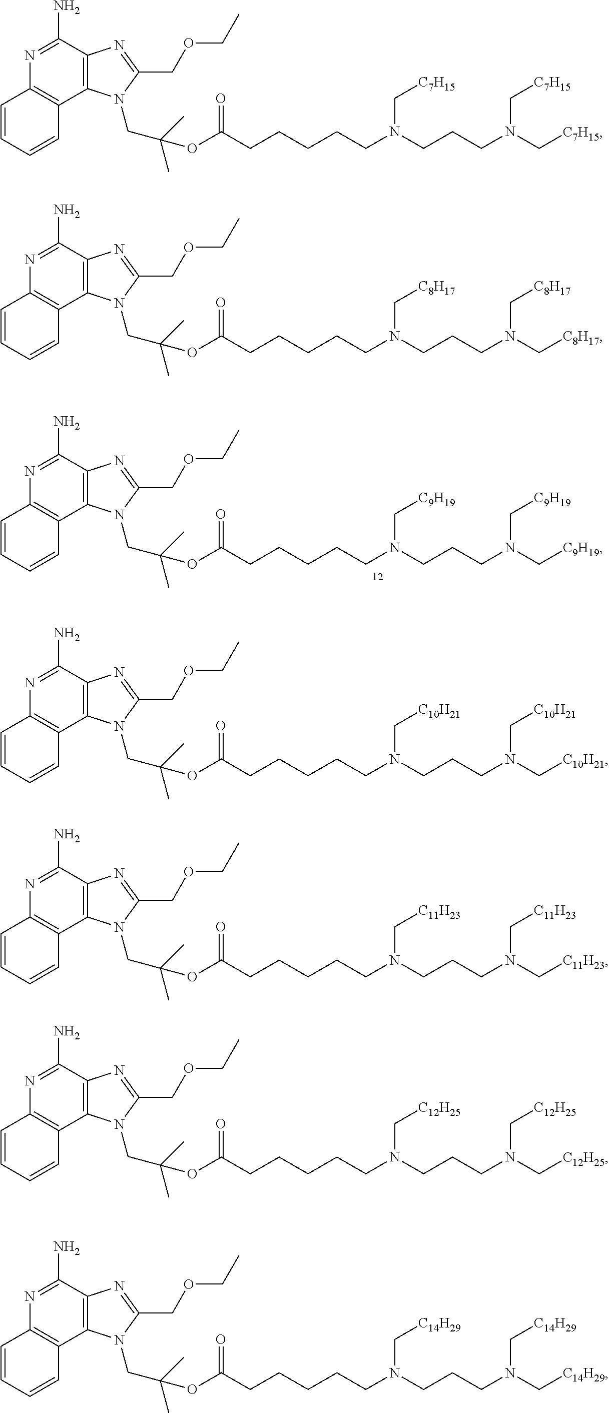 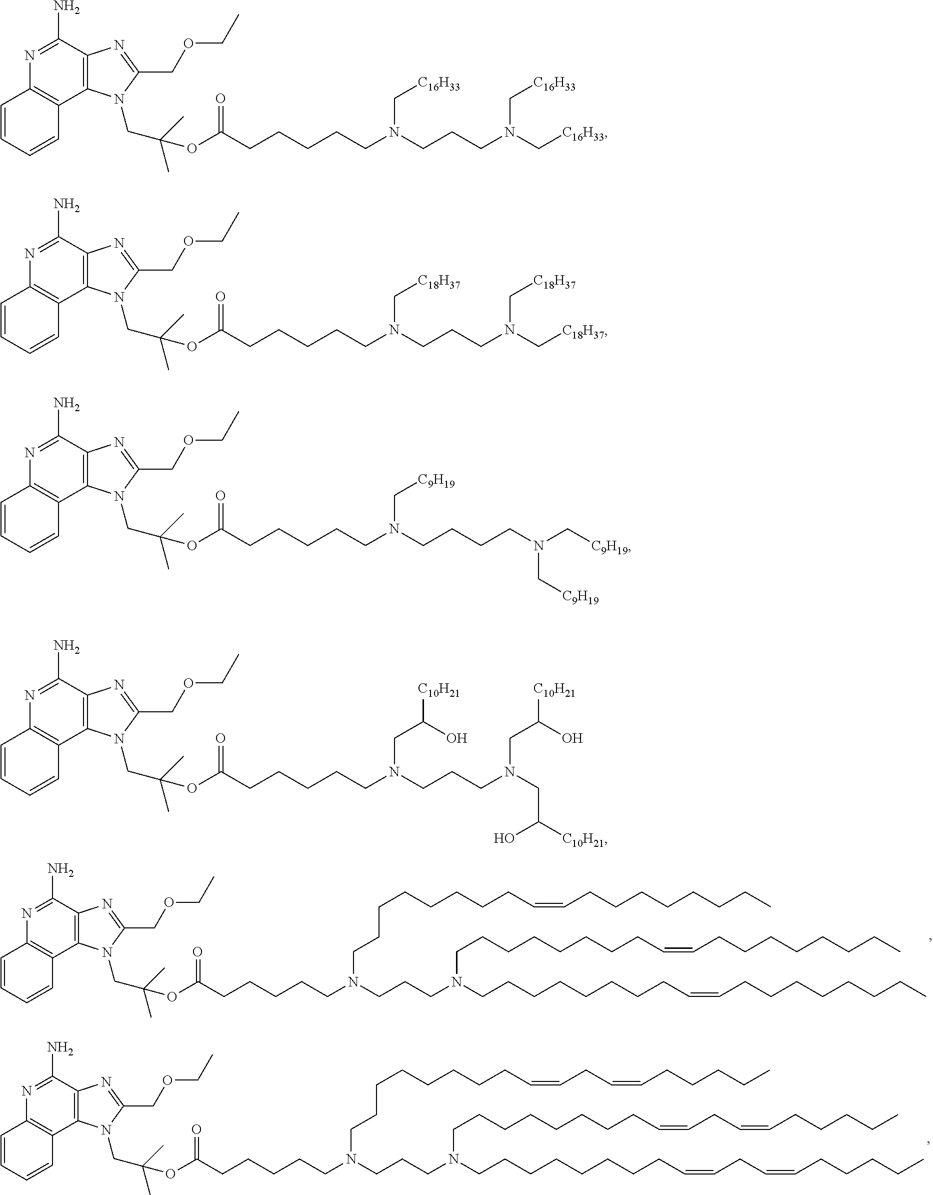 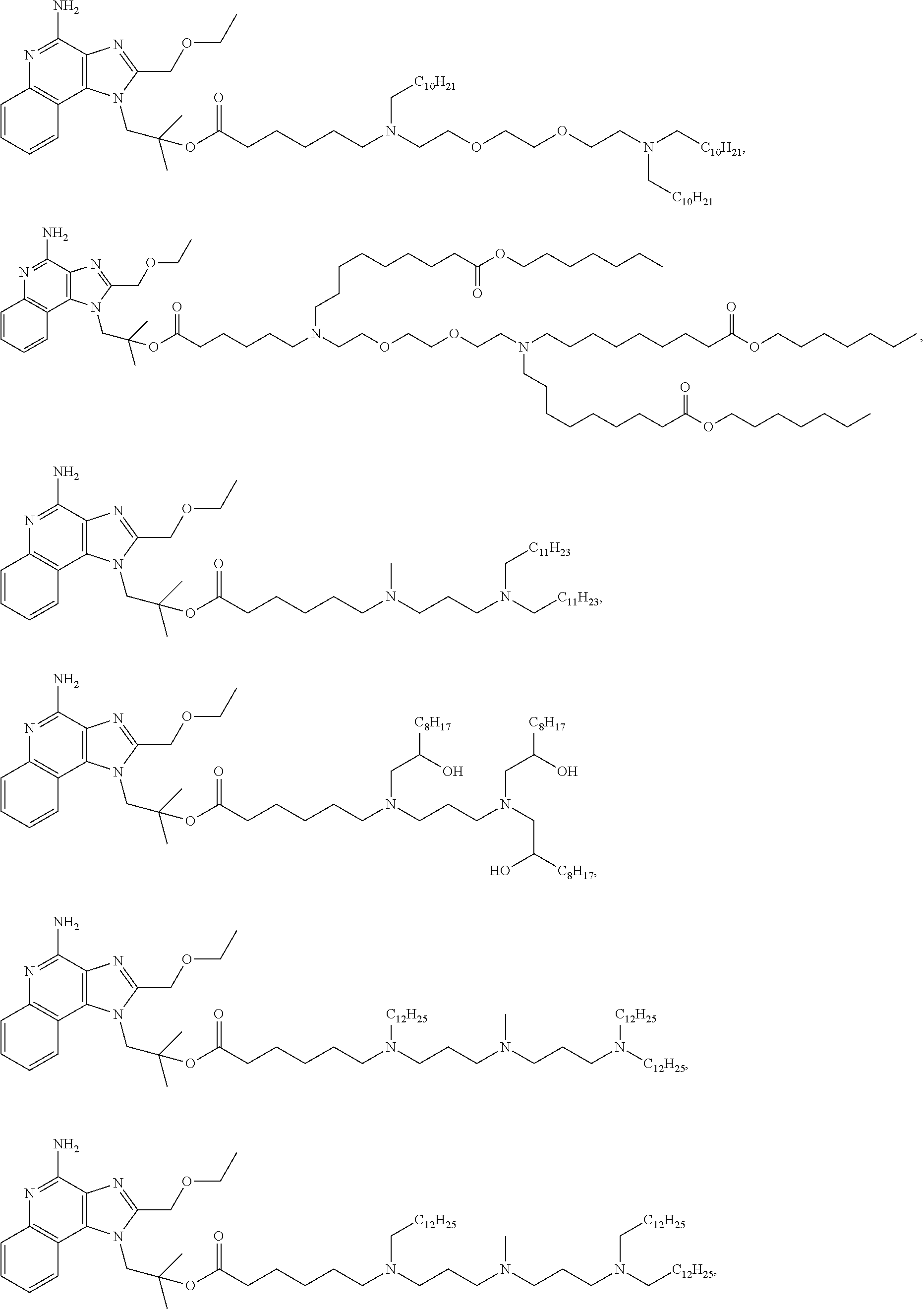 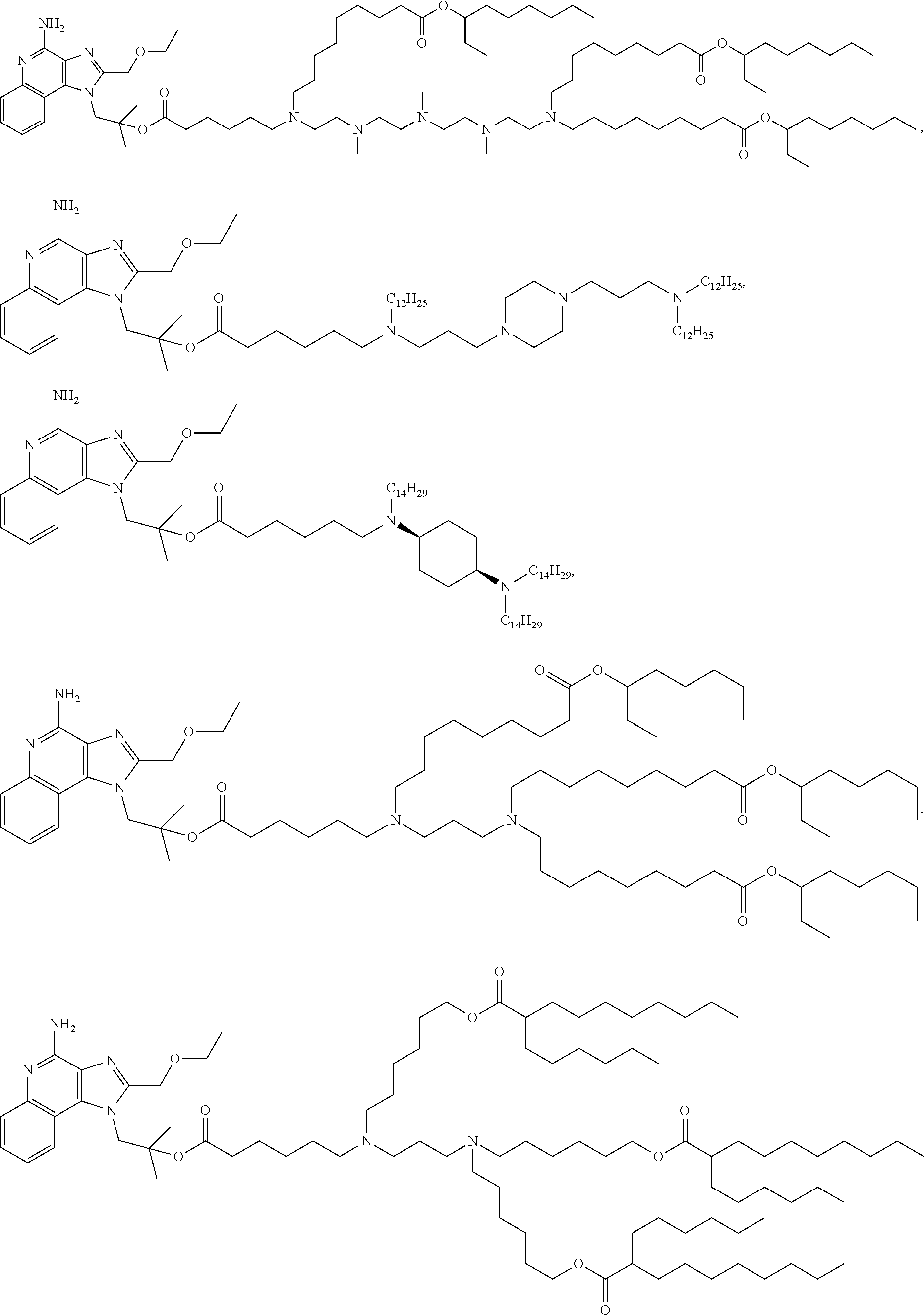 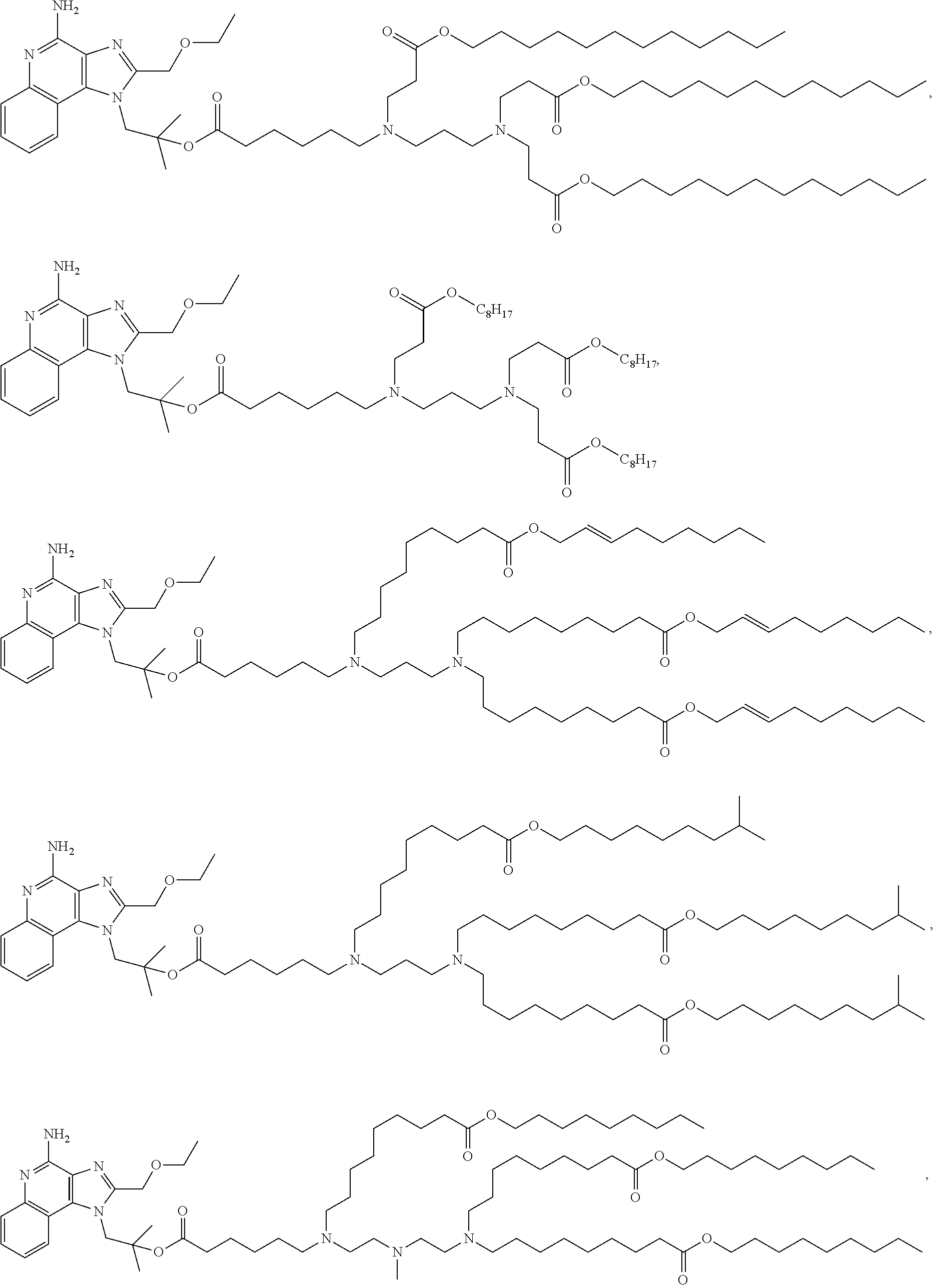 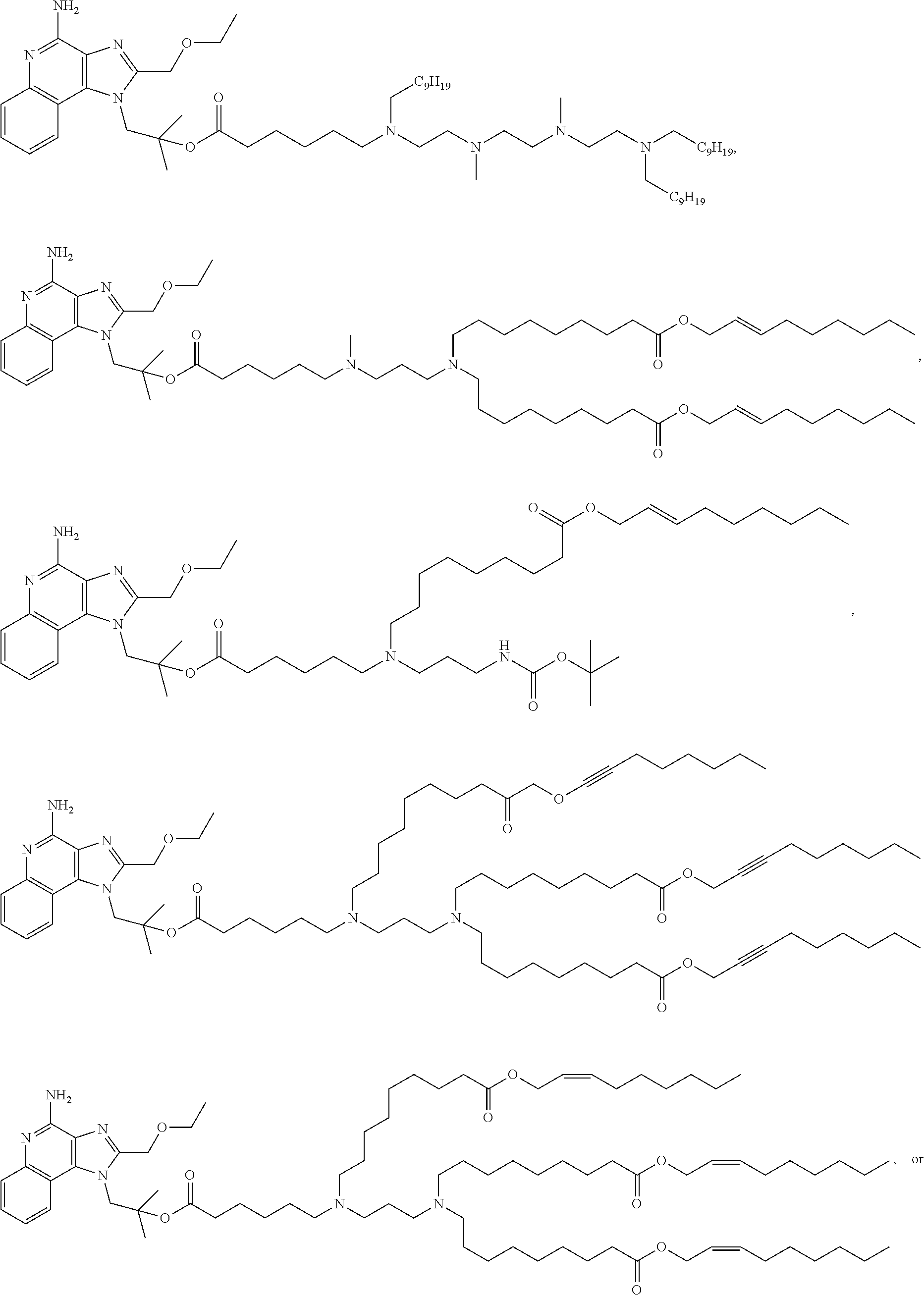 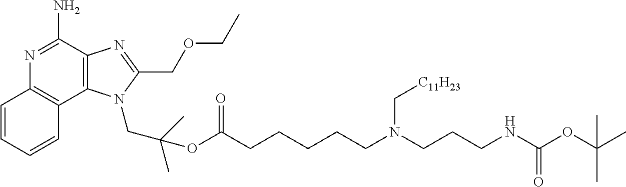
or a salt thereof.

14. The compound of claim 1, wherein the compound is:

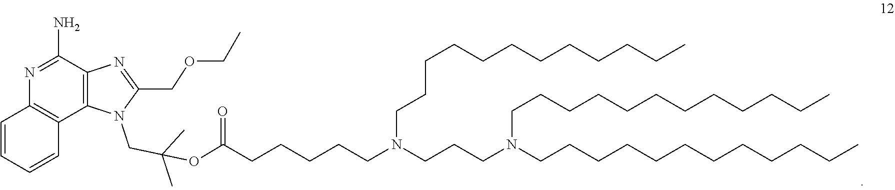 [SMILES: CCCCCCCCCCCCN(CCCCCCCCCCCC)CCCN(CCCCCCCCCCCC)CCCCCC(=O)OC(C)(C)Cn1c(COCC)nc2c(N)nc3ccccc3c21]

15. The compound of claim 1, wherein the compound is:

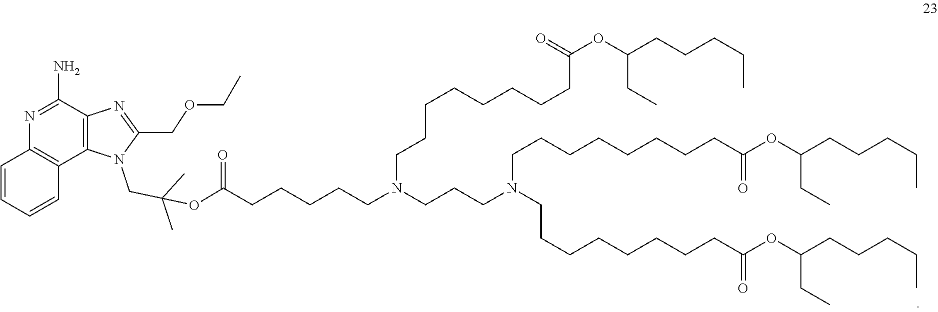 [SMILES: CCCCCC(CC)OC(=O)CCCCCCCCN(CCCCCCCCC(=O)OC(CC)CCCCC)CCCN(CCCCCCCCC(=O)OC(CC)CCCCC)CCCCCC(=O)OC(C)(C)Cn1c(COCC)nc2c(N)nc3ccccc3c21]

16. A composition comprising:
a compound of claim 1; and
an agent.

17-48. (canceled)

49. A nanoparticle comprising:
a compound of claim 1; and
a non-cationic lipid;
a polyethylene glycol-lipid; and
a sterol.

50-97. (canceled)

98. A composition comprising:
an antibody, a ligand, or an antigen binding fragment thereof that specifically binds a co-stimulatory molecule; and
a nanoparticle of claim 49, comprising an mRNA encoding the co-stimulatory molecule.

99-106. (canceled)

107. A pharmaceutical composition comprising a pharmaceutically acceptable carrier and an effective amount of a compound of claim 1.

108. A method for delivering an agent into a cell, comprising:
introducing into the cell a composition of claim 16.

109-133. (canceled)


SEQUENCE LISTING (24 sequences)

SEQ ID NO: 1 - RNA, 918 nt, Homo sapiens
gggaaaaguagaaagaaagaaagaagagaaaauaaagacaaagagccaccaugugcgugggagcacggagacugggaaggggaccuugcgccgcccugcugcugcugggccugggccuguccaccgugacaggccugcacugcgugggcgacaccuacccuucuaacgauaggugcugucacgagugucgcccaggcaauggcaugguguccaggugcucccgcucucagaacaccgugugccggccuuguggcccaggcuucuauaaugacguggugagcuccaagcccugcaagccuuguacauggugcaaccugcggagcggcuccgagagaaagcagcugugcaccgccacacaggauaccgugugccgguguagagccggcacacagccacuggacucuuacaagccaggaguggauugugcaccuugcccaccuggccacuuuagcccaggcgacaaccaggccuguaagcccuggaccaauugcacacuggcaggcaagcacacccugcagccagcaucuaauucuagcgaugccaucugcgaggacagagauccaccagcaacccagccucaggagacacagggaccuccagccaggccaaucaccgugcagccaacagaggcauggccucggaccucucagggaccaagcacaagacccguggaggugccuggaggaagggcaguggcagcuaucuuggggcucggguugguacugggacugcuuggcccacuugcuaucuugcuggcucuguaucugcugaggcgcgaccagcgccugcccccugaugcacacaagccaccaggaggaggaagcuuccggaccccaauccaggaggagcaggcagacgcacacuccacacuggccaagaucugauuguguaugcguuaauaaaaagaaggaacucgua

SEQ ID NO: 2 - RNA, 903 nt, Mus musculus
gggaaaaguagaaagaaagaaagaagagaaaauaaagacaaagagccaccauguauguguggguucagcagcccacagcccuucugcugcugggacucacacuuggaguuacagcaaggcggcucaacuguguuaaacauaccuaccccaguggucacaagugcugucgugagugccagccaggccaugguauggugagccgcugugaucauaccagggauacucuaugucauccgugugagacuggcuucuacaaugaagcugucaauuaugauaccugcaagcaguguacacagugcaaccaucgaaguggaagugaacucaagcagaauugcacaccuacucaggauacugucugcagauguagaccaggcacccaaccucggcaggacagcggcuacaagcuuggaguugacuguguucccugcccuccuggccacuuuucuccaggcaacaaccaggccugcaagcccuggaccaauuguaccuuaucuggaaagcagacccgccacccagccagugacagcuuggacgcagucugugaggacagaagccuccuggccacacugcucugggagacccagcgcccuacauucaggccaaccacuguccaauccaccacagucuggcccaggacuucugaguugcccucuccacccaccuuggugacuccugagggcccugcauuugcuguucuccuaggccugggccugggccugcuggcucccuugacuguccugcuggccuuguaccugcuccggaaggcuuggagauugccuaacacucccaaaccuuguuggggaaacagcuucaggaccccgauccaggaggaacacacagacgcacacuuuacucuggccaagaucugauuguguaugcguuaauaaaaagaaggaacucgua

SEQ ID NO: 3 - RNA, 50 nt, Artificial Sequence
  synthetic construct
gggaaaaguagaaagaaagaaagaagagaaaauaaagacaaagagccacc

SEQ ID NO: 4 - RNA, 34 nt, Artificial Sequence
  synthetic construct
uuguguaugcguuaauaaaaagaaggaacucgua

SEQ ID NO: 5 - RNA, 834 nt, Homo sapiens
augugcgugggagcacggagacugggaaggggaccuugcgccgcccugcugcugcugggccugggccuguccaccgugacaggccugcacugcgugggcgacaccuacccuucuaacgauaggugcugucacgagugucgcccaggcaauggcaugguguccaggugcucccgcucucagaacaccgugugccggccuuguggcccaggcuucuauaaugacguggugagcuccaagcccugcaagccuuguacauggugcaaccugcggagcggcuccgagagaaagcagcugugcaccgccacacaggauaccgugugccgguguagagccggcacacagccacuggacucuuacaagccaggaguggauugugcaccuugcccaccuggccacuuuagcccaggcgacaaccaggccuguaagcccuggaccaauugcacacuggcaggcaagcacacccugcagccagcaucuaauucuagcgaugccaucugcgaggacagagauccaccagcaacccagccucaggagacacagggaccuccagccaggccaaucaccgugcagccaacagaggcauggccucggaccucucagggaccaagcacaagacccguggaggugccuggaggaagggcaguggcagcuaucuuggggcucggguugguacugggacugcuuggcccacuugcuaucuugcuggcucuguaucugcugaggcgcgaccagcgccugcccccugaugcacacaagccaccaggaggaggaagcuuccggaccccaauccaggaggagcaggcagacgcacacuccacacuggccaagaucuga

SEQ ID NO: 6 - RNA, 819 nt, Mus musculus
auguauguguggguucagcagcccacagcccuucugcugcugggacucacacuuggaguuacagcaaggcggcucaacuguguuaaacauaccuaccccaguggucacaagugcugucgugagugccagccaggccaugguauggugagccgcugugaucauaccagggauacucuaugucauccgugugagacuggcuucuacaaugaagcugucaauuaugauaccugcaagcaguguacacagugcaaccaucgaaguggaagugaacucaagcagaauugcacaccuacucaggauacugucugcagauguagaccaggcacccaaccucggcaggacagcggcuacaagcuuggaguugacuguguucccugcccuccuggccacuuuucuccaggcaacaaccaggccugcaagcccuggaccaauuguaccuuaucuggaaagcagacccgccacccagccagugacagcuuggacgcagucugugaggacagaagccuccuggccacacugcucugggagacccagcgcccuacauucaggccaaccacuguccaauccaccacagucuggcccaggacuucugaguugcccucuccacccaccuuggugacuccugagggcccugcauuugcuguucuccuaggccugggccugggccugcuggcucccuugacuguccugcuggccuuguaccugcuccggaaggcuuggagauugccuaacacucccaaaccuuguuggggaaacagcuucaggaccccgauccaggaggaacacacagacgcacacuuuacucuggccaagaucuga

SEQ ID NO: 7 - DNA, 918 bp, Homo sapiens
gggaaaagtagaaagaaagaaagaagagaaaataaagacaaagagccaccatgtgcgtgggagcacggagactgggaaggggaccttgcgccgccctgctgctgctgggcctgggcctgtccaccgtgacaggcctgcactgcgtgggcgacacctacccttctaacgataggtgctgtcacgagtgtcgcccaggcaatggcatggtgtccaggtgctcccgctctcagaacaccgtgtgccggccttgtggcccaggcttctataatgacgtggtgagctccaagccctgcaagccttgtacatggtgcaacctgcggagcggctccgagagaaagcagctgtgcaccgccacacaggataccgtgtgccggtgtagagccggcacacagccactggactcttacaagccaggagtggattgtgcaccttgcccacctggccactttagcccaggcgacaaccaggcctgtaagccctggaccaattgcacactggcaggcaagcacaccctgcagccagcatctaattctagcgatgccatctgcgaggacagagatccaccagcaacccagcctcaggagacacagggacctccagccaggccaatcaccgtgcagccaacagaggcatggcctcggacctctcagggaccaagcacaagacccgtggaggtgcctggaggaagggcagtggcagctatcttggggctcgggttggtactgggactgcttggcccacttgctatcttgctggctctgtatctgctgaggcgcgaccagcgcctgccccctgatgcacacaagccaccaggaggaggaagcttccggaccccaatccaggaggagcaggcagacgcacactccacactggccaagatctgattgtgtatgcgttaataaaaagaaggaactcgta

SEQ ID NO: 8 - DNA, 903 bp, Mus musculus
gggaaaagtagaaagaaagaaagaagagaaaataaagacaaagagccaccatgtatgtgtgggttcagcagcccacagcccttctgctgctgggactcacacttggagttacagcaaggcggctcaactgtgttaaacatacctaccccagtggtcacaagtgctgtcgtgagtgccagccaggccatggtatggtgagccgctgtgatcataccagggatactctatgtcatccgtgtgagactggcttctacaatgaagctgtcaattatgatacctgcaagcagtgtacacagtgcaaccatcgaagtggaagtgaactcaagcagaattgcacacctactcaggatactgtctgcagatgtagaccaggcacccaacctcggcaggacagcggctacaagcttggagttgactgtgttccctgccctcctggccacttttctccaggcaacaaccaggcctgcaagccctggaccaattgtaccttatctggaaagcagacccgccacccagccagtgacagcttggacgcagtctgtgaggacagaagcctcctggccacactgctctgggagacccagcgccctacattcaggccaaccactgtccaatccaccacagtctggcccaggacttctgagttgccctctccacccaccttggtgactcctgagggccctgcatttgctgttctcctaggcctgggcctgggcctgctggctcccttgactgtcctgctggccttgtacctgctccggaaggcttggagattgcctaacactcccaaaccttgttggggaaacagcttcaggaccccgatccaggaggaacacacagacgcacactttactctggccaagatctgattgtgtatgcgttaataaaaagaaggaactcgta

SEQ ID NO: 9 - DNA, 50 bp, Artificial Sequence
  synthetic construct
gggaaaagtagaaagaaagaaagaagagaaaataaagacaaagagccacc

SEQ ID NO: 10 - DNA, 34 bp, Artificial Sequence
  synthetic construct
ttgtgtatgcgttaataaaaagaaggaactcgta

SEQ ID NO: 11 - DNA, 834 bp, Homo sapiens
atgtgcgtgggagcacggagactgggaaggggaccttgcgccgccctgctgctgctgggcctgggcctgtccaccgtgacaggcctgcactgcgtgggcgacacctacccttctaacgataggtgctgtcacgagtgtcgcccaggcaatggcatggtgtccaggtgctcccgctctcagaacaccgtgtgccggccttgtggcccaggcttctataatgacgtggtgagctccaagccctgcaagccttgtacatggtgcaacctgcggagcggctccgagagaaagcagctgtgcaccgccacacaggataccgtgtgccggtgtagagccggcacacagccactggactcttacaagccaggagtggattgtgcaccttgcccacctggccactttagcccaggcgacaaccaggcctgtaagccctggaccaattgcacactggcaggcaagcacaccctgcagccagcatctaattctagcgatgccatctgcgaggacagagatccaccagcaacccagcctcaggagacacagggacctccagccaggccaatcaccgtgcagccaacagaggcatggcctcggacctctcagggaccaagcacaagacccgtggaggtgcctggaggaagggcagtggcagctatcttggggctcgggttggtactgggactgcttggcccacttgctatcttgctggctctgtatctgctgaggcgcgaccagcgcctgccccctgatgcacacaagccaccaggaggaggaagcttccggaccccaatccaggaggagcaggcagacgcacactccacactggccaagatctga

SEQ ID NO: 12 - DNA, 819 bp, Mus musculus
atgtatgtgtgggttcagcagcccacagcccttctgctgctgggactcacacttggagttacagcaaggcggctcaactgtgttaaacatacctaccccagtggtcacaagtgctgtcgtgagtgccagccaggccatggtatggtgagccgctgtgatcataccagggatactctatgtcatccgtgtgagactggcttctacaatgaagctgtcaattatgatacctgcaagcagtgtacacagtgcaaccatcgaagtggaagtgaactcaagcagaattgcacacctactcaggatactgtctgcagatgtagaccaggcacccaacctcggcaggacagcggctacaagcttggagttgactgtgttccctgccctcctggccacttttctccaggcaacaaccaggcctgcaagccctggaccaattgtaccttatctggaaagcagacccgccacccagccagtgacagcttggacgcagtctgtgaggacagaagcctcctggccacactgctctgggagacccagcgccctacattcaggccaaccactgtccaatccaccacagtctggcccaggacttctgagttgccctctccacccaccttggtgactcctgagggccctgcatttgctgttctcctaggcctgggcctgggcctgctggctcccttgactgtcctgctggccttgtacctgctccggaaggcttggagattgcctaacactcccaaaccttgttggggaaacagcttcaggaccccgatccaggaggaacacacagacgcacactttactctggccaagatctga

SEQ ID NO: 13 - DNA, 597 bp, Mus musculus
atggaaggggaaggggttcaacccctggatgagaatctggaaaacggatcaaggccaagattcaagtggaagaagacgctaaggctggtggtctctgggatcaagggagcagggatgcttctgtgcttcatctatgtctgcctgcaactctcttcctctccggcaaaggaccctccaatccaaagactcagaggagcagttaccagatgtgaggatgggcaactattcatcagctcatacaagaatgagtatcaaactatggaggtgcagaacaattcggttgtcatcaagtgcgatgggctttatatcatctacctgaagggctcctttttccaggaggtcaagattgaccttcatttccgggaggatcataatcccatctctattccaatgctgaacgatggtcgaaggattgtcttcactgtggtggcctctttggctttcaaagataaagtttacctgactgtaaatgctcctgatactctctgcgaacacctccagataaatgatggggagctgattgttgtccagctaacgcctggatactgtgctcctgaaggatcttaccacagcactgtgaaccaagtaccactgtga

SEQ ID NO: 14 - DNA, 552 bp, Homo sapiens
atggaaagggtccaacccctggaagagaatgtgggaaatgcagccaggccaagattcgagaggaacaagctattgctggtggcctctgtaattcagggactggggctgctcctgtgcttcacctacatctgcctgcacttctctgctcttcaggtatcacatcggtatcctcgaattcaaagtatcaaagtacaatttaccgaatataagaaggagaaaggtttcatcctcacttcccaaaaggaggatgaaatcatgaaggtgcagaacaactcagtcatcatcaactgtgatgggttttatctcatctccctgaagggctacttctcccaggaagtcaacattagccttcattaccagaaggatgaggagcccctcttccaactgaagaaggtcaggtctgtcaactccttgatggtggcctctctgacttacaaagacaaagtctacttgaatgtgaccactgacaatacctccctggatgacttccatgtgaatggcggagaactgattcttatccatcaaaatcctggtgaattctgtgtcctttga

SEQ ID NO: 15 - DNA, 603 bp, Mus musculus
atgaagccgtacttctgccgtgtctttgtcttctgcttcctaatcagacttttaacaggagaaatcaatggctcggccgatcataggatgttttcatttcacaatggaggtgtacagatttcttgtaaataccctgagactgtccagcagttaaaaatgcgattgttcagagagagagaagtcctctgcgaactcaccaagaccaagggaagcggaaatgcggtgtccatcaagaatccaatgctctgtctatatcatctgtcaaacaacagcgtctcttttttcctaaacaacccagacagctcccagggaagctattacttctgcagcctgtccatttttgacccacctccttttcaagaaaggaaccttagtggaggatatttgcatatttatgaatcccagctctgctgccagctgaagctctggctacccgtagggtgtgcagctttcgttgtggtactcctttttggatgcatacttatcatctggttttcaaaaaagaaatacggatccagtgtgcatgaccctaatagtgaatacatgttcatggcggcagtcaacacaaacaaaaagtctagacttgcaggtgtgacctcataa

SEQ ID NO: 16 - DNA, 599 bp, Homo sapiens
atgaagtcaggcctctggtatttctttctcttctgcttgcgcattaaagttttaacaggagaaatcaatggttctgccaattatgagatgtttatatttcacaacggaggtgtacaaattttatgcaaatatcctgacattgtccagcaatttaaaatgcagttgctgaaaggggggcaaatactctgcgatctcactaagacaaaaggaagtggaaacacagtgtccattaagagtctgaaattctgccattctcagttatccaacaacagtgtctctttttttctatacaacttggaccattctcatgccaactattacttctgcaacctatcaatttttgatcctcctccttttaaagtaactcttacaggaggatatttgcatatttatgaatcacaactttgttgccagctgaagttcggttacccataggatgtgcagcctttgttgtagtctgcattttgggatgcatacttatttgttggcttacaaaaaagaagtattcatccagtgtgcacgaccctaacggtgaatacatgttcatgagagcagtgaacacagccaaaaaatctagactcacagatgtgaccctataa

SEQ ID NO: 17 - DNA, 771 bp, Mus musculus
atgggaaacaactgttacaacgtggtggtcattgtgctgctgctagtgggctgtgagaaggtgggagccgtgcagaactcctgtgataactgtcagcctggtactttctgcagaaaatacaatccagtctgcaagagctgccctccaagtaccttctccagcataggtggacagccgaactgtaacatctgcagagtgtgtgcaggctatttcaggttcaagaagttttgctcctctacccacaacgcggagtgtgagtgcattgaaggattccattgcttggggccacagtgcaccagatgtgaaaaggactgcaggcctggccaggagctaacgaagcagggttgcaaaacctgtagcttgggaacatttaatgaccagaacggtactggcgtctgtcgaccctggacgaactgctctctagacggaaggtctgtgcttaagaccgggaccacggagaaggacgtggtgtgtggaccccctgtggtgagcttctctcccagtaccaccatttctgtgactccagagggaggaccaggagggcactccttgcaggtccttaccttgttcctggcgctgacatcggctttgctgctggccctgatcttcattactctcctgttctctgtgctcaaatggatcaggaaaaaattcccccacatattcaagcaaccatttaagaagaccactggagcagctcaagaggaagatgcttgtagctgccgatgtccacaggaagaagaaggaggaggaggaggctatgagctgtga

SEQ ID NO: 18 - DNA, 768 bp, Homo sapiens
atgggaaacagctgttacaacatagtagccactctgttgctggtcctcaactttgagaggacaagatcattgcaggatccttgtagtaactgcccagctggtacattctgtgataataacaggaatcagatttgcagtccctgtcctccaaatagtttctccagcgcaggtggacaaaggacctgtgacatatgcaggcagtgtaaaggtgttttcaggaccaggaaggagtgttcctccaccagcaatgcagagtgtgactgcactccagggtttcactgcctgggggcaggatgcagcatgtgtgaacaggattgtaaacaaggtcaagaactgacaaaaaaaggttgtaaagactgttgctttgggacatttaacgatcagaaacgtggcatctgtcgaccctggacaaactgttctttggatggaaagtctgtgcttgtgaatgggacgaaggagagggacgtggtctgtggaccatctccagccgacctctctccgggagcatcctctgtgaccccgcctgcccctgcgagagagccaggacactctccgcagatcatctccttctttcttgcgctgacgtcgactgcgttgctcttcctgctgttcttcctcacgctccgtttctctgttgttaaacggggcagaaagaaactcctgtatatattcaaacaaccatttatgagaccagtacaaactactcaagaggaagatggctgtagctgccgatttccagaagaagaagaaggaggatgtgaactgtga

SEQ ID NO: 19 - DNA, 930 bp, Mus musculus
atggaccagcacacacttgatgtggaggataccgcggatgccagacatccagcaggtacttcgtgcccctcggatgcggcgctcctcagagataccgggctcctcgcggacgctgcgctcctctcagatactgtgcgccccacaaatgccgcgctccccacggatgctgcctaccctgcggttaatgttcgggatcgcgaggccgcgtggccgcctgcactgaacttctgttcccgccacccaaagctctatggcctagtcgctttggttttgctgcttctgatcgccgcctgtgttcctatcttcacccgcaccgagcctcggccagcgctcacaatcaccacctcgcccaacctgggtacccgagagaataatgcagaccaggtcacccctgtttcccacattggctgccccaacactacacaacagggctctcctgtgttcgccaagctactggctaaaaaccaagcatcgttgtgcaatacaactctgaactggcacagccaagatggagctgggagctcatacctatctcaaggtctgaggtacgaagaagacaaaaaggagttggtggtagacagtcccgggctctactacgtatttttggaactgaagctcagtccaacattcacaaacacaggccacaaggtgcagggctgggtctctcttgttttgcaagcaaagcctcaggtagatgactttgacaacttggccctgacagtggaactgttcccttgctccatggagaacaagttagtggaccgttcctggagtcaactgttgctcctgaaggctggccaccgcctcagtgtgggtctgagggcttatctgcatggagcccaggatgcatacagagactgggagctgtcttatcccaacaccaccagctttggactctttcttgtgaaacccgacaacccatgggaatga

SEQ ID NO: 20 - DNA, 765 bp, Homo sapiens
atggaatacgcctctgacgcttcactggaccccgaagccccgtggcctcccgcgccccgcgctcgcgcctgccgcgtactgccttgggccctggtcgcggggctgctgctgctgctgctgctcgctgccgcctgcgccgtcttcctcgcctgcccctgggccgtgtccggggctcgcgcctcgcccggctccgcggccagcccgagactccgcgagggtcccgagctttcgcccgacgatcccgccggcctcttggacctgcggcagggcatgtttgcgcagctggtggcccaaaatgttctgctgatcgatgggcccctgagctggtacagtgacccaggcctggcaggcgtgtccctgacggggggcctgagctacaaagaggacacgaaggagctggtggtggccaaggctggagtctactatgtcttctttcaactagagctgcggcgcgtggtggccggcgagggctcaggctccgtttcacttgcgctgcacctgcagccactgcgctctgctgctggggccgccgccctggctttgaccgtggacctgccacccgcctcctccgaggctcggaactcggccttcggtttccagggccgcttgctgcacctgagtgccggccagcgcctgggcgtccatcttcacactgaggccagggcacgccatgcctggcagcttacccagggcgccacagtcttgggactcttccgggtgacccccgaaatcccagccggactcccttcaccgaggtcggaataa

SEQ ID NO: 21 - DNA, 687 bp, Mus musculus
atgggggcatgggccatgctgtatggagtctcgatgctctgtgtgctggacctaggtcagccgagtgtagttgaggagcctggctgtggccctggcaaggttcagaacggaagtggcaacaacactcgctgctgcagcctgtatgctccaggcaaggaggactgtccaaaagaaaggtgcatatgtgtcacacctgagtaccactgtggagaccctcagtgcaagatctgcaagcactacccctgccaaccaggccagagggtggagtctcaaggggatattgtgtttggcttccggtgtgttgcctgtgccatgggcaccttctccgcaggtcgtgacggtcactgcagactttggaccaactgttctcagtttggatttctcaccatgttccctgggaacaagacccacaatgctgtgtgcatcccggagccactgcccactgagcaatacggccatttgactgtcatcttcctggtcatggctgcatgcattttcttcctaaccacagtccagctcggcctgcacatatggcagctgaggaggcaacacatgtgtcctcgagagacccagccattcgcggaggtgcagttgtcagctgaggatgcttgcagcttccagttccctgaggaggaacgcggggagcagacagaagaaaagtgtcatctggggggtcggtggccatga

SEQ ID NO: 22 - DNA, 726 bp, Homo sapiens
atggcacagcacggggcgatgggcgcgtttcgggccctgtgcggcctggcgctgctgtgcgcgctcagcctgggtcagcgccccaccgggggtcccgggtgcggccctgggcgcctcctgcttgggacgggaacggacgcgcgctgctgccgggttcacacgacgcgctgctgccgcgattacccgggcgaggagtgctgttccgagtgggactgcatgtgtgtccagcctgaattccactgcggagacccttgctgcacgacctgccggcaccacccttgtcccccaggccagggggtacagtcccaggggaaattcagttttggcttccagtgtatcgactgtgcctcggggaccttctccgggggccacgaaggccactgcaaaccttggacagactgcacccagttcgggtttctcactgtgttccctgggaacaagacccacaacgctgtgtgcgtcccagggtccccgccggcagagccgcttgggtggctgaccgtcgtcctcctggccgtggccgcctgcgtcctcctcctgacctcggcccagcttggactgcacatctggcagctgaggagtcagtgcatgtggccccgagagacccagctgctgctggaggtgccgccgtcgaccgaagacgccagaagctgccagttccccgaggaagagcggggcgagcgatcggcagaggagaaggggcggctgggagacctgtgggtgtga

SEQ ID NO: 23 - DNA, 870 bp, Mus musculus
atggtgtctttgcctcggctgtgcgcgctatggggctgcttgttgacagcggtccatctagggcagtgtgttacgtgcagtgacaaacagtacctccacgatggccagtgctgtgatttgtgccagccaggaagccgactgacaagccactgcacagctcttgagaagacccaatgccacccatgtgactcaggcgaattctcagcccagtggaacagggagattcgctgtcaccagcacagacactgtgaacccaatcaagggcttcgggttaagaaggagggcaccgcagaatcagacactgtctgtacctgtaaggaaggacaacactgcaccagcaaggattgcgaggcatgtgctcagcacacgccctgtatccctggctttggagttatggagatggccactgagaccactgataccgtctgtcatccctgcccagtcggcttcttctccaatcagtcatcacttttcgaaaagtgttatccctggacaagctgtgaggataagaacttggaggtcctacagaaaggaacgagtcagactaatgtcatctgtggtttaaagtcccggatgcgagccctgctggtcattcctgtcgtgatgggcatcctcatcaccattttcggggtgtttctctatatcaaaaaggtggtcaagaaaccaaaggataatgagatcttaccccctgcggctcgacggcaagatccccaggagatggaagattatcccggtcataacaccgctgctccagtgcaggagacactgcacgggtgtcagcctgtcacacaggaggatggtaaagagagtcgcatctcagtgcaggagcggcaggtgacagacagcatagccttgaggcccctggtctaa

SEQ ID NO: 24 - DNA, 783 bp, Mus musculus
atgatagaaacatacagccaaccttcccccagatccgtggcaactggacttccagcgagcatgaagatttttatgtatttacttactgttttccttatcacccaaatgattggatctgtgctttttgctgtgtatcttcatagaagattggataaggtcgaagaggaagtaaaccttcatgaagattttgtattcataaaaaagctaaagagatgcaacaaaggagaaggatctttatccttgctgaactgtgaggagatgagaaggcaatttgaagaccttgtcaaggatataacgttaaacaaagaagagaaaaaagaaaacagctttgaaatgcaaagaggtgatgaggatcctcaaattgcagcacacgttgtaagcgaagccaacagtaatgcagcatccgttctacagtgggccaagaaaggatattataccatgaaaagcaacttggtaatgcttgaaaatgggaaacagctgacggttaaaagagaaggactctattatgtctacactcaagtcaccttctgctctaatcgggagccttcgagtcaacgcccattcatcgtcggcctctggctgaagcccagcagtggatctgagagaatcttactcaaggcggcaaatacccacagttcctcccagctttgcgagcagcagtctgttcacttgggcggagtgtttgaattacaagctggtgcttctgtgtttgtcaacgtgactgaagcaagccaagtgatccacagagttggcttctcatcttttggcttactcaaactctga